SEQUENCE LISTING (162 sequences)

SEQ ID NO: 1 - DNA, 131 bp, synthetic construct
  source 1..131: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 2 - DNA, 115 bp, synthetic construct
  source 1..115: mol_type = other DNA
aaataattgagccttaaagaagaaattctttaagtggatgctctcaaactcagggaaacctaaatctagttatagacaaggcaatcctgagccaagccgaagtagtaattagtaa

SEQ ID NO: 3 - DNA, 97 bp, synthetic construct
  source 1..97: mol_type = other DNA
tgcgccgatgaaggtgtagagactagacggcacccacctaaggcaaacgctatggtgaaggcatagtccagggagtggcgaaagtcacacaaaccgg

SEQ ID NO: 4 - DNA, 108 bp, synthetic construct
  source 1..108: mol_type = other DNA
atttcgatgtgccttgcgccgggaaaccacgcaagggatggtgtcaaattcggcgaaacctaagcgcccgcccgggcgtatggcaacgccgagccaagcttcggcgcc

SEQ ID NO: 5 - DNA, 219 bp, synthetic construct
  source 1..219: mol_type = other DNA
aaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatg

SEQ ID NO: 6 - DNA, 166 bp, synthetic construct
  source 1..166: mol_type = other DNA
taattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatacttt

SEQ ID NO: 7 - skipped

SEQ ID NO: 8 - DNA, 12 bp, synthetic construct
  source 1..12: mol_type = other DNA
cgctacggactt

SEQ ID NO: 9 - DNA, 12 bp, synthetic construct
  source 1..12: mol_type = other DNA
aatccgccggtg

SEQ ID NO: 10 - DNA, 15 bp, synthetic construct
  source 1..15: mol_type = other DNA
gagcggcggactcat

SEQ ID NO: 11 - DNA, 15 bp, synthetic construct
  source 1..15: mol_type = other DNA
ctaccgtttaatatt

SEQ ID NO: 12 - DNA, 15 bp, synthetic construct
  source 1..15: mol_type = other DNA
gatgttttcttgggt

SEQ ID NO: 13 - DNA, 12 bp, synthetic construct
  source 1..12: mol_type = other DNA
aaaatccgttga

SEQ ID NO: 14 - DNA, 12 bp, synthetic construct
  source 1..12: mol_type = other DNA
aaaaaggcatga

SEQ ID NO: 15 - DNA, 12 bp, synthetic construct
  source 1..12: mol_type = other DNA
aaaatctgttga

SEQ ID NO: 16 - DNA, 12 bp, synthetic construct
  source 1..12: mol_type = other DNA
aaagtccgttga

SEQ ID NO: 17 - DNA, 14 bp, synthetic construct
  source 1..14: mol_type = other DNA
aaaatccgttgcga

SEQ ID NO: 18 - DNA, 16 bp, synthetic construct
  source 1..16: mol_type = other DNA
aaaatccgttgcgtca

SEQ ID NO: 19 - DNA, 12 bp, synthetic construct
  source 1..12: mol_type = other DNA
aaaaaaaaaagg

SEQ ID NO: 20 - DNA, 12 bp, synthetic construct
  source 1..12: mol_type = other DNA
aaaaccccctga

SEQ ID NO: 21 - DNA, 12 bp, synthetic construct
  source 1..12: mol_type = other DNA
aaaatgggggga

SEQ ID NO: 22 - DNA, 12 bp, synthetic construct
  source 1..12: mol_type = other DNA
aaaaccccccca

SEQ ID NO: 23 - DNA, 12 bp, synthetic construct
  source 1..12: mol_type = other DNA
tttttccgttga

SEQ ID NO: 24 - DNA, 10 bp, synthetic construct
  source 1..10: mol_type = other DNA
aatccgttga

SEQ ID NO: 25 - skipped

SEQ ID NO: 26 - DNA, 12 bp, synthetic construct
  source 1..12: mol_type = other DNA
gaaatccgttga

SEQ ID NO: 27 - DNA, 12 bp, synthetic construct
  source 1..12: mol_type = other DNA
taaatccgttga

SEQ ID NO: 28 - DNA, 12 bp, synthetic construct
  source 1..12: mol_type = other DNA
caaatccgttga

SEQ ID NO: 29 - DNA, 121 bp, synthetic construct
  source 1..121: mol_type = other DNA
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacaca

SEQ ID NO: 30 - DNA, 84 bp, synthetic construct
  source 1..84: mol_type = other DNA
aaaatccgttgaccttaaacggtcgtgtgggttcaagtccctccacccccagaaaccaactttattactatattccccacaacc

SEQ ID NO: 31 - DNA, 51 bp, synthetic construct
  source 1..51: mol_type = other DNA
aaaatccgttgaccttaaacggtcgtgtgggttcaagtccctccaccccca

SEQ ID NO: 32 - DNA, 12 bp, synthetic construct
  source 1..12: mol_type = other DNA
aatccgttggtg

SEQ ID NO: 33 - DNA, 12 bp, synthetic construct
  source 1..12: mol_type = other DNA
aatccgccggtg

SEQ ID NO: 34-48 - skipped (15 entries)

SEQ ID NO: 49 - DNA, 10 bp, synthetic construct
  source 1..10: mol_type = other DNA
aagtccgttg

SEQ ID NO: 50 - skipped

SEQ ID NO: 51 - DNA, 11 bp, synthetic construct
  source 1..11: mol_type = other DNA
tccgttgcgtc

SEQ ID NO: 52-55 - skipped (4 entries)

SEQ ID NO: 56 - DNA, 15 bp, synthetic construct
  source 1..15: mol_type = other DNA
agacgctacggactt

SEQ ID NO: 57 - DNA, 15 bp, synthetic construct
  source 1..15: mol_type = other DNA
agacgctacagactt

SEQ ID NO: 58 - DNA, 15 bp, synthetic construct
  source 1..15: mol_type = other DNA
agacgcttgcctctt

SEQ ID NO: 59 - DNA, 15 bp, synthetic construct
  source 1..15: mol_type = other DNA
agacgcaacggactt

SEQ ID NO: 60 - DNA, 15 bp, synthetic construct
  source 1..15: mol_type = other DNA
agacccttttttctt

SEQ ID NO: 61 - DNA, 15 bp, synthetic construct
  source 1..15: mol_type = other DNA
agacgctgggggctt

SEQ ID NO: 62 - DNA, 15 bp, synthetic construct
  source 1..15: mol_type = other DNA
agacgccccccactt

SEQ ID NO: 63 - DNA, 15 bp, synthetic construct
  source 1..15: mol_type = other DNA
agacggggggggctt

SEQ ID NO: 64 - DNA, 55 bp, synthetic construct
  source 1..55: mol_type = other DNA
aaaaaacaaaaaacaaaacggctattatgcgttaccggcgagacgctacggactt

SEQ ID NO: 65 - DNA, 15 bp, synthetic construct
  source 1..15: mol_type = other DNA
agacgctacggactt

SEQ ID NO: 66 - DNA, 15 bp, synthetic construct
  source 1..15: mol_type = other DNA
agacgctacggacaa

SEQ ID NO: 67 - DNA, 15 bp, synthetic construct
  source 1..15: mol_type = other DNA
agacgctacggactc

SEQ ID NO: 68 - DNA, 15 bp, synthetic construct
  source 1..15: mol_type = other DNA
agacgctacggacta

SEQ ID NO: 69 - DNA, 15 bp, synthetic construct
  source 1..15: mol_type = other DNA
agacgctacggactg

SEQ ID NO: 70 - DNA, 15 bp, synthetic construct
  source 1..15: mol_type = other DNA
gagcagcggactcat

SEQ ID NO: 71 - DNA, 15 bp, synthetic construct
  source 1..15: mol_type = other DNA
gagcggcggactcat

SEQ ID NO: 72-86 - skipped (15 entries)

SEQ ID NO: 87 - DNA, 10 bp, synthetic construct
  source 1..10: mol_type = other DNA
caacggactt

SEQ ID NO: 88 - skipped

SEQ ID NO: 89 - DNA, 11 bp, synthetic construct
  source 1..11: mol_type = other DNA
gacgcaacgga

SEQ ID NO: 90-93 - skipped (4 entries)

SEQ ID NO: 94 - DNA, 25 bp, synthetic construct
  source 1..25: mol_type = other DNA
ccgattagttgtaagtcatctattg

SEQ ID NO: 95 - DNA, 27 bp, synthetic construct
  source 1..27: mol_type = other DNA
tcaacggattttaagtccgtagcgtct

SEQ ID NO: 96 - DNA, 10 bp, synthetic construct
  source 1..10: mol_type = other DNA
attttaagtc

SEQ ID NO: 97 - DNA, 20 bp, synthetic construct
  source 1..20: mol_type = other DNA
aacggattttaagtccgtag

SEQ ID NO: 98 - DNA, 23 bp, synthetic construct
  source 1..23: mol_type = other DNA
caacggattttaagtccgtagcg

SEQ ID NO: 99 - DNA, 25 bp, synthetic construct
  source 1..25: mol_type = other DNA
tcaacggattttaagtccgtagcgt

SEQ ID NO: 100 - DNA, 27 bp, synthetic construct
  source 1..27: mol_type = other DNA
tcaacggattttaagtccgtagcgtct

SEQ ID NO: 101 - DNA, 29 bp, synthetic construct
  source 1..29: mol_type = other DNA
atcaacggattttaagtccgtagcgtctt

SEQ ID NO: 102 - DNA, 27 bp, synthetic construct
  source 1..27: mol_type = other DNA
tgcgaggaaccatccggccagctcctg

SEQ ID NO: 103 - DNA, 25 bp, synthetic construct
  source 1..25: mol_type = other DNA
gatagtcgttaggcatttatgtaga

SEQ ID NO: 104 - DNA, 27 bp, synthetic construct
  source 1..27: mol_type = other DNA
tattaaacggtagacccaagaaaacat

SEQ ID NO: 105 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgtattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 106 - DNA, 537 bp, synthetic construct
  source 1..537: mol_type = other DNA
ttttgcggctctgccgccgttcgggttttacctgttttcacagagcaaaacaggacctctagtttcgtgcttaaacgagatcatgctcgaactagaactataacgctggtcactggacccgtgccgcgccttgcggatctttgcgggaatggtggctagtgggctgtggaagtgactctaaccacacgcccctcaagtgtgggaaaacacgaactggtgtagcgacgacgataggccttgggacaccctctccagtgatggagacccaaggggccaaaagccacgccttgtgccctgtcgttcacaaccccagtgcagttcgtgccagtacctgcttttgggaagtgtgctttggacagctgaaaacagtcctagtgggagactaaggatgcccaggaggtacccggaggtaacaagtgacactctggatctgacttggggagagcgggtctgctttacagacgccactctttaaaaaacttctatgtctcgtcaggcaccggaggccgggccttttcctttaaaacaatacacttt

SEQ ID NO: 107 - DNA, 438 bp, synthetic construct
  source 1..438: mol_type = other DNA
ataacgctggtcactggacccgtgccgcgccttgcggatctttgcgggaatggtggctagtgggctgtggaagtgactctaaccacacgcccctcaagtgtgggaaaacacgaactggtgtagcgacgacgataggccttgggacaccctctccagtgatggagacccaaggggccaaaagccacgccttgtgccctgtcgttcacaaccccagtgcagttcgtgccagtacctgcttttgggaagtgtgctttggacagctgaaaacagtcctagtgggagactaaggatgcccaggaggtacccggaggtaacaagtgacactctggatctgacttggggagagcgggtctgctttacagacgccactctttaaaaaacttctatgtctcgtcaggcaccggaggccgggccttttcctttaaaacaatacacttt

SEQ ID NO: 108 - DNA, 643 bp, synthetic construct
  source 1..643: mol_type = other DNA
cgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcata

SEQ ID NO: 109 - DNA, 750 bp, synthetic construct
  source 1..750: mol_type = other DNA
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgc

SEQ ID NO: 110 - DNA, 750 bp, synthetic construct
  source 1..750: mol_type = other DNA
ttaaaacagcctgtgggttgcacccacccacagggcccactgggcgctagcactctggtatcacgatacctttgtgcgcctgttttatatccccaccccgagtaaacgttagaagttacgcaaccccgatcaatagtaggtgtagcactccagctgcatcgagatcaagcacttctgtctccccggaccgagtatcaatagactgctaacgcggttgaaggagaaaacgttcgttacccggccaattacttcgagaagcccagtagtgccgtgaaagttgcggagtgtttcgctcagcacttcccccgtgtagatcaggctgatgagtcaccgcgatccccacaggtgactgtggcggtggctgcgttggcggcctgcctatggggcaacccataggacgctctaatacagacatggtgcgaagagcctattgagctaattggtagtcctccggcccctgaatgcggctaatcctaactgcggagcacatgccctcaaaccagggggtggtgtgtcgtaacgggtaactctgcagcggaaccgactactttgggtgtccgtgtttctttttattcttataatggctgcttatggtgacaattaaagaattgttaccatatagctattggattggccatccggtgactaacaaatcgctcatataccagtttgttggttttgttcccttatcacatacagctcataacaccctcttatatttactacaattgaatagcaagaaatgggggc

SEQ ID NO: 111 - DNA, 643 bp, synthetic construct
  source 1..643: mol_type = other DNA
gagtaaacgttagaagttacgcaaccccgatcaatagtaggtgtagcactccagctgcatcgagatcaagcacttctgtctccccggaccgagtatcaatagactgctaacgcggttgaaggagaaaacgttcgttacccggccaattacttcgagaagcccagtagtgccgtgaaagttgcggagtgtttcgctcagcacttcccccgtgtagatcaggctgatgagtcaccgcgatccccacaggtgactgtggcggtggctgcgttggcggcctgcctatggggcaacccataggacgctctaatacagacatggtgcgaagagcctattgagctaattggtagtcctccggcccctgaatgcggctaatcctaactgcggagcacatgccctcaaaccagggggtggtgtgtcgtaacgggtaactctgcagcggaaccgactactttgggtgtccgtgtttctttttattcttataatggctgcttatggtgacaattaaagaattgttaccatatagctattggattggccatccggtgactaacaaatcgctcatataccagtttgttggttttgttcccttatcacatacagctcataacaccctcttatatttactacaattgaatagcaagaa

SEQ ID NO: 112 - DNA, 435 bp, synthetic construct
  source 1..435: mol_type = other DNA
taagtagggaacatattcaattcatattgttcatctcactgaacccgcatgaaggactgcattgcatatcctggacgaggtgacgtggaatatttggacatttatggattggacactataacgctttgtgcctctacggagatgtaaccataatcttaagtagtagtaccccagcacaagaggataaagtggcatacacgacaacgggtgttgctcgcaccttagtaatgtggatgtccacccttggagcgtgctgaaactctgtgggtaaagacacatattagtacaaatgtgggggaactcactgaaagggcatgtcccgtgtactggtgtgccggaaagtgggggtcgctttctggagaacttagtagttcttgttattgggtgatagccttgcggcggatcaacccacagttttaatccgttgttttgcat

SEQ ID NO: 113 - DNA, 643 bp, synthetic construct; identical to SEQ ID NO: 111
  source 1..643: mol_type = other DNA

SEQ ID NO: 114 - DNA, 444 bp, synthetic construct
  source 1..444: mol_type = other DNA
cctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 115 - DNA, 446 bp, synthetic construct
  source 1..446: mol_type = other DNA
tgacatacaccgtgcaatttgaaactccgcctggtctttccaggtctagaggggtaacactttgtactgtgcttgactccacgctcggtccactggcgagtgttagtaacagcactggtgcttcgtagcggagcatggtggccgtgggaactcctccttggtaacaaggacccacggggccgaaagccacgtcctgacggacccaccatgtgtgcaaccccagcacggcaacttttctgtgaaactcactctaaggtgacactgatactggtattcaagtactggtgacaggctaaggatgcccttcaggtaccccgaggtaacacgcgacactcgggatctgagaaggggactggggcttctgtaaaagcgcccagtttaaaaagcttctatgcctggataggtgaccggaggccggcgcctttccattataactactgacttta

SEQ ID NO: 116 - DNA, 298 bp, synthetic construct
  source 1..298: mol_type = other DNA
cctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcacc

SEQ ID NO: 117 - DNA, 339 bp, synthetic construct
  source 1..339: mol_type = other DNA
acctgcctcttacgaggcgacactccaccatggatcactcccctgtgaggaacttctgtcttcacgcggaaagcgcctagccatggcgttagtacgagtgtcgtgcagcctccaggaccccccctcccgggagagccatagtggtctgcggaaccggtgagtacaccggaatcgctggggtgaccgggtcctttcttggagcaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaac

SEQ ID NO: 118 - DNA, 279 bp, synthetic construct
  source 1..279: mol_type = other DNA
ttcacgcagaaagcgtctagccatggcgttagtatgagtgttgtacagcctccaggaccccccctcccgggagagccatagtggtcttcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgcggtactgcctgatagggtgcttgcgagtgccccgggaggtctcgtagaccgtgcacc

SEQ ID NO: 119 - DNA, 279 bp, synthetic construct
  source 1..279: mol_type = other DNA
ttcacgcagaaagcgtctagccatggcgttagtatgagtgtcgaacagcctccaggaccccccctcccgggagagccatagtggtctgcggaaccggtgagtacaccggaattgccgggatgaccgggtcctttcttggataaacccgctcaatgcccggagatttgggcgtgcccccgcgagactgctagccgagtagtgttgggtcgcgaaaggccttgtggtactgcctgatagggtgcttgcgagtgccccgggaggtctcgtagaccgtgcacc

SEQ ID NO: 120 - DNA, 339 bp, synthetic construct
  source 1..339: mol_type = other DNA
gccagccccttacggggcgacactccgccatgaatcactcccctgcgaggaaccactgtcctcacgcagaaagcgtctagccatgacgttagtatgagtgtcgtacagcctccaggaccccccctcccgggagagccatagtggtctgcggaaccggtgagtacaccggaattgccgggaagactgggtcctttcttggatcaacccactctatgcccggagatttgggcgtgcccccgcgagactgctagccgagtagtgttgggtcgcgaaaggccttgtggtactgcctgatagggtgcttgcgagtgccccgggaggtctcgtagaccgtgcacc

SEQ ID NO: 121 - DNA, 373 bp, synthetic construct
  source 1..373: mol_type = other DNA
gtatacgaggttagttcattctcgtatgcatgattggacaaattaaaatttcaatttggatcagggcctccctccagcgacggccgaactgggctagccatgcccacagtaggactagcaaacggagggactagccgtagtggcgagctccctgggtggtctaagtcctgagtacaggacagtcgtcagtagttcgacgtgagcagaagcccacctcgatatgctatgtggacgagggcatgcccaagacacaccttaaccctagcgggggtcgctagggtgaaatcacaccacgtgatgggagtacgacctgatagggtgctgcagaggcccactattaggctagtataaaaatctctgctgtacatggcac

SEQ ID NO: 122 - DNA, 367 bp, synthetic construct
  source 1..367: mol_type = other DNA
taagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaat

SEQ ID NO: 123 - DNA, 443 bp, synthetic construct
  source 1..443: mol_type = other DNA
acactcatttcccccctccacccttaaggtggttgtatcccctacaccctaccctcccttccacataggacgaataaacggacttgagattaaggcaagtacataaggtatggtttttggatacacttaaatggcagtagcgtggcgagctatggaaaaatcgcaattgtcgatagccatgttagcgacgcgcttcggcgtgctcctttggtgattcggcgactggttacaggagagtagacagtgagctatgggcaaacccctacagtattacttaggggaatgtgcaattgagacttgacgagcgtctctttgagatgtggcgcatgctcttggcattaccatagtgagcttccaggttgggaaacctggactgggtctatactgcctgatagggtcgcggctggccgcctgtaactagtatagtcagttgaaaacccccc

SEQ ID NO: 124 - DNA, 193 bp, synthetic construct
  source 1..193: mol_type = other DNA
aaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 125 - DNA, 189 bp, synthetic construct
  source 1..189: mol_type = other DNA
gttaagatgtgatcttgcttccttatacaattttgagaggttaataagaaggaagtagtgctatcttaataattaggttaactatttagttttactgttcaggatgcctattggcagccccataatatccaggacaccctctctgcttcttatatgattaggttgtcatttagaataagaaaataacct

SEQ ID NO: 126 - DNA, 207 bp, synthetic construct
  source 1..207: mol_type = other DNA
atagcaccacccgatcgtaaactccatgtattggttacccatctgcatcgaaaactctccgaacactaggtgcagtaaggctttcatggagtggtttgctatttagcgtacgtgtaccataggcagccccaaaaacacgtgtgaggagaaagtcccagtcactttgggcaaagtagacagccgcgcttgcgtggtgggacttaatta

SEQ ID NO: 127 - DNA, 210 bp, synthetic construct
  source 1..210: mol_type = other DNA
agtatagtgttctggaggcatcattctatggttacccatcattagaggaaatttccaataaactctggtgtaaggcttagagtgatggtcgaggtgccctatttagggtgaggagcctcggtggcagccccaccaaatcctctattggataggaacagctgtactgggcagttacagcagtcgtatggtaacacatgcggcgttccgaaa

SEQ ID NO: 128 - DNA, 626 bp, synthetic construct
  source 1..626: mol_type = other DNA
ttaaaactgggtgtgggttgttcccacccacaccacccaatgggtgttgtactctgttattccggtaactttgtacgccagtttttccctcccctccccatccttttacgtaacttagaagttttaaatacaagaccaatagtaggcaactctccaggttgtctaaggtcaagcacttctgtttccccggttgatgttgatatgctccaacagggcaaaaacaacagataccgttatccgcaaagtgcctacacagagcttagtaggattctgaaagatctttggttggtcgttcagctgcatacccagcagtagaccttgcagatgaggctggacattccccactggtaacagtggtccagcctgcgtggctgcctgcgcacctctcatgaggtgtgaagccaaagatcggacagggtgtgaagagccgcgtgtgctcactttgagtcctccggcccctgaatgcggctaaccttaaacctgcagccatggctcataagccaatgagtttatggtcgtaacgagtaattgcgggatgggaccgactactttgggtgtccgtgtttcactttttcctttattaattgcttatggtgacaatatatatattgatatatattggcatc

SEQ ID NO: 129 - DNA, 675 bp, synthetic construct
  source 1..675: mol_type = other DNA
cccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 130 - DNA, 508 bp, synthetic construct
  source 1..508: mol_type = other DNA
gtataagagacaggtgtttgccttgtcttcggactggcatcttgggaccaaccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 131 - DNA, 730 bp, synthetic construct
  source 1..730: mol_type = other DNA
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatg

SEQ ID NO: 132 - DNA, 744 bp, synthetic construct
  source 1..744: mol_type = other DNA
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 133 - DNA, 709 bp, synthetic construct
  source 1..709: mol_type = other DNA
tatggcaggcgggcttgtggacggcttcggcccacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 134 - DNA, 626 bp, synthetic construct
  source 1..626: mol_type = other DNA
ttaaaacagcggatgggtatcccaccatccgacccacagggtgtagtgctctggtattttgtacctttgcacgcctgtttccccattgtacccctccttaaatttcctccccaagtaacgttagaagtttaaggaaacaaatgtacaataggaagcatcacatccagtggtgttatgtacaagcacttctgtttccccggagcgaggtataagtggtacccaccgccgaaagcctttaaccgttatccgccaatcaactacgtaatggctagtattaccatgtttgtgacttggtgttcgatcaggtggttccccccactagtttggtcgatgaggctaggaactccccacgggtgaccgtgtcctagcctgcgtggcggccaacccagcttttgctgggacgcctttttacagacatggtgtgaagacctgcatgtgcttgattgtgagtcctcggcccctgaatgcggctaaccttaaccccggagccttgcaacataatccaatgttgttgaggtcgtaatgagtaattctgggatgggaccgactactttgggtgtccgtgtttccttttattctttatattgtcttatggtcacagcatatatagcatatatactgtgatc

SEQ ID NO: 135 - DNA, 416 bp, synthetic construct
  source 1..416: mol_type = other DNA
cttccttttaattcgtaactgataagtgatagtccttggaagctaggtatttgttacgctagttttggattatcttgtgcccaacatttgttttcgaacatatgttgtgtttaaacacagaaatctagtttctttggttatgagtttaatggaatatccttttgaaagacttgccttggcgcgggctagagcgcaattgtcaccaggtattgcaccaatggtggcgacagggtacagaagagcaagtactcctgactgggtaatgggactgcattgcatatccctaggcatctattgagatttctctggagcccaccagcatggagttcctgtatgggaatgcaggactggacttgtgctgcctgacagggtcgcggctggccgtctgtactttgtatagtcagttgaaactcatt

SEQ ID NO: 136 - DNA, 262 bp, synthetic construct
  source 1..262: mol_type = other DNA
cggacttaaataattgagccttaaagaagaaattctttaagtggatgctctcaaactcagggaaacctaaatctagttatagacaaggcaatcctgagccaagccgaagtagtaattagtaaaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 137 - DNA, 232 bp, synthetic construct
  source 1..232: mol_type = other DNA
gagcggcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaaacgctatggtgaaggcatagtccagggagtggcgaaagtcacacaaaccggaatccgccggtg

SEQ ID NO: 138 - DNA, 321 bp, synthetic construct
  source 1..321: mol_type = other DNA
ctttcggacaggggttcgattcaaaagagtcgccttatgaagtgattcataagtgaaaacttggctttatcggtgaaaccgaaacgtaagacgtcggcaataccgagtggtatgtggggaaacccaacagccgtatcgactcataggtttctaaccttaagaaatgaaattttcttaagtaagaaatactaggatagaagttttaaactataatatagcttctataacatgccaaggtacttaaaacttataagttttaagcacgagatatagtcagtgccattagagataatggaatagcatgtcccctcgcctccacca

SEQ ID NO: 139 - DNA, 351 bp, synthetic construct
  source 1..351: mol_type = other DNA
agacgctacggacttaattggattgagccttggtatggaaacctactaagtgataactttcaaattcagagaaacccgggaattaacaatgggcaatcctgagccaaatcctggtttacgcgaacaaaccaaagtgtagaaagcgagaaagaagggataggtgcagagactcaatggaagctgttctaacaaatggagttcactaccttgtattgatcaaatgattcacttcatagtctgatagatccttggtggaactgattaatcggacgagaataaagatagagtcccattctacatgtcaatactgacaacaatgaaatttatagtaagatgaaaatccgttgactt

SEQ ID NO: 140 - DNA, 351 bp, synthetic construct; identical to SEQ ID NO: 139
  source 1..351: mol_type = other DNA

SEQ ID NO: 141 - DNA, 351 bp, synthetic construct; identical to SEQ ID NO: 139
  source 1..351: mol_type = other DNA

SEQ ID NO: 142 - DNA, 440 bp, synthetic construct
  source 1..440: mol_type = other DNA
ctacggacttaattggattgagccttggtatggaaacctaccaagtgataactttcaaattcagagaaaccctggaattaaaaatgggcaatcctgagccaaatcccgttttatgaaaacaaacaagggtttcagaaagcgagaataaataaaggataggtgcagagactcaatggaagctgttctaacaaatggagttggctgcattgtgttcgtagtaaaggaatcgaaacttccgaaaggatgaaagataaacctatatacatatgtatacttactgaaatactatcgccaaataattcaaaatgattaatgacgactccgtccgctaaatctttttatttttaaaaatttttaaatcggataagaataaagatagagtcccattctacatgtcaatatcgacaacaatgaaatttatagtaagaggaaaatccgtc

SEQ ID NO: 143 - DNA, 867 bp, synthetic construct
  source 1..867: mol_type = other DNA
aatcgcagtgtggtttaacattcgggccacactataataaactccttgaattcggtaaacctctcaacgaataaagttcgcagacaataccgagctaagcttaatatgctaagattttaaatgtgtttactccaaccaaacatgctataataaacctaagaggtgatagcaatggagcattggaaatggatagatggttttgagggcaaatacgaagtcagtgatttaggcagagtaaggtcttatgcaactggcaaatgcgcttatctatctgttaatcgcctaactcgcgatggctatagccatattgctttgcgaaaaaatggcaaagcatatgagtttagactaaacagactggtggcagcagcttttattggccaaccatctaaagagaaaaacacagttaatcacattaatggcatcaaaactgataatcgagcagttaaccttgaatgggcctcacgatcagagcaaatgtatcatgcttatcaacaccatttaaaattacccaggaaaggaaaacgcagaacatttgacgacttcagctttgaagaaaagcaagatatctggaataattatcagccatataaaaaagggcacagtaaaagatatttttgcattaaatataatacccactattcagtgattgacaaaatcttagcagaaaagaaagtgtaacgactatcgaaacacaaaaagcatcagctgattagctgatgctttttgaatggagtagagtagtccccaagcgggggcgaagcagggagcatcagcaatatgatgaagagctagtctgaacttgatagaaatatcaagagcatgtatggacgcgatacatgcgcaacaacattacgaagaatttgcaaa

SEQ ID NO: 144 - DNA, 345 bp, synthetic construct
  source 1..345: mol_type = other DNA
agacgctgcggacttaatttaattgagctttagttgagaaatttactaaatgattgttttcaaattcagggaaacctaggttgcaaaaaaaagataaaaattaggtaatcctgagccaaattttgtttactaaaacaaaagaggtgcagagactcaaagaaaactatcctaacgaaattttttatcatttttataaaaaattggattaatatattaattaataataataaaattattaaatcattttttcattttaaatatagacgaggataaagatagagtccgtttttacaagttaattttaacaacaatgcaaattgtagtaaaatgaaaatccgttggctt

SEQ ID NO: 145 - DNA, 508 bp, synthetic construct
  source 1..508: mol_type = other DNA
ctacggacttgatcgaattgagccttggtatggaaacctaccaagtgatagcttccaaatccagggaaccctgggatattttgaatgggtaatcctgagccaaatccggttcatgaagacaatgtttcttctcctaagataggaaggggataggtgcagagactcaatggaagctattctaacgaatgaatctcatttggtccaatactgtagttactgtagttatagaacgatctatttacacctcaaaaatggagagagatgttatataacatcagacaaaactggcgatcagaactcgttccaagcattttattcataagatagatgccagattcgagttgaagtactgatttgacattaagtaatccaattatgaacttatctactctagatgaataatttaattattttttggaataaatggttggacgaggataaagatagagtccaattctacatgtcaatgtaaacaacaatgcaaattgcagtaggaggaaaatccgtt

SEQ ID NO: 146 - DNA, 479 bp, synthetic construct
  source 1..479: mol_type = other DNA
agacgctacggacttgattgtattgagccttggtatggaaacctgctaagtggtaacttccaaattcagagaaaccctggaatgaaaaatgggcaatcctgagccaaatccacttttttcaaaaaagtggttctcaaactagaacccaaaggaaaaggataggtgcagagactcaatggaagctgttctaacgaatcgaagtaataacgattaatcacagaacccatattataatataggttctttattttattttgagaatgaaattaggaatgattatgaaatagaaaattcataatttttttagaattattgtgaatctattccaatcgaatattgagtaatcaaatccttcaattcattgttttcgagatcttttaaaaagtggattaatcggacgaggataaagagagagtcccattctacatgtcaatactgacaacaatgaaatttctagtaaaaggaaaatccgtcgactt

SEQ ID NO: 147 - DNA, 403 bp, synthetic construct
  source 1..403: mol_type = other DNA
aacaggttgacgacaacaggcctgtgacagcggggcttcgaactttccgtgtttttttttcttccgctagtcgatcatccgtgacggccgggcaagcgcccgagtaccaggaccgtcgggcccccgagaagggggggcctaggatgcggcaagacgacccggttcggggaacgccagcgtgcgctggccgatcccgaggcgaggtgccgtagcgggcacccgtcgtaacgcgcggtagggcgtcggtcccccctcccacggcggaggggaggcttaaggtacgtgctaaacccccagcgatggggcctgtaggaaaagccctggtacggcgaagcctacggggaccgcccgatggcggtcggatgccggcgggccacggagcccccggcgtcgattgctgtta

SEQ ID NO: 148 - DNA, 270 bp, synthetic construct
  source 1..270: mol_type = other DNA
agacgcagcggacttagaaaactgggcctcgatcgcgaaagggatcgagtggcagctctcaaactcagggaaacctaaaactttaaacattaaagtcatggcaatcctgagccaagctaaagctaaacaactaacagctttagaaggtgcagagactagacgggagctaccctaacggattcagccgagggtaaagggatagtccaattctcaacatcgcgattgttgatggcagcgaaagttgcagagagaatgaaaatccgctgactg

SEQ ID NO: 149 - DNA, 1046 bp, synthetic construct
  source 1..1046: mol_type = other DNA
gatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaaatcgttcaggtgaaaataattcattctttaatcataagcattcagacataactaaatctaaaatatcagaaaagatgaaaggtaaaaagcctagtaatattaaaaagatttcatgtgatggggttatttttgattgtgcagcagatgcagctagacattttaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatatt

SEQ ID NO: 150 - DNA, 488 bp, synthetic construct
  source 1..488: mol_type = other DNA
agacgctacggacttgattgtattgagccttggtatggaaacctgctaagtggtaacttccaaattcagagaaaccctggaatgaaaaatgggcaatcctgagccaaatcccttttttgaaaaacaagtggttctcaaactagaacccaaaggaaaaggataggtgcagagactcaatggaagctgttctaacgaatcgaagtaataacgattaatcacagaacccatattataatataggttctttattttatttttagaatgaaattaggaatgattatgaaatagaaaattcataatttttttttagaattattgtgaatctattccaatcaaatattgagtaatcaaatccttcaattcattgttttcgagatcttttaattttaaaaagtggattaatcggacgaggataaagagagagtcccattctacatgtcaatactgacaacaatgaaatttctagtaaaaggaaaatccgtcgactt

SEQ ID NO: 151 - DNA, 20 bp, synthetic construct
  source 1..20: mol_type = other DNA
ccgtcgattgtccactggtc

SEQ ID NO: 152 - DNA, 40 bp, synthetic construct
  source 1..40: mol_type = other DNA
ccgtcgattgtccactggtcccgtcgattgtccactggtc

SEQ ID NO: 153 - DNA, 20 bp, synthetic construct
  source 1..20: mol_type = other DNA
aaaaaaaaaaaaaaaaaaaa

SEQ ID NO: 154 - DNA, 20 bp, synthetic construct
  source 1..20: mol_type = other DNA
tttttttttttttttttttt

SEQ ID NO: 155 - DNA, 40 bp, synthetic construct
  source 1..40: mol_type = other DNA
aaaaaaaaaaaaaaaaaaaaaaaaaaaaaaaaaaaaaaaa

SEQ ID NO: 156 - DNA, 40 bp, synthetic construct
  source 1..40: mol_type = other DNA
tttttttttttttttttttttttttttttttttttttttt

SEQ ID NO: 157 - DNA, 20 bp, synthetic construct
  source 1..20: mol_type = other DNA
cccccccccccccccccccc

SEQ ID NO: 158 - DNA, 20 bp, synthetic construct
  source 1..20: mol_type = other DNA
gggggggggggggggggggg

SEQ ID NO: 159 - DNA, 40 bp, synthetic construct
  source 1..40: mol_type = other DNA
gggggggggggggggggggggggggggggggggggggggg

SEQ ID NO: 160 - DNA, 40 bp, synthetic construct
  source 1..40: mol_type = other DNA
cccccccccccccccccccccccccccccccccccccccc

SEQ ID NO: 161 - DNA, 20 bp, synthetic construct
  source 1..20: mol_type = other DNA
gaccagtggacaatcgacgg

SEQ ID NO: 162 - DNA, 40 bp, synthetic construct
  source 1..40: mol_type = other DNA
gaccagtggacaatcgacgggaccagtggacaatcgacgg

(19) United States
(12) Patent Application Publication
(10) Pub. No.: US 2024/0392304 A1
(43) Pub. Date: Nov. 28, 2024

(54) CIRCULAR RNA AND PREPARATION METHOD THEREOF

(71) Applicant: Center for Excellence in Molecular Cell Science, Chinese Academy of Sciences, Shanghai (CN)

(72) Inventors: Siqi LI, Shanghai (CN); Yifeng XU, Shanghai (CN); Chuxiao LIU, Shanghai (CN); Lingling CHEN, Shanghai (CN)

(73) Assignee: Center for Excellence in Molecular Cell Science, Chinese Academy of Sciences, Shanghai (CN)

(21) Appl. No.: 18/695,345

(22) PCT Filed: Sep. 26, 2022
(86) PCT No.: PCT/CN2022/121279

§ 371 Date: Mar. 25, 2024

(30) Foreign Application Priority Data
Sep. 26, 2021 (CN) 202111131281.0
Sep. 27, 2021 (CN) 202111136958.X
Sep. 27, 2021 (CN) 202111138732.3

(51) Int. Cl. C12N 15/67

(52) U.S. Cl. CPC C12N 15/67; C12N 2830/42; C12N 2830/60; C12N 2840/203

(57) ABSTRACT

The present invention relates to the field of biomedicine, in particular, to an improved circular RNA and a preparation method thereof, wherein the improved circular RNA has high generation efficiency and reduced immunogenicity. The present invention also relates to a vector for the preparation of the improved circular RNA, and the use of the improved circular RNA.

### TECHNICAL FIELD

The present invention relates to the field of biomedicine, in particular, to an improved circular RNA and preparation method thereof, wherein the improved circular RNA can be prepared with high efficiency and has reduced immunogenicity. The present invention also relates to a vector for the preparation of the improved circular RNA, and the use of the improved circular RNA.

### BACKGROUND

Circular RNA is a common type of RNA in eukaryotes. Naturally occurring circular RNAs are primarily produced by a molecular mechanism within cells called “back splicing”. Eukaryotic circular RNAs have been found to have a variety of molecular and cellular regulatory functions. For example, circular RNAs can regulate the expression of target genes by binding to microRNAs; circular RNAs can regulate gene expression by directly binding to target proteins.

Due to its circular nature, circular RNA has a longer half-life than linear mRNA, so it is speculated that circular RNA synthesized in vitro may have higher stability. Methods of forming circular RNAs in vitro include chemical method, enzymatic catalysis method, and ribozyme catalysis method. Chemical methods are expensive and the size of the circular RNA molecules that can be produced is limited. The enzymatic method mainly utilizes T4 RNA ligase to catalyze the circularization of linear RNA, and the size of RNA payload that can achieve circularization is also limited. Ribozyme catalysis (e.g., based on Group I introns) is a promising method for the preparation of circular RNAs.

The natural Group I intron system can undergo cleavage and ligation reactions to form circular intronic RNAs. A specific cleavage site conserved sequence located in the 5′ exon E1 is cleaved by the nucleophilic attack of the free 3′ hydroxyl group of guanosine triphosphate, resulting in a naked 3′ hydroxyl group, while the guanylate binds on the cleaved 5′ exon E1. After that, the exposed 3′ hydroxyl group at the 5′ end of the intron attacks the conserved sequence between the 3′ end of the intron and the exon E2, the exon E2 is excised, and the intron undergoes a circularizing reaction to obtain a circular intronic RNA. An improved ribozyme-catalyzed approach derived from Anabaena tRNA introns has been reported for the formation of circular RNAs in vitro, termed the “Group I permuted intron-exon self-splicing system, PIE system”. This method can excise the intron to form circular RNA containing exons. Therefore, this method has the potential to form expressible circular RNAs. The basic design principle of the PIE system is to connect the exon E1 sequence and E2 sequence end to end by molecular cloning to form a continuous circular plasmid. The intron is cleaved by restriction endonuclease to obtain a linear plasmid. Then, in vitro transcription is performed through the T7 promoter upstream of the permuted 3′ intron to obtain a linear RNA containing a 3′ intron-E2-E1-5′ intron structure. Similar to the natural Group I intron system, the specific cleavage site conserved sequence of exon E1 is cleaved by the nucleophilic attack of the free 3′ hydroxyl of guanylate, and the exon E1 produces a naked 3′ hydroxyl, while the guanylate binds to the cleaved 5′ intron. Subsequently, the exposed 3′ hydroxyl of exon E1 attacks the conserved sequence between the 3′ intron and exon E2, the 3′ intron is excised, and exon E2 and E1 undergo a circularizing reaction to obtain circular E1-E2 RNA.

However, there is a need in the art for improved circular RNAs and preparation methods thereof.

### SUMMARY OF THE INVENTION

In one aspect, the present invention provides a circular RNA precursor comprising the following elements from 5′ to 3′ direction in the following order:
a) a 3′ self-splicing intron fragment;
b) a first residual circularizing element;
c) a nucleotide sequence of interest;
d) a second residual circularizing element; and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor. In some preferred embodiments, the total length of the first residual circularizing element and the second residual circularizing element is about 5 to about 100 nucleotides.

In one aspect, provided herein is a nucleic acid vector for generating a circular RNA molecule, said vector comprises a coding sequence of the circular RNA precursor of the present invention.

In another aspect, the present invention provides a circular RNA, which is prepared from the circular RNA precursor or the nucleic acid vector of the present invention.

In another aspect, the present invention provides a circular RNA, which comprising a first residual circularizing element, a nucleotide sequence of interest, and a second residual circularizing element. In some preferred embodiments, the total length of the first residual circularizing element and the second residual circularizing element is about 5 to about 100 nucleotides.

In another aspect, the present invention also provides the use of the circular RNA precursor and/or circular RNA of the present invention as an expression vector.

In another aspect, the present invention provides a pharmaceutical composition comprising the nucleic acid vector of the present invention and/or the circular RNA precursor and/or circular RNA of the present invention, and a pharmaceutically acceptable carrier.

In another aspect, the present invention provides a method for preparing a circular RNA, the method comprises:
1) providing a circular RNA precursor of the present invention or obtaining a circular RNA precursor by transcribing from the nucleic acid vector of the present invention;
2) incubating the circular RNA precursor in the presence of a divalent metal cation at a temperature at which RNA circularization occurs; and
3) harvesting the circular RNA obtained in step 2).

In another aspect, the present invention provides a method for preparing a circular RNA, the method comprises
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.

In one aspect, the present invention provides a method for purifying a circular RNA, the method comprises:
a) contacting a mixture comprising circular RNA and uncircularized linear circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.

In one aspect, the present invention provides a method for purifying circular RNA, the method comprises:
i) contacting a mixture comprising circular RNA and uncircularized linear circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and optionally, steps i)-iii) are performed multiple times, e.g., 2 times, 3 times, 4 times or more.

In one aspect, the present invention provides a method for purifying circular RNA, the method comprises:
i) adding a linear RNA-specific tag to the linear RNA in a mixture comprising circular RNA and linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii),
optionally, steps ii)-iv) are performed multiple times, e.g., 2 times, 3 times, 4 times or more.

In one aspect, the present invention provides a circular RNA produced or purified by the method of the invention.

In one aspect, the present invention provides an in vitro transcription method comprising:
a) providing a nucleic acid vector as a template for in vitro transcription;
b) incubating the nucleic acid vector in an in vitro transcription system comprising a divalent metal cation and an RNA polymerase for a first period of time; and
c) i) adding an additional amount of metal cation to the system, or ii) changing the buffer of the system and adding a metal cation to the system, and incubating the system for a second period of time.

In one aspect, the present invention provides an RNA produced by the in vitro transcription method of the invention.

### BRIEF DESCRIPTION OF THE DRAWINGS

FIG. 1. Different methods for RNA circularization.

FIG. 2. RNA circularization by T4 ligase.

FIG. 3. RNA circularization through splint and T4 ligase.

FIG. 4. RNA circularization by Td Group I introns.

FIG. 5. RNA circularization by Ana Group I introns.

FIG. 6. RNA circularization by Tornado system.

FIG. 7. Schematic diagram of the AnaX system and components for RNA circularization.

FIG. 8. Circular POLR2A generated by Group I intron splicing stimulates immune responses.

FIG. 9. Secondary structures of circular POLR2A_Lig, circular POLR2A_TD and circular POLR2A_Ana predicted by in vitro circSHAPE-MaP.

FIG. 10. Shortened the alien sequence in circular RNA reduce immunogenicity.

FIG. 11. The translation efficiency of AnaX-circRNA is higher than that of mRNA.

FIG. 12. The immunogenicity of AnaX-circRNA is lower than that of mRNA.

FIG. 13. AnaX circularizes RNAs of different lengths.

FIG. 14. AnaX-catalyzed circular RNAs are less immunogenic.

FIG. 15. AnaX catalyzed circular RBD and Luciferase RNAs with higher translation efficiency as compared to the corresponding mRNAs.

FIG. 16. Modification of base pairs in the stem-loop of the residual sequences.

FIG. 17. Increasing the number of base pairs in the stem-loop increases the efficiency of RNA circularization.

FIG. 18. Replacement of base pairs in stem-loop of residual sequences with AU pairs or GC pairs.

FIG. 19. Strong pairing ability in stem-loop structure increases RNA circularization efficiency (different time periods for circularization).

FIG. 20. Strong pairing ability in stem-loop structure increases RNA circularization efficiency (encoding for mCherry and Luciferase).

FIG. 21. Engineering the base pairs in the stem-loop of the residual sequence does not affect immunogenicity.

FIG. 22. Replacing base pairs in stem-loop of residual sequences with GC pairs increases circular RNA translation efficiency.

FIG. 23. Effect of the loop in the stem-loop of the residual sequence on RNA circularization.

FIG. 24. Shortened alien sequences in circular RNA maintain high circularization efficiency.

FIG. 25. Modification of the homology arms of the AnaX Group I intron.

FIG. 26. Increasing the homology arm length of AnaX Group I intron can improve the efficiency of RNA circularization.

FIG. 27. Improvement of Azoarcus Group I Intron and its stem-loop structure.

FIG. 28. IRES did not affect protein translation either upstream or downstream of the protein coding region.

FIG. 29. Simplified and optimized RNA circularization method.

FIG. 30. The addition of Mg²⁺ improves the efficiency of IVT and circularization.

FIG. 31. Adding a large amount of Mg²⁺ after 3 h of IVT can significantly improve the efficiency of IVT.

FIG. 32. Adding NaOAc can improve in vitro transcription efficiency.

FIG. 33. Adding KCl can improve in vitro transcription efficiency.

FIG. 34. Mg(OAc)₂ can improve in vitro transcription efficiency.

FIG. 35. High temperature affects in vitro transcription efficiency without promoting circularization.

FIG. 36. Prolonged in vitro transcription time increases RNA yield and promotes RNA circularization.

FIG. 37. Effect of buffer on in vitro transcription and RNA circularization efficiency.

FIG. 38. Effects of different MnCl₂ concentrations on RNA circularization efficiency.

FIG. 39. Effect of circularization temperature and time on RNA circularization efficiency.

FIG. 40. Effects of different buffers and different pH on RNA circularization efficiency.

FIG. 41. Purifying AnaX-circRNA by using a ligand.

FIG. 42. Comparing the enrichment of circular RNAs by ligands of different lengths.

FIG. 43. Ligand lengths were shortened to compare the specificity of ligand binding to circular RNAs.

FIG. 44. Translation efficiency of circular mCherry RNA purified by affinity oligo-ligand magnetic beads.

FIG. 45. Low immunogenicity of circular mCherry purified by affinity oligo-ligand magnetic beads.

FIG. 46. Affinity oligo-ligand-purified circular Luciferase RNA.

FIG. 47. Affinity oligo-ligand purified circular POLR2A cyclized by T4 ligase.

FIG. 48. Purification of Td class I intron-cyclized circular POLR2A by affinity oligo-ligands.

FIG. 49. Efficiency of circular RNA purification by Ligand Intron and Ligand Feature columns.

FIG. 50 CircRNA purified by double affinity chromatography.

FIG. 51. Tailing of linear RNA to purify circular RNA.

FIG. 52. Circular RNAs are more stable in cells than linear RNAs.

FIG. 53. Naked circular RNAs are stable at RT or 4° C.

FIG. 54. Comparison of expression and stability of Luciferase mRNA and circular Luciferase RNA in cells.

FIG. 55. Comparison of the immunogenicity of Luciferase mRNA and circular Luciferase RNA in cells.

FIG. 56. Delivery of naked circular RNA by intradermal injection.

FIG. 57. Delivery of circular RNA by liposomes.

FIG. 58. m5C modification affects RNA circularization.

FIG. 59. Ψ modification affects RNA circularization.

FIG. 60. m1Ψ modification affects RNA circularization.

FIG. 61. m6A modification affects RNA circularization.

FIG. 62. Nucleotide modifications affect circular RNA expression.

FIG. 63. Efficiency of different types of viral IRES detected by fluorescence.

FIG. 64. Efficiency of different types of viral IRES detected by Western Blot.

FIG. 65. BRAV1_L and PV1_L show translation efficiencies comparable to CVB3.

FIG. 66. Detection of the activity of different IRES in different cells.

### DETAILED DESCRIPTION OF THE INVENTION

In the present invention, unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, Efland Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. Although the nucleotide sequences herein may be represented as DNA sequences (comprising T(s)), when referring to RNA, one skilled in the art can readily determine the corresponding RNA sequence (i.e., replacing T with U).

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). There are many methods for determining sequence identity. An example of algorithms suitable for determining percent sequence identity is the algorithm used in the Basic Local Alignment Search Tool (hereinafter “BLAST”), see e.g., Altschul et al., J. Mol. Biol. 215:403-410, 1990 and Altschul et al, Nucleic Acids Res., 15:3389-3402, 1997. Software for performing BLAST analysis is publicly available through the National Center for Biotechnology Information (hereafter “NCBI”). Default parameters used to determine sequence identity using software available from NCBI (such as BLASTN for nucleic acid sequences) are described in McGinnis et al. Nucleic Acids Res., 32: W20-W25, 2004.

### I. Circular RNA, Precursor, and Vector

In one aspect, the present invention provides a circular RNA precursor comprising the following elements from 5′ to 3′ direction in the following order:
a) a 3′ self-splicing intron fragment,
b) a first residual circularizing element,
c) a nucleotide sequence of interest,
d) a second residual circularizing element, and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor.

“Circular RNA precursor” herein refers to a linear RNA molecule capable of forming a covalently linked closed circular RNA molecule, e.g., by self-splicing. The circular RNA precursor may be produced by transcription from a nucleic acid vector comprising a coding sequence of the circular RNA precursor. Alternatively, the circular RNA precursor may also be obtained by chemical synthesis.

In some embodiments, the circular RNA precursor is capable of forming a covalently linked closed circular RNA molecule by self-splicing under the action of the self-splicing intron fragments and the residual circularizing elements.

As used herein, the term “self-splicing intron” refers to an intron having self-splicing ribozyme activity and capable of excising itself and joining two flanking exons. In some embodiments, the splicing is autocatalytic splicing.

“Self-splicing introns” include, but are not limited to, Group I introns and Group II introns. Group I introns contain 14 subgroups, while most of the Group I introns belong to the IC3 subgroup. For example, the Group I intron may be a Group I intron of the cyanobacterium Anabaena belonging to the IC3 subgroup or a Group I intron from a T4 phage Group I intron belonging to the IA2 subgroup or a Group I intron from Azoarcus sp. BH72 belonging to the IC3 subgroup. Additional examples of self-splicing introns useful in the present invention include, but are not limited to, self-splicing introns derived from the following organisms: Enterobacteriophage T4, Bacteriophage Twort, Bacteriophage SPO1, Bacteriophage S3b, Bacillus anthracis, Clostridium botulinum, Tetrahymena thermophila, Dunaliella parva, Pneumocystis carinii, Physarum polycephalum, Anabaena sp. PCC7120, Scytonema hofmanni, Agrobacterium tumefaciens, Synechocystis PCC 6803, Synechococcus elongatus PCC 6301, Neurospora crassa, Candida albicans, Scytalidium cerradiumydiaces, Pediadiaces Chlamydomonas nivalis, Chlorella vulgaris, Amoebidium parasiticum, Neurospora crassa, Emericella nidulans, Saccharomyces cerevisiae, Schizosaccharomyces pombe, Neochloris aquatica, Dunaliella parva, Symkania negevensis, Emericella nidulans. See e.g., Vicens, Q., et al., (2008). Toward predicting self-splicing and protein-facilitated splicing of group I introns. RNA 14: 2013-2029; Tanner, A. M., et al., (1996). Activity and thermostability of the small self-splicing group I intron in the pre-tRNAIIe of the purple bacterium Azoarcus. RNA 2:74-83.

In some embodiments, the 3′ self-splicing intron fragment and the 5′ self-splicing intron fragment are derived from a same self-splicing intron. In some embodiments, the 3′ self-splicing intron fragment is derived from or contains a 3′ terminal portion of the self-splicing intron (a native self-splicing intron), and accordingly the 5′ self-splicing intron fragment is derived from or contains a 5′ terminal portion of the self-splicing intron (a native self-splicing intron). In some embodiments, the 3′ self-splicing intron fragment and the 5′ self-splicing intron fragment in combination retain the self-splicing activity of the self-splicing intron (a native self-splicing intron).

In some embodiments, the 3′ self-splicing intron fragment is derived from a 3′ terminal portion of a native self-splicing intron starting from an internal split site to the 3′ end of the native self-splicing intron, and accordingly the 5′ self-splicing intron fragment is derived from a 5′ terminal portion of the native self-splicing intron starting from the internal split site to the 5′ end of the native self-splicing intron, and, the 3′ self-splicing intron fragment and the 5′ self-splicing intron fragment in combination retain the self-splicing activity of the native self-splicing intron.

In some embodiments, the self-splicing intron is a Group I intron. In some embodiments, the self-splicing intron is a Group I intron of the IA2 or IC3 subgroup, preferably IC3 subgroup.

In some embodiments, the 3′ self-splicing intron fragment is a 3′ Group I intron fragment. In some embodiments, the 5′ self-splicing intron fragment is a 5′ Group I intron fragment.

As used herein, a 3′ self-splicing intron fragment (e.g., a 3′ Group I intron fragment) is a sequence that is at least 75%, e.g., at least 80%, at least 85%, at least 90%, at least 95%, 100% identical to the 3′ terminal portion of a native self-splicing intron (e.g., a Group I intron). A 5′ self-splicing intron fragment (e.g., a 5′ Group I intron fragment) is a sequence that is at least 75%, e.g., at least 80%, at least 85%, at least 90%, at least 95%, 100% identical to the 5′ terminal portion of a native self-splicing intron (e.g., a Group I intron).

It is generally believed that in order to achieve circularization, the native Group I intron needs to be split at an internal site to form a so called “Group I permuted intron-exon self-splicing system, PIE system”. The internal split site is selected to allow generating two separate portions of native Group I intron (the 3′ terminal portion and the 5′ terminal portion) which together can maintain ribozyme activity necessary for the self-splicing, even after permutation. It is believed that the two separate portions of native Group I intron maintaining the overall conformation of the native Group I intron can maintain ribozyme activity necessary for the self-splicing. In some embodiments, the 3′ terminal portion of the native Group I intron is the portion from the internal split site to the 3′ end of the native Group I intron, correspondingly, the 5′ terminal portion of the native Group I intron is the portion from the internal split site to the 5′ end of the native Group I intron.

The internal split site of a Group I intron can be determined by a person skilled in the art, e.g., by referenced to Puttaraju M., et al., (1992) Group I permuted intron-exon (PIE) sequences self-splice to produce circular exons; and/or Puttaraju M., et al., (1996) Circular ribozymes generated in Escherichia coli using group I self-splicing permuted intron-exon sequences. For example, for a Group I intron, especially an Anabaena Group I intron, it can usually be split at a specific site in its P6 region to form the PIE system. Thus, in some embodiments, the 3′ terminal portion of the native Group I intron (e.g., Anabaena Group I intron) is the portion starting from a specific site in the P6 region to the 3′ end of the native Group I intron (e.g., Anabaena Group I intron). In some embodiments, the 5′ terminal portion of the native Group I intron (e.g., Anabaena Group I intron) is the portion starting from a specific site in the P6 region to the 5′ end of the Group I intron (e.g., Anabaena Group I intron). For a Group I introns, especially an Anabaena Group I intron, the split site can also be located within its P2, P5, P8 or P9 region to form the PIE system, as can be seen in WO2021236855A1. For a Group II intron, the split site can be located within its D4 region, as can be seen in Roth A., et al., (2021) Natural circularly permuted group II introns I bacteria produce RNA circles; Pyle M. A., et al., (2016) Group II intron self-splicing.

The 3′ terminal portion of a native Group I intron may have a length of about 5% to about 95%, such as about 5%, about 10%, about 20%, about 30%, about 40%, about 50%, about 60%, about 70%, about 80%, about 90%, about 95% of the full length of the native Group I intron. Accordingly, the 5′ terminal portion of a native Group I intron may have a length of about 5% to about 90%, such as about 5%, about 10%, about 20%, about 30%, about 40%, about 50%, about 60%, about 70%, about 80%, about 90%, about 95% of the full length of the native Group I intron.

In some embodiments, the combination of the 3′ Group I intron fragment and the 5′ Group I intron fragment substantially has the self-splicing activity of the corresponding native Group I intron.

In some embodiments, the self-splicing intron is a Group I intron of cyanobacterial Anabaena, for example, the Group I intron of the pre-tRNA-Leu gene of cyanobacterial Anabaena. Accordingly, in some embodiments, the 3′ self-splicing intron fragment (3′ Group I intron fragment) and the 5′ self-splicing intron fragment (5′ Group I intron fragment) are derived from a Group I intron of cyanobacterium Anabaena, for example, the Group I intron of the pre-tRNA-Leu gene of cyanobacterial Anabaena. In some embodiments, the native Group I intron of the Anabaena pre-tRNA-Leu gene has the nucleotide sequence of SEQ ID NO:136. The P6 region corresponds to position 98 to position 157 of SEQ ID NO:136. The split site may be any position from position 122 to position 138 of SEQ ID NO:136.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1 or a nucleotide sequence having at least 75%, e.g., at least 80%, at least 85%, at least 90%, at least 95%, 100% identity to SEQ ID NO: 1. In some embodiments, the 5′ self-splicing intron fragment (5′ Group I intron fragment) is derived from the Group I intron of the Anabaena pre-tRNA-Leu gene and comprises or consists of a nucleotide sequence of SEQ ID NO:2 or a nucleotide sequence having at least 75%, e.g., at least 80%, at least 85%, at least 90%, at least 95%, 100% identity to SEQ ID NO: 2.

In some embodiments, the self-splicing intron is a Group I intron of T4 phage, for example, the Group I intron of the td gene of T4 phage. Accordingly, in some embodiments, the 3′ self-splicing intron fragment (3′ Group I intron fragment) and the 5′ self-splicing intron fragment (5′ Group I intron fragment) are derived from a T4 phage Group I intron, for example, the Group I intron of the td gene of T4 phage. In some embodiments, the native Group I intron of the td gene of T4 phage has the nucleotide sequence of SEQ ID NO: 149. The P6 region corresponds to position 100 to position 246 of SEQ ID NO:149. The split site may be any position from position 109 to position 125 of SEQ ID NO:149.

In some embodiments, the 3′ self-splicing intron fragment (3′ Group I intron fragment) is derived from the Group I intron of td gene of T4 phage and comprises a nucleotide sequence of SEQ ID NO: 5 or a nucleotide sequence having at least 75%, e.g., at least 80%, at least 85%, at least 90%, at least 95%, 100% identity to SEQ ID NO: 5. In some embodiments, the 5′ self-splicing intron fragment (5′ Group I intron fragment) is derived from the Group I intron of td gene of T4 phage and comprises the nucleotide sequence of SEQ ID NO:6 or a nucleotide sequence having at least 75%, e.g., at least 80%, at least 85%, at least 90%, at least 95%, 100% identity to SEQ ID NO: 6.

In some embodiments, the self-splicing intron may be a Group I intron of Azoarcus sp. BH72, for example, the Group I intron of the pre-tRNA-Ile gene of Azoarcus sp. BH72. Accordingly, in some embodiments, the 3′ self-splicing intron fragment (3′ Group I intron fragment) and the 5′ self-splicing intron fragment (5′ Group I intron fragment) are derived from a Group I intron of Azoarcus sp. BH72, for example, the Group I intron of the pre-tRNA-Ile gene of Azoarcus sp. BH72. In some embodiments, the native Group I intron of the pre-tRNA-Ile gene of Azoarcus sp. BH72 has the nucleotide sequence of SEQ ID NO:137. The P6 region corresponds to position 108 to position 138 of SEQ ID NO:137. The split site may be any position from position 121 to position 125 of SEQ ID NO:137.

In some embodiments, the 3′ self-splicing intron fragment (3′ Group I intron fragment) is derived from the Group I intron of pre-tRNA-Ile gene of Azoarcus sp. BH72 and comprises a nucleotide sequence of SEQ ID NO: 3 or a nucleotide sequence having at least 75%, e.g., at least 80%, at least 85%, at least 90%, at least 95%, 100% identity to SEQ ID NO: 3. In some embodiments, the 5′ self-splicing intron fragment (5′ Group I intron fragment) is derived from the Group I intron of pre-tRNA-Ile gene of Azoarcus sp. BH72 and comprises a nucleotide sequence of SEQ ID NO:4 or a nucleotide sequence having at least 75%, e.g., at least 80%, at least 85%, at least 90%, at least 95%, 100% identity to SEQ ID NO: 4.

As used herein, a “residual circularizing element” refers to a sequence that is involved in or required for circularization by the self-splicing intron and participates in circularization together with the self-splicing intron but is retained in the final circular RNA. A “residual circularizing element” can also be referred as an “intra-circle circularizing element” herein.

The inventors have surprisingly found that when self-splicing introns are used for RNA circularization, the introduction of an additional circularizing element into the circular RNA may result in increased immunogenicity of the circular RNA molecule, and after truncating and mutating the elements remained in the circular RNA (residual circularizing elements), the circularization efficiency can be retained or even improved, and the immunogenicity of the circular RNA can be significantly reduced, which is of great significance for the potential application of circular RNA as a drug or drug carrier.

In some embodiments, the total length of the first residual circularizing element and the second residual circularizing element is no greater than about 500 nucleotides, e.g., no greater than about 500, about 400, about 300, about 200, about 100, about 90, about 80, about 70, about 60, about 50, about 40, about 30, about 20, about 15, about 10, about 5 nucleotides.

In some embodiments, the total length of the first residual circularizing element and the second residual circularizing element is from about 2 to about 500 nucleotides, or any integer number therebetween. In some embodiments, the total length of the first residual circularizing element and the second residual circularizing element is from about 2 to about 400 nucleotides, or any integer number therebetween. In some embodiments, the total length of the first residual circularizing element and the second residual circularizing element is from about 2 to about 300 nucleotides, or any integer number therebetween. In some embodiments, the total length of the first residual circularizing element and the second residual circularizing element is from about 2 to about 200 nucleotides, or any integer number therebetween. In some embodiments, the total length of the first residual circularizing element and the second residual circularizing element is from about 2 to about 100 nucleotides, or any integer number therebetween. In some embodiments, the total length of the first residual circularizing element and the second residual circularizing element is from about 2 to about 90 nucleotides, or any integer number therebetween. In some embodiments, the total length of the first residual circularizing element and the second residual circularizing element is from about 2 to about 80 nucleotides, or any integer number therebetween. In some embodiments, the total length of the first residual circularizing element and the second residual circularizing element is from about 2 to about 70 nucleotides, or any integer number therebetween. In some embodiments, the total length of the first residual circularizing element and the second residual circularizing element is from about 2 to about 60 nucleotides, or any integer number therebetween. In some embodiments, the total length of the first residual circularizing element and the second residual circularizing element is about 2 to about 50 nucleotides, or any integer number therebetween. In some embodiments, the total length of the first residual circularizing element and the second residual circularizing element is from about 2 to about 40 nucleotides, or any integer number therebetween. In some embodiments, the total length of the first residual circularizing element and the second residual circularizing element is about 2 to about 30 nucleotides, or any integer number therebetween. In some embodiments, the total length of the first residual circularizing element and the second residual circularizing element is from about 2 to about 20 nucleotides, or any integer number therebetween. In some embodiments, the total length of the first residual circularizing element and the second residual circularizing element is from about 2 to about 15 nucleotides, or any integer number therebetween. In some embodiments, the total length of the first residual circularizing element and the second residual circularizing element is about 2 to about 10 nucleotides, or any integer number therebetween.

In some embodiments, the total length of the first residual circularizing element and the second residual circularizing element is at least 5 nucleotides in length. In some embodiments, the total length of the first residual circularizing element and the second residual circularizing element is at least 10 nucleotides in length. In some embodiments, the total length of the first residual circularizing element and the second residual circularizing element is at least 15 nucleotides in length.

In some embodiments, the total length of the first residual circularizing element and the second residual circularizing element is from about 5 to about 100 nucleotides, or any integer number therebetween. In some embodiments, the total length of the first residual circularizing element and the second residual circularizing element is from about 10 to about 100 nucleotides, or any integer number therebetween. In some embodiments, the total length of the first residual circularizing element and the second residual circularizing element is from about 15 to about 100 nucleotides, or any integer number therebetween. In some embodiments, the total length of the first residual circularizing element and the second residual circularizing element is from about 15 to about 30 nucleotides, or any integer number therebetween. In some embodiments, the total length of the first residual circularizing element and the second residual circularizing element is from about 20 to about 35 nucleotides, or any integer number therebetween. In some embodiments, the combined length of the first residual circularizing element and the second residual circularizing element is from about 25 to about 40 nucleotides, or any integer number therebetween. In some embodiments, the total length of the first residual circularizing element and the second residual circularizing element is from about 30 to about 45 nucleotides, or any integer number therebetween. In some embodiments, the total length of the first residual circularizing element and the second residual circularizing element is from about 35 to about 50 nucleotides, or any integer number therebetween. In some embodiments, the total length of the first residual circularizing element and the second residual circularizing element is from about 40 to about 55 nucleotides, or any integer number therebetween. In some embodiments, the combined length of the first residual circularizing element and the second residual circularizing element is from about 45 to about 60 nucleotides, or any integer number therebetween. In some embodiments, the total length of the first residual circularizing element and the second residual circularizing element is from about 50 to about 65 nucleotides, or any integer number therebetween. In some embodiments, the total length of the first residual circularizing element and the second residual circularizing element is from about 55 to about 70 nucleotides, or any integer number therebetween. In some embodiments, the total length of the first residual circularizing element and the second residual circularizing element is about 60 to about 75 nucleotides, or any integer number therebetween. In some embodiments, the total length of the first residual circularizing element and the second residual circularizing element is from about 65 to about 80 nucleotides, or any integer number therebetween. In some embodiments, the total length of the first residual circularizing element and the second residual circularizing element is from about 70 to about 85 nucleotides, or any integer number therebetween. In some embodiments, the total length of the first residual circularizing element and the second residual circularizing element is from about 75 to about 90 nucleotides, or any integer number therebetween. In some embodiments, the combined length of the first residual circularizing element and the second residual circularizing element is from about 80 to about 95 nucleotides, or any integer number therebetween. In some embodiments, the total length of the first residual circularizing element and the second residual circularizing element is from about 85 to about 100 nucleotides, or any integer number therebetween.

In some embodiments, the total length of the first residual circularizing element and the second residual circularizing element is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500 nucleotides.

In some embodiments, the first residual circularizing element and the second residual circularizing element are configured such that the circular RNA comprising them has reduced immunogenicity relative to a control circular RNA comprising the circularizing elements of SEQ ID NO:29 and SEQ ID NO:64 (Ana 3.0, with residual circularizing elements having a total length of 176 nucleotides).

In some embodiments, the first residual circularizing element and the second residual circularizing element are configured such that the circular RNA comprising them has reduced immunogenicity relative to a control linear RNA, for example, a corresponding linear RNA which contains the same nucleotide sequence of interest but has no chemically modified nucleotide.

“Reduced immunogenicity” as used herein may refer to that the circular RNA, upon contacted with cells, elicits a reduced immune response, i.e., an immune response at a level lower than a control circular RNA or control linear RNA. For example, reduced immune response refers to reduced expression of cytokines. The cytokines include, but are not limited to, IFNβ, TNFα, IL6 and/or RIG-I. In some embodiments, the immunogenicity of the circular RNA is reduced by at least about 10%, at least about 20%, at least about 30%, at least about 40%, at least about 50%, at least about 60%, at least about 70%, at least about 80%, at least about 90% or more. The reduced immunogenicity can be determined by methods well known in the art, such as those described in Example 10 of the present application.

In some embodiments, the first residual circularizing element and the second residual circularizing element are configured such that the circular RNA or circular RNA precursor comprising them has comparable or increased circularization efficiency relative to a control circular RNA or circular RNA precursor comprising the circularizing elements of SEQ ID NO:29 and SEQ ID NO:64 (Ana 3.0).

“Circularization efficiency” as used herein may refer to the ratio of outcome circular RNA to input precursor in a given time period. Alternatively, “Circularization efficiency” as used herein may refer to the ratio of desired circular RNA to linear RNAs in the final product in a given time period. The circularizing efficiency can be determined by methods well known in the art, such as those described in Example 17, 19 and 20 of the present application.

In some embodiments, the circularizing efficiency of the circular RNA is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or more.

In some embodiments, the first residual circularizing element comprises or consists of from 5′ to 3′ direction a 3′ exon region and optionally a first spacer. In the circular RNA precursor, the 5′ end of the 3′ exon region is directly connected to the 3′ end of the 3′ self-splicing intron fragment.

As used herein, “exon region” in the residual circularizing element is a sequence derived from the native exon of the self-splicing intron (the exon flanking the self-splicing intron) and capable of being recognized and/or spliced by the self-splicing intron (or a combination of the first self-splicing intron fragment and the second self-splicing intron fragment), and thus is required for circularization. An “exon region” can also be referred as a “splicing site sequence” herein.

In some embodiments, the 3′ exon region is derived from the native 3′ exon of the self-splicing intron (the exon flanking (downstream of) the 3′ end of the self-splicing intron) or a contiguous fragment thereof starting from the 5′ terminal nucleotide.

In some embodiments, the 3′ exon region is the entire native 3′ exon of the self-splicing intron, or has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the entire native 3′ exon of the self-splicing intron, or has 1, 2, 3, 4, 5, 6, 7, 8, 9, or 10 or more nucleotide substitutions, deletions or additions compared to the entire native 3′ exon of the self-splicing intron.

In some embodiments, the 3′ exon region is a contiguous fragment starting from the 5′ terminal nucleotide of the native 3′ exon. In some embodiments, the 3′ exon region has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with a contiguous fragment starting from the 5′ terminal nucleotide of the native 3′ exon. In some embodiments, the 3′ exon region has 1, 2, 3, 4, 5, 6, 7, 8, 9, or 10 or more nucleotide substitutions, deletions or additions compared to a continuous fragment starting from the 5′ terminal nucleotide of the native 3′ exon.

In some embodiments, the contiguous fragment starting from the 5′ terminal nucleotide of the native 3′ exon comprises or consists of at least 1%, at least 5%, at least 10%, at least 20%, at least 30%, at least 40%, at least 50%, at least 60%, at least 70%, at least 80%, at least 90% nucleotides of the native 3′ exon. In some embodiments, the contiguous fragment starting from the 5′ terminal nucleotide of the native 3′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 5′ terminal nucleotide of the native 3′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 3′ exon.

In some embodiments, where the self-splicing intron is a Group I intron, the 3′ exon region at least comprises a sequence (such as a sequence of about 1 to about 20 nucleotides) at it 5′ terminus which can pair with the P1 region of the corresponding Group I intron to form a P10 duplex region.

It is believed that for self-splicing of Group I introns, the consecutive one or more nucleotides (such as at least about 1 to about 7 nucleotides) from the 5′ end of the native 3′ exon can pair with the P1 region to form a P10 duplex region, and thus plays an important role in self-splicing. Definitions of the P1 and P10 regions of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the second residual circularizing element comprises or consists of from 3′ to 5′ direction a 5′ exon region and optionally a second spacer. In the circular RNA precursor, the 3′ end of the 5′ exon region is directly connected to the 5′ end of the second self-splicing intron fragment.

In some embodiments, the 5′ exon region is derived from the native 5′ exon of the self-splicing intron (the exon flanking (downstream of) the 5′ end of the self-splicing intron) or a contiguous fragment thereof starting from the 3′ terminal nucleotide.

In some embodiments, the 5′ exon region is the entire native 5′ exon of the self-splicing intron, or has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the entire native 5′ exon of the Group I intron, or has 1, 2, 3, 4, 5, 6, 7, 8, 9, or 10 or more nucleotide substitutions, deletions or additions compared to the entire native 5′ exon of the self-splicing intron.

In some embodiments, the 5′ exon region is a contiguous fragment starting from the 3′ terminal nucleotide of the native 5′ exon. In some embodiments, the 5′ exon region has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with a contiguous fragment starting from the 3′ terminal nucleotide of the native 5′ exon. In some embodiments, the 5′ exon region has 1, 2, 3, 4, 5, 6, 7, 8, 9, or 10 or more nucleotide substitutions, deletions or additions compared to a continuous fragment starting from the 3′ terminal nucleotide of the native 5′ exon.

In some embodiments, the contiguous fragment starting from the 3′ terminal nucleotide of the native 5′ exon comprises or consists of at least 1%, at least 5%, at least 10%, at least 20%, at least 30%, at least 40%, at least 50%, at least 60%, at least 70%, at least 80%, at least 90% nucleotides of the native 5′ exon. In some embodiments, the contiguous fragment starting from the 3′ terminal nucleotide of the native 5′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 3′ terminal nucleotide of the native 5′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 5′ exon.

In some embodiments, where the self-splicing intron is a Group I intron, the 5′ exon region comprises a sequence (for example, a sequence of about 3 to about 8 consecutive nucleotides) at its 3′ terminus which can pair with the internal guide sequence (IGS) of the corresponding Group I intron to form a P1 double-stranded region.

It is believed that for self-splicing of Group I introns, about 3 to about 8 consecutive nucleotides from the 3′ end of the native 5′ exon can pair with the internal guide sequence (IGS) of the intron to form the P1 double-stranded region, thus playing an important role in self-splicing. Definitions of IGS and/or P1 region of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the 3′ exon region is derived from the 3′ native exon of the Group I intron of the Anabaena pre-tRNA-Leu gene. The 3′ native exon of the Group I intron of the Anabaena pre-tRNA-Leu gene comprises the nucleotide sequence of SEQ ID NO:7. In some embodiments, the 5′ exon region is derived from the 5′ native exon of the Group I intron of the Anabaena pre-tRNA-Leu gene. The 5′ native exon of the Group I intron of the Anabaena pre-tRNA-Leu gene comprises the nucleotide sequence of SEQ ID NO:8.

In some embodiments, the 3′ exon region is derived from the 3′ native exon of the Group I intron of the td gene of T4 phage. The 3′ native exon of the td gene of T4 phage comprises the nucleotide sequence of SEQ ID NO:11. In some embodiments, the 5′ exon region is derived from the 5′ native exon of the Group I intron of the td gene of T4 phage. The 5′ native exon of the td gene of T4 phage comprises the nucleotide sequence of SEQ ID NO:12.

In some embodiments, the 3′ exon region is derived from the 3′ native exon of the Group I intron of pre-tRNA-Ile gene of Azoarcus sp. BH72. The 3′ native exon of pre-tRNA-Ile gene of Azoarcus sp. BH72 comprises the nucleotide sequence of SEQ ID NO:9. In some embodiments, the 5′ exon region is derived from the 5′ native exon of the Group I intron of pre-tRNA-Ile gene of Azoarcus sp. BH72. The 5′ native exon of pre-tRNA-Ile gene of Azoarcus sp. BH72 comprises the nucleotide sequence of SEQ ID NO:10.

During the circularization of the circular RNA precursor, the 3′ self-splicing intron fragment (e.g., 3′ Group I intron fragment) and the sequence upstream of its 5′ end (if present), and the 5′ self-splicing intron fragment (e.g., 5′ Group I intron fragment) and the sequence downstream of its 3′ end (if present) are excised, and the 5′ end of the first residual circularizing element and the 3′ end of the second residual circularizing element are covalently linked to achieve circularization of the RNA.

In some embodiments, the first residual circularizing element and the second residual circularizing element comprise spacers of different sequences, or one of them comprises a spacer and the other does not.

As used herein, “spacer” refers to any contiguous nucleotide sequence that at least does not negatively interfere with the function of the elements connected by it. Generally, if it is desired to avoid the interaction of two near or adjacent elements, a spacer can be inserted between the two elements. The spacer sequences described herein can serve two functions: (1) to facilitate circularization and (2) to facilitate functionality by allowing correct folding of the residual circularizing element and the nucleotide sequence of interest (e.g., IRES). In some embodiments, the spacer is no more than 150, no more than 100, no more than 50, no more than 30, no more than 10, no more than 5, or no more than 3 nucleotides in length. In some embodiments, the spacer is 5 nucleotides in length. In some embodiments, the spacer is 4 nucleotides in length. In some embodiments, the spacer is 3 nucleotides in length. In some embodiments, the first spacer may be absent. In some embodiments, the second spacer may be absent. In some embodiments, the first spacer and the second spacer may be absent.

In some embodiments, the first residual circularizing element and the second residual circularizing element are configured to be capable of forming a stem-loop structure. In some embodiments, the loop of the stem-loop structure comprises the splicing junction.

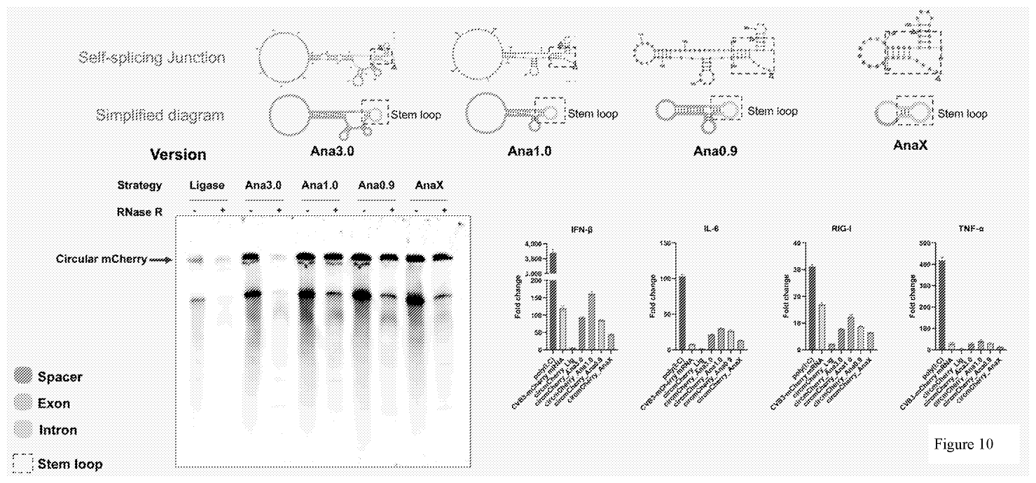

In some embodiments, the presence of the stem-loop structure can be predicted and/or determined by the nucleotide sequences of the 3′ self-splicing intron fragment (e.g., 3′ Group I intron fragment), the first residual circularizing element, the second residual circularizing element, and the 5′ self-splicing intron fragment (e.g., 5′ Group I intron fragment) involved in circularization. In some embodiments, the presence of a stem-loop structure can be predicted and/or determined from the nucleotide sequence by RNA structure prediction tools such as RNAfold (http://rna.tbi.univie.ac.at/cgi-bin/RNAWebSuite/RNAfold.cgi) or RNAstructure (https://rna.urmc.rochester.edu/RNAstructureWeb/index.html). The presence of a stem-loop structure can be predicted and/or determined by reference to the method described in Example 3 and FIG. 10.

In some embodiments, the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure, e.g., through self-splicing for circularization.

Typically, the sequences forming the loop of the stem-loop structure are derived from the 3′ exon region and/or the 5′ exon region.

In some embodiments, where the self-splicing intron is a Group I intron, the first loop sequence comprises or consists of one or more nucleotides (for example, about 1 to about 20 nucleotides) which can pair with the P1 region of the corresponding Group I intron (or the structure formed by the 3′ self-splicing intron fragment and the 5′ self-splicing intron fragment) to form a P10 duplex region during the circularization.

In some embodiments, the first loop sequence may comprise or consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20. In some specific embodiments, n is 2, 4, or 5.

In some embodiments, the first loop sequence comprises or consists of the about 1 to about 7 consecutive nucleotides starting from the 5′ terminal nucleotide of the native 3′ exon of the Group I intron.

In some embodiments, the first loop sequence for example comprises or consists of AAAA, AA, UUUU, UAAA, CAAAA, or GAAAA.

In some embodiments, where the self-splicing intron is a Group I intron, the second loop sequence comprises or consists of one or more nucleotides (about 3 to about 8 nucleotides) which can pair with the internal guide sequence (IGS) of the corresponding Group I intron (or the structure formed by the 3′ self-splicing intron fragment and the 5′ self-splicing intron fragment) to form a P1 duplex region during the circularization.

In some embodiments, the second loop sequence comprises or consists of the about 3 to about 8 consecutive nucleotides starting from the 3′ terminal nucleotide of the native 5′ exon of the Group I intron.

In some embodiments, the second loop sequence for example comprises or consists of CUU or CUC.

In some specific embodiments, a loop with a sequence of CUUAAAA, CUUUUUU, CUUAA, CUUGAAA, CUUUAAA, CUUCAAA or CUCAAAA can be formed after circularization.

In some embodiments, the first loop sequence comprises or consists of AAAA and the second loop sequence comprises or consists of CUU. In some specific embodiments, a loop with a sequence of CUUAAAA is formed after circularization.

The pairing sequences forming the stem of the stem-loop structure may be derived from the exon regions, however, it may also be derived from the spacer sequences. Alternatively, the pairing sequence may be derived from an exon region and a spacer sequence, i.e., the pairing sequence comprises at least a portion of an exon region and at least a portion of the spacer.

Without being bound by any theory, the RNA circularization efficiency based on intron self-splicing (e.g., Group I intron self-splicing) is related to the number of base pairs or the type or composition of base pairs in the stem portion of the stem-loop structure formed by the residual circularizing element. The stability of the stem-loop structure (e.g., as can be predicted from calculated free energies) may affect the circularization efficiency.

In some embodiments, the stem portion of the stem-loop structure comprises at least 2 base pairs, such as at least 3, at least 4, at least 5, at least 6, at least 7, at least 8, at least 9, at least 10, at least 11, at least 12, at least 13, at least 14, at least 15 or more base pairs, preferably consecutive matched base pairs. In some embodiments, the stem portion of the stem-loop structure comprises 2-15 or more consecutive matched base pairs. In some embodiments, the stem portion of the stem-loop structure comprises 3-15 or more consecutive matched base pairs. In some embodiments, the stem portion of the stem-loop structure comprises 4-15 or more consecutive matched base pairs. In some embodiments, the stem portion of the stem-loop structure comprises 5-15 or more consecutive matched base pairs. In some embodiments, the stem portion of the stem-loop structure comprises 6-15 or more consecutive matched base pairs. In some embodiments, the stem portion of the stem-loop structure comprises 7-15 or more consecutive matched base pairs. In some embodiments, the stem portion of the stem-loop structure comprises 8-15 or more consecutive matched base pairs. In some embodiments, the stem portion of the stem-loop structure comprises 9-15 or more consecutive matched base pairs. In some embodiments, the stem portion of the stem-loop structure comprises 10-15 or more consecutive matched base pairs. In some embodiments, the stem portion of the stem-loop structure comprises 11-15 or more consecutive matched base pairs. In some embodiments, the stem portion of the stem-loop structure comprises 12-15 or more consecutive matched base pairs. In some embodiments, the stem portion of the stem-loop structure comprises 13-15 or more consecutive matched base pairs. In some embodiments, the stem portion of the stem-loop structure comprises 14-15 or more consecutive matched base pairs. In some embodiments, the stem portion of the stem-loop structure comprises 2, 3, 4, 5, 6, 7, 8, 9, 10, 11, 12, 13, 14, or 15 base pairs, preferably consecutive matched base pairs. In some embodiments, the stem portion of the stem-loop structure comprises 5 base pairs, preferably consecutive matched base pairs. In some embodiments, the stem portion of the stem-loop structure comprises 6 base pairs, preferably consecutive matched base pairs. In some embodiments, the stem portion of the stem-loop structure comprises 7 base pairs, preferably consecutive matched base pairs.

In some embodiments, the stem portion in the stem-loop structure comprises up to 2 base mismatches, or up to 1 base mismatch, preferably, the stem portion comprises no base mismatches.

In some embodiments, the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower. In some embodiments, the predicted free energy of the stem-loop structure is from about −1 kal/mol to about −10 kal/mol. In some embodiments, the predicted free energy of the stem-loop structure is from about −2 kal/mol to about −10 kal/mol. In some embodiments, the predicted free energy of the stem-loop structure is from about −3 kal/mol to about −10 kal/mol. In some embodiments, the predicted free energy of the stem-loop structure is from about −4 kal/mol to about −10 kal/mol. In some embodiments, the predicted free energy of the stem-loop structure is from about −5 kal/mol to about −10 kal/mol. In some embodiments, the predicted free energy of the stem-loop structure is from about −6 kal/mol to about −10 kal/mol. In some embodiments, the predicted free energy of the stem-loop structure is from about −7 kal/mol to about −10 kal/mol. In some embodiments, the predicted free energy of the stem-loop structure is from about −8 kal/mol to about −10 kal/mol. In some embodiments, the predicted free energy of the stem-loop structure is from about −9 kal/mol to about −10 kal/mol. The free energy can be determined, for example, by RNAfold (http://ma.tbi.univie.ac.at/cgi-bin/RNAWebSuite/RNAfold.cgi) or RNAstructure (https://rna.urmc.rochester.edu/RNAstructureWeb/index.html) Structure Prediction Tool.

In some embodiments, the first pairing sequence comprises only Gs and the second pairing sequence comprises only Cs. In some embodiments, the first pairing sequence comprises only Cs and the second pairing sequence comprises only Gs. In some embodiments, the first pairing sequence includes only A and the second pairing sequence includes only U.

In some embodiments, the first pairing sequence comprises or consists of the sequence of any one of SEQ ID NO:42-55.

In some embodiments, the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55. In some embodiments, the first residual circularizing element comprises or consists of a nucleotide sequence selected from SEQ ID NOs: 13-55.

In some embodiments, the second pairing sequence comprises or consists of the sequence of any one of SEQ ID NO:78-93.

In some embodiments, the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93. In some embodiments, the second residual circularizing element comprises or consists of a nucleotide sequence selected from SEQ ID NOs: 56-93.

In some embodiments, the first residual circularizing element comprises or consists of a nucleotide sequence of any one of SEQ ID NOs: 13-55 and the second residual circularizing element comprises or consists of a nucleotide sequence of any one of SEQ ID NOs: 56-93.

In some embodiments, the first residual circularizing element comprises or consists of a nucleotide sequence of SEQ ID NO: 15 and the second residual circularizing element comprises or consists of a nucleotide sequence of SEQ ID NO: 57.

In some embodiments, the first residual circularizing element comprises or consists of a nucleotide sequence of SEQ ID NO: 14 and the second residual circularizing element comprises or consists of a nucleotide sequence of SEQ ID NO: 58.

In some embodiments, the first residual circularizing element comprises or consists of a nucleotide sequence of SEQ ID NO: 18 and the second residual circularizing element comprises or consists of a nucleotide sequence of SEQ ID NO: 59.

In some embodiments, the first residual circularizing element comprises or consists of a nucleotide sequence of SEQ ID NO: 19 and the second residual circularizing element comprises or consists of a nucleotide sequence of SEQ ID NO: 60.

In some embodiments, the first residual circularizing element comprises or consists of a nucleotide sequence of SEQ ID NO: 20 and the second residual circularizing element comprises or consists of a nucleotide sequence of SEQ ID NO: 61.

In some embodiments, the first residual circularizing element comprises or consists of a nucleotide sequence of SEQ ID NO: 21 and the second residual circularizing element comprises or consists of a nucleotide sequence of SEQ ID NO: 62.

In some embodiments, the first residual circularizing element comprises or consists of a nucleotide sequence of SEQ ID NO: 23 and the second residual circularizing element comprises or consists of a nucleotide sequence of SEQ ID NO: 56.

In some embodiments, the first residual circularizing element comprises or consists of a nucleotide sequence of SEQ ID NO: 24 and the second residual circularizing element comprises or consists of a nucleotide sequence of SEQ ID NO: 56.

In some embodiments, the first residual circularizing element comprises or consists of a nucleotide sequence of SEQ ID NO: 26 and the second residual circularizing element comprises or consists of a nucleotide sequence of SEQ ID NO: 56.

In some embodiments, the first residual circularizing element comprises or consists of a nucleotide sequence of SEQ ID NO: 27 and the second residual circularizing element comprises or consists of a nucleotide sequence of SEQ ID NO: 56.

In some embodiments, the first residual circularizing element comprises or consists of a nucleotide sequence of SEQ ID NO: 28 and the second residual circularizing element comprises or consists of a nucleotide sequence of SEQ ID NO: 56.

In some embodiments, the first residual circularizing element comprises or consists of a nucleotide sequence of SEQ ID NO: 13 and the second residual circularizing element comprises or consists of a nucleotide sequence of SEQ ID NO: 67.

In some preferred embodiments, the first residual circularizing element comprises or consists of a nucleotide sequence of SEQ ID NO: 13 and the second residual circularizing element comprises or consists of a nucleotide sequence of SEQ ID NO: 59.

In some preferred embodiments, the first residual circularizing element comprises or consists of a nucleotide sequence of SEQ ID NO: 13 and the second residual circularizing element comprises or consists of a nucleotide sequence of SEQ ID NO: 56.

In some preferred embodiments, the first residual circularizing element comprises or consists of a nucleotide sequence of SEQ ID NO: 17 and the second residual circularizing element comprises or consists of a nucleotide sequence of SEQ ID NO: 59.

In some preferred embodiments, the first residual circularizing element comprises or consists of a nucleotide sequence of SEQ ID NO: 22 and the second residual circularizing element comprises or consists of a nucleotide sequence of SEQ ID NO: 63.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1 or a nucleotide sequence having at least 75%, e.g., at least 80%, at least 85%, at least 90%, at least 95%, 100% identity to SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2 or a nucleotide sequence having at least 75%, e.g., at least 80%, at least 85%, at least 90%, at least 95%, 100% identity to SEQ ID NO: 2;
the total length of the first residual circularizing element and the second residual circularizing element is from about 5 to about 100 nucleotides, or any integer number therebetween;
the first residual circularizing element and the second residual circularizing element are configured to be capable of forming a stem-loop structure upon self-splicing for circularization, the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent, the first pairing sequence and the second pairing sequence can complementarily pair to each other to form the stem of the stem-loop structure, wherein the first loop sequence and the second loop sequence can form the loop of the stem-loop structure, e.g., through self-splicing for circularization,
wherein the first loop sequence comprises or consists of AAAA, AA, UUUU, UAAA, CAAAA, or GAAAA, and the second loop sequence comprises or consists of CUU or CUC; and
wherein the first pairing sequence comprises or consists of the sequence of any one of SEQ ID NO:42-55; and the second pairing sequence comprises or consists of the sequence of any one of SEQ ID NO:78-93.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1 or a nucleotide sequence having at least 75%, e.g., at least 80%, at least 85%, at least 90%, at least 95%, 100% identity to SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2 or a nucleotide sequence having at least 75%, e.g., at least 80%, at least 85%, at least 90%, at least 95%, 100% identity to SEQ ID NO: 2;
the total length of the first residual circularizing element and the second residual circularizing element is from about 5 to about 100 nucleotides, or any integer number therebetween;
the first residual circularizing element and the second residual circularizing element are configured to be capable of forming a stem-loop structure upon self-splicing for circularization, the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55, and the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2;
the first residual circularizing element and the second residual circularizing element are configured to be capable of forming a stem-loop structure upon self-splicing for circularization, wherein
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 13 and the second residual circularizing element comprises or consists of a nucleotide sequence of SEQ ID NO: 56;
15) the first residual circularizing element comprises or consists of a nucleotide sequence of SEQ ID NO: 17 and the second residual circularizing element comprises or consists of a nucleotide sequence of SEQ ID NO: 59; or
16) the first residual circularizing element comprises or consists of a nucleotide sequence of SEQ ID NO: 22 and the second residual circularizing element comprises or consists of a nucleotide sequence of SEQ ID NO: 63.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2;
the first residual circularizing element and the second residual circularizing element are configured to be capable of forming a stem-loop structure upon self-splicing for circularization, wherein the first residual circularizing element comprises or consists of a nucleotide sequence of SEQ ID NO: 13 and the second residual circularizing element comprises or consists of a nucleotide sequence of SEQ ID NO: 59.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2;
the first residual circularizing element and the second residual circularizing element are configured to be capable of forming a stem-loop structure upon self-splicing for circularization, wherein the first residual circularizing element comprises or consists of a nucleotide sequence of SEQ ID NO: 17 and the second residual circularizing element comprises or consists of a nucleotide sequence of SEQ ID NO: 59.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2;
the first residual circularizing element and the second residual circularizing element are configured to be capable of forming a stem-loop structure upon self-splicing for circularization, wherein the first residual circularizing element comprises or consists of a nucleotide sequence of SEQ ID NO: 22 and the second residual circularizing element comprises or consists of a nucleotide sequence of SEQ ID NO: 63.

In some embodiments, the 3′ self-splicing intron fragment (3′ Group I intron fragment) is derived from the Group I intron of the Anabaena pre-tRNA-Leu gene and comprises or consists of a nucleotide sequence of SEQ ID NO: 1;
the 5′ self-splicing intron fragment (5′ Group I intron fragment) is derived from the Group I intron of the Anabaena pre-tRNA-Leu gene and comprises or consists of a nucleotide sequence of SEQ ID NO:2;
the first residual circularizing element and the second residual circularizing element are configured to be capable of forming a stem-loop structure upon self-splicing for circularization, wherein the first residual circularizing element comprises or consists of a nucleotide sequence of SEQ ID NO: 13 and the second residual circularizing element comprises or consists of a nucleotide sequence of SEQ ID NO: 56.

In some embodiments, the circular RNA precursor further comprises a 5′ homology arm sequence and a 3′ homology arm sequence capable of complementary pairing to form a homology arm double-stranded region. In some embodiments, the 5′ homology arm sequence is upstream of the 5′ end of the 3′ self-splicing intron fragment and the 3′ homology arm sequence is downstream of the 3′ end of the 5′ self-splicing intron fragment.

The homology arm can be, for example, about 5-50 nucleotides in length, for example, about 5-50, about 10-50, about 20-50, about 30-50, or about 40-50 nucleotides in length. In some embodiments, the homology arm may be 20 nucleotides in length. In some embodiments, the homology arm may be 40 nucleotides in length. In certain embodiments, the homology arm is at least 5, 6, 7, 8, 9, 10, 11, 12, 13, 14, 15, 16, 17, 18, 19, or 20 nucleotides in length. In certain embodiments, the homology arm is no more than 50, 45, 40, 35, 30, 25, or 20 nucleotides in length. In certain embodiments, the homology arm is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. The two homology arm sequences may be polyA and polyT, respectively, or polyG and polyC, respectively.

In some embodiments, one of the homology arm sequence may have the nucleotide sequence of any one of SEQ ID NO: 151-162 or a nucleotide sequence having at least 50%, at least 60%, at least 70%, at least 80%, at least 90%, at least 95%, or at least 99% sequence identity with any one of SEQ ID NO:151-162, while the other homology arm sequence may have the corresponding complementary sequence. In some preferred embodiments, the 5′ homology arm sequence the nucleotide sequence of SEQ ID NO:152, and the 5′ homology arm sequence the nucleotide sequence of SEQ ID NO:162.

In some embodiments, the nucleotide sequence of interest comprises at least one protein-coding sequence and a translation initiation element such as an internal ribosome entry site (IRES) operably linked thereto. Here, “operably linked” refers to that the translation initiation element such as IRES can mediate translation of the encoded protein. In some embodiments, in the circular RNA precursor, the translation initiation element, such as an IRES, is located upstream of the 5′ end of the at least one protein-coding sequence, or the translation initiation element, such as an IRES, is located downstream of the 3′ end of the at least one protein-coding sequence.

Protein-coding sequences can encode proteins of eukaryotic, prokaryotic or viral origin. In certain embodiments, the protein can be any protein for therapeutic or diagnostic use. For example, the protein coding region can encode human proteins, antigens, antibodies, gene editing enzymes such as CRISPR nucleases, and the like. For example, the encoded protein can be a chimeric antigen receptor, an immunomodulatory protein, and/or a transcription factor, and the like. Some specific examples include, but are not limited to, EGF, FGF1, RBD, G6PC, PAH, HGF, and the like.

The IRES sequence may be selected from, but is not limited to, the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2). Wild-type IRES sequences can also be modified and used in the present invention. Preferably, the IRES is CVB3, BRAV-1_L, PV1_L, CAV2_L, BRAV-1, PV1, or CAV2.

Exemplary IRESs comprise a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.

In some embodiments, the nucleotide sequence of interest is a non-protein coding sequence. For example, the non-protein-coding sequence can be antisense RNA, aptamer, guide RNA, or non-protein-coding RNA existing in any organism, and the like. The non-protein coding sequence may or may not contain a specific secondary structure.

In some embodiments, the nucleotide sequence of interest is at least 10, 20, 40, 60, 80, 100, 200, 300, 400, 500, 600, 700, 800, 900, 1000, 1500, 2000, 2500, 3000, 3500, 4000, 4500, 10000, 20000 nucleotides in length. In some embodiments, the nucleotide sequence of interest is about 10-about 20000 nucleotides in length.

The circular RNA precursor may be (e.g., chemically) unmodified, partially modified or fully modified. In some embodiments, the circular RNA precursor comprises at least one nucleotide modification. In some embodiments, up to 100% of the nucleotides of the circular RNA precursor are modified. In some embodiments, the at least one nucleotide modification is a cytidine modification, a uridine modification, or an adenosine modification. In some embodiments, the at least one nucleoside modification is selected from the group consisting of 5-methylcytosine (m5C), N6-methyladenosine (m6A), pseudouridine (W), N1-methylpseudouridine (m1ψ) and 5-methoxyuridine (5moU). In some embodiments, the circular RNA precursor comprises less than 100%, less than 90%, less than 80%, less than 70%, less than 60%, less than 50%, less than 40%, less than 30%, less than 20%, less than 15%, less than 10%, less than 5%, less than 1% of a specific nucleotide modification. As used herein, the percentage of a particular nucleotide modification refers to the ratio of nucleotides in the sequence that have undergone that particular modification to nucleotides that can undergo that particular modification.

In some preferred embodiments, the circular RNA precursor is unmodified. In some embodiments, the circular RNA precursor does not contain nucleotide chemical modification.

In one aspect, provided herein is a nucleic acid vector for generating a circular RNA molecule, said vector comprises a coding sequence of the circular RNA precursor of the present invention.

As used herein, “vector” refers to a DNA derived from a virus, plasmid or cell of a higher organism into which a foreign DNA fragment can be or has been inserted for cloning and/or expression purposes. In certain embodiments, the vector can be stably maintained in the organism. The vector may contain, for example, an origin of replication, a selectable marker or a reporter gene, such as antibiotic resistance or GFP, and/or a multiple cloning site (MCS). The term includes linear DNA fragments (e.g., PCR products, linear plasmid fragments), plasmid vectors, viral vectors, cosmids, bacterial artificial chromosomes (BACs), yeast artificial chromosomes (YACs), and the like.

In some embodiments, the nucleic acid vector further comprises an RNA polymerase promoter sequence operably linked to the coding sequence of the circular RNA precursor. The operably linked promoter allows in vivo and/or in vitro transcription of the circular RNA precursor. The promoter is, for example, a T7 RNA polymerase promoter, a T6 viral RNA polymerase promoter, a SP6 viral RNA polymerase promoter, a T3 viral RNA polymerase promoter or a T4 viral RNA polymerase promoter.

In another aspect, the present invention provides a circular RNA, which is prepared from the circular RNA precursor or the nucleic acid vector of the present invention.

In another aspect, the present invention provides a circular RNA, which comprising a first residual circularizing element, a nucleotide sequence of interest, and a second residual circularizing element.

In some embodiments, the first residual circularizing element, the nucleotide sequence of interest, and the second residual circularizing element may have the definitions mentioned above.

In some embodiments, the first residual circularizing element and the second residual circularizing element are involved in or required for RNA circularization by a self-splicing intron (e.g., a Group I intron). The first residual circularizing element and the second residual circularizing element participate in circularization together with the self-splicing intron (e.g., a Group I intron) but are retained in the final circular RNA.

In some embodiments, the first residual circularizing element and the second residual circularizing element are covalently linked. In some embodiments, 5′ end of the first residual circularizing element is covalently linked to 3′ end of the second residual circularizing element.

As used herein, a “residual circularizing element” refers to a sequence that is involved in or required for circularization by the self-splicing intron. The residual circularizing elements participate in circularization together with the self-splicing intron but are retained in the final circular RNA.

The inventors have surprisingly found that when self-splicing introns are used for RNA circularization, the introduction of an additional circularizing element into the circular RNA may result in increased immunogenicity of the circular RNA molecule, and after truncating and mutating the elements remained in the circular RNA (residual circularizing elements), the circularization efficiency can be retained or even improved, and the immunogenicity of the circular RNA can be significantly reduced, which is of great significance for the potential application of circular RNA as a drug or drug carrier.

In some embodiments, the total length of the first residual circularizing element and the second residual circularizing element is no greater than about 500 nucleotides, e.g., no greater than about 500, about 400, about 300, about 200, about 100, about 90, about 80, about 70, about 60, about 50, about 40, about 30, about 20, about 15, about 10, about 5 nucleotides.

In some embodiments, the total length of the first residual circularizing element and the second residual circularizing element is from about 2 to about 500 nucleotides, or any integer number therebetween. In some embodiments, the total length of the first residual circularizing element and the second residual circularizing element is from about 2 to about 400 nucleotides, or any integer number therebetween. In some embodiments, the total length of the first residual circularizing element and the second residual circularizing element is from about 2 to about 300 nucleotides, or any integer number therebetween. In some embodiments, the total length of the first residual circularizing element and the second residual circularizing element is from about 2 to about 200 nucleotides, or any integer number therebetween. In some embodiments, the total length of the first residual circularizing element and the second residual circularizing element is from about 2 to about 100 nucleotides, or any integer number therebetween. In some embodiments, the total length of the first residual circularizing element and the second residual circularizing element is from about 2 to about 90 nucleotides, or any integer number therebetween. In some embodiments, the total length of the first residual circularizing element and the second residual circularizing element is from about 2 to about 80 nucleotides, or any integer number therebetween. In some embodiments, the total length of the first residual circularizing element and the second residual circularizing element is from about 2 to about 70 nucleotides, or any integer number therebetween. In some embodiments, the total length of the first residual circularizing element and the second residual circularizing element is from about 2 to about 60 nucleotides, or any integer number therebetween. In some embodiments, the total length of the first residual circularizing element and the second residual circularizing element is about 2 to about 50 nucleotides, or any integer number therebetween. In some embodiments, the total length of the first residual circularizing element and the second residual circularizing element is from about 2 to about 40 nucleotides, or any integer number therebetween. In some embodiments, the total length of the first residual circularizing element and the second residual circularizing element is about 2 to about 30 nucleotides, or any integer number therebetween. In some embodiments, the total length of the first residual circularizing element and the second residual circularizing element is from about 2 to about 20 nucleotides, or any integer number therebetween. In some embodiments, the total length of the first residual circularizing element and the second residual circularizing element is from about 2 to about 15 nucleotides, or any integer number therebetween. In some embodiments, the total length of the first residual circularizing element and the second residual circularizing element is about 2 to about 10 nucleotides, or any integer number therebetween.

In some embodiments, the total length of the first residual circularizing element and the second residual circularizing element is at least 5 nucleotides in length. In some embodiments, the total length of the first residual circularizing element and the second residual circularizing element is at least 10 nucleotides in length. In some embodiments, the total length of the first residual circularizing element and the second residual circularizing element is at least 15 nucleotides in length.

In some embodiments, the total length of the first residual circularizing element and the second residual circularizing element is from about 5 to about 100 nucleotides, or any integer number therebetween. In some embodiments, the total length of the first residual circularizing element and the second residual circularizing element is from about 10 to about 100 nucleotides, or any integer number therebetween. In some embodiments, the total length of the first residual circularizing element and the second residual circularizing element is from about 15 to about 100 nucleotides, or any integer number therebetween. In some embodiments, the total length of the first residual circularizing element and the second residual circularizing element is from about 15 to about 30 nucleotides, or any integer number therebetween. In some embodiments, the total length of the first residual circularizing element and the second residual circularizing element is from about 20 to about 35 nucleotides, or any integer number therebetween. In some embodiments, the combined length of the first residual circularizing element and the second residual circularizing element is from about 25 to about 40 nucleotides, or any integer number therebetween. In some embodiments, the total length of the first residual circularizing element and the second residual circularizing element is from about 30 to about 45 nucleotides, or any integer number therebetween. In some embodiments, the total length of the first residual circularizing element and the second residual circularizing element is from about 35 to about 50 nucleotides, or any integer number therebetween. In some embodiments, the total length of the first residual circularizing element and the second residual circularizing element is from about 40 to about 55 nucleotides, or any integer number therebetween. In some embodiments, the combined length of the first residual circularizing element and the second residual circularizing element is from about 45 to about 60 nucleotides, or any integer number therebetween. In some embodiments, the total length of the first residual circularizing element and the second residual circularizing element is from about 50 to about 65 nucleotides, or any integer number therebetween. In some embodiments, the total length of the first residual circularizing element and the second residual circularizing element is from about 55 to about 70 nucleotides, or any integer number therebetween. In some embodiments, the total length of the first residual circularizing element and the second residual circularizing element is about 60 to about 75 nucleotides, or any integer number therebetween. In some embodiments, the total length of the first residual circularizing element and the second residual circularizing element is from about 65 to about 80 nucleotides, or any integer number therebetween. In some embodiments, the total length of the first residual circularizing element and the second residual circularizing element is from about 70 to about 85 nucleotides, or any integer number therebetween. In some embodiments, the total length of the first residual circularizing element and the second residual circularizing element is from about 75 to about 90 nucleotides, or any integer number therebetween. In some embodiments, the combined length of the first residual circularizing element and the second residual circularizing element is from about 80 to about 95 nucleotides, or any integer number therebetween. In some embodiments, the total length of the first residual circularizing element and the second residual circularizing element is from about 85 to about 100 nucleotides, or any integer number therebetween.

In some embodiments, the total length of the first residual circularizing element and the second residual circularizing element is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500 nucleotides.

In some embodiments, the first residual circularizing element and the second residual circularizing element are configured such that the circular RNA comprising them has reduced immunogenicity relative to a control circular RNA comprising the circularizing elements of SEQ ID NO:29 and SEQ ID NO:64 (Ana 3.0, with residual circularizing elements having a total length of 176 nucleotides).

In some embodiments, the circular RNA exhibits reduced immunogenicity relative to a control circular RNA comprising the circularizing elements of SEQ ID NO:29 and SEQ ID NO:64 (Ana 3.0, with residual circularizing elements having a total length of 176 nucleotides).

In some embodiments, the first residual circularizing element and the second residual circularizing element are configured such that the circular RNA comprising them has reduced immunogenicity relative to a control linear RNA, for example, a corresponding linear RNA without chemically modified nucleotides.

In some embodiments, the circular RNA exhibits reduced immunogenicity relative to a control linear RNA, for example, a corresponding linear RNA without chemically modified nucleotides.

“Reduced immunogenicity” as used herein may refer to that the circular RNA, upon contacted with cells, elicits a reduced immune response, i.e., an immune response at a level lower than a control circular RNA or control linear RNA. For example, reduced immune response refers to reduced expression of cytokines. The cytokines include, but are not limited to, IFNβ, TNFα, IL6 and/or RIG-I. In some embodiments, the immunogenicity of the circular RNA is reduced by at least about 10%, at least about 20%, at least about 30%, at least about 40%, at least about 50%, at least about 60%, at least about 70%, at least about 80%, at least about 90% or more. The reduced immunogenicity can be determined by methods well known in the art, such as those described in Example 10 of the present application.

In some embodiments, the first residual circularizing element and the second residual circularizing element are configured such that the circular RNA comprising them can be generated with a comparable or increased circularization efficiency relative to a circular RNA comprising the residual circularizing elements of SEQ ID NO:29 and SEQ ID NO:64 (Ana 3.0).

“Circularization efficiency” as used herein may refer to the ratio of outcome circular RNA to input precursor in a given time period. Alternatively, “Circularization efficiency” as used herein may refer to the ratio of desired circular RNA to linear RNAs in the final product in a given time period. The circularizing efficiency can be determined by methods well known in the art, such as those described in Example 17, 19 and 20 of the present application.

In some embodiments, the circularizing efficiency of the circular RNA is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or more.

In some embodiments, the first residual circularizing element comprises or consists of from 5′ to 3′ direction a 3′ exon region and optionally a spacer. In the circular RNA precursor, the 5′ end of the 3′ exon region is directly connected to the 3′ end of the 3′ self-splicing intron fragment.

As used herein, “exon region” in the residual circularizing element is a sequence derived from the native exon of the self-splicing intron (the exon flanking the self-splicing intron) and capable of being recognized and/or spliced by the self-splicing intron (or a combination of the first self-splicing intron fragment and the second self-splicing intron fragment), and thus is required for circularization.

In some embodiments, the 3′ exon region is derived from the native 3′ exon of the self-splicing intron (the exon flanking (downstream of) the 3′ end of the self-splicing intron) or a contiguous fragment thereof starting from the 5′ terminal nucleotide.

In some embodiments, the 3′ exon region is the entire native 3′ exon of the self-splicing intron, or has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the entire native 3′ exon of the self-splicing intron, or has 1, 2, 3, 4, 5, 6, 7, 8, 9, or 10 or more nucleotide substitutions, deletions or additions compared to the entire native 3′ exon of the self-splicing intron.

In some embodiments, the 3′ exon region is a contiguous fragment starting from the 5′ terminal nucleotide of the native 3′ exon. In some embodiments, the 3′ exon region has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with a contiguous fragment starting from the 5′ terminal nucleotide of the native 3′ exon. In some embodiments, the 3′ exon region has 1, 2, 3, 4, 5, 6, 7, 8, 9, or 10 or more nucleotide substitutions, deletions or additions compared to a continuous fragment starting from the 5′ terminal nucleotide of the native 3′ exon.

In some embodiments, the contiguous fragment starting from the 5′ terminal nucleotide of the native 3′ exon comprises or consists of at least 1%, at least 5%, at least 10%, at least 20%, at least 30%, at least 40%, at least 50%, at least 60%, at least 70%, at least 80%, at least 90% nucleotides of the native 3′ exon. In some embodiments, the contiguous fragment starting from the 5′ terminal nucleotide of the native 3′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 5′ terminal nucleotide of the native 3′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 3′ exon.

In some embodiments, where the self-splicing intron is a Group I intron, the 3′ exon region at least comprises a sequence (such as a sequence of about 1 to about 20 nucleotides) at it 5′ terminus which can pair with the P1 region of the corresponding Group I intron to form a P10 duplex region.

It is believed that for self-splicing of Group I introns, the consecutive one or more nucleotides (such as at least about 1 to about 7 nucleotides) from the 5′ end of the native 3′ exon can pair with the P1 region to form a P10 duplex region, and thus plays an important role in self-splicing. Definitions of the P1 and P10 regions of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the second residual circularizing element comprises or consists of from 3′ to 5′ direction a 5′ exon region and optionally a spacer. In the circular RNA precursor, the 3′ end of the 5′ exon region is directly connected to the 5′ end of the second self-splicing intron fragment.

In some embodiments, the 5′ exon region is derived from the native 5′ exon of the self-splicing intron (the exon flanking (downstream of) the 5′ end of the self-splicing intron) or a contiguous fragment thereof starting from the 3′ terminal nucleotide.

In some embodiments, the 5′ exon region is the entire native 5′ exon of the self-splicing intron, or has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the entire native 5′ exon of the Group I intron, or has 1, 2, 3, 4, 5, 6, 7, 8, 9, or 10 or more nucleotide substitutions, deletions or additions compared to the entire native 5′ exon of the self-splicing intron.

In some embodiments, the 5′ exon region is a contiguous fragment starting from the 3′ terminal nucleotide of the native 5′ exon. In some embodiments, the 5′ exon region has at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with a contiguous fragment starting from the 3′ terminal nucleotide of the native 5′ exon. In some embodiments, the 5′ exon region has 1, 2, 3, 4, 5, 6, 7, 8, 9, or 10 or more nucleotide substitutions, deletions or additions compared to a continuous fragment starting from the 3′ terminal nucleotide of the native 5′ exon.

In some embodiments, the contiguous fragment starting from the 3′ terminal nucleotide of the native 5′ exon comprises or consists of at least 1%, at least 5%, at least 10%, at least 20%, at least 30%, at least 40%, at least 50%, at least 60%, at least 70%, at least 80%, at least 90% nucleotides of the native 5′ exon. In some embodiments, the contiguous fragment starting from the 3′ terminal nucleotide of the native 5′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 3′ terminal nucleotide of the native 5′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 5′ exon.

In some embodiments, where the self-splicing intron is a Group I intron, the 5′ exon region comprises a sequence (for example, a sequence of about 3 to about 8 consecutive nucleotides) at its 3′ terminus which can pair with the internal guide sequence (IGS) of the corresponding Group I intron to form a P1 double-stranded region.

It is believed that for self-splicing of Group I introns, about 3 to about 8 consecutive nucleotides from the 3′ end of the native 5′ exon can pair with the internal guide sequence (IGS) of the intron to form the P1 double-stranded region, thus playing an important role in self-splicing. Definitions of IGS and/or P1 region of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the 3′ exon region is derived from the 3′ native exon of the Group I intron of the Anabaena pre-tRNA-Leu gene. The 3′ native exon of the Group I intron of the Anabaena pre-tRNA-Leu gene comprises the nucleotide sequence of SEQ ID NO:7. In some embodiments, the 5′ exon region is derived from the 5′ native exon of the Group I intron of the Anabaena pre-tRNA-Leu gene. The 5′ native exon of the Group I intron of the Anabaena pre-tRNA-Leu gene comprises the nucleotide sequence of SEQ ID NO:8.

In some embodiments, the 3′ exon region is derived from the 3′ native exon of the Group I intron of the td gene of T4 phage. The 3′ native exon of the td gene of T4 phage comprises the nucleotide sequence of SEQ ID NO:11. In some embodiments, the 5′ exon region is derived from the 5′ native exon of the Group I intron of the td gene of T4 phage. The 5′ native exon of the td gene of T4 phage comprises the nucleotide sequence of SEQ ID NO:12.

In some embodiments, the 3′ exon region is derived from the 3′ native exon of the Group I intron of pre-tRNA-Ile gene of Azoarcus sp. BH72. The 3′ native exon of pre-tRNA-Ile gene of Azoarcus sp. BH72 comprises the nucleotide sequence of SEQ ID NO:9. In some embodiments, the 5′ exon region is derived from the 5′ native exon of the Group I intron of pre-tRNA-Ile gene of Azoarcus sp. BH72. The 5′ native exon of pre-tRNA-Ile gene of Azoarcus sp. BH72 comprises the nucleotide sequence of SEQ ID NO:10.

During the circularization of the circular RNA precursor, the 3′ self-splicing intron fragment (e.g., 3′ Group I intron fragment) and the sequence upstream of its 5′ end (if present), and the 5′ self-splicing intron fragment (e.g., 5′ Group I intron fragment) and the sequence downstream of its 3′ end (if present) are excised, and the 5′ end of the first residual circularizing element and the 3′ end of the second residual circularizing element are covalently linked to achieve circularization of the RNA.

In some embodiments, the first residual circularizing element and the second residual circularizing element comprise spacers of different sequences, or one of them comprises a spacer and the other does not.

As used herein, “spacer” refers to any contiguous nucleotide sequence that at least does not negatively interfere with the function of the elements connected by it. Generally, if it is desired to avoid the interaction of two near or adjacent elements, a spacer can be inserted between the two elements. The spacer sequences described herein can serve two functions: (1) to facilitate circularization and (2) to facilitate functionality by allowing correct folding of the residual circularizing element and the nucleotide sequence of interest (e.g., IRES). In some embodiments, the spacer is no more than 150, no more than 100, no more than 50, no more than 30, no more than 10, no more than 5, or no more than 3 nucleotides in length. In some embodiments, the spacer is 5 nucleotides in length. In some embodiments, the spacer is 4 nucleotides in length. In some embodiments, the spacer is 3 nucleotides in length. In some embodiments, the spacer may be absent.

In some embodiments, the first residual circularizing element and the second residual circularizing element are configured to be capable of forming a stem-loop structure. In some embodiments, the loop of the stem-loop structure comprises the splicing junction.

In some embodiments, the presence of the stem-loop structure can be predicted and/or determined by the nucleotide sequences of the 3′ self-splicing intron fragment (e.g., 3′ Group I intron fragment), the first residual circularizing element, the second residual circularizing element, and the 5′ self-splicing intron fragment (e.g., 5′ Group I intron fragment) involved in circularization. In some embodiments, the presence of a stem-loop structure can be predicted and/or determined from the nucleotide sequence by RNA structure prediction tools such as RNAfold (http://rna.tbi.univie.ac.at/cgi-bin/RNAWebSuite/RNAfold.cgi) or RNAstructure (https://rna.urmc.rochester.edu/RNAstructureWeb/index.html). The presence of a stem-loop structure can be predicted and/or determined by reference to the method described in Example 3 and FIG. 10.

In some embodiments, the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to form the stem of the stem-loop structure, wherein the first loop sequence and the second loop sequence can form the loop of the stem-loop structure, e.g., through self-splicing.

Typically, the sequences forming the loop of the stem-loop structure are derived from the 3′ exon region and/or the 5′ exon region.

In some embodiments, where the self-splicing intron is a Group I intron, the first loop sequence comprises or consists of one or more nucleotides (for example, about 1 to about 20 nucleotides) which can pair with the P1 region of the corresponding Group I intron to form a P10 duplex region during the circularization.

In some embodiments, the first loop sequence may comprise or consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.

In some embodiments, the first loop sequence comprises or consists of the about 1 to about 7 consecutive nucleotides starting from the 5′ terminal nucleotide of the native 3′ exon of the Group I intron.

In some embodiments, the first loop sequence for example comprises or consists of AAAA, AA, UUUU, UAAA, CAAAA, or GAAAA.

In some embodiments, where the self-splicing intron is a Group I intron, the second loop sequence comprises or consists of one or more nucleotides (about 3 to about 8 nucleotides) which can pair with the internal guide sequence (IGS) of the Group I intron to form a P1 duplex region during the circularization.

In some embodiments, the second loop sequence comprises or consists of the about 3 to about 8 consecutive nucleotides starting from the 3′ terminal nucleotide of the native 5′ exon of the Group I intron.

In some embodiments, the second loop sequence for example comprises or consists of CUU or CUC.

In some specific embodiments, a loop with a sequence of CUUAAAA, CUUUUUU, CUUAA, CUUGAAA, CUUUAAA, CUUCAAA or CUCAAAA can be formed after circularization.

In some embodiments, the first loop sequence comprises or consists of AAAA and the second loop sequence comprises or consists of CUU. In some specific embodiments, a loop with a sequence of CUUAAAA is formed after circularization.

The pairing sequences forming the stem of the stem-loop structure may be derived from the exon regions, however, it may also be derived from the spacer sequences. Alternatively the pairing sequence may be derived from an exon region and a spacer sequence, i.e., the pairing sequence comprises at least a portion of an exon region and at least a portion of the spacer.

Without being bound by any theory, the RNA circularization efficiency based on intron self-splicing (e.g., Group I intron self-splicing) is related to the number of base pairs or the type or composition of base pairs in the stem portion of the stem-loop structure formed by the residual circularizing element. The stability of the stem-loop structure (e.g., as can be predicted from calculated free energies) may affect the circularization efficiency.

In some embodiments, the stem portion of the stem-loop structure comprises at least 2 base pairs, such as at least 3, at least 4, at least 5, at least 6, at least 7, at least 8, at least 9, at least 10, at least 11, at least 12, at least 13, at least 14, at least 15 or more base pairs, preferably consecutive base pairs. In some embodiments, the stem portion of the stem-loop structure comprises 2-15 or more consecutive base pairs. In some embodiments, the stem portion of the stem-loop structure comprises 3-15 or more consecutive base pairs. In some embodiments, the stem portion of the stem-loop structure comprises 4-15 or more consecutive base pairs. In some embodiments, the stem portion of the stem-loop structure comprises 5-15 or more consecutive base pairs. In some embodiments, the stem portion of the stem-loop structure comprises 6-15 or more consecutive base pairs. In some embodiments, the stem portion of the stem-loop structure comprises 7-15 or more consecutive base pairs. In some embodiments, the stem portion of the stem-loop structure comprises 8-15 or more consecutive base pairs. In some embodiments, the stem portion of the stem-loop structure comprises 9-15 or more consecutive base pairs. In some embodiments, the stem portion of the stem-loop structure comprises 10-15 or more consecutive base pairs. In some embodiments, the stem portion of the stem-loop structure comprises 11-15 or more consecutive base pairs. In some embodiments, the stem portion of the stem-loop structure comprises 12-15 or more consecutive base pairs. In some embodiments, the stem portion of the stem-loop structure comprises 13-15 or more consecutive base pairs. In some embodiments, the stem portion of the stem-loop structure comprises 14-15 or more consecutive base pairs. In some embodiments, the stem portion of the stem-loop structure comprises 2, 3, 4, 5, 6, 7, 8, 9, 10, 11, 12, 13, 14, or 15 base pairs, preferably consecutive base pairs. In some embodiments, the stem portion of the stem-loop structure comprises 5 base pairs, preferably consecutive base pairs. In some embodiments, the stem portion of the stem-loop structure comprises 6 base pairs, preferably consecutive base pairs. In some embodiments, the stem portion of the stem-loop structure comprises 7 base pairs, preferably consecutive base pairs.

In some embodiments, the stem portion in the stem-loop structure comprises up to 2 base mismatches, or up to 1 base mismatch, preferably, the stem portion comprises no base mismatches.

In some embodiments, the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower. In some embodiments, the predicted free energy of the stem-loop structure is from about −1 kal/mol to about −10 kal/mol. In some embodiments, the predicted free energy of the stem-loop structure is from about −2 kal/mol to about −10 kal/mol. In some embodiments, the predicted free energy of the stem-loop structure is from about −3 kal/mol to about −10 kal/mol. In some embodiments, the predicted free energy of the stem-loop structure is from about −4 kal/mol to about −10 kal/mol. In some embodiments, the predicted free energy of the stem-loop structure is from about −5 kal/mol to about −10 kal/mol. In some embodiments, the predicted free energy of the stem-loop structure is from about −6 kal/mol to about −10 kal/mol. In some embodiments, the predicted free energy of the stem-loop structure is from about −7 kal/mol to about −10 kal/mol. In some embodiments, the predicted free energy of the stem-loop structure is from about −8 kal/mol to about −10 kal/mol. In some embodiments, the predicted free energy of the stem-loop structure is from about −9 kal/mol to about −10 kal/mol. The free energy can be determined, for example, by RNAfold (http://ma.tbi.univie.ac.at/cgi-bin/RNAWebSuite/RNAfold.cgi) or RNAstructure (https://rna.urmc.rochester.edu/RNAstructureWeb/index.html) Structure Prediction Tool.

In some embodiments, the first pairing sequence comprises only Gs and the second pairing sequence comprises only Cs. In some embodiments, the first pairing sequence comprises only Cs and the second pairing sequence comprises only Gs. In some embodiments, the first pairing sequence includes only A and the second pairing sequence includes only U.

In some embodiments, the first pairing sequence comprises or consists of the sequence of any one of SEQ ID NO:42-55. In some embodiments, the second pairing sequence comprises or consists of the sequence of any one of SEQ ID NO:78-93.

In some embodiments, the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55. In some embodiments, the first residual circularizing element comprises or consists of a nucleotide sequence selected from SEQ ID NOs: 13-55.

In some embodiments, the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93. In some embodiments, the second residual circularizing element comprises or consists of a nucleotide sequence selected from SEQ ID NOs: 56-93.

In some embodiments, the first residual circularizing element comprises or consists of a nucleotide sequence of any one of SEQ ID NOs: 13-55 and the second residual circularizing element comprises or consists of a nucleotide sequence of any one of SEQ ID NOs: 56-93.

In some embodiments, the total length of the first residual circularizing element and the second residual circularizing element is from about 5 to about 100 nucleotides, or any integer number therebetween;
the first residual circularizing element and the second residual circularizing element are configured to be capable of forming a stem-loop structure upon self-splicing for circularization, the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent, the first pairing sequence and the second pairing sequence can complementarily pair to each other to form the stem of the stem-loop structure, wherein the first loop sequence and the second loop sequence can form the loop of the stem-loop structure, e.g., through self-splicing for circularization,
wherein the first loop sequence comprises or consists of AAAA, AA, UUUU, UAAA, CAAAA, or GAAAA, and the second loop sequence comprises or consists of CUU or CUC; and
wherein the first pairing sequence comprises or consists of the sequence of any one of SEQ ID NO:42-55; and the second pairing sequence comprises or consists of the sequence of any one of SEQ ID NO:78-93.

In some embodiments, the total length of the first residual circularizing element and the second residual circularizing element is from about 5 to about 100 nucleotides, or any integer number therebetween;
the first residual circularizing element and the second residual circularizing element are configured to be capable of forming a stem-loop structure upon self-splicing for circularization, the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55, and the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.

In some embodiments, the first residual circularizing element and the second residual circularizing element are configured to be capable of forming a stem-loop structure upon self-splicing for circularization, wherein
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 13 and the second residual circularizing element comprises or consists of a nucleotide sequence of SEQ ID NO: 56;
15) the first residual circularizing element comprises or consists of a nucleotide sequence of SEQ ID NO: 17 and the second residual circularizing element comprises or consists of a nucleotide sequence of SEQ ID NO: 59; or
16) the first residual circularizing element comprises or consists of a nucleotide sequence of SEQ ID NO: 22 and the second residual circularizing element comprises or consists of a nucleotide sequence of SEQ ID NO: 63.

In some embodiments, the first residual circularizing element comprises or consists of a nucleotide sequence of SEQ ID NO: 15 and the second residual circularizing element comprises or consists of a nucleotide sequence of SEQ ID NO: 57.

In some embodiments, the first residual circularizing element comprises or consists of a nucleotide sequence of SEQ ID NO: 14 and the second residual circularizing element comprises or consists of a nucleotide sequence of SEQ ID NO: 58.

In some embodiments, the first residual circularizing element comprises or consists of a nucleotide sequence of SEQ ID NO: 18 and the second residual circularizing element comprises or consists of a nucleotide sequence of SEQ ID NO: 59.

In some embodiments, the first residual circularizing element comprises or consists of a nucleotide sequence of SEQ ID NO: 19 and the second residual circularizing element comprises or consists of a nucleotide sequence of SEQ ID NO: 60.

In some embodiments, the first residual circularizing element comprises or consists of a nucleotide sequence of SEQ ID NO: 20 and the second residual circularizing element comprises or consists of a nucleotide sequence of SEQ ID NO: 61.

In some embodiments, the first residual circularizing element comprises or consists of a nucleotide sequence of SEQ ID NO: 21 and the second residual circularizing element comprises or consists of a nucleotide sequence of SEQ ID NO: 62.

In some embodiments, the first residual circularizing element comprises or consists of a nucleotide sequence of SEQ ID NO: 23 and the second residual circularizing element comprises or consists of a nucleotide sequence of SEQ ID NO: 56.

In some embodiments, the first residual circularizing element comprises or consists of a nucleotide sequence of SEQ ID NO: 24 and the second residual circularizing element comprises or consists of a nucleotide sequence of SEQ ID NO: 56.

In some embodiments, the first residual circularizing element comprises or consists of a nucleotide sequence of SEQ ID NO: 26 and the second residual circularizing element comprises or consists of a nucleotide sequence of SEQ ID NO: 56.

In some embodiments, the first residual circularizing element comprises or consists of a nucleotide sequence of SEQ ID NO: 27 and the second residual circularizing element comprises or consists of a nucleotide sequence of SEQ ID NO: 56.

In some embodiments, the first residual circularizing element comprises or consists of a nucleotide sequence of SEQ ID NO: 28 and the second residual circularizing element comprises or consists of a nucleotide sequence of SEQ ID NO: 56.

In some embodiments, the first residual circularizing element comprises or consists of a nucleotide sequence of SEQ ID NO: 13 and the second residual circularizing element comprises or consists of a nucleotide sequence of SEQ ID NO: 67.

In some preferred embodiments, the first residual circularizing element comprises or consists of a nucleotide sequence of SEQ ID NO: 13 and the second residual circularizing element comprises or consists of a nucleotide sequence of SEQ ID NO: 59.

In some preferred embodiments, the first residual circularizing element comprises or consists of a nucleotide sequence of SEQ ID NO: 13 and the second residual circularizing element comprises or consists of a nucleotide sequence of SEQ ID NO: 56.

In some preferred embodiments, the first residual circularizing element comprises or consists of a nucleotide sequence of SEQ ID NO: 17 and the second residual circularizing element comprises or consists of a nucleotide sequence of SEQ ID NO: 59.

In some preferred embodiments, the first residual circularizing element comprises or consists of a nucleotide sequence of SEQ ID NO: 22 and the second residual circularizing element comprises or consists of a nucleotide sequence of SEQ ID NO: 63.

In some embodiments, the nucleotide sequence of interest comprises at least one protein-coding sequence and a translation initiation element such as an IRES (the translation initiation element such as IRES is as defined above) operably linked thereto. Here, “operably linked” refers to that the translation initiation element such as IRES is capable of directing translation of the encoded protein.

In some embodiments, the circular RNA comprises in order: a first residual circularizing element, a translation initiation element such as an IRES, at least one protein coding sequence, and a second residual circularizing element. In some embodiments, the circular RNA comprises in order: a first residual circularizing element, at least one protein coding sequence, a translation initiation element such as an IRES, and a second residual circularizing element.

Protein-coding sequences can encode proteins of eukaryotic, prokaryotic or viral origin. In certain embodiments, the protein can be any protein for therapeutic or diagnostic use. For example, the protein coding region can encode human proteins, antigens, antibodies, gene editing enzymes such as CRISPR nucleases, and the like. For example, the encoded protein can be a chimeric antigen receptor, an immunomodulatory protein, and/or a transcription factor, and the like. Some specific examples include, but are not limited to, EGF, FGF1, RBD, G6PC, PAH, HGF, and the like.

The IRES sequence may be selected from, but is not limited to, the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2). Wild-type IRES sequences can also be modified and used in the present invention. Preferably, the IRES is CVB3, BRAV-1_L, PV1_L, CAV2_L, BRAV-1, PV1, or CAV2.

Exemplary IRESs comprise a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.

In some embodiments, the nucleotide sequence of interest is a non-protein coding sequence. For example, the non-protein-coding sequence can be antisense RNA, aptamer, guide RNA, or non-protein-coding RNA existing in any organism, and the like. The non-protein coding sequence may or may not contain a specific secondary structure.

In some embodiments, the nucleotide sequence of interest is at least 10, 20, 40, 60, 80, 100, 200, 300, 400, 500, 600, 700, 800, 900, 1000, 1500, 2000, 2500, 3000, 3500, 4000, 4500, 10000, 20000 nucleotides in length.

In some embodiments, the circular RNA is at least 10, 20, 40, 60, 80, 100, 200, 300, 400, 500, 600, 700, 800, 900, 1000, 1500, 2000, 2500, 3000, 3500, 4000, 4500, 5000, 10000, 20000 nucleotides in length. In some embodiments, the circular RNA is at least about 10 nucleotides in length. In some embodiments, the circular RNA is about 500 nt or less. In some embodiments, the circular RNA is at least about 1 knt.

The circular RNA may be unmodified, partially modified or fully modified. In some embodiments, the circular RNA comprises at least one nucleotide modification. In some embodiments, up to 100% of the nucleotides of the circular RNA are modified. In some embodiments, the at least one nucleotide modification is a cytidine modification, a uridine modification, or an adenosine modification. In some embodiments, the at least one nucleotide modification is selected from the group consisting of 5-methylcytosine (m5C), N6-methyladenosine (m6A), pseudouridine (ψ), N1-methylpseudouridine (m1ψ) and 5-methoxyuridine (5moU). In one embodiment, the circular RNA comprises less than 100%, less than 90%, less than 80%, less than 70%, less than 60%, less than 50%, less than 40%, less than 30%, less than 20%, less than 15%, less than 10%, less than 5%, less than 1% of a specific nucleotide modification. As used herein, the percentage of a particular nucleotide modification refers to the ratio of nucleotides in the sequence that have undergone that particular modification to nucleotides that can undergo that particular modification.

In some preferred embodiments, the circular RNA is unmodified. In some embodiments, the circular RNA does not contain nucleotide modification.

In another aspect, the present invention also provides the use of the circular RNA precursor and/or circular RNA of the present invention as an expression vector.

In another aspect, the present invention provides a pharmaceutical composition comprising the nucleic acid vector of the present invention and/or the circular RNA precursor and/or circular RNA of the present invention, and a pharmaceutically acceptable carrier. The specific use of the composition may depend on the nucleotide sequence of interest.

In some embodiment, the pharmaceutical composition is for use in treating a disease in a subject. The specific disease to be treated may depend on the specific nucleotide sequence of interest.

Pharmaceutically acceptable carriers may include, but are not limited to, buffers, excipients, stabilizers or preservatives. Examples of pharmaceutically acceptable carriers are physiologically compatible solvents, dispersion media, coatings, antibacterial and antifungal agents, isotonic and absorption delaying agents, and the like, such as salts, buffers, carbohydrates, antioxidants, aqueous or non-aqueous carriers, preservatives, wetting agents, surfactants or emulsifying agents or combinations thereof. The amount of a pharmaceutically acceptable carrier in a pharmaceutical composition can be determined experimentally based on the activity of the carrier and the desired properties of the formulation, such as stability and/or minimal oxidation.

### II. Method for Preparation of Circular RNA

In another aspect, the present invention provides a method for preparing a circular RNA, the method comprises:
1) providing a circular RNA precursor of the present invention or obtaining a circular RNA precursor by transcribing from the nucleic acid vector of the present invention;
2) incubating the circular RNA precursor in the presence of a divalent metal cation at a temperature at which RNA circularization occurs; and
3) harvesting the circular RNA obtained in step 2).

In some embodiments, the divalent metal cation is Mg²⁺ and/or Mn²⁺.

In some embodiments, the concentration of the divalent metal cation is at least about 5 mM, e.g., about 5 mM to about 550 mM, e.g., at least about 5 mM, about 10 mM, about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In another aspect, the present invention provides a method for preparing a circular RNA, the method comprises
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.

In some embodiments, the nucleic acid vector is the nucleic acid vector described in Section I of the present invention.

In some embodiments, the divalent metal cation in the in vitro transcription system is Mg²⁺.

In some embodiments, the in vitro transcription system further comprises a monovalent metal cation and/or a monovalent anion.

In some embodiments, the monovalent metal cation is Na⁺ or K⁺.

In some embodiments, the monovalent metal anion is Cl⁻ or CH3COO⁻ (OAc).

In some embodiments, the concentration of the divalent metal cation in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM. In some embodiments, the concentration of the divalent metal cation in the system during the first time period is 30 mM.

In some embodiments, the concentration of the monovalent metal cation in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM. In some embodiments, the monovalent metal cation is Na⁺, the concentration in the system during the first time period is 15 mM. In some embodiments, the monovalent metal cation is K⁺, the concentration in the system during the first time period is 90 mM.

In some embodiments, the concentration of the monovalent anion in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM, about 100 mM, about 150 mM. In some embodiments, the monovalent anion is Cl⁻, the concentration in the system during the first time period is 90 mM. In some embodiments, the monovalent anion is CH3COO⁻ (OAc), the concentration in the system during the first time period is 125 mM.

In some embodiments, the in vitro transcription and self-circularization occur in the same reaction system.

In some embodiments, the method does not include a step of isolating and/or purifying the linear RNA produced by the in vitro transcription.

Those skilled in the art would know that the in vitro transcription system also comprises various components required for transcription, such as buffers, rATP, rCTP, rUTP, rGTP and the like.

In some embodiments, the buffer of the in vitro transcription system is Tris-HCl buffer, or HEPES buffer, or MES buffer, or citrate buffer, or phosphate buffer. In some embodiments, the buffer of the in vitro transcription system is HEPES buffer.

In some embodiments, the in vitro transcription system has a pH of about 5-about 8, such as a pH of about 5, about 5.5, about 6, about 6.5, about 7, about 7.5, or about 8. In some embodiments, the in vitro transcription system has a pH of about 7.5.

The RNA polymerase depends on the promoter used in the nucleic acid vector to drive transcription. The RNA polymerase may include, but is not limited to, a T7 RNA polymerase, a T6 viral RNA polymerase, a SP6 viral RNA polymerase, a T3 viral RNA polymerase, or a T4 viral RNA polymerase. In some embodiments, the RNA polymerase is T7 RNA polymerase.

In some embodiments, the first time period is at least 0.5 hours, such as about 0.5 hours to about 24 hours, such as about 0.5 hours, about 1 hour, about 1.5 hours, about 2 hours, about 2.5 hours, about 3 hours, about 3.5 hours, about 4 hours, about 5 hours, about 10 hours, or about 24 hours. In some embodiments, the first time period is 3 hours.

In some embodiments, the incubation for the first time period is carried out at about 16° C. to about 60° C., such as at about 16° C., about 17° C., about 18° C., about 19° C., about 20° C. ° C., about 21° C., about 22° C., about 23° C., about 24° C., about 25° C., about 26° C., about 27° C., about 28° C., about 29° C., about 30° C. ° C., about 31° C., about 32° C., about 33° C., about 34° C., about 35° C., about 36° C., about 37° C., about 38° C., about 39° C., about 40° C. ° C., about 41° C., about 42° C., about 43° C., about 44° C., about 45° C., about 46° C., about 47° C., about 48° C., about 49° C., about 50° C. ° C., about 51° C., about 52° C., about 53° C., about 54° C., about 55° C., about 56° C., about 57° C., about 58° C., about 59° C., or about 60° C. In some embodiments, the incubation for the first time period is carried out at about 37° C.

In some embodiments, after incubation for the first time period in step b), the method further comprises step c):
adding an additional amount of metal cation to the in vitro transcription system and incubating for a second time period; or
changing the buffer of the system, adding a metal cation and incubating for a second time period.

In some embodiments, the metal cation added for the incubation of the second time period is a divalent metal cation, such as Mg²⁺ or Mn²⁺.

In some embodiments, during the incubation of the second time period, the metal cation is added to a final concentration of at least about 5 mM, such as about 5 mM to about 550 mM, such as at least about 5 mM, at least about 10 mM, at least about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In some embodiments, the buffer of the system is changed to Tris-HCl buffer, or HEPES buffer, or MES buffer, or citrate buffer, or phosphate buffer during the second time period.

In some embodiments, the pH of the system in the second time period is 5-8, such as pH 5, pH 5.5, pH 6, pH 6.5, pH 7, pH 7.5, or pH 8.

In some embodiments, the second time period is at least 5 minutes, such as about 5 minutes to about 2 hour, such as about 5 minutes, about 10 minutes, about 15 minutes, about 20 minutes, about 25 minutes, about 30 minutes, about 45 minutes, about 50 minutes, about 55 minutes, about 60 minutes, about 120 minutes or more.

In some embodiments, wherein the reaction for the second time period is carried out at about 25° C. to about 75° C., such as at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 50° C., about 55° C., about 60° C., about 65° C., about 70° C., or about 75° C.

In some embodiments, the method further comprises a step of recovering or purifying the circular RNA as produced.

In one aspect, the present invention provides a circular RNA produced by the method of the invention.

In one aspect, the present invention provides an in vitro transcription method comprising:
a) providing a nucleic acid vector as a template for in vitro transcription;
b) incubating the nucleic acid vector in an in vitro transcription system comprising a divalent metal cation and an RNA polymerase for a first period of time; and
c) i) adding an additional amount of metal cation to the system, or ii) changing the buffer of the system and adding a metal cation to the system, and incubating the system for a second period of time.

In some embodiments, the in vitro transcription system during the first time period further comprises a monovalent metal cation and/or a monovalent anion.

In some embodiments, the divalent metal cation in the in vitro transcription system during the first time period is Mg²⁺.

In some embodiments, the monovalent metal cation during the first time period is Na⁺ or K⁺.

In some embodiments, the monovalent metal anion during the first time period is Cl⁻ or CH3COO⁻ (OAc).

The RNA polymerase depends on the promoter used in the nucleic acid vector to drive transcription. The RNA polymerase may include, but is not limited to, a T7 RNA polymerase, a T6 viral RNA polymerase, a SP6 viral RNA polymerase, a T3 viral RNA polymerase, or a T4 viral RNA polymerase. In some embodiments, the RNA polymerase is T7 RNA polymerase.

In some embodiments, the concentration of the divalent metal cation in the system for the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM.

In some embodiments, the concentration of the monovalent metal cation in the system for the first time period is from about 5 mM to about 100 mM, e.g., about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM.

In some embodiments, the concentration of the monovalent anion in the system for the first time period is from about 5 mM to about 150 mM, e.g., about 5 mM, about 10 mM, about 15 mM, about 20 mM, about 25 mM, about 30 mM, about 35 mM, about 40 mM, about 45 mM, about 50 mM, about 100 mM, about 150 mM.

In some embodiments, the buffer of the in vitro transcription system for the first time period is Tris-HCl buffer, or HEPES buffer, or MES buffer, or citrate buffer, or phosphate buffer.

In some embodiments, the pH of the in vitro transcription system for the first time period is 5-8, such as pH 5, pH 5.5, pH 6, pH 6.5, pH 7, pH 7.5, or pH 8.

In some embodiments, the first time period is at least 0.5 hours, such as about 0.5 hours to about 24 hours, such as about 0.5 hours, about 1 hour, about 1.5 hours, about 2 hours, about 2.5 hours, about 3 hours, about 3.5 hours, about 4 hours, about 5 hours, about 10 hours, or about 24 hours.

In some embodiments, the incubation for the first time period is carried out at about 16° C. to about 60° C., such as at about 16° C., about 17° C., about 18° C., about 19° C., about 20° C. ° C., about 21° C., about 22° C., about 23° C., about 24° C., about 25° C., about 26° C., about 27° C., about 28° C., about 29° C., about 30° C. ° C., about 31° C., about 32° C., about 33° C., about 34° C., about 35° C., about 36° C., about 37° C., about 38° C., about 39° C., about 40° C. ° C., about 41° C., about 42° C., about 43° C., about 44° C., about 45° C., about 46° C., about 47° C., about 48° C., about 49° C., about 50° C. ° C., about 51° C., about 52° C., about 53° C., about 54° C., about 55° C., about 56° C., about 57° C., about 58° C., about 59° C., or about 60° C.

In some embodiments, the added metal cation in the system for the second time period is a divalent metal cation, such as Mg²⁺ or Mn²⁺.

In some embodiments, during the second time period, the metal cation is added to a final concentration of at least about 5 mM, such as about 5 mM to about 550 mM, such as at least about 5 mM, at least about 10 mM, at least about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In some embodiments, the second time period is at least 5 minutes, such as about 5 minutes to about 2 hour, such as about 5 minutes, about 10 minutes, about 15 minutes, about 20 minutes, about 25 minutes, about 30 minutes, about 45 minutes, about 50 minutes, about 55 minutes, about 60 minutes, about 120 minutes or more.

In some embodiments, the incubation for the second time period is carried out at about 25° C. to about 75° C., such as at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 50° C., about 55° C., about 60° C., about 65° C., about 70° C., or about 75° C.

In some embodiments, the method further comprises a step of recovering and/or purifying the RNA obtained in step c).

In one aspect, the present invention provides an RNA produced by the method of the invention.

### III. Circular RNA Purification Method

In one aspect, the present invention provides a method for purifying a circular RNA, the method comprises:
a) contacting a mixture comprising circular RNA and uncircularized linear circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.

In some embodiments, the circular RNA is prepared by circularizing a linear circular RNA precursor. In some embodiments, the circular RNA is prepared by ligating both ends of a linear circular RNA precursor with an RNA ligase, such as a T4 RNA ligase. In some embodiments, the circular RNA is prepared by the self-splicing ribozyme activity of Group I intron-based circularizing elements contained in the linear circular RNA precursor, e.g., the circular RNA is the circular RNA described in Section I herein or any clause below and/or prepared by the method described in Section II herein or any clause below.

In some embodiments, the circular RNA-specific probe is a single-stranded DNAprobe.

In some embodiments, the circular RNA-specific probe is a single-stranded RNAprobe.

In some embodiments, the circular RNA-specific probe specifically hybridizes to a region flanking the circularization junction of the circular RNA.

In some embodiments, the circular RNA-specific probe is at least 10 nucleotides, at least 12 nucleotides, at least 14 nucleotides, at least 16 nucleotides, at least 18 nucleotides in length acid, at least 20 nucleotides, at least 25 nucleotides, at least 30 nucleotides, at least 35 nucleotides in length or longer, for example, the circular RNA-specific probe is 16, 17, 18, 19, 20, 21, 22, 23, 24, 25, 25, 27, 28, 29 or 30 nucleotides in length.

In some embodiments, the circular RNA-specific probe is immobilized on a support such as a solid support, e.g., the circular RNA-specific probe is immobilized on the support after binding to the circular RNA or the circular RNA-specific probe is pre-immobilized on the support.

In some embodiments, the condition in step a) include denaturing the RNA at between about 60° C. and about 95° C. (e.g., about 60° C., about 62° C., about 64° C., about 66° C., about 68° C., about 70° C., about 75° C., about 80° C., about 85° C., about 90° C., about 95° C.) for about 2 minutes to about 10 minutes (e.g., about 2, 3, 4, 5, 6, 7, 8, 9, or 10 minutes), then gradually reducing the temperature to below about 40° C. (e.g., below about 35° C., below about 30° C., below about 25° C., below about 20° C. or less) to allow the circular RNA annealing to the circular RNA-specific probe.

In some embodiments, the condition in step a) include a high salt concentration range of 0.25M-2M, e.g., 0.25M, 0.5M, 0.75M, 1M, 1.25M, 1.5M, 1.75M or 2M. In some embodiments, the salt is NaCl or a guanidine salt (e.g., Guanidine hydrochloride).

In some embodiments, in step b), the one or more components are removed by washing the complex with a washing buffer.

In some embodiments, step c) is performed by increasing the temperature to about 60° C. to about 95° C. (e.g., about 60° C., about 62° C., about 64° C., about 66° C., about 68° C.) C, about 70° C., about 75° C., about 80° C., about 85° C., about 90° C., about 95° C.) to release the circular RNA.

In some embodiments, in step c) the circular RNA is released by elution with an elution buffer. In some embodiments, the elution buffer is a low salt buffer, e.g., a buffer with salt concentration lower than 0.5M. In some embodiment, the elution buffer is Tris-EDTA buffer (TE buffer) or water.

In some embodiments, the method further includes the following steps:
i) contacting the mixture comprising circular RNA and uncircularized linear circular RNA precursor to a linear circular RNA precursor-specific probe in a condition that allows the linear circular RNA precursor-specific probe to specifically bind to and form a complex with the linear circular RNA precursor;
ii) removing the complex formed by the linear circular RNA precursor-specific probe with the linear circular RNA precursor from the mixture, and
iii) collecting the circular RNA-containing mixture obtained in step ii).

In some embodiments, steps i)-iii) are performed before step a), for example, steps i)-iii) may be performed multiple times before step a), e.g., 2, 3, 4 or more times. In some embodiments, steps i)-iii) are performed concurrently with steps a)-c).

In one aspect, the present invention provides a method for purifying circular RNA, the method comprises:
i) contacting a mixture comprising circular RNA and uncircularized linear circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and
optionally, steps i)-iii) are performed multiple times, e.g., 2 times, 3 times, 4 times or more.

In some embodiments, the linear circular RNA precursor-specific probe specifically binds to the linear circular RNA precursor and does not substantially bind to the circular RNA.

In some embodiments, the linear circular RNA precursor-specific probe is immobilized on a support, such as a solid support, for example, the linear circular RNA precursor-specific probe is then immobilized on the support after binding to the linear circular RNA precursor, or the linear circular RNA precursor-specific probe is pre-immobilized on the support.

In some embodiments of the method for purifying circular RNA of the invention, the linear circular RNA precursor comprises the following elements arranged in the following order from the 5′ to 3′ direction:
a) a 3′ self-splicing intron fragment;
b) a first residual circularizing element;
c) a nucleotide sequence of interest;
d) a second residual circularizing element; and
e) a 5′ self-splicing intron fragment;
wherein the linear circular RNA precursor is capable of removing the 3′ self-splicing intron fragment and the 5′ self-splicing intron fragment by self-splicing, generating a circular RNA comprising the first residual circularizing element, the nucleotide sequence of interest and the second residual circularizing element. In some embodiments, the elements of the linear circular RNA precursor are as defined in Section I herein or any clause below.

In some embodiments, the circular RNA-specific probe specifically hybridizes to at least a portion of the first residual circularizing element and a portion of the second residual circularizing element.

In some embodiments, the linear precursor RNA-specific probe hybridizes to a portion of the linear circular RNA precursor outside the first residual circularizing element, the nucleotide sequence of interest, and the second residual circularizing element.

In some embodiments, the linear precursor RNA-specific probe hybridizes to the 3′ self-splicing intron fragment or a portion thereof or a 5′ flanking sequence thereof, or the 5′ self-splicing intron fragment or a portion thereof or a 3′ flanking sequence thereof.

In some specific embodiments, the linear circular RNA precursor contains a sequence selected from SEQ ID NOs:96-101 or a complement sequence thereof, preferably, SEQ ID NO:100 or a complement sequence thereof outside the first residual circularizing element, the nucleotide sequence of interest, and the second residual circularizing element, to which the linear precursor RNA-specific probe specifically hybridizes.

In some embodiments of the method for purifying circular RNA of the invention, the molar ratio of the probe to the RNA molecules in the mixture is from about 1:1 to about 100,000:1.

In some embodiments, the linear circular RNA precursor-specific probe hybridizes to a 3′ homology arm sequence or portion thereof on the linear circular RNA precursor, or a 5′ homology arm sequence or portion thereof on the linear circular RNA precursor. In some embodiments, the homology arm sequence comprises polyA, polyU, polyC or polyG. In some embodiments, the homology arm sequence is about 10-about 200 nt in length.

Accordingly, in some embodiments, the linear precursor RNA-specific probe comprises polyT, poly A, polyG or polyC. In some embodiments, the linear precursor RNA-specific probe is about 10-about 200 nt in length.

In one aspect, the present invention provides a method for purifying circular RNA, the method comprises:
i) adding a linear RNA-specific tag to the linear RNA in a mixture comprising circular RNA and linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii),
optionally, steps ii)-iv) are performed multiple times, e.g., 2 times, 3 times, 4 times or more.

In some embodiments, the tag comprises a polyA, polyG, polyU, or polyC sequence. Accordingly, the probe includes a polyT/polyU, polyC, polyA, or polyG sequence. Alternatively, the tag can be a random sequence, and the probe comprises a sequence complementally pair with said random sequence.

In some embodiments, the tag may be about 10-200 nt in length. In some embodiments, the probe may be about 10-200 nt in length.

In some embodiments, the tag is added to the linear RNA by adding a PolyA/T/C/G polymerase or a ligase to the mixture. In some embodiments, adding rATP, rGTP, rUTP, rCTP or rNTP, or a random tag sequence of about 10-200 nt to the mixture is also included.

In some embodiments, the linear RNA probe specifically binds to the added tag without substantially binding to the circular RNA. In some embodiments, the linear RNA probe is a single-stranded DNA probe, or a single-stranded RNA probe.

In some embodiments, the linear RNA probe is immobilized on a support such as a solid support, for example, the linear RNA probe is immobilized on the support after binding to the linear RNA, or, the linear RNA probe is pre-immobilized on the support.

### CLAUSES

The subsequent clauses are part of the disclosure and shall further illustrate the invention.
Clause 1. A circular RNA precursor comprising the following elements from 5′ to 3′ direction in the following order:
a) a 3′ self-splicing intron fragment;
b) a first residual circularizing element;
c) a nucleotide sequence of interest;
d) a second residual circularizing element; and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 5 to about 100 nucleotides.
Clause 2. The circular RNA precursor of clause 1, wherein the self-splicing intron is selected from Group I introns and Group II introns, for example, the self-splicing intron is Group I introns.
Clause 3. The circular RNA precursor of clause 1 or 2, wherein the self-splicing intron is selected from IC3 subgroup of Group I introns.
Clause 4. The circular RNA precursor of any one of clauses 1-3, wherein the self-splicing intron is selected from a Group I intron of the cyanobacterium Anabaena, a Group I intron from a T4 phage or a Group I intron from Azoarcus sp. BH72, for example, the self-splicing intron is a Group I intron of the cyanobacterium Anabaena.
Clause 5. The circular RNA precursor of any one of clauses 1-4, wherein the self-splicing intron is selected from the Group I intron of the Anabaena pre-tRNA-Leu gene, the Group I intron of the td gene of T4 phage, or the Group I intron of the pre-tRNA-Ile gene of Azoarcus sp. BH72, for example, the self-splicing intron is the Group I intron of the Anabaena pre-tRNA-Leu gene.
Clause 6. The circular RNA precursor of any one of clauses 1-5, wherein the 3′ self-splicing intron fragment is derived from a 3′ terminal portion of a native self-splicing intron starting from an internal split site to the 3′ end of the native self-splicing intron, and wherein the 5′ self-splicing intron fragment is derived from a 5′ terminal portion of the native self-splicing intron starting from the internal split site to the 5′ end of the native self-splicing intron, and, the 3′ self-splicing intron fragment and the 5′ self-splicing intron fragment in combination retain the self-splicing activity of the native self-splicing intron.
Clause 7. The circular RNA precursor of clause 6, wherein the internal split site is located within the P6, P2, P5, P8 or P9 region of the Group I intron or the D4 region of the Group II intron, preferably, the internal split site is located within the P6 region of the Group I intron.
Clause 8. The circular RNA precursor of clause 6 or 7, wherein the 3′ self-splicing intron fragment is a sequence having at least 75%, at least 80%, at least 85%, at least 90%, at least 95%, 100% sequence identity to the 3′ terminal portion of a native self-splicing intron, and the 5′ self-splicing intron fragment is a sequence having at least 75%, at least 80%, at least 85%, at least 90%, at least 95%, 100% sequence identity to the 5′ terminal portion of a native self-splicing intron.
Clause 9. The circular RNA precursor of any one of clauses 1-8, wherein self-splicing intron is Group I intron of the Anabaena pre-tRNA-Leu gene, and the 3′ self-splicing intron fragment comprises or consists of a nucleotide sequence of SEQ ID NO: 1 or a nucleotide sequence having at least 75%, at least 80%, at least 85%, at least 90%, at least 95%, 100% identity to SEQ ID NO: 1, and the 5′ self-splicing intron fragment comprises or consists of a nucleotide sequence of SEQ ID NO: 2 or a nucleotide sequence having at least 75%, at least 80%, at least 85%, at least 90%, at least 95%, 100% identity to SEQ ID NO: 2.
Clause 10. The circular RNA precursor of any one of clauses 1-8, wherein the self-splicing intron is the Group I intron of the td gene of T4 phage, and the 3′ self-splicing intron fragment comprises or consists of a nucleotide sequence of SEQ ID NO: 5 or a nucleotide sequence having at least 75%, at least 80%, at least 85%, at least 90%, at least 95%, 100% identity to SEQ ID NO: 5, and the 5′ self-splicing intron fragment comprises or consists of a nucleotide sequence of SEQ ID NO: 6 or a nucleotide sequence having at least 75%, at least 80%, at least 85%, at least 90%, at least 95%, 100% identity to SEQ ID NO: 6.
Clause 11. The circular RNA precursor of any one of clauses 1-8, wherein the self-splicing intron is the Group I intron of the pre-tRNA-Ile gene of Azoarcus sp. BH72, and the 3′ self-splicing intron fragment comprises or consists of a nucleotide sequence of SEQ ID NO: 3 or a nucleotide sequence having at least 75%, at least 80%, at least 85%, at least 90%, at least 95%, 100% identity to SEQ ID NO: 3, and the 5′ self-splicing intron fragment comprises or consists of a nucleotide sequence of SEQ ID NO: 4 or a nucleotide sequence having at least 75%, at least 80%, at least 85%, at least 90%, at least 95%, 100% identity to SEQ ID NO: 4.
Clause 12. The circular RNA precursor of any one of clauses 1-11, wherein the first residual circularizing element and the second residual circularizing element are configured such that the circular RNA comprising them has reduced immunogenicity relative to a control RNA, such as a control circular RNA comprising the circularizing elements of SEQ ID NO:29 and SEQ ID NO:64 or a control linear RNA comprising the same nucleotide sequence of interest.
Clause 13. The circular RNA precursor of any one of clauses 1-12, wherein the first residual circularizing element and the second residual circularizing element are configured such that the circular RNA or circular RNA precursor comprising them has comparable or increased circularization efficiency relative to a control circular RNA or circular RNA precursor, such as a control circular RNA or circular RNA precursor comprising a first residual circularizing element and a second residual circularizing element of SEQ ID NO:29 and SEQ ID NO:64 (Ana 3.0), respectively.
Clause 14. The circular RNA precursor of any one of clauses 1-13, wherein the total length of the first residual circularizing element and the second residual circularizing element is about 20 to about 35 nucleotides.
Clause 15. The circular RNA precursor of any one of clauses 1-14, wherein the first residual circularizing element and the second residual circularizing element are configured to be capable of forming a stem-loop structure, e.g., upon self-splicing for circularization.
Clause 16. The circular RNA precursor of clause 15, wherein the loop of the stem-loop structure comprises the splicing junction.
Clause 17. The circular RNA precursor of any one of clauses 15-16, wherein the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure, e.g., through self-splicing for circularization.
Clause 18. The circular RNA precursor of clause 17, wherein the first loop sequence comprises or consists of one or more nucleotides which can pair with the P1 region of the corresponding self-splicing intron to form a P10 duplex region during the circularization.
Clause 19. The circular RNA precursor of clause 17 or 18, wherein the first loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.
Clause 20. The circular RNA precursor of any one of clauses 17-19, wherein the first loop sequence comprises or consists of the sequence of AAAA, AA, UUUU, UAAA, CAAAA, or GAAAA, preferably AAAA.
Clause 21. The circular RNA precursor of any one of clauses 17-20, wherein the second loop sequence comprises or consists of one or more nucleotides which can pair with the internal guide sequence (IGS) of the corresponding self-splicing intron to form a P1 duplex region during the circularization.
Clause 22. The circular RNA precursor of any one of clauses 17-21, wherein the second loop sequence comprises or consists of CUU or CUC, preferably CUU.
Clause 23. The circular RNA precursor of any one of clauses 17-22, wherein the loop of the stem-loop structure has a sequence of CUUAAAA, CUUUUUU, CUUAA, CUUGAAA, CUUUAAA, CUUCAAA or CUCAAAA, preferably CUUAAAA.
Clause 24. The circular RNA precursor of any one of clauses 17-23, wherein the stem portion of the stem-loop structure comprises 2-15 or more consecutive matched base pairs, preferably, the stem portion of the stem-loop structure comprises 5, 6 or 7 consecutive matched base pairs.
Clause 25. The circular RNA precursor of any one of clauses 17-24, wherein the stem portion in the stem-loop structure comprises up to 2 base pair mismatches, or the stem portion comprises only 1 base pair mismatch, preferably, the stem portion comprises no base pair mismatches.
Clause 26. The circular RNA precursor of any one of clauses 17-25, wherein the first pairing sequence comprises only Gs and the second pairing sequence comprises only Cs.
Clause 27. The circular RNA precursor of any one of clauses 17-25, wherein the first pairing sequence comprises only Cs and the second pairing sequence comprises only Gs.
Clause 28. The circular RNA precursor of any one of clauses 17-25, wherein the first pairing sequence includes only A and the second pairing sequence includes only U.
Clause 29. The circular RNA precursor of any one of clauses 17-25, wherein the first pairing sequence comprises or consists of the sequence of any one of SEQ ID NO:42-55, and, the second pairing sequence comprises or consists of the sequence of any one of SEQ ID NO:78-93.
Clause 30. The circular RNA precursor of any one of clauses 15-29, wherein the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower.
Clause 31. The circular RNA precursor of any one of clauses 1-30, wherein the first residual circularizing element comprises or consists of from 5′ to 3′ direction a 3′ exon region and optionally a first spacer, the second residual circularizing element comprises or consists of from 3′ to 5′ direction a 5′ exon region and optionally a second spacer.
Clause 32. The circular RNA precursor of clause 31, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
Clause 33. The circular RNA precursor of clause 32, wherein 3′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 5′ exon or a contiguous fragment of about 1-about 50 nucleotides starting from the 3′ terminal nucleotide of the native 5′ exon.
Clause 34. The circular RNA precursor of any one of clauses 1-33, wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55.
Clause 35. The circular RNA precursor of any one of clauses 1-34, wherein the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.
Clause 36. The circular RNA precursor of any one of clauses 1-35, wherein the first residual circularizing element comprises or consists of a nucleotide sequence of any one of SEQ ID NOs: 13-55 and the second residual circularizing element comprises or consists of a nucleotide sequence of any one of SEQ ID NOs: 56-93.
Clause 37. The circular RNA precursor of any one of clauses 1-36, wherein,
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 17 and the second residual circularizing element comprises or consists of a nucleotide sequence of SEQ ID NO: 59; or
15) the first residual circularizing element comprises or consists of a nucleotide sequence of SEQ ID NO: 22 and the second residual circularizing element comprises or consists of a nucleotide sequence of SEQ ID NO: 63.
Clause 38. A circular RNA precursor comprising the following elements from 5′ to 3′ direction in the following order:
a) a 3′ self-splicing intron fragment;
b) a first residual circularizing element;
c) a nucleotide sequence of interest;
d) a second residual circularizing element; and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 5 to about 100 nucleotides,
wherein the self-splicing intron is selected from the Group I intron of the Anabaena pre-tRNA-Leu gene,
wherein the 3′ self-splicing intron fragment comprises or consists of a nucleotide sequence of SEQ ID NO: 1 or a nucleotide sequence having at least 75%, at least 80%, at least 85%, at least 90%, at least 95%, 100% identity to SEQ ID NO: 1, and the 5′ self-splicing intron fragment comprises or consists of a nucleotide sequence of SEQ ID NO: 2 or a nucleotide sequence having at least 75%, at least 80%, at least 85%, at least 90%, at least 95%, 100% identity to SEQ ID NO: 2,
wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55, and the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.
Clause 39. The circular RNA precursor of clause 38, wherein the 3′ self-splicing intron fragment comprises or consists of a nucleotide sequence of SEQ ID NO: 1 and 5′ self-splicing intron fragment comprises or consists of a nucleotide sequence of SEQ ID NO: 2.
Clause 40. The circular RNA precursor of clause 38 or 39, wherein
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 17 and the second residual circularizing element comprises or consists of a nucleotide sequence of SEQ ID NO: 59; or
15) the first residual circularizing element comprises or consists of a nucleotide sequence of SEQ ID NO: 22 and the second residual circularizing element comprises or consists of a nucleotide sequence of SEQ ID NO: 63.
Clause 41. The circular RNA precursor of any one of clauses 1-40, wherein the circular RNA precursor further comprises a 5′ homology arm sequence and a 3′ homology arm sequence capable of complementary pairing to each other to form a homology arm double-stranded region.
Clause 42. The circular RNA precursor of clause 41, wherein the 5′ homology arm sequence is upstream of the 3′ self-splicing intron fragment and the 3′ homology arm sequence is downstream of the 5′ self-splicing intron fragment.
Clause 43. The circular RNA precursor of any one of clauses 41-42, wherein the homology arm is about 5-50 nucleotides in length, preferably, about 40 nucleotides in length.
Clause 44. The circular RNA precursor of any one of clauses 41-43, wherein the two homology arm sequences may be polyA and polyT, respectively, or polyG and polyC, respectively.
Clause 45. The circular RNA precursor of any one of clauses 41-44, wherein one of the homology arm sequence has the nucleotide sequence of any one of SEQ ID NO: 151-161 or a nucleotide sequence having at least 50%, at least 60%, at least 70%, at least 80%, at least 90%, at least 95%, or at least 99% sequence identity with any one of SEQ ID NO:151-161, while the other homology arm sequence has the corresponding complementary sequence.
Clause 46. The circular RNA precursor of any one of clauses 1-45, wherein the nucleotide sequence of interest comprises at least one protein-coding sequence and a translation initiation element such as an internal ribosome entry site (IRES) operably linked thereto.
Clause 47. The circular RNA precursor of clause 46, wherein the translation initiation element, such as an IRES, is located upstream to the 5′ end of the at least one protein-coding sequence.
Clause 48. The circular RNA precursor of clause 46, wherein the translation initiation element, such as an IRES, is located downstream to the 3′ end of the at least one protein-coding sequence.
Clause 49. The circular RNA precursor of any one of clauses 46-48, wherein the protein-coding sequence encodes a protein of eukaryotic, prokaryotic or viral origin, for example, a protein for therapeutic or diagnostic use.
Clause 50. The circular RNA precursor of any one of clauses 46-49, wherein the translation initiation element is an internal ribosome entry site (TRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is CVB3, BRAV-1_L, PV1_L, CAV2_L, BRAV-1, PV1, or CAV2.
Clause 51. The circular RNA precursor of any one of clauses 46-50, wherein the IRES comprises a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.
Clause 52. The circular RNA precursor of any one of clauses 1-45, wherein the nucleotide sequence of interest is a non-protein coding sequence, for example, the non-protein-coding sequence is selected from antisense RNA, aptamer, guide RNA, or a non-protein-coding RNA naturally existing in an organism.
Clause 53. The circular RNA precursor of any one of clauses 1-52, wherein the nucleotide sequence of interest is about 10-about 20000 nucleotides in length.
Clause 54. The circular RNA precursor of any one of clauses 1-53, wherein the circular RNA precursor does not contain nucleotide chemical modification.
Clause 55. A nucleic acid vector for generating a circular RNA molecule, said vector comprises a coding sequence of the circular RNA precursor of any one of clauses 1-54.
Clause 56. The nucleic acid vector of clause 55, which further comprises an RNA polymerase promoter sequence operably linked to the coding sequence of the circular RNA precursor.
Clause 57. The nucleic acid vector of clause 56, wherein the promoter is a T7 RNA polymerase promoter, a T6 viral RNA polymerase promoter, a SP6 viral RNA polymerase promoter, a T3 viral RNA polymerase promoter or a T4 viral RNA polymerase promoter, preferably a T7 RNA polymerase promoter.
Clause 58. A circular RNA, which is prepared from the circular RNA precursor of any one of clauses 1-54 or from the nucleic acid vector of any one of clauses 55-57.
Clause 59. A circular RNA, which comprises a first residual circularizing element, a nucleotide sequence of interest, and a second residual circularizing element, wherein the first residual circularizing element, the nucleotide sequence of interest, and the second residual circularizing element are defined in any one of clauses 1-54.
Clause 60. A circular RNA, which comprises a first residual circularizing element, a nucleotide sequence of interest, and a second residual circularizing element, wherein the total length of the first residual circularizing element and the second residual circularizing element is about 5 to about 100 nucleotides.
Clause 61. The circular RNA of clause 60, wherein the first residual circularizing element and the second residual circularizing element are involved in RNA circularization with a self-splicing intron, such as a Group I intron, preferably a Group I intron of the Anabaena pre-tRNA-Leu gene.
Clause 62. The circular RNA of clause 60 or 61, wherein the first residual circularizing element and the second residual circularizing element are covalently linked, for example, 5′ end of the first residual circularizing element is covalently linked to 3′ end of the second residual circularizing element.
Clause 63. The circular RNA of any one of clauses 60-62, wherein the first residual circularizing element and the second residual circularizing element are configured such that the circular RNA comprising them has reduced immunogenicity relative to a control RNA, such as a control circular RNA comprising the circularizing elements of SEQ ID NO:29 and SEQ ID NO:64 or a control linear RNA comprising the same nucleotide sequence of interest.
Clause 64. The circular RNA of any one of clauses 60-63, wherein the first residual circularizing element and the second residual circularizing element are configured such that the circular RNA comprising them can be generated with a comparable or increased circularization efficiency relative to a control circular RNA, such as a control circular RNA comprising the residual circularizing elements of SEQ ID NO:29 and SEQ ID NO:64 (Ana 3.0).
Clause 65. The circular RNA of any one of clauses 60-64, wherein the total length of the first residual circularizing element and the second residual circularizing element is about 20 to about 35 nucleotides.
Clause 66. The circular RNA of any one of clauses 60-65, wherein the first residual circularizing element and the second residual circularizing element are configured to be capable of forming a stem-loop structure.
Clause 67. The circular RNA of clause 66, wherein the loop of the stem-loop structure comprises the splicing junction between the first residual circularizing element and the second residual circularizing element.
Clause 68. The circular RNA of any one of clauses 60-67, wherein the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure.
Clause 69. The circular RNA of clause 68, wherein the first loop sequence comprises or consists of one or more nucleotides which can pair with the P1 region of the corresponding self-splicing intron to form a P10 duplex region during the circularization.
Clause 70. The circular RNA of clause 68 or 69, wherein the first loop sequence comprises or consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.
Clause 71. The circular RNA of any one of clauses 68-70, wherein the first loop sequence comprises or consists of the sequence of AAAA, AA, UUUU, UAAA, CAAAA, or GAAAA, preferably AAAA.
Clause 72. The circular RNA of any one of clauses 68-71, wherein the second loop sequence comprises or consists of one or more nucleotides which can pair with the internal guide sequence (IGS) of the corresponding self-splicing intron to form a P1 duplex region during the circularization.
Clause 73. The circular RNA of any one of clauses 68-72, wherein the second loop sequence comprises or consists of CUU or CUC, preferably CUU.
Clause 74. The circular RNA of any one of clauses 68-73, wherein the loop of the stem-loop structure has a sequence of CUUAAAA, CUUUUUU, CUUAA, CUUGAAA, CUUUAAA, CUUCAAA or CUCAAAA, preferably CUUAAAA.
Clause 75. The circular RNA of any one of clauses 68-74, wherein the stem portion of the stem-loop structure comprises 2-15 or more consecutive base pairs, preferably, the stem portion of the stem-loop structure comprises 5, 6 or 7 consecutive base pairs.
Clause 76. The circular RNA of any one of clauses 68-75, wherein the stem portion in the stem-loop structure comprises up to 2 base mismatches, or the stem portion comprises only 1 base mismatch, preferably, the stem portion comprises no base mismatches.
Clause 77. The circular RNA of any one of clauses 68-76, wherein the first pairing sequence comprises only Gs and the second pairing sequence comprises only Cs.
Clause 78. The circular RNA of any one of clauses 68-76, wherein the first pairing sequence comprises only Cs and the second pairing sequence comprises only Gs.
Clause 79. The circular RNA of any one of clauses 68-76, wherein the first pairing sequence includes only A and the second pairing sequence includes only U.
Clause 80. The circular RNA of any one of clauses 68-76, wherein the first pairing sequence comprises or consists of the sequence of any one of SEQ ID NO:42-55, and, the first pairing sequence comprises or consists of the sequence of any one of SEQ ID NO:78-93.
Clause 81. The circular RNA of any one of clauses 67-80, wherein the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower.
Clause 82. The circular RNA of any one of clauses 60-81, wherein the first residual circularizing element comprises or consists of from 5′ to 3′ direction a 3′ exon region and optionally a spacer, the second residual circularizing element comprises or consists of from 3′ to 5′ direction a 5′ exon region and optionally a spacer.
Clause 83. The circular RNA of clause 82, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
Clause 84. The circular RNA of clause 83, wherein 3′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 5′ exon or a contiguous fragment of about 1-about 50 nucleotides starting from the 3′ terminal nucleotide of the native 5′ exon.
Clause 85. The circular RNA of any one of clauses 60-84, wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55.
Clause 86. The circular RNA precursor of any one of clauses 60-85, wherein the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.
Clause 87. The circular RNA of any one of clauses 60-86, wherein the first residual circularizing element comprises or consists of a nucleotide sequence of any one of SEQ ID NOs: 13-55 and the second residual circularizing element comprises or consists of a nucleotide sequence of any one of SEQ ID NOs: 56-93.
Clause 88. The circular RNA of any one of clauses 60-87, wherein,
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 17 and the second residual circularizing element comprises or consists of a nucleotide sequence of SEQ ID NO: 59; or
15) the first residual circularizing element comprises or consists of a nucleotide sequence of SEQ ID NO: 22 and the second residual circularizing element comprises or consists of a nucleotide sequence of SEQ ID NO: 63.
Clause 89. The circular RNA of any one of clauses 60-88, wherein the nucleotide sequence of interest comprises at least one protein-coding sequence and a translation initiation element such as an internal ribosome entry site (IRES) operably linked thereto.
Clause 90. The circular RNA of clause 89, wherein the translation initiation element, such as an IRES, is located upstream to the 5′ end of the at least one protein-coding sequence.
Clause 91. The circular RNA of clause 89, wherein the translation initiation element, such as an IRES, is located downstream to the 3′ end of the at least one protein-coding sequence.
Clause 92. The circular RNA of any one of clauses 89-91, wherein the protein-coding sequence encodes a protein of eukaryotic, prokaryotic or viral origin, for example, a protein for therapeutic or diagnostic use.
Clause 93. The circular RNA of any one of clauses 89-92, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is CVB3, BRAV-1_L, PV1_L, CAV2_L, BRAV-1, PV1, or CAV2.
Clause 94. The circular RNA of any one of clauses 89-93, wherein the IRES comprises a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.
Clause 95. The circular RNA of any one of clauses 60-88, wherein the nucleotide sequence of interest is a non-protein coding sequence, for example, the non-protein-coding sequence is selected from antisense RNA, aptamer, guide RNA, or a non-protein-coding RNA naturally existing in an organism.
Clause 96. The circular RNA of any one of clauses 60-95, wherein the nucleotide sequence of interest is about 10-about 20000 nucleotides in length.
Clause 97. The circular RNA of any one of clauses 60-96, wherein the circular RNA does not contain nucleotide chemical modification.
Clause 98. Use of the circular RNA precursor any one of clauses 1-54 and/or circular RNA of any one of clauses 58-97 as an expression vector.
Clause 99. A pharmaceutical composition comprising the circular RNA precursor of any one of clauses 1-54 and/or the nucleic acid vector of any one of clauses 55-57 and/or circular RNA of any one of clauses 58-97, and a pharmaceutically acceptable carrier.
Clause 100. The pharmaceutical composition of clause 99, which is for use in treating a disease in a subject.
Clause 101. A method for preparing a circular RNA, the method comprises:
1) providing a circular RNA precursor of any one of clauses 1-54 or obtaining a circular RNA precursor by transcribing from the nucleic acid vector of any one of clauses 55-57;
2) incubating the circular RNA precursor in the presence of a divalent metal cation at a temperature at which RNA circularization occurs; and
3) harvesting the circular RNA obtained in step 2).
Clause 102. The method of clause 101, wherein the divalent metal cation is Mg²⁺ and/or Mn²⁺.
Clause 103. The method of clause 101 or 102, wherein the concentration of the divalent metal cation is about 5 mM to about 550 mM.
Clause 104. A method for preparing a circular RNA, the method comprises
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.
Clause 105. The method of clause 104, wherein the nucleic acid vector is the nucleic acid vector of any one of clauses 55-57.
Clause 106. The method of clause 104 or 105, wherein the divalent metal cation in the in vitro transcription system is Mg²⁺.
Clause 107. The method of any one of clauses 104-106, wherein the in vitro transcription system further comprises a monovalent metal cation and/or a monovalent anion.
Clause 108. The method of clause 107, wherein the monovalent metal cation is Na⁺ or K⁺.
Clause 109. The method of clause 107, wherein the monovalent metal anion is Cl⁻ or CH3COO⁻ (OAc).
Clause 110. The method of any one of clauses 104-109, wherein the concentration of the divalent metal cation in the system during the first time period is from about 5 mM to about 50 mM.
Clause 111. The method of any one of clauses 107-110, wherein the concentration of the monovalent metal cation in the system during the first time period is from about 5 mM to about 100 mM.
Clause 112. The method of any one of clauses 107-111, wherein the concentration of the monovalent anion in the system during the first time period is from about 5 mM to about 150 mM.
Clause 113. The method of any one of clauses 107-112, wherein the method does not include a step of isolating and/or purifying the linear RNA produced by the in vitro transcription.
Clause 114. The method of any one of clauses 107-113, wherein the buffer of the in vitro transcription system is selected from Tris-HCl buffer, or HEPES buffer, or MES buffer, or citrate buffer, or phosphate buffer, preferably, the buffer of the in vitro transcription system is HEPES buffer.
Clause 115. The method of any one of clauses 107-114, wherein the in vitro transcription system has a pH of about 5-about 8, preferably, the in vitro transcription system has a pH of about 7.5.
Clause 116. The method of any one of clauses 107-115, wherein the RNA polymerase is selected from a T7 RNA polymerase, a T6 viral RNA polymerase, a SP6 viral RNA polymerase, a T3 viral RNA polymerase, or a T4 viral RNA polymerase, preferably, the RNA polymerase is T7 RNA polymerase.
Clause 117. The method of any one of clauses 107-116, wherein the first time period is about 0.5 hours to about 24 hours, preferably, the first time period is 3 hours.
Clause 118. The method of any one of clauses 107-117, wherein the incubation for the first time period is carried out at about 16° C. to about 60° C., preferably, the incubation for the first time period is carried out at about 37° C.
Clause 119. The method of any one of clauses 107-118, wherein after incubation for the first time period in step b), the method further comprises step c):
adding an additional amount of metal cation to the in vitro transcription system and incubating for a second time period; or
changing the buffer of the system, adding a metal cation and incubating for a second time period.
Clause 120. The method of clause 119, wherein the metal cation added for the incubation of the second time period is a divalent metal cation, such as Mg²⁺ or Mn²⁺.
Clause 121. The method of clause 119 or 120, wherein during the incubation of the second time period, the metal cation is added to a final concentration of about 5 mM to about 550 mM.
Clause 122. The method of any one of clauses 119-121, wherein the buffer of the system is changed to Tris-HCl buffer, or HEPES buffer, or MES buffer, or citrate buffer, or phosphate buffer during the second time period.
Clause 123. The method of any one of clauses 119-122, wherein the pH of the system in the second time period is 5-8.
Clause 124. The method of any one of clauses 119-123, wherein the second time period is about 5 minutes to about 2 hour.
Clause 125. The method of any one of clauses 119-124, wherein the incubation for the second time period is carried out at about 25° C. to about 75° C.
Clause 126. The method of any one of clauses 104-125, wherein the method further comprises a step of recovering or purifying the circular RNA as produced.
Clause 127. A method for purifying a circular RNA, the method comprises:
a) contacting a mixture comprising circular RNA and uncircularized linear circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.
Clause 128. The method of clause 127, wherein the circular RNA is prepared by circularizing a linear circular RNA precursor.
Clause 129. The method of clause 127 or 128, wherein the circular RNA is prepared by ligating both ends of a linear circular RNA precursor with an RNA ligase, or, the circular RNA is prepared by the self-splicing ribozyme activity of self-splicing intron-based circularizing elements contained in the linear circular RNA precursor.
Clause 130. The method of any one of clauses 127-129, wherein the circular RNA-specific probe is a single-stranded DNA or RNA probe.
Clause 131. The method of any one of clauses 127-130, wherein the circular RNA-specific probe specifically hybridizes to a region flanking the circularization junction of the circular RNA.
Clause 132. The method of any one of clauses 127-131, wherein the circular RNA-specific probe is about 10 nucleotides-about 35 nucleotides in length or longer.
Clause 133. The method of any one of clauses 127-132, wherein the circular RNA-specific probe is immobilized on a support such as a solid support, e.g., the circular RNA-specific probe is immobilized on the support after binding to the circular RNA or the circular RNA-specific probe is pre-immobilized on the support.
Clause 134. The method of any one of clauses 127-133, wherein the condition in step a) include denaturing the RNA at between about 60° C. and about 95° C. for about 2 minutes to about 10 minutes, then gradually reducing the temperature to below about 40° C. to allow the circular RNA annealing to the circular RNA-specific probe.
Clause 135. The method of clause 134, wherein step c) is performed by increasing the temperature to about 60° C. to about 95° C. (e.g., about 60° C., about 62° C., about 64° C., about 66° C., about 68° C.) C, about 70° C., about 75° C., about 80° C., about 85° C., about 90° C., about 95° C.) to release the circular RNA.
Clause 136. The method of any one of clauses 127-133, wherein the condition in step a) include a high salt concentration range of 0.25M-2M.
Clause 137. The method of clause 136, wherein the salt is NaCl or a guanidine salt.
Clause 138. The method of clause 136 or 137, wherein in step c) the circular RNA is released by elution with an elution buffer, such as a low salt buffer.
Clause 139. The method of clause 138, wherein the elution buffer is Tris-EDTA buffer (TE buffer) or water.
Clause 140. The method of any one of clauses 127-139, wherein in step b), the one or more components are removed by washing the complex with a washing buffer.
Clause 141. The method of any one of clauses 127-140, the method further includes the following steps:
i) contacting the mixture comprising circular RNA and uncircularized linear circular RNA precursor to a linear circular RNA precursor-specific probe in a condition that allows the linear circular RNA precursor-specific probe to specifically bind to and form a complex with the linear circular RNA precursor;
ii) removing the complex formed by the linear circular RNA precursor-specific probe with the linear circular RNA precursor from the mixture, and
iii) collecting the circular RNA-containing mixture obtained in step ii).
Clause 142. The method of clause 141, wherein steps i)-iii) are performed before step a), for example, steps i)-iii) may be performed multiple times before step a), e.g., 2, 3, 4 or more times.
Clause 143. The method of clause 141, wherein steps i)-iii) are performed concurrently with steps a)-c).
Clause 144. A method for purifying circular RNA, the method comprises:
i) contacting a mixture comprising circular RNA and uncircularized linear circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and
optionally, steps i)-iii) are performed multiple times, e.g., 2 times, 3 times, 4 times or more.
Clause 145. The method of clause 144, wherein the linear circular RNA precursor-specific probe specifically binds to the linear circular RNA precursor and does not substantially bind to the circular RNA.
Clause 146. The method of clause 144 or 145, wherein the linear circular RNA precursor-specific probe is immobilized on a support, such as a solid support, for example, the linear circular RNA precursor-specific probe is then immobilized on the support after binding to the linear circular RNA precursor, or the linear circular RNA precursor-specific probe is pre-immobilized on the support.
Clause 147. The method of any one of clauses 127-146, wherein the linear circular RNA precursor comprises the following elements arranged in the following order from the 5′ to 3′ direction:
a) a 3′ self-splicing intron fragment;
b) a first residual circularizing element;
c) a nucleotide sequence of interest;
d) a second residual circularizing element; and
e) a 5′ self-splicing intron fragment;
wherein the linear circular RNA precursor is capable of removing the 3′ self-splicing intron fragment and the 5′ self-splicing intron fragment by self-splicing, generating a circular RNA comprising the first residual circularizing element, the nucleotide sequence of interest and the second residual circularizing element.
Clause 148. The method of clause 147, wherein the circular RNA-specific probe specifically hybridizes to at least a portion of the first residual circularizing element and a portion of the second residual circularizing element.
Clause 149. The method of clause 147 or 148, wherein the linear precursor RNA-specific probe hybridizes to a portion of the linear circular RNA precursor outside the first residual circularizing element, the nucleotide sequence of interest, and the second residual circularizing element.
Clause 150. The method of any one of clauses 147-149, wherein the linear precursor RNA-specific probe hybridizes to the 3′ self-splicing intron fragment or a portion thereof or a 5′ flanking sequence thereof, or the 5′ self-splicing intron fragment or a portion thereof or a 3′ flanking sequence thereof.
Clause 151. The method of any one of clauses 147-150, wherein the linear circular RNA precursor contains a sequence selected from SEQ ID NOs:96-101 or a complement sequence thereof, preferably, SEQ ID NO:100 or a complement sequence thereof outside the first residual circularizing element, the nucleotide sequence of interest, and the second residual circularizing element, to which the linear precursor RNA-specific probe specifically hybridizes.
Clause 152. The method of any one of clauses 147-151, the molar ratio of the probe to the RNA molecules in the mixture is from about 1:1 to about 100,000:1.
Clause 153. A method for purifying circular RNA, the method comprises:
i) adding a linear RNA-specific tag to the linear RNA in a mixture comprising circular RNA and linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii), optionally, steps ii)-iv) are performed multiple times, e.g., 2 times, 3 times, 4 times or more.
Clause 154. The method of clause 153, wherein the tag comprises a polyA, polyG, polyU, or polyC sequence.
Clause 155. The method of clause 153 or 154, wherein the tag is about 10-200 nt in length or the probe is about 10-200 nt in length.
Clause 156. The method of any one of clauses 153-155, wherein the tag is added to the linear RNA by adding a PolyA/T/C/G polymerase or a ligase to the mixture.
Clause 157. The method of any one of clauses 153-156, wherein the linear RNA probe specifically binds to the added tag without substantially binding to the circular RNA.
Clause 158. The method of any one of clauses 153-157, wherein the linear RNA probe is a single-stranded DNA probe, or a single-stranded RNA probe.
Clause 159. The method of any one of clauses 153-158, wherein the linear RNA probe is immobilized on a support such as a solid support, for example, the linear RNA probe is immobilized on the support after binding to the linear RNA, or, the linear RNA probe is pre-immobilized on the support.
Clause 160. A nucleic acid vector for generating a circular RNA molecule, which comprises a coding sequence of a circular RNA precursor comprising the following elements operably linked and arranged in the following order from 5′ to 3′ direction:
a) a 3′ Group I intron fragment;
b) a first residual circularizing element;
c) a nucleotide sequence of interest;
d) a second residual circularizing element; and
e) a 5′ Group I intron fragment;
wherein, when the circular RNA precursor is generated by transcription from the nucleic acid vector, the circular RNA precursor can generate a circular RNA comprising the first residual circularizing element, the nucleotide sequence of interest and the second residual circularizing element by self-splicing;
wherein the total length of the first residual circularizing element and the second residual circularizing element is at least 15 nucleotides, and the first residual circularizing element and the second residual circularizing element form a stem-loop structure in the circular RNA.
Clause 161. The nucleic acid vector of clause 160, wherein the first residual circularizing element comprises from the 5′ to 3′ direction: b1) a 3′ exon region; and/or b2) a 5′ spacer.
Clause 162. The nucleic acid vector of clause 160 or 161, wherein the second residual circularizing element comprises from the 5′ to 3′ direction: d1) a 3′ spacer; and/or d2) a 5′ exon region.
Clause 163. The nucleic acid carrier of any one of clauses 160-162, wherein the 3′ Group I intron fragment and the 5′ Group I intron fragment are from a cyanobacteria Anabaena (Anabaena) Group I intron or from a T4 phage Group I intron.
Clause 164. The nucleic acid vector of any one of clauses 160-163, wherein the stem portion in the stem-loop structure comprises at least 3 base pairs, or at least 5 base pairs, or at least 7 base pairs.
Clause 165. The nucleic acid vector of any one of clauses 160-164, wherein the stem portion in the stem-loop structure comprises up to 2 base pair mismatches, or at most 1 base pair mismatch, or does not comprise base pair mismatches.
Clause 166. The nucleic acid vector of any one of clauses 160-165, wherein the first residual circularizing element comprises a nucleotide sequence having at least 80%, at least 90%, at least 95% or 100% sequence identity to the nucleotide sequence selected from the group consisting of SEQ ID NOs: 13, 16, 30 and 31.
Clause 167. The nucleic acid vector of any one of clauses 160-166, wherein the second residual circularizing element comprises a nucleotide sequence having at least 80%, at least 90%, at least 95% or 100% sequence identity to the nucleotide sequence selected from the group consisting of SEQ ID NOs: 56, 57, 59 and 65.
Clause 168. The nucleic acid vector of any one of clauses 160-167, wherein the first residual circularizing element and the second residual circularizing element comprise respectively
SEQ ID NO: 13 and SEQ ID NO: 56; or
SEQ ID NO: 31 and SEQ ID NO: 65; or
SEQ ID NO: 30 and SEQ ID NO:65; or
SEQ ID NO:13 and SEQ ID NO:59; or
SEQ ID NO: 16 and SEQ ID NO: 56; or
SEQ ID NO: 13 and SEQ ID NO: 57.
Clause 169. The nucleic acid vector of any one of clauses 160-168, wherein the 3′ Group I intron fragment comprises the nucleotide sequence shown in SEQ ID NO: 1, and the 5′ Group I intron fragment comprises the nucleotide sequence shown in SEQ ID NO:2.
Clause 170. The nucleic acid vector of any one of clauses 160-169, further comprising a 5′ homology arm and a 3′ homology arm.
Clause 171. The nucleic acid vector of clauses 170, wherein the 5′ homology arm is located to the 5′ terminus of the 3′ Group I intron fragment, and the 3′ homology arm is located to 3′ terminus of the 5′ Group I intron fragment.
Clause 172. The nucleic acid vector of any one of clauses 160-171, wherein the nucleotide sequence of interest comprises a protein-coding sequence and an IRES (e.g., a CVB3 IRES) operably linked thereto.
Clause 173. The nucleic acid vector of any one of clauses 160-171, wherein the nucleotide sequence of interest is a non-protein coding sequence.
Clause 174. The nucleic acid vector of any one of clauses 160-173, further comprising a promoter sequence, such as a T7 promoter, operably linked to the coding sequence of the circular RNA precursor.
Clause 175. A circular RNA, which
1) is prepared from the nucleic acid vector according to one of the preceding claims; and/or
2) comprises a first residual circularizing element, a nucleotide sequence of interest, and a second residual circularizing element, wherein the first and second residual circularizing elements have a total length of at least 15 nucleotides, and the first residual circularizing element and the second residual circularizing element form a stem-loop structure in the circular RNA.
Clause 176. The circular RNA of clause 175, wherein the first residual circularizing element comprises from the 5′ to 3′ direction: b1) a 3′ exon region; and/or b2) a 5′ spacer.
Clause 177. The circular RNA of clause 176, wherein the second residual circularizing element comprises from the 5′ to 3′ direction: d1) a 3′ spacer; and/or d2) a 5′ exon region.
Clause 178. The circular RNA of any one of clauses 175-177, wherein the stem portion in the stem-loop structure comprises at least 3 base pairs, or at least 5 base pairs, or at least 7 base pairs.
Clause 179. The circular RNA of any one of clauses 175-178, wherein the stem portion in the stem-loop structure comprises up to 2 base pair mismatches, or up to 1 base pair mismatch, or does not comprise base pair mismatches.
Clause 180. The circular RNA of any one of clauses 175-179, wherein the first residual circularizing element comprises a nucleotide sequence having at least 80%, at least 90%, at least 95% or 100% sequence identity to the nucleotide sequence selected from the group consisting of SEQ ID NOs: SEQ ID NOs: 13, 16, 30 and 31.
Clause 181. The circular RNA of any one of clauses 175-180, wherein the second residual circularizing element comprises a nucleotide sequence having at least 80%, at least 90%, at least 95% or 100% sequence identity to the nucleotide sequence selected from the group consisting of SEQ ID NOs: 56, 57, 59 and 65.
Clause 182. The circular RNA of any one of clauses 175-181, wherein the first residual circularizing element and the second residual circularizing element respectively comprise
SEQ ID NO: 13 and SEQ ID NO: 56; or
SEQ ID NO: 31 and SEQ ID NO: 65; or
SEQ ID NO: 30 and SEQ ID NO:65; or
SEQ ID NO:13 and SEQ ID NO:59; or
SEQ ID NO: 16 and SEQ ID NO: 56; or
SEQ ID NO: 13 and SEQ ID NO: 57.
Clause 183. A pharmaceutical composition comprising the nucleic acid carrier of any one of clauses 1-174 and/or the circular RNA of any one of clauses 175-182, and a pharmaceutically acceptable carrier.

### EXAMPLES

The following examples are included to further illustrate the invention described herein and to demonstrate embodiments of the invention. It should be appreciated by those of skill in the art that the techniques disclosed in the examples which follow represent techniques discovered by the inventors to function well in the practice of the invention, and thus can be considered to constitute modes for its practice. However, those of skill in the art should, in light of the present disclosure, appreciate that many changes can be made in the specific embodiments which are disclosed and still obtain a like or similar result which are within the spirit and scope of the invention.

### Example 1. Circular RNA Circularization Method

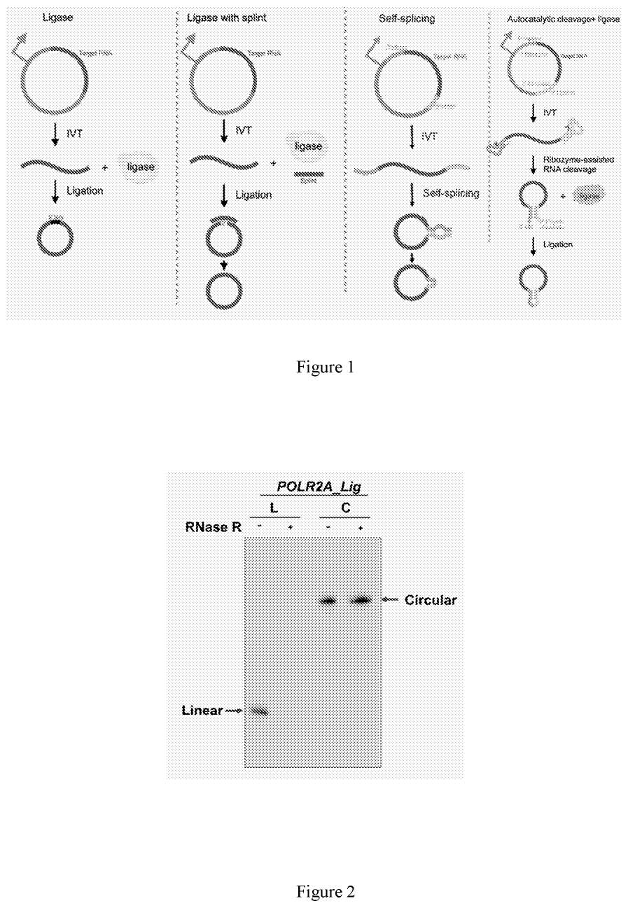

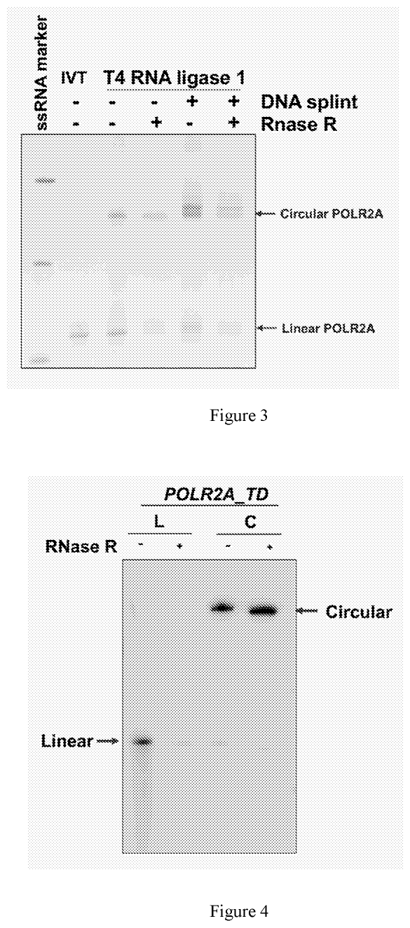

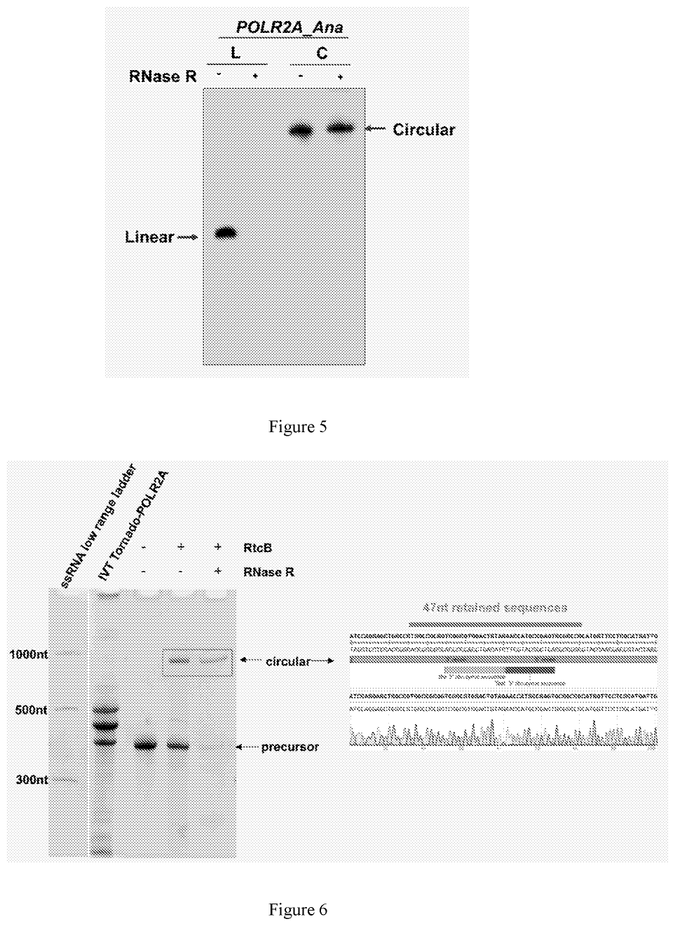

This Example illustrates how different circular RNAs can be circularized in vitro (FIG. 1), including:
1) Through ligase, such as RNA ligase, DNA ligase, and etc., ligating the 3′-OH terminal and 5′-phosphate terminal of linear RNA produced by in vitro transcription to form phosphodiester bond to generate circular RNA (FIG. 2).
2) By using DNA or RNA splint with complementary sequence with the both termini of linear RNA produced by in vitro transcription, under the catalysis of ligase (such as RNA ligase, DNA ligase, etc.), the 3′-OH end of linear RNA and 5′-phosphate termini are linked by phosphodiester bonds to form circular RNA (FIG. 3).
3) Through self-splicing ribozymes, such as group I introns, group II introns, and etc., a cleavage and ligation reaction occur to form a circular RNA. The basic principle is to connect exons E1 and E2 end to end by molecular cloning to generate a continuous circular plasmid. The introns are cleaved by restrictive endonucleases to obtain linearized plasmids. Then, in vitro transcription was performed through the promoter upstream of the inverted 3′ intron to obtain a linear RNA containing the structure of 3′ intron-E2-E1-5′ intron. The conserved sequence of specific cleavage site of exon E1 is cleaved by the nucleophilic attack of the free 3′ hydroxyl of guanosine, exon E1 produces an exposed 3′ hydroxyl, and guanosine binds to the cleaved 5′ intron. Subsequently, the exposed 3′ hydroxyl of exon E1 attacks the conserved sequence between the 3′ intron and exon E2, the 3′ intron is excised, and exon E2 and E1 get connected. The reaction resulted in circular E1-E2 RNA (FIG. 4, 5).
4) Through the combined reaction of self-cleaving ribozymes (such as HDV, HHR, Twister, etc.) and ligases (such as RtcB, etc.), the ends of linear RNAs are joined to form circular RNAs. The basic principle is that the 5′ and 3′ self-cleaving ribozyme sequences are placed at the end of the linear plasmid through molecular cloning, and after in vitro transcription is initiated, the resulted linear RNA precursor will be automatically cleaved by the ribozymes placed at 5′ and 3′ end. The cleavage leads to the formation of 5′-OH and 2′, 3′-cyclic phosphate, respectively, and then under the action of ligase (RtcB, and etc.), the ends are linked by phosphodiester bonds to form circular RNA (FIG. 6).

### Example 2. Residual Circularizing Elements of Group I Intron Causes the Immunogenicity of Circular RNA

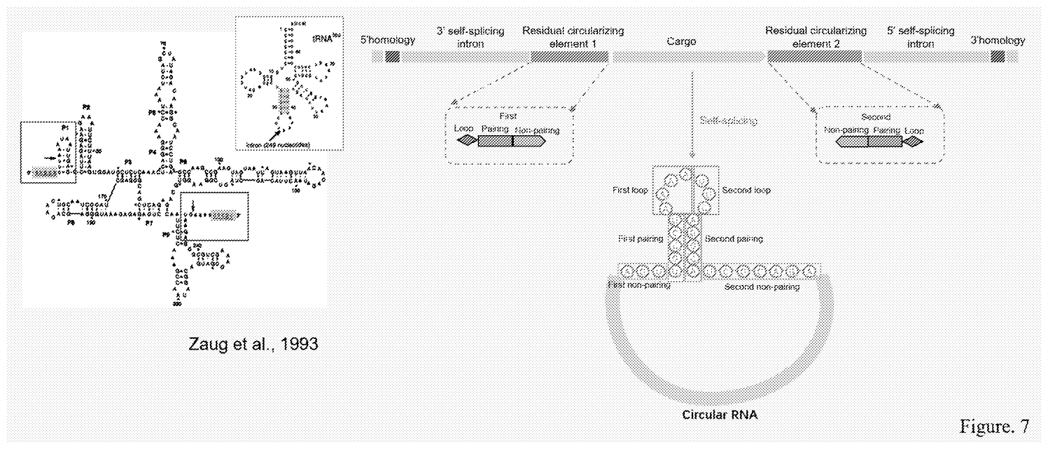

Circular RNA synthesized in vitro mainly includes two elements, 1) the residual circularizing element, that is, the additional sequence introduced to the final product of the circular RNA during the circularization reaction; 2) the sequence of interest (Cargo) (FIG. 7). The residual circularizing elements are additional sequences retained on the circular RNA introduced by in vitro circularization methods (mainly via ribozyme methods). Taking the circularization through Anabaena pre-tRNA group I intron self-splicing (Wesselhoeft A. R., et al., (2018) Engineering circular RNA for potent and stable translation in eukaryotic cells.) as an example, additional 176 nucleotide sequence were introduced and retained in the circular RNA final product. This embodiment illustrates that the residual of additional unnecessary nucleotide sequences in the circular RNA final product can negatively affect the corresponding circular RNA product, at least including possibly interfering with the insertion of the sequence of interest, or potentially leading to a certain degree of innate immune responses in the cells.

### 1. Circular RNA Generation and Purification

The purified circular RNA circular POLR2A was prepared in vitro in three ways (FIGS. 2, 4, and 5):
Method 1: Circular RNA was prepared by in vitro transcription and T4 RNA ligase via intramolecular ligation, and purified by gel excision of denature polyacrylamide gel (denature PAGE) to obtain circular POLR2A with high purity (FIG. 2).
Method 2: Circular RNA was prepared by in vitro transcription RNA and td group I intron self-cleavage (Chen G. Y et al., (2017) Sensing Self and Foreign Circular RNAs by Intron Identity; Wesselhoeft A. R., et al., (2018) Engineering circular RNA for potent and stable translation in eukaryotic cells.), and purified in vitro by gel excision of denature PAGE to obtain circular POLR2A with high purity (FIG. 4).
Method 3: Circular RNA was successfully prepared by in vitro RNA transcription and Anabaena group I intron circularization (Wesselhoeft A. R., et al., (2018) Engineering circular RNA for potent and stable translation in eukaryotic cells.), and purified in vitro by gel excision of denature PAGE to obtain circular POLR2A with high purity (FIG. 5).

### 2. Comparison of the Immunogenicity of Circular POLR2A Obtained in Three Different Ways at the Cellular Level

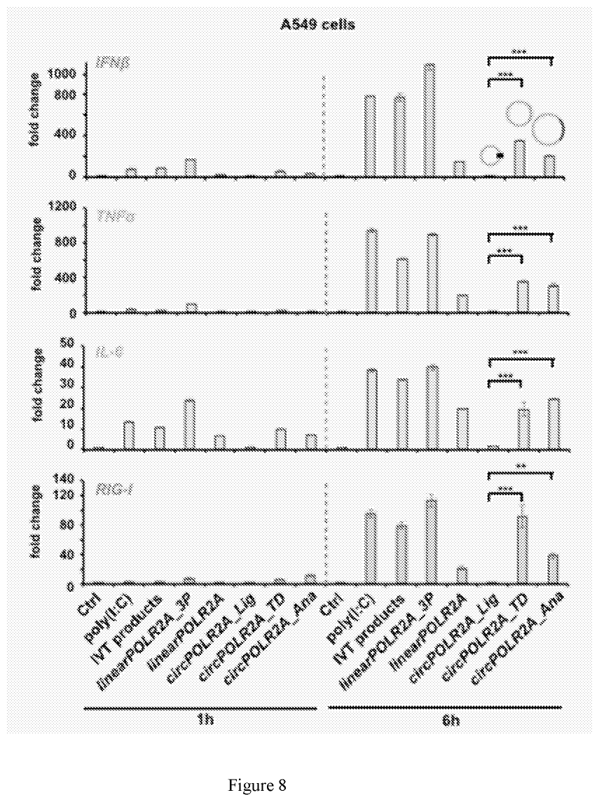

Circular RNA was successfully prepared by the above three circularization methods, and purified in vitro by denature PAGE gel to obtain circular POLR2A with high purity. Then, the circular POLR2A (200 ng/well) were transfected into human A549 cells in 12-well plate using lipofectamine. After 1 hour or 6 hours of transfection, the cells were harvested, and the expression level of the cytokines, include IFNβ, TNFα, IL6 and RIG-I, were determined using quantitative RT-PCR and normalized to 18S RNA. The results showed that compared to double strand RNA Poly(I:C), unpurified circular RNA, and linear POLR2A, the purified circular POLR2A prepared via Method 1 did not lead to the elevated expression level of IFNβ, TNFα, IL6 and RIG-I (FIG. 8). These data demonstrated that the circular POLR2A prepared and purified in vitro using Method 1 did not cause obvious immune responses. In contrast, the circular POLR2A prepared and purified in vitro using Method 2 and Method 3 led to elevated expression level of IFNβ, TNFα, IL6 and RIG-I (FIG. 8). The data demonstrated that circular RNA prepared using methods 2 and 3 elicited augmented immune responses.

### 3. Potential Mechanistic Explanation

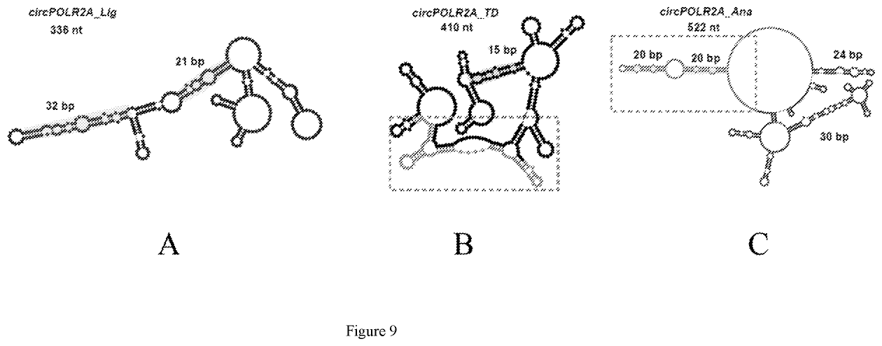

The secondary structures of circular RNAs prepared by the three methods were predicted. As shown in FIG. 9, the circular RNA prepared by Method 2 and Method 3 retained additional nucleotide sequence introduced via the circularization methods, the additional introduced sequence will form a stable double-stranded stem-loop structure (in-frame part of FIG. 9), and it is speculated that the additional introduced sequence will form a stable double-stranded stem-loop structure. The stable double-stranded stem-loop structure elicits an innate immune response within the cell.

### Example 3. Improved Residual Circularizing Elements for Group I Introns

As previously described, through circularization via in vitro transcription and self-splicing by Anabaena pre-tRNA group I intron, additional 176 nucleosides were introduced and retained in the circular RNA final product (Wesselhoeft et al., 2018 A. R., et al., (2018) Engineering circular RNA for potent and stable translation in eukaryotic cells.). We hypothesized that the potential adverse factors of the additionally introduced sequences may, in particular, lead to a certain degree of innate immune responses in the cells. In this embodiment, a series of modifications were explored on the self-splicing circularization method by Anabaena pre-tRNA class I introns (named Ana3.0) in Wesselhoeft et al., 2018.

### 1. Shortening the Sequence of Residual Circularizing Element in Circular RNA Generated by Anabaena Group I Intron Remain the Circularization Efficiency

Preliminary prediction of RNA secondary structure by RNAstructure (https://rna.urmc.rochester.edu/RNAstructureWeb/index.html) on the retained additional nucleotide sequence indicated that the retained additional nucleotide sequence tends to form a relatively long stem-loop structure. Under the premise of not changing the overall stem-loop structure around the splicing site, the residual sequences were subjected to a series of truncations based on the distance from the splice site (FIG. 10), and the effect of truncations on the circularization efficiency of Anabaena group I intron has been tested. The truncated versions of the residual sequences retained 99 nucleotides (Ana1.0), 66 nucleotides (Ana0.9) and 27 nucleotides (AnaX). Taking the circular mCherry generated in vitro as an example, the result of Northern Blot showed that if 27 nucleotides or above were retained, and its circularization efficiency was similar to the original version Ana3.0 (FIG. 10). RNase R was used as a tool enzyme for verifying the circular structure of circular RNAs, and it can effectively degrade linear RNAs with free ends, but not circular RNAs.

### 2. Shortening the Residual Circularizing Elements of Circular RNA Generated by Anabaena Group I Intron Leads to Lowered Immunogenicity at Cellular Level

In order to compare the immunogenicity of a series of truncated Anabaena group I intron self-splicing circular RNAs, circular mCherry of different Anabaena group I intron truncated versions were synthesized in vitro, and the purity of different truncated versions were checked by Northern Blot (FIG. 10). The same amount (200 ng) of linear mCherry mRNA and circular mCherry RNA of different truncated versions were transfected into A549 cells. At 6 hours post-transfection, the cells were collected, and Trizol reagent was added to extract the total RNA from the cells according to the manufacture's instructions and subjected to RT-qPCR detection for the expression IFNβ, TNFα, IL6 and RIG-I at mRNA level. The results showed that the circular mCherry generated via AnaX retaining 27 additional nucleotides was less immunogenic than other Anabaena group I intron truncated versions. In addition, the immunogenicity of different truncated versions of circular mCherry is lower than that of linear mCherry mRNA, which is consistent with the conclusion that circular RNA has lower immunogenicity than linear mRNA in Embodiment 2. Meanwhile, the circular mCherry formed by T4 ligase was the least immunogenic (FIG. 10). IFNβ, TNFα, IL6 and RIG-I are commonly used as marker genes for the measurement of cellular immunogenicity, and the positive control Poly (I:C) is a commonly used long double-stranded RNA to mimic virus infection at circular level.

3. AnaX-Circular RNA has Higher Translation Efficiency than mRNA

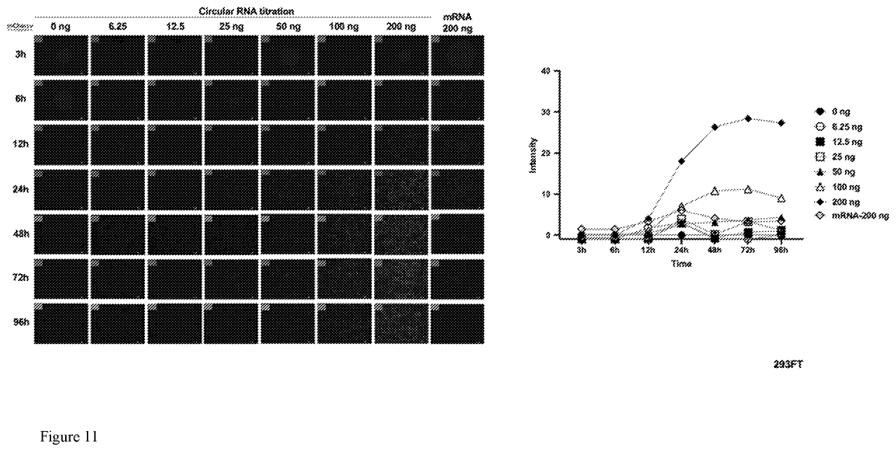

In order to measure the translation efficiency, 200 ng linear mCherry mRNA and various amount (6.25 ng-200 ng) of circular mCherry RNA containing residual circularizing elements generated via AnaX circularization method were transfected into human HEK293FT cells using Lipofectamine MessengerMAX™ (Thermo Fisher). The red fluorescent signal of the translational product mCherry was detected by fluorescence microscope at different time points (FIG. 11). As shown in FIG. 11, 24 hours post transfection, the red fluorescence signal of 200 ng mRNA transfected cells was similar to that of 50 ng circular mCherry (FIG. 11, red fluorescence quantitative statistical results), indicating that AnaX synthesizes circular mCherry with higher intracellular translation efficiency than linear mCherry mRNA.

4. AnaX-circRNA Exhibits Lower Immunogenicity than mRNA

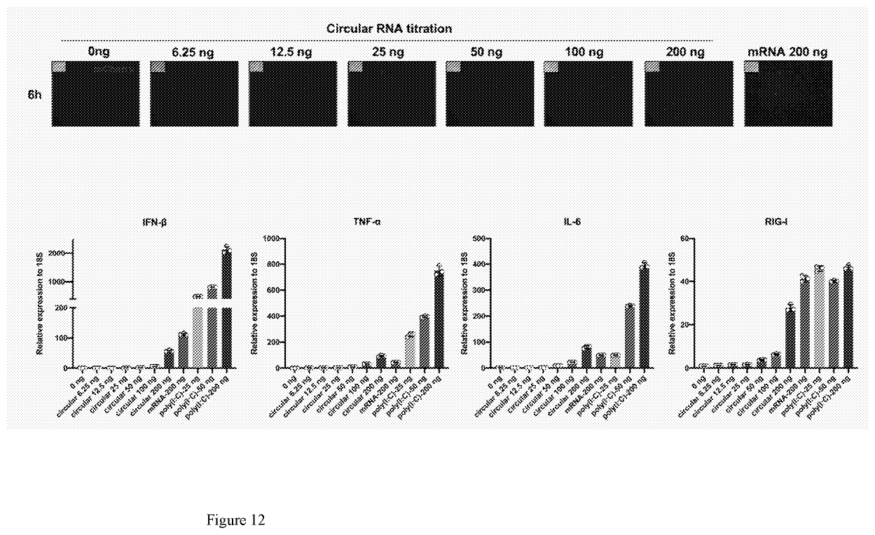

In order to measure the intracellular innate immune response caused by the transfection of linear mRNA, 200 ng linear mCherry mRNA and various amount (6.25 ng-200 ng) of circular mCherry RNA containing residual circularizing elements generated via AnaX circularization method were transfected into human A549 cells using Lipofectamine MessengerMAX™ (Thermo Fisher). After 6 hours of transfection, cells were collected, and Trizol reagent was added to extract the total RNA of cells for RT-qPCR to detect the mRNA expression levels of cytokines IFNβ, TNFα, IL6 and RIG-I, respectively. The results showed that the expression levels of corresponding cytokines in A549 cells transfected with 200 ng of linear mCherry mRNA were higher than those in A549 cells transfected with circular RNA generated via AnaX (FIG. 12). Moreover, under the same translation level, the A549 cells transfected with the circular mCherry almost did not show the elevated level of the corresponding cytokines, indicating that under the same translation efficiency, the circular mCherry generated via AnaX exhibits lower immunogenicity than the linear mCherry mRNA (FIG. 12). IFNβ, TNFα, IL6 and RIG-I are commonly used marker genes for evaluating intracellular immunogenicity, and the positive control Poly (I:C) is a commonly used long double-stranded RNA virus mimic.

### Example 4. Circularization of RNAs with Different Lengths by AnaX System

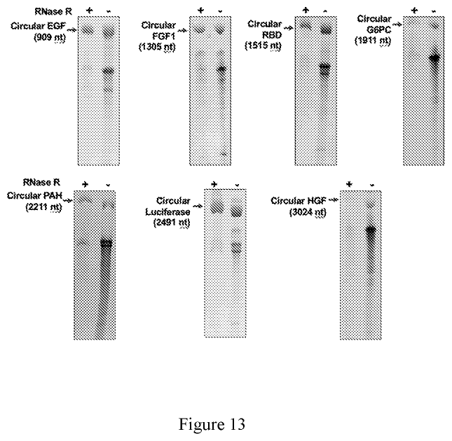

In order to test the circularization of RNAs with different length of sequences of interest (cargos), various circular RNAs, include EGF, FGF1, RBD, G6PC, PAH, Luciferase and HGF, were generated in vitro using the AnaX circularization method and the circularization efficiency has been visualized on denature PAGE gel (FIG. 13). The results showed that the RNA with different length can be circularized by the AnaX system with high efficiency. However, the circularization efficiency decreases along with the increasing size of the cargos (FIG. 13). RNase R is a tool enzyme for verifying the circular structure of circular RNAs, and it can effectively degrade linear RNAs with bare ends, but not circular RNAs.

In order to compare the immunogenicity of the circular RNAs generated via AnaX carrying different sizes of cargos, 200 ng RNAs generated above has been transfected into A549 cells and equal amount of mRNAs with same coding sequences have been used as control. After 6 hours of transfection, the cells were collected, and Trizol reagent was added to extract the total RNA and the RT-qPCR was performed to determine the expressions level of IFNβ, TNFα, IL6 and RIG-I at mRNA level. As shown in FIG. 14, the circular RNAs are less immunogenic than their corresponding linear mRNAs with the same coding sequence, and the data are consistent with the conclusions in Example 2. IFNβ, TNFα, IL6 and RIG-I are commonly used marker genes for measurement of intracellular immunogenicity, and the positive control Poly (I:C) is a commonly used mimic of long double-stranded RNA virus stimulation.

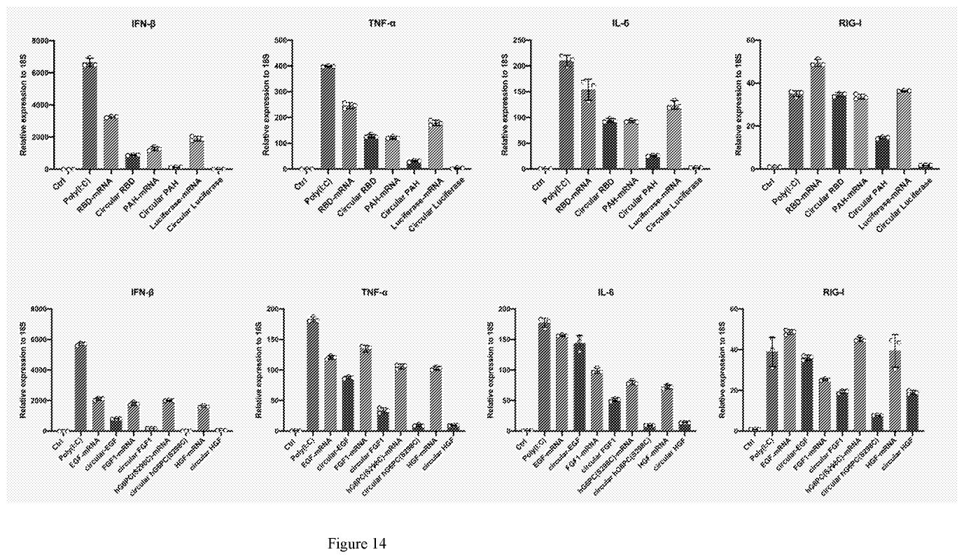

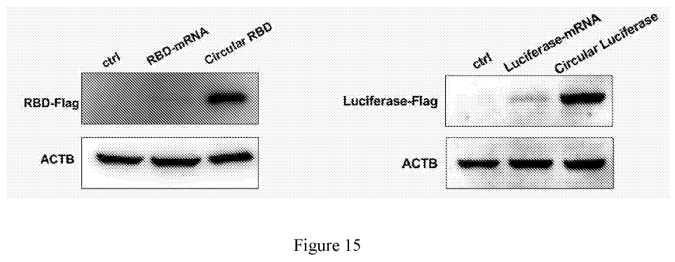

In order to test the intracellular protein translation efficiency of the circular RNA and linear RNA, the circular RBD and circular Luciferase synthesized by AnaX and their corresponding mRNAs with the same coding sequence were transfected with Lipofectamine MessengerMAX™ (Thermo Fisher) RNA transfection reagent into HEK293FT cells. The total cell protein was collected after 24 hours, and the level of translated protein was detected by Western Blot. The results showed that the circular RNA generated via AnaX had higher translation efficiency than the corresponding linear mRNA of the same coding sequence (FIG. 15).

In conclusion, this series of embodiments demonstrate that circular RNAs generated via AnaX has higher translation efficiency and lower immunogenicity than their linear mRNA counterparts. More importantly, compared with the circular RNA generated by Ana3.0 in Wesselhoeft et al., 2018 A. R., et al., (2018) Engineering circular RNA for potent and stable translation in eukaryotic cells., the circular RNA generated via AnaX retains fewer residual nucleotides and has lower immunogenicity.

### Example 5. Further Improvements to Residual Circularization Element Sequences in Circular RNA Generated by AnaX System

It has been reported that the stem-loop paired region of the exon anticodon region of natural Anabaena tRNALeu group I intron is necessary for the self-splicing reaction of Group I intron (Zaug, A. J., et al. (1993). Self-splicing of the group I intron from Anabaena pre-tRNA: requirement for base-pairing of the exons in the anticodon stem. Biochemistry 32(31): 7946-7953.). The stem-loop structure formed by the AnaX residual circularizing elements is proximal to the stem-loop structure in the natural anticodon region of tRNALeu and was a rationale design based on the principle that such structure is important in self-splicing reaction.

1. Interfering with the Pairing in the Stem of the Stem-Loop Structure of the Residual Circularizing Elements Leads to Weakened Circularization Efficiency

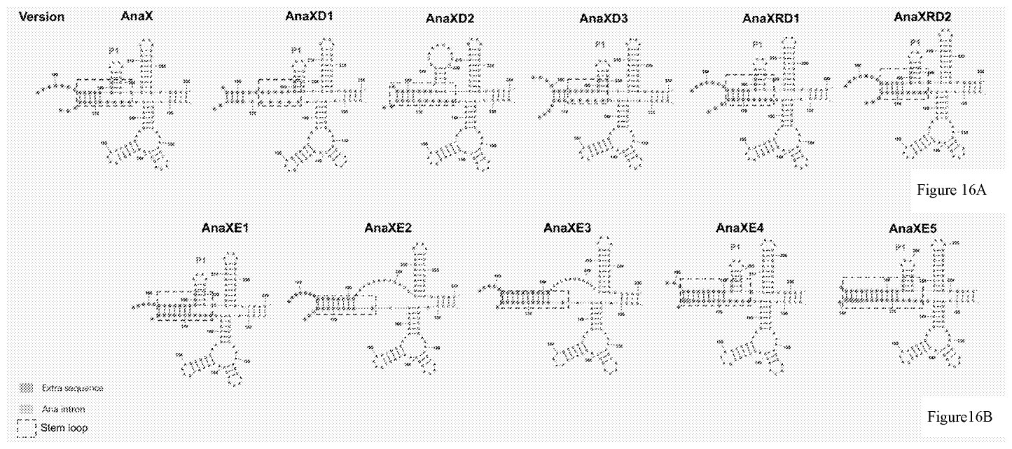

There are 5 base pairs (5 bp) in the stem-loop structure of the AnaX residual circularizing element. The mutant AnaXD1 introduced a G-A single-base mutation in the middle of the paired region of the first residual circularizing element at the 3′ end, and its corresponding rescuing mutant AnaXRD1 introduced a C-T mutation at the corresponding position of the second residual circularizing element at the 5′ end; the 5 nucleotides of the first residual circularizing element pairing region at 3′, namely, ACGGA are mutated to its complimentary counterpart UGCCU in AnaXD2 aiming to destroy the pairing region of the stem; the 5 nucleotides of the second residual circularizing element pairing region at 5′, namely, UCCGU are mutated to its complimentary counterpart AGGCA in AnaXD3 aiming to destroy the pairing region of the stem. The rescuing mutant AnaXRD2 carries the complimentary mutations simultaneously to form a pairing stem again in the stem-loop structure. According to the preliminary RNA secondary structure prediction, the mutations carried by AnaXD1 and AnaXD2 completely destroy the stem-loop structure of the feature element, AnaXD3 disrupts the original pairing (5 base pairs) to produce weaker structure with 3 base pairs at the stem. The rescuing mutants AnaXRD1 and AnaXRD2 can restore the residual circularizing element stem-loop structure (FIG. 16A).

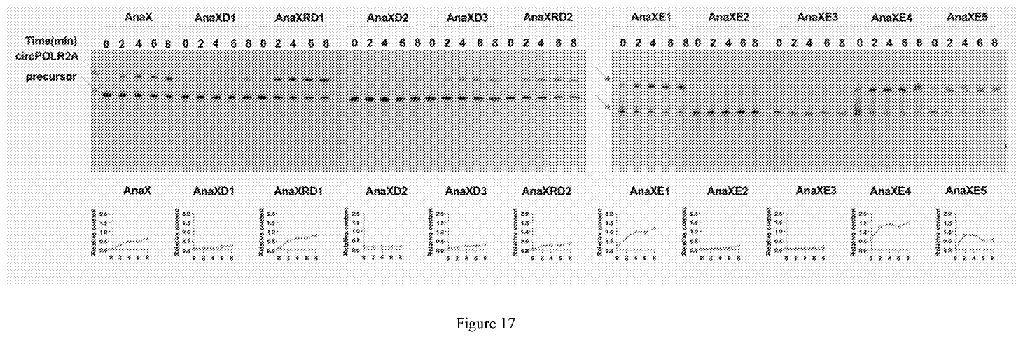

Taking the circularization of POLR2A as an example, the precursor RNA was generated by in vitro transcription in a buffer with low magnesium ion concentration (5 mM MgCl₂), and then in a self-splicing reaction buffer (50 mM Tris-HCl, 10 mM MgCl₂, 1 mM DTT, pH 7.5). The reaction was stopped by the addition of RNA denaturing loading buffer after incubation at 55° C. for certain amount of time. Denature PAGE showed the trend of the precursor and the circular RNA generated in the self-splicing reaction over time. The circular RNAs generated by the AnaXD1 and AnaXD3 mutants were reduced to about 30% of the AnaX version within 8 minutes of the circularization reaction. AnaXD2 mutant led to almost no circular RNA generated, while the circular RNA generation efficiency in the AnaXRD1 and AnaXRD2 mutants was rescued to various degrees compared with the above three mutants (FIG. 17).

In summary, base pairing at the stem of the stem-loop structure region of the AnaX residual circularizing element is necessary for the generation of circular RNAs from the circularization reaction.

### 2. Increasing the Number of Base Pairings in the Stem-Loop Structure of the Residual Circularizing Element Leads to Improved Circularization Efficiency

The mutant AnaXE1 carries a single mutation at the position distal from splicing site within the 3′ first residual circularizing element to increase the number of base pairs of the stem in the stem-loop structure from 5 to 7; the mutant AnaXE2 carries a single mutation at the position distal from splicing site within the 5′ second residual circularizing element to increase the number of base pairs of the stem of the stem-loop structure from 5 to 7; the mutant AnaXE3 carries more than 2 mutations to increase the base pairs of the stem at the stem-loop structure to 9 pairs; the mutant AnaXE4 carries the insertion of two additional nucleotides at the position distal from splicing site within the 5′ second residual circularizing element on top of AnaXE1 mutant to increase the base pairs of the stem of the stem-loop structure to 9 pairs; the mutant AnaXE5 carries the insertion of additional four nucleotides at the distal position away from splicing site within the 5′ second residual circularizing element on top of AnaXE1 mutant to increase the base pairs of the stem of the stem-loop structure to 11 pairs. AnaXE1, AnaXE4, and AnaXE5 enhanced pairing while maintaining the pairing of the first residual circularizing element proximal to the splice site with the intron, while AnaXE2 and AnaXE3 disrupted this pairing (FIG. 16B).

Taking the circularization of POLR2A as an example, the precursor RNA was generated by in vitro transcription in a buffer with low magnesium ion concentration (5 mM MgCl₂), and then incubated in a self-splicing reaction buffer (50 mM Tris-HCl, 10 mM MgCl₂, 1 mM DTT, pH 7.5). The reaction was stopped by the addition of RNA denaturing loading buffer after incubation at 55° C. for certain amount of time. The results of denature PAGE showed that the trend of dynamic changes of precursors and circular RNAs generated in the self-splicing reaction. The speed of circular RNA generation of AnaXE1, AnaXE4, and AnaXE5 was significantly faster than that of AnaX, while the speed of circular RNA generation of AnaXE2 and AnaXE3 was significantly reduced (FIG. 17).

To further verify the concept that increasing the number of base pairs at the position distal from the splice site within the residual circularizing element leads to improved circularization efficiency, firefly Luciferase RNAs with residual circularizing element of AnaX, AnaXE1 and AnaXE4 were generated via in vitro transcription and further subjected to circularization reaction. The circularization efficiency was then compared among these residual circularizing elements. Denature PAGE gel results showed that the amount of circular RNA products of AnaXE1 and AnaXE4 is more than AnaX with the same reaction condition and same reaction time (FIG. 19A, B).

In conclusion, increasing the number of complementary base pairs at the stem in the stem-loop structure of the residual circularizing elements can lead to improved circularization efficiency.

3. Replace the Sequence of Paired Bases of the Stem-Loop Structure within the Residual Circularizing Element to Artificially Designed Sequences to Enhance the Circularization Efficiency

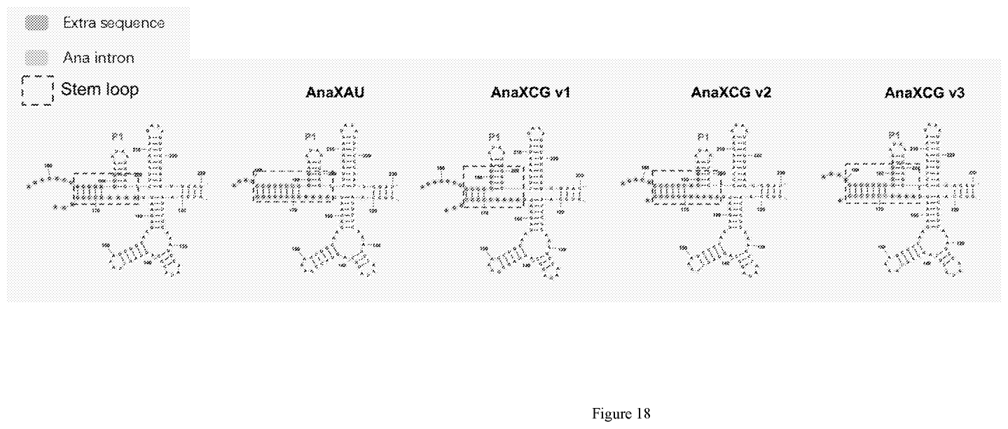

The stability of stem within the stem loop structure of residual circularizing element could be possibly increased with the increasing G/C ratio in the stem of the stem-loop structure. The AnaXAU mutants contain the majority of AU pairs in the stem and the minimal base pairs have to be at least 7 pairs to achieve the lower free energy in comparison to AnaX (FIG. 18). Base pairs at stem region of mutants AnaXCGv1, AnaXCGv2, and AnaXCGv3 were all changed to CG pairs. AnaXCGv1 is with 5 GC pairs, AnaXCGv2 and AnaXCGv3 are with 7 GC pairs. The 3′ first residual circularizing elements of AnaXCGv2 are all G, and AnaXCGv1 and AnaXCGv3 are all C. (FIG. 18).

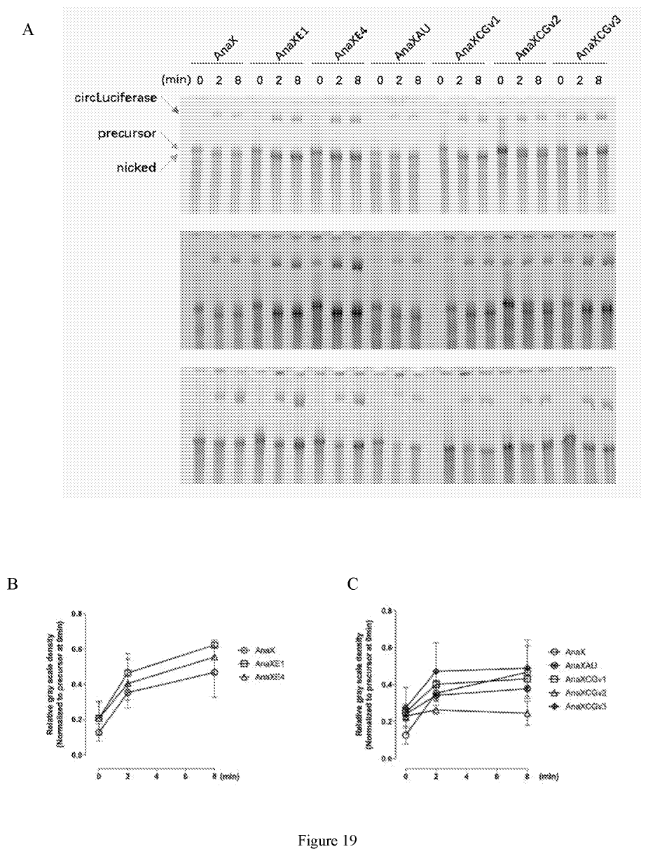

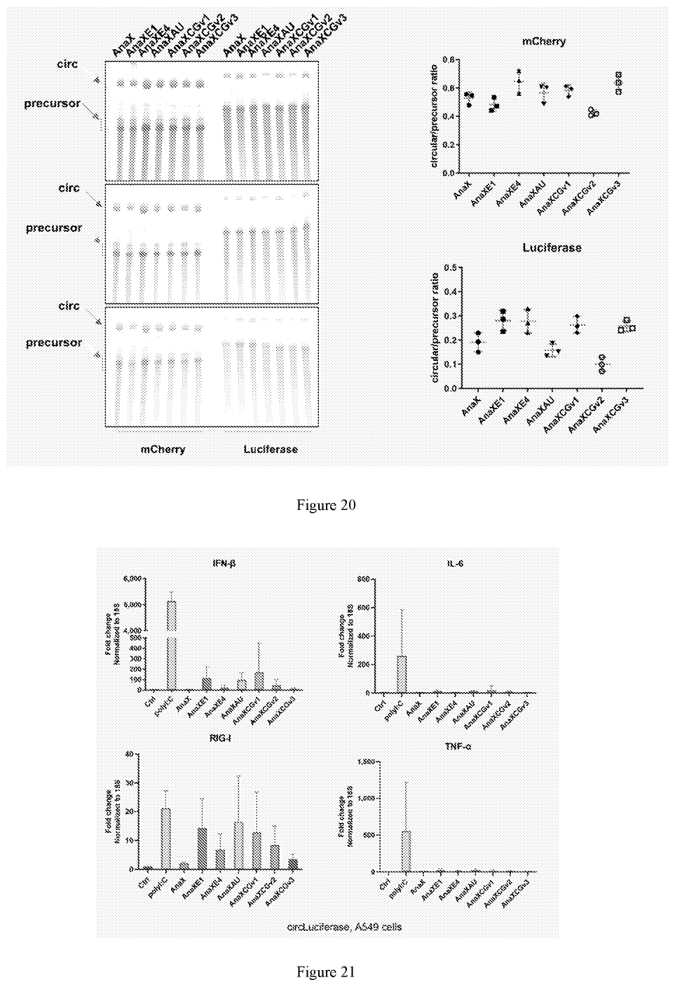

Taking the circularization of Luciferase RNA as an example, the precursor RNA was in vitro transcribed and then the self-splicing circularization reaction was performed. Denature PAGE gel results showed that AnaXCGv3 produced more circular RNA products than AnaX after 2 minutes and 8 minutes of reaction. The amount of circular RNA products of AnaXAU is similar to that of AnaX. The amount of circular RNA products generated by AnaXAU and AnaXCGv2 was less than that of AnaX (FIG. 19A, C). AnaXCGv1 and AnaXCGv3 led to higher circularization efficiency demonstrated by higher amount of circular RNA compared to AnaX, AnaXAU and AnaXCGv2 led to lower circularization efficiency demonstrated by lower amount of circular RNA compared to AnaX (FIG. 20) on denature PAGE gel. These data are consistent with the results described above.

The above rationale design of residual circularizing elements has also been applied to generate circular mCherry RNA. Denature PAGE gel results showed that the circularization efficiency of AnaXAU, AnaXCGv1 and AnaXCGv3 was higher than AnaX, and the efficiency of AnaXCGv2 was lower than AnaX (FIG. 20), indicating that the paired base composition of the stem-loop structure of the residual circularizing element has a significant effect on the circularization efficiency. The circularization efficiency may also be influenced by the length and base composition of the sequence of interest (cargo), but all the data still supported that AnaXCGv1 and AnaXCGv3 improves circularization efficiency.

In conclusion, the pairing sequence of the stem at the stem-loop structure of the AnaX residual circularizing element can be replaced with a rationally designed pairing sequence, and the AnaXCGv1 and AnaXCGv3 with all G/C pairs in the stem region of the stem-loop structure resulted in improved circularization efficiency in RNAs carrying different cargos in base composition and length.

### 4. Effects of Mutated AnaX Residual Circularizing Elements on Circular RNA Immunogenicity

In order to compare the immunogenicity of AnaX and the mutants. Luciferase RNAs containing AnaX, AnaXE1, AnaXE4, AnaXAU, AnaXCGv1, AnaXCGv2 and AnaXCGv3 were generated using in vitro transcription and circularization. The same amount (100 ng) of circular Luciferase containing various residual circularizing elements was transfected into human A549 cells. After 6 hours of transfection, the cells were collected and Trizol reagent was added to extract the total RNA of the cells for RT-qPCR detection of IFNβ, TNFα, IL6 and RIG-I at mRNA level. The results showed that the circular Luciferases containing AnaX, AnaXE4, and AnaXCGv3 had low immunogenicity, and the average up-regulation of IFNβ mRNA expression was within 25-fold, and the average up-regulation of RIG-I mRNA expression was within 7-fold. IL6 and TNFα stayed at very low level without any significant changes (FIG. 21). IFNβ, TNFα, IL6 and RIG-I are commonly used marker genes for measurement of intracellular immunogenicity, and the positive control Poly (I:C) is a commonly used mimic of double-stranded RNA virus stimulation.

### 5. Effects of Mutated AnaX Residual Circularizing Elements on the Translation Efficiency of Circular RNA

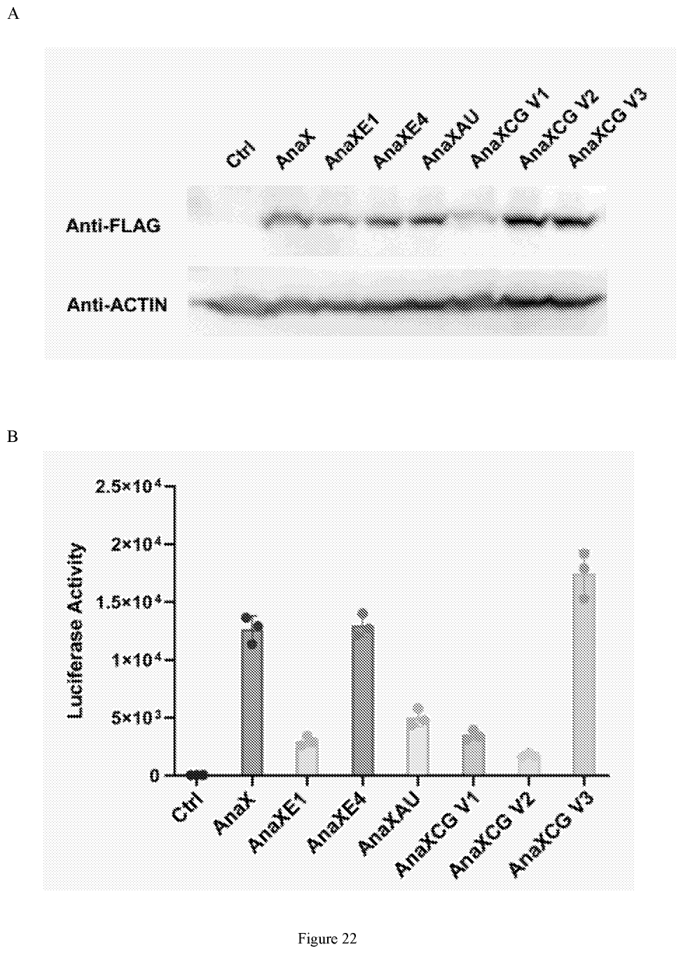

In order to compare the translation efficiency of the in vitro synthesized circular RNAs with AnaX residual circularizing element or versions of mutated AnaX residual circularizing elements, luciferase RNAs were produced via in vitro transcription and further subjected to circularization reactions. Circular Luciferase RNAs with residual circularizing elements of AnaX, AnaXE1, AnaXE4, AnaXAU, AnaXCGv1, AnaXCGv2 and AnaXCGv3 were tested for translation in HEK293FT cells, respectively. The same amount (200 ng) of in vitro circularized circular Luciferase was transfected into human HEK293FT cells. After 24 hours, the cells were collected to extract total protein samples. The Western Blot results showed that the Luciferase protein expression of AnaXCGv1 is lower than that of AnaX, the Luciferase protein expression of AnaXCGv2 and AnaXCGv3 is higher than that of AnaX, and the Luciferase protein expression of AnaXE1, AnaXE4, and AnaXAU was close to AnaX (FIG. 22A). Actin was used as a reference of Western Blot. In addition, the enzymatic activity of luciferase was measured using luminescence signal elicited by adding the corresponding substrate. The luminescence signal reflects the expression level of luciferase in the cells. The results showed that luminescence signal of circular Luciferase containing residual circularizing elements from AnaXE4 and AnaX were similar. The luminescence signal intensity of AnaXCGv3 circular Luciferase was higher than that of AnaX, and the luminescence signal intensity of the other constructs was significantly lower than that of AnaX (FIG. 22B).

In summary, the stem-loop structure formed by the residual circularizing elements of circular RNA, namely, AnaX and the mutations are important for a high circularization efficiency. Keeping the loop structure conformation and increasing the number of base pairs of the stem/or strengthening the base pairing (i.e. G/C pairs v.s. A/T pairs) would improve the circularization efficiency. In addition, such residual circularizing elements would also lead to improved translation efficiency and lowered cellular immunogenicity.

### 6. Changing the Specific Sequence of the Loop in the Stem-Loop Structure of the Residual Circularizing Element can Inhibit Circularization

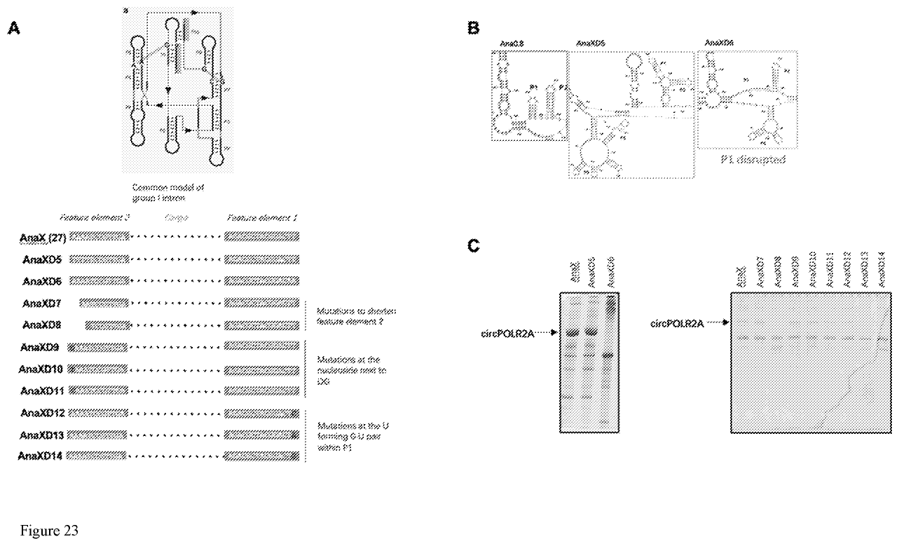

The 1st residual circularizing element of Anabaena Group I intron, the 3 nucleotides proximal to the splicing site and the internal guide sequence (IGS) form the P1 double-stranded region of the ribozyme, and determine the 5′ splicing site. The second residual circularizing element and several bases proximal to the splice site are involved in forming the P10 duplex region of the ribozyme and determine the 3′ splicing site. These two partial sequences form the loop region in the stem-loop structure of the residual circularizing element. In order to explore the effect of the loop region in the stem-loop structure of the residual circularizing element on RNA circularization, different mutants (AnaXD5-AnaXD14) were designed for the loop region to determine the effect on circularization efficiency. Mutant AnaXD 5 carries the nucleotide changes from AAAA to UUUU at loop region of the stem loop structure of the second residual circularizing element; mutant AnaXD 6 carries the nucleotide changes from CUU to CAA at the at loop region of the stem loop structure of the first residual circularizing element; mutant AnaXD 7 is with the changes of AAAA at the second residual circularizing element loop region truncated to AA; mutant AnaXD 8 is with the changes of removal of AAAA at the loop region of the second residual circularizing element; mutant AnaXD 9 is with the change of AAAA at the loop region of the second residual circularizing element to GAAA; mutant AnaXD 10 is with the change of AAAA at the loop of the second residual circularizing element to UAAA; mutant AnaXD 11 is with the change from AAAA to CAAA at the second residual circularizing element loop region; mutant AnaXD 12 is with the change of CUU to at the first residual circularizing element loop region CUC; mutant AnaXD 13 is with the change of CUU at the first residual circularizing element loop region to CUA; mutant AnaXD 14 is with the change of CUU at the first residual circularizing element loop region to CUG (FIG. 23A).

Taking the circularization of POLR2A as an example, if the loop of the stem-loop structure of AnaX P1 region has been destroyed in the mutants AnaXD 6, AnaXD 13 and AnaXD14, the RNAs cannot be circularized. In the mutant AnaXD12, CUU in the loop region of the first residual circularizing element was changed to CUC, and the GU pairing at the stem loop of the AnaX system P1 was converted into a GC pairing, therefore, maintained the original conformation without influencing the circularization. The mutants AnaXD 5, AnaXD 7, AnaXD9, AnaXD10 and AnaXD11 with mutated or truncated the bases in the loop region of the second residual circularizing element still kept the circularization efficiency. However, complete removal of all nucleotides in the loop region of the second residual circularizing element (mutants AnaXD8) leads to significant reduction of circularization efficiency (FIG. 23 B, C).

In summary, the P1 structure destroyed by the mutations in the loop region of the first residual circularizing element leads to the failure of circularization of RNA. The nucleotides in the loop region of the second residual circularizing element function as linkers. Mutation or truncation does not affect circularization, but nucleotides cannot be completely deleted in the loop region.

### Example 6. Influence of the Stem Length of AnaX Residual Circularizing Element on Circularization

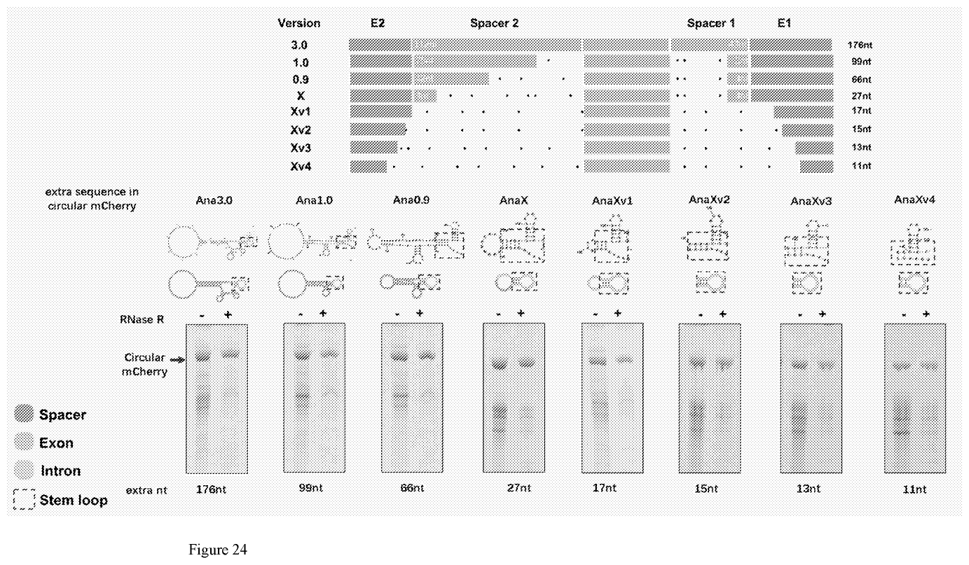

The minimum number of base pairings required for circularization has been explored in the stem region of the stem-loop structure of AnaX residual circularizing element. The loop structure of the stem-loop region around the splicing site has been kept with a serial of truncations of the residual circularizing element to 17, 15, 13 and 11 nucleotides and the stem base pairs are 5 pairs, 4 pairs, 3 pairs, and 2 pairs, respectively. The truncations are named as AnaXv1, AnaXv2, AnaXv3, and AnaXv4, respectively. Taking the circularization of mCherry RNAs as an example, the above truncated versions all retain the circularization capability (FIG. 24). It is worth noting that the 5′ and 3′ ends of the sequence of interest form a structure with 3 base pairs, which may compensate for the truncation of the stem of the residual circularizing element. The above results indicate that the circularization capability of the AnaX can still be kept, if keeping the loop conformation in the stem-loop structure and minimizing the number of base pairings in the stem.

### Example 7. The Alterations of Homology Arm Sequence Outside the Self-Splicing Intron Fragment of the AnaX System

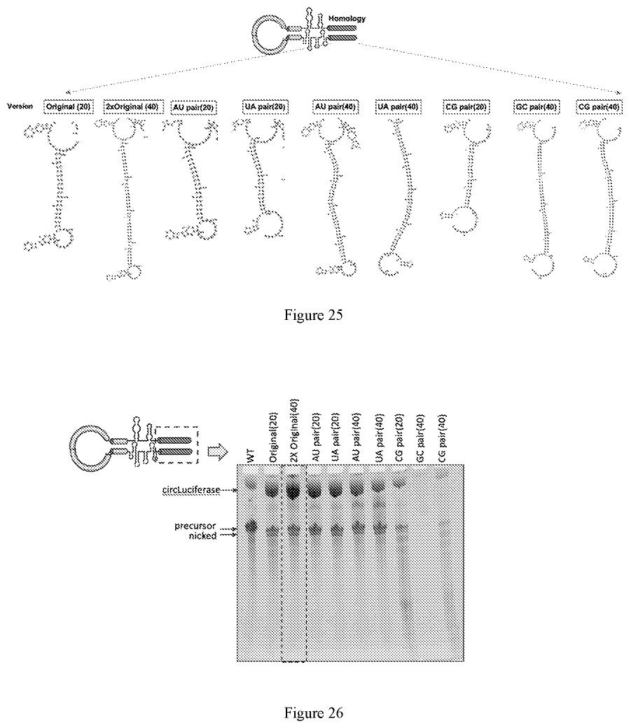

As shown in FIG. 25, as the length of the sequence of interest (cargo) increases, the circularization efficiency of the AnaX will be decreased. In order to improve the circularization efficiency of larger RNA cargos, we changed the homology arm sequence outside the intron fragment of the AnaX system, including 1) increasing the length of the homology arm sequence; 2) replacing the random sequence of the homology arm with AU bases pairing sequence; 3) replacing the random sequence of homology arm with UA base pairing sequence; 4) replacing the random sequence of homology arm with AU base pairing sequence and increase the number of base pairs; 5) replacing the random sequence of homology arm with UA base pairing sequence and increase the number of base pairs; 6) replacing the homology arm random sequence with a CG base pairing sequence; 7) replacing the homology arm random sequence with a GC base pairing sequence and increase the number of base pairs; 8) replacing the homology arm random sequence with a GC base pairing sequence and increase the number of base pairs; 9) removal of the homology arm sequences (FIG. 25).

In order to examine the effect of the retrofitted homology arm sequence outside the intron fragment on the circularization efficiency of the AnaX system, taking the circularization of luciferase RNAs as an example, and detected the circularization efficiency by denature PAGE gel. The result showed that increasing the length of the random base-pairing sequence of the homology arm significantly improved the RNA circularization efficiency. However, when the homology arm sequence was changed to AU/UA pairing, RNA circularization efficiency had no obvious effect. On the contrary, increasing the number of AU/UA pairings will reduce the RNA circularization efficiency. While the homology arm sequence is changed to GC/CG pairing, it will affect the in vitro transcription efficiency, and inhibit the RNA circularization efficiency (FIG. 26).

In conclusion, increasing the length of homology arm sequence outside the intron fragment can significantly improve the RNA circularization efficiency of the AnaX catalytic intron, but if the homology arm sequence on the outside is the poly GC pairing, the in vitro transcription efficiency will be seriously affected, thereby reducing the amount of circRNA generated.

### Example 8. Circularization Efficiency and Improved Residual Circularizing Element of Azoarcus Group I Intron

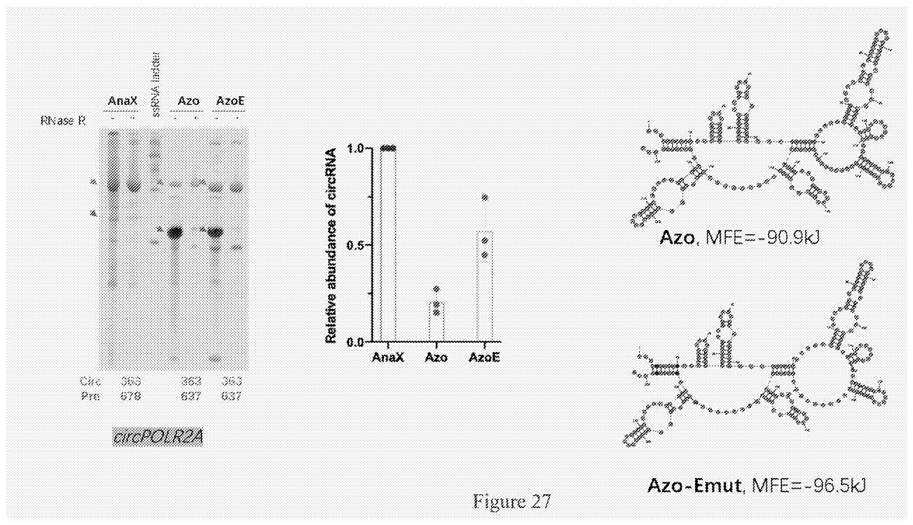

To further verify the importance of the stem-loop in the residual circularizing elements on the circularization of Group I Intron, we tested the circularization efficiency of another wild-type Group I Intron. The sequence of residual circularizing elements has been mutated to verify the effect on the circularization efficiency. Taking circular POLR2A as an example, the results of denature PAGE gel showed that the wild-type Azoarcus Group I Intron could lead to circularization and generate the circular RNA, but its circularization efficiency was lower than that of the improved AnaX. However, changing nucleotides of the stem region in the stem-loop of the residual circularizing elements of Azoarcus Group I Intron, that is, changing the original AU pairing to GC pairing, enhanced the stability of the stem structure and significantly improved the circularization efficiency of Azoarcus Group I Intron (FIG. 27). These results indicated that the stem-loop structure in the residual circularizing element of Group I Introns plays an important role in circularization, and improving the stability of the stem structure can improve the circularization efficiency.

### Example 9. IRES can be Located Downstream of Protein Coding Sequences in the AnaX System

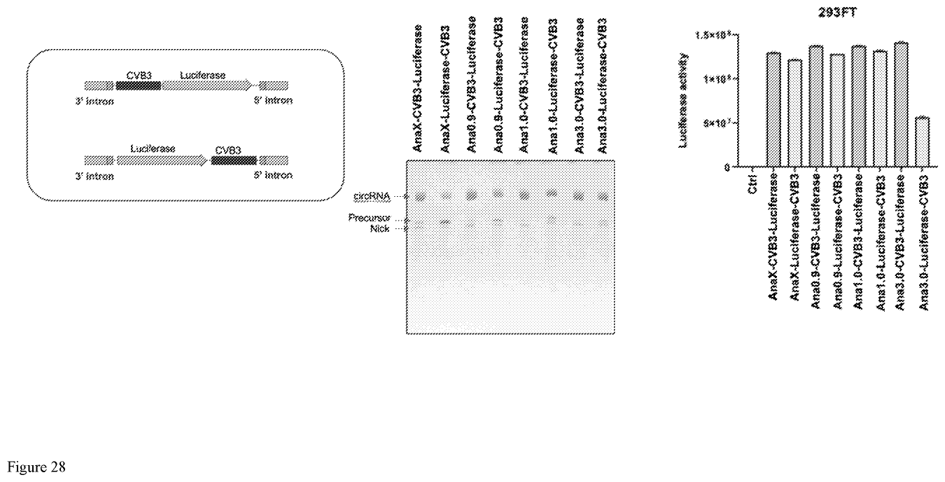

In order to detect the effect of placing IRES downstream of the protein coding sequence on protein translation in circular RNA, taking Luciferase as an example, IRES (CVB3) was located either upstream or downstream of the Luciferase coding sequence in the AnaX, Ana0.9, Ana1.0 and Ana3.0, respectively. Denature PAGE gel results showed that all the RNAs could be circularized with similar efficiency regardless of location of IRES at either upstream or downstream of the protein coding region. Subsequently, the circular Luciferases of different Anabaena group I introns were purified via gel excision from denature PAGE. Equal amount (200 ng) of circular Luciferase RNA was transfected into human HEK293FT cells using Lipofectamine MessengerMAX™ (Thermo Fisher) RNA transfection reagent. The intensity of the luciferase protein reacting with the substrate was used to reflect the luciferase protein expression level, thus, RNA translation efficiency. The results showed that with the AnaX, Ana0.9 and Ana1.0, IRES did not affect protein translation either placed upstream or downstream of the protein coding region. However, for Ana3.0, IRES placed downstream of the protein coding sequence can significantly inhibit protein translation. It is possible that the strong structure formed by the residual circularizing feature elements in the Ana3.0 system hindered the rate of ribosome movement (FIG. 28), thus, leading to low protein translation efficiency.

### Example 10. Improvement of Circular RNA Preparation Method

This Example demonstrated that an improved circular RNA preparation method which generates the circular RNA mCherry efficiently when synthesized in vitro via in vitro transcription followed by the self-splicing method of AnaX, and the circular RNA prepared by this method can be properly translated to protein (FIG. 29).

Taking the circular RNA mCherry synthesized in vitro as an example, the circular mCherry was prepared by in vitro transcription and self-splicing of the AnaX. The improved circular RNA preparation method specifically includes the following steps:
1. Plasmid construction and in vitro transcription: The template of circular RNA mCherry was obtained by PCR. The complete linear sequence with the T7 promoter at the 5′ terminus was inserted into the pUT7 vector through a multiple cloning site, and the recombinant plasmid was verified by Sanger-sequencing.
2. 1 μg of PCR-amplified linear template sequence with T7 promoter at the 5′ terminal, was incubated with 2 μl of T7 RNA Polymerase (Promega), rATP, rCTP, rUTP, rGTP (1 mM each) and buffer containing 20 mM Mg²⁺ at 37° C. for 3.5 hours to obtain a primary RNA mix product containing the linear precursor RNA with an Anabaena I intron self-splicing sequence at the 5′ and 3′ termini and the circular mCherry after self-splicing.
3. The circular RNA was analyzed and then purified by denaturing PAGE gel.

In order to determine the circularization efficiency of the improved circular RNA preparation method, taking the mCherry RNA as the example, we used denature PAGE gel to analyze the circularization efficiency of mCherry RNA primary product obtained by improved circular RNA preparation method or mCherry RNA primary product obtained by original circularization method. The results indicating that the circularization efficiency (25.5%) of circular RNA obtained by the improved circular RNA preparation method was basically the same as the original method (27.9%) (FIG. 29).

In order to further test the translation efficiency of the circular RNA prepared by improved method described above in cells, 200 ng of the circular mCherry prepared by improved method was introduced into the human HEK293FT cells using Lipofectamine MessengerMAX™ (Thermo Fisher) RNA transfection reagent. Fluorescence imaging microscope was used to detect the red fluorescent signal of mCherry protein (FIG. 29) to confirm the translational level of the circular mCherry prepared by improved method. The result showed the circular mCherry product prepared by the improved method can translate red fluorescent protein in cells, and the translation efficiency of red fluorescent protein is the similar as that of the original method.

In conclusion, the improved method for preparing circular RNA, is more convenient than the original method while keeping substantially the same circularization efficiency. The improved method has a significant application potential for large-scale preparation of circular RNA.

### Example 11. Improved In Vitro Transcription Conditions can Effectively Improve the Circularization Efficiency

This Example demonstrated that improving the in vitro transcription conditions can effectively increase the total RNA yield and RNA circularization efficiency.

### 1. Increasing the Concentration of Mg²⁺ can Promote In Vitro Transcription Efficiency and Circularization Efficiency

To further optimize the circularization efficiency, taking the circular mCherry generated by AnaX system as an example, after in vitro transcription (IVT) reaction was incubated at 37° C. for 3.5 hours, a gradient concentration of Mg²⁺ (0-500 mM) was added to the IVT product and further incubated at 37° C. for 0.5 hours. The total RNA yield and RNA circularization efficiency was analyzed by native agarose gel and denature PAGE. The results indicated that the amount of total RNA has a positive correlation with the additional Mg²⁺ concentration (FIG. 30). Furthermore, the circular mCherry and corresponding linear precursor RNA was also significantly increased (FIG. 30). In addition, using two other circular RNAs with different sequences, circular HGF and EGF, the similar results have been generated and confirmed that both total RNA yield and the circular RNA yield and circularization efficiency can be significantly increased under the condition of gradually increasing Mg²⁺ concentration.

In summary, by increasing the concentration of Mg²⁺, not only the yield of total RNA generation was improved, but also increased the circularization efficiency for generating circular RNAs.

### 2. Monovalent Metal Cations Promote In Vitro Transcription Efficiency and Increase the Total Yield of Circular RNA

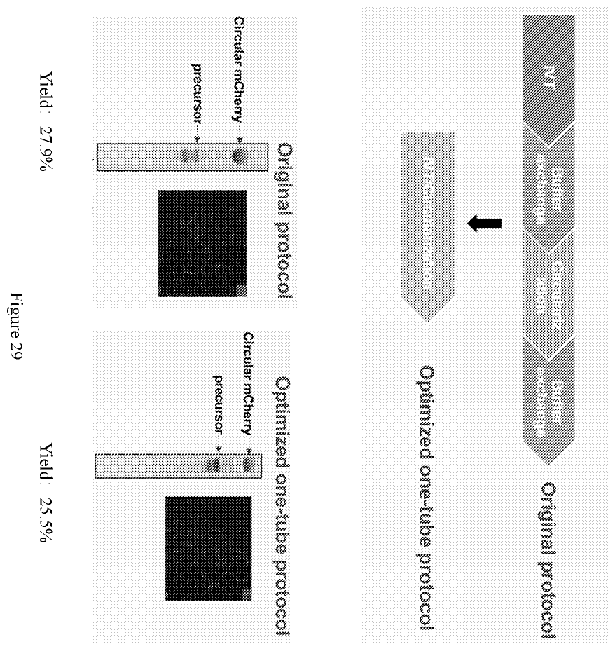

In order to further optimize the in vitro transcription and circularization efficiency, taking circular Luciferase RNA generated by AnaX as an example, during the in vitro transcription reaction, additional sodium ions (Na⁺) or potassium ions (K⁺) were added in a gradient concentration, and native agarose gel was used to analyze their effect on RNA in vitro transcription and circularization efficiency. The results showed that with the increase of Na⁺ concentration, the total RNA yield of in vitro transcription increased gradually. When the Na⁺ concentration reached 15 mM, the total RNA yield reached a plateau. While, elevated Na⁺ concentration did not affect the circularization efficiency of Luciferase RNA, but there was a significant increase in the total production of circular Luciferase RNA (FIG. 32). Like Na⁺, adding additional K⁺ can promote the efficiency of in vitro transcription without affecting RNA circularization. When the concentration of K⁺ reaches 90 mM, the in vitro transcription efficiency reaches a plateau (FIG. 33). These results indicated that monovalent metal cations, like Na⁺ and K⁺, can promote the efficiency of in vitro transcription without affecting RNA circularization, thereby increasing the total yield of circular RNA.

### 3. Monovalent Anions can Promote In Vitro Transcription Efficiency and Increase the Total Yield of Circular RNA

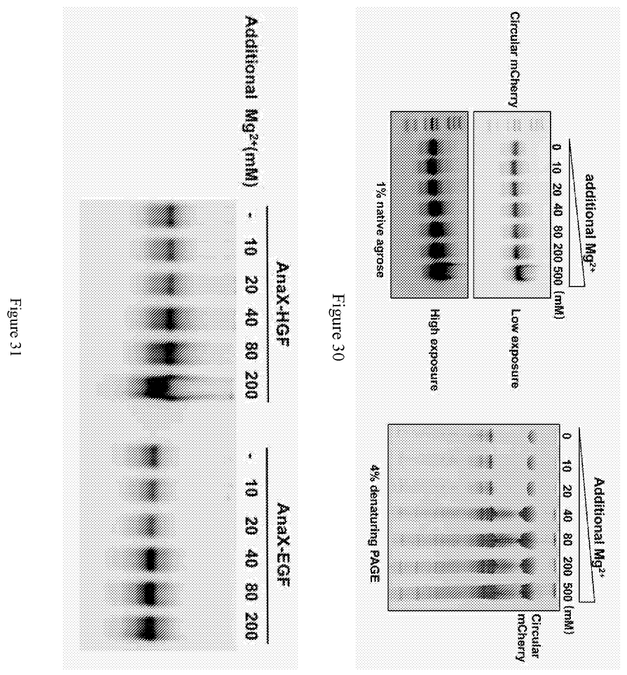

In order to further optimize the in vitro transcription and circularization efficiency, taking circular Luciferase RNA generated by AnaX as an example, during the in vitro transcription reaction, chloride ions (Cl⁻) or acetate ions (OAc⁻) in gradient concentrations were additionally added and detected the effects on the in vitro transcription and circularization efficiency by native agarose gel. The results showed that with the increase of Cl⁻ concentration, the total RNA yield of in vitro transcription increased gradually. When the Cl⁻ concentration reached 90 mM, the total RNA yield reached a plateau. While, elevated Cl⁻ concentration did not affect the circularization efficiency of Luciferase RNA, but there was a significant increase in the total production of circular Luciferase RNA (FIG. 33). Like Cl⁻, adding additional OAc⁻ can promote the efficiency of in vitro transcription without affecting RNA circularization (FIGS. 33 and 34). These results indicated that monovalent metal cations, like Cl⁻ and OAc, can promote the efficiency of in vitro transcription without affecting RNA circularization, thereby increasing the total yield of circular RNA.

### 4. The Effect of In Vitro Transcription Temperature on RNA Transcription and Circularization

As known in the art, 55° C. is a suitable temperature for RNA circularization. Therefore, in order to further simplify the RNA circularization step, we explored the effect of increasing the temperature on RNA in vitro transcription and whether there is a positive effect on enhancing circularization efficiency. Taking circular Luciferase RNA generated by AnaX as an example, we used thermostable T7 polymerase to perform in vitro transcription at different temperatures and subsequent circularization reactions, and detected the total RNA yield and circularization efficiency of Luciferase RNA produced by in vitro transcription by native agarose gel. The results showed that compared with the general temperature of 37° C., in vitro transcription could be performed at high temperatures of 50° C. and 55° C., but the transcription efficiency was significantly reduced, and it did not play a role in promoting the efficiency of RNA circularization (FIG. 35). These results showed that in vitro transcription and circularization reaction at the temperature that is suitable for RNA circularization cannot further improve the efficiency of RNA circularization, but inhibit the RNA in vitro transcription efficiency.

### 5. The Effect of In Vitro Transcription Duration on RNA Transcription and Circularization

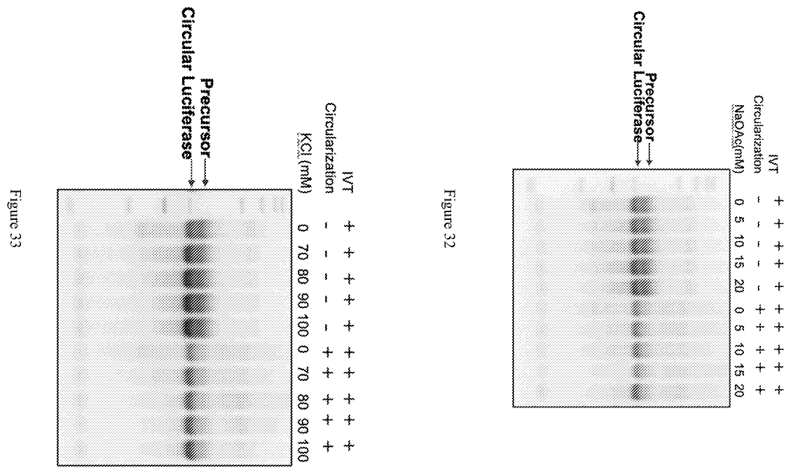

To examine the effect of in vitro transcription duration on transcription and circularization efficiency, circular Luciferase and circular IL2 RNAs were used as examples to compare the in vitro transcription efficiency and circularization efficiency at different time points by native agarose gel electrophoresis. The results showed that with the increase of in vitro transcription time, the circularization efficiency of circular Luciferase and circular IL2 was positively correlated with the length of in vitro transcription duration, but the transcription yields of circular Luciferase and circular IL2 were optimal at 5-7.5 hours (FIG. 36). Prolonged transcription may cause RNA degradation, resulting in a significant decrease in RNA transcription yield. These results indicated that extending the in vitro transcription time to a certain extent can promote RNA circularization and obtain high yields of RNA, but too long in vitro transcription may cause RNA degradation and reduce the total RNA yield.

### 6. Effects of In Vitro Transcription Buffer on RNA Transcription and Circularization

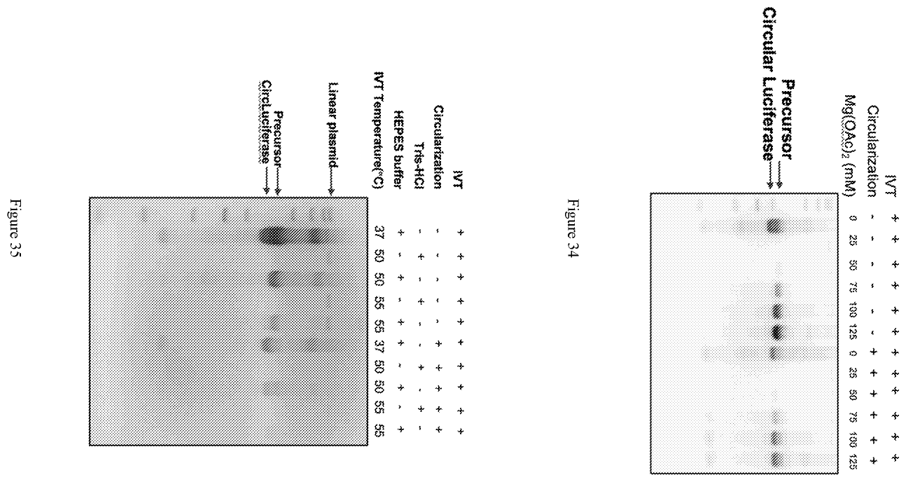

In order to examine the effects of different buffers on in vitro transcription and circularization efficiency, taking circular Luciferase RNA generated by AnaX as an example, during the in vitro transcription, HEPES and Tris-HCl buffers were tested for in vitro transcription and subsequent circularization reactions, respectively. The native agarose gel was used to compare the effects of different buffers on in vitro transcription efficiency and circularization efficiency. The results showed that HEPES and Tris-HCl buffer systems had the same in vitro transcription efficiency, but during in vitro transcription, circular Luciferase could achieve a higher circularization rate in HEPES buffer system than Tris-HCl buffer. These results indicated that HEPES and Tris-HCl buffer system did not affect the efficiency of in vitro transcription, but HEPES buffer was more favorable for circularization during in vitro transcription (FIG. 37).

### Example 12. Improved Circularization Condition can Effectively Improve Circularization Efficiency

This Example demonstrated that improving the circularization conditions in the circular RNA preparation process can effectively improve the circularization efficiency.

### 1. Divalent Metal Ions can Promote the Efficiency of Circularization

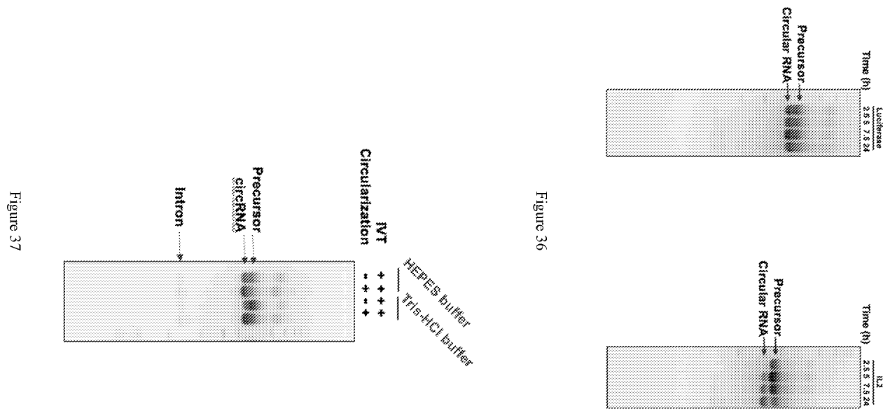

In order to further optimize the circularization efficiency, taking circular Luciferase RNA generated by AnaX as an example, during the circularization step, manganese (Mn²⁺) in gradient concentrations (0-50 mM) were tested. The corresponding circularization efficiency was analyzed the by native agarose gel. As shown in FIG. 38, increasing the concentration of manganese within a certain range improved the RNA circularization efficiency. Among them, the best concentration of Mn²⁺ is 20 mM, and further increasing the concentration beyond 20 mM cannot further improve the circularization efficiency. These results indicated that in the process of circularization, Mn²⁺ can promote the splicing of Group I Introns and improve the circularization efficiency, and the best Mn²⁺ concentration is 20 mM.

### 2. Influence of Temperature and Reaction Time on Circularization Efficiency

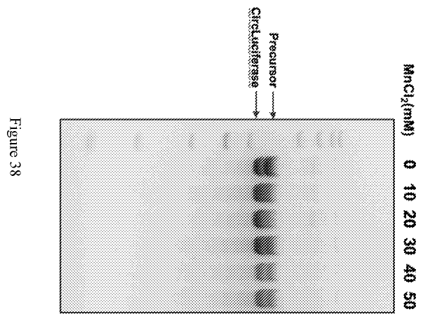

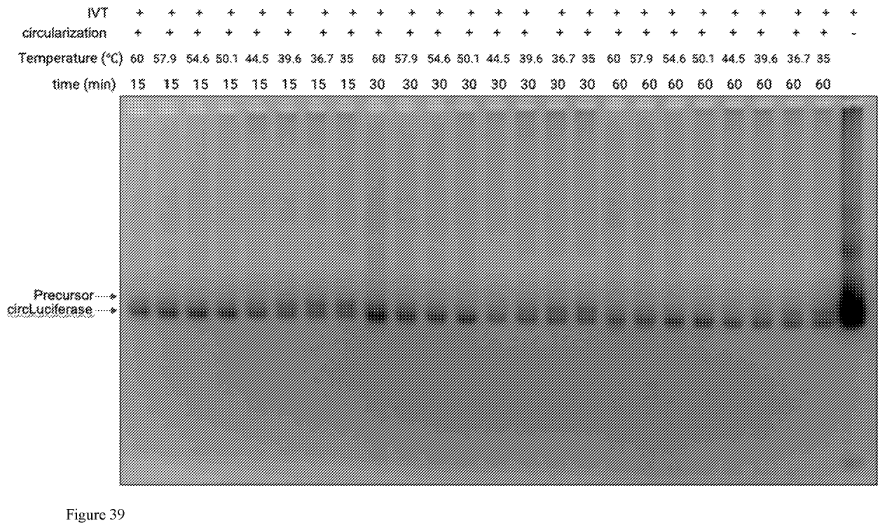

In order to explore the effect of reaction temperature and time on the circularization efficiency, taking circular Luciferase RNA generated by AnaX as an example, during the circularization step, different temperatures (35° C.-60° C.) and times (15-60 min) were tested. The corresponding circularization efficiency was analyzed by native agarose gel. As shown in FIG. 39, with increasing temperature, the circularization efficiency gradually increased, and began to reach the plateau at 50° C. When the temperature reached 60° C., the circularization efficiency began to decline. Different reaction time showed no significant difference for the circularization efficiency, that is, 15-30 min was sufficient for a complete circularization reaction. The above results showed that 50° C.-60° C. is the optimal temperature for the circularization reaction, and the reaction time is not the main factor affecting the circularization efficiency.

### 3. Influence of Buffer and pH on Circularization Efficiency

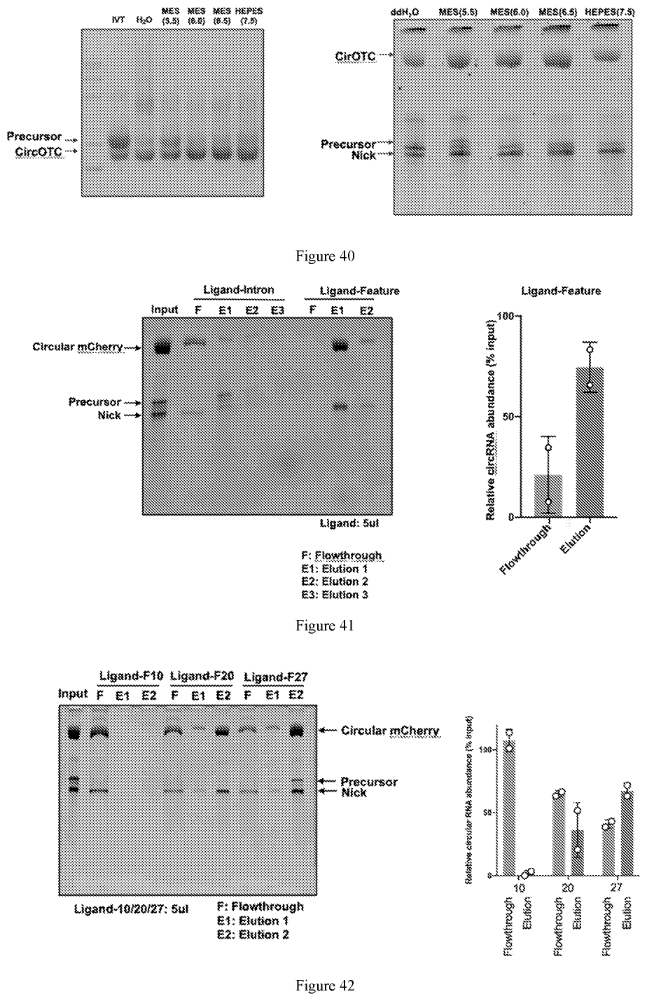

In order to explore the effect of different buffers and pH on the circularization efficiency, taking the circular OTC RNA as an example, HEPES and MES buffer with different pH (5.5-7.5) were tested for circularization. Native agarose gel and denaturing PAGE gel were used to analyze the effects of different buffers and different pH on the circularization efficiency. The results showed that the HEPES and MES buffer systems had similar in vitro transcription efficiencies at the PH above 6.0, however, at pH 5.5, the circularization efficiencies were significantly reduced. The above results indicate that both HEPES and MES buffers are suitable for in vitro circularization above pH 6.0, but low pH is not suitable to circularization (FIG. 40).

### Example 13. Novel Circular RNA Purification Method can Effectively Enrich Circular RNA

This Example demonstrated that the novel circular RNA purification method can effectively remove the linear precursor RNAs and cleaved-intronic RNAs, enrich and purify the circular RNA synthesized in vitro by the Group I self-splicing introns.

Taking the circular mCherry RNA as an example, the circular mCherry was prepared by in vitro transcription and further circularized using the AnaX self-splicing intron with 27 nucleotides as the residual circularizing element. The primary RNA product prepared by this circularization method comprises circular mCherry and a series of linear precursor RNAs and the cleaved-intronic RNAs during self-splicing and circularization. In order to efficiently enrich and purify the circular mCherry from the primary RNA product and remove the linear precursor RNAs and the cleaved-intronic RNAs, a complementary base paired DNA probe was designed to remove the linear precursor RNAs and intronic RNAs. The strategy of this purification method is different from the purification strategy of the existing technology. This method uses complementary paired DNA probes that only specifically bind to linear precursor RNAs and cleaved-intronic RNAs, then uses streptavidin beads to bind DNA probes to specifically remove linear precursor RNAs and intronic RNAs. Next, the RNA sample was incubated with the complementary base paired DNA probe that specific target to residual circularizing element of circular RNA, and enriched with streptavidin beads. Finally, eluted the circular RNA with elution buffer, and achieve the purpose of enriching and purifying the circular RNA.

Complementary base pairing DNA probes were designed for the intron in the linear precursor RNA and the residual circularizing element of circRNA, named as Ligand-Intron (SEQ ID NO:94), and Ligand-Feature (SEQ ID NO:95), respectively. The probes were synthesized by Shanghai Sangon Biotech and modified with Biotin (biotin). 20 μg of the primary circularization mCherry RNA product was placed to a 1.5 ml RNase-free centrifuge tube, and 5 μL (100 mM) of the above biotin-modified Ligand-Intron probe (the molar ratio of DNA probe to target RNA is 10⁰˜10⁵:1) was add, incubated at 68° C. for 10 minutes, and then placed at room temperature (˜25° C.) to cool down naturally for annealing, so that the biotin-modified DNA probe can effectively bind to the target precursor RNAs and cleaved-intronic RNAs. 200 μL of streptavidin beads (1 mg of beads can bind 200 pmol of biotin-modified DNA probes) was added to the above mixture, placed on a rotating shaker and incubated at room temperature (˜25° C.) for 15 minutes, and the supernatant after the incubation was collected. The supernatant was then placed in a new RNase-free centrifuge tube and collected as the flow-through fraction. Streptavidin beads was washed three times with BW buffer (5 mM Tris-HCl, 0.5 mM EDTA, 1M NaCl, 0.01% Tween-20), 100 μL of RNase-free ddH₂O added to the tube and the tube was placed in a water bath at 68° C. for 2 minutes to elute and collect the elution (three eluates were named E1, E2 and E3, respectively).

After the Ligand-Intron probe was used to remove the precursor RNAs and cleaved-intronic RNAs, the previously collected flow-through fractions were taken to further enrich and purify the circular RNA. 5 μL (100 mM) of the biotin-modified Ligand-Feature probes were added to the above flow-through fractions, incubated at 68° C. for 10 minutes, and cooled down at room temperature (˜25° C.) naturally to allow the biotin-modified DNA probes binds efficiently to the target circular RNA. 200 μL of streptavidin beads was added to the mixture, placed on a rotating shaker and incubated at room temperature (˜25° C.) for 15 minutes. After incubation, the supernatant was collected and placed in a new RNase-free centrifuge tube as the flow-through fractions. Streptavidin beads was washed three times with BW buffer (5 mM Tris-HCl, 0.5 mM EDTA, 1M NaCl, 0.01% Tween-20), 100 μL of RNase-free ddH₂O added to the tube, and the tube was placed in a water bath at 68° C. for 2 minutes to collect the eluate as the elution fraction (two eluates were named E1 and E2, respectively).

In order to test the purification efficiency of the novel circular RNA purification method, the above-mentioned circular RNA preparation primary product (Input), as well as the flow-through and elution fractions after enrichment and elution with different probes were collected, and were further subjected to the analysis on the denaturing PAGE gel. As the results shown in FIG. 41, the Ligand-Intron probe can effectively bind and remove the linear precursor RNAs and cleaved-intronic RNAs in the primary circularization RNA product. Thus, the efficiency of Ligand-Feature probe-specific enrichment and purification of circular mCherry is improved.

In summary, the novel circular RNA purification method described above can effectively remove the linear precursor RNA and the cleaved-intronic RNA, and then achieve the enrichment and purification of circular RNA.

### Example 14. Influence of the Length of Different Probes on Circular RNA Purification

As mentioned above, the Ligand-Intron probe can effectively bind and remove the linear precursor RNA and cleaved-intron RNA mixed in the initial circularization RNA product, while the Ligand-Feature probe can specifically enrich and purify the circular RNA. In order to further explore the length of probes for effective purification of circular RNAs, probes with different lengths, contained biotin labels, were designed and synthesized for purifying circular RNAs (Ligand-F10, SEQ ID NO. 96; Ligand-F20, SEQ ID NO. 97; Ligand-F23, SEQ ID NO. 98; Ligand-F25, SEQ ID NO. 99; Ligand-F27, SEQ ID NO. 100; Ligand-F29, SEQ ID NO. 101.). Using these series of probes with different lengths, the efficiency of these probes for purifying circular RNA was tested by taking circular mCherry RNA as an example.

First, the effect of three probes (Ligand-F10, Ligand-F20, and Ligand-F27) on circular RNA enrichment was examined. The experimental procedure was as follows: 20 μg of the primary circularization mCherry RNA product was placed into a 1.5 ml RNase-free centrifuge tube and 5 μL (100 mM) of the biotin-modified DNA probes was added. The tube was then heated to 68° C. for 10 minutes, and placed at room temperature (˜25° C.) for natural cooling down, so that the biotin-modified DNA probe can effectively bind to the target circular RNA. 200 μL of streptavidin beads were added to the above mixture and incubated on a rotating shaker at room temperature (˜25° C.) for 15 minutes. After incubation, the supernatant was collected and transferred to a new RNase-free centrifuge tube as the flow-through fraction. The streptavidin beads were wash three times with BW buffer (5 mM Tris-HCl, 0.5 mM EDTA, 1 M NaCl, 0.01% Tween-20), and 100 μL RNase-free ddH₂O water was add to the mixture and the tube was placed in a water bath at 68° C. for 2 minutes to collect the elution fraction (two eluates were named E1 and E2, respectively). The collected circular RNA preparation primary product (Input), flow-through and elution fractions were analyzed by denaturing PAGE gel electrophoresis. As shown in FIG. 42, two sets of probes, Ligand-F20 and Ligand-F27, can specifically enrich and purify circular mCherry, but the shorter Ligand-F10 cannot effectively enrich circular RNA. In addition, Ligand-F27 has a stronger efficiency of enriching circular RNA than Ligand-F20, but its enrichment and purification product also contained a certain linear precursor RNA and nick RNA.

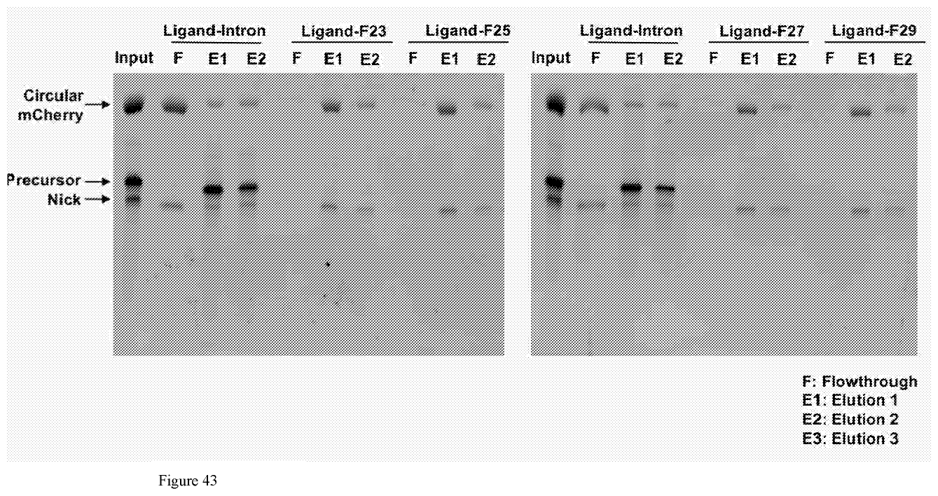

Further probe lengths (Ligand-F23, Ligand-F25, Ligand-F27 and Ligand-F29) were examined for their efficiency on circular RNA purification. Taking circular mCherry as an example, the specific experimental procedure is as described above. The above-mentioned circular RNA preparation primary product (Input), as well as the flow-through and elution fractions after enrichment and elution with different probes were collected, and analyzed by denaturing PAGE gel electrophoresis. As shown in FIG. 43, the Oligo-27 probe can specifically enrich and purify circular mCherry, Ligand-F23, Ligand-F25 and Ligand-F29 can specifically enrich and purify circular mCherry, indicating that probes with different lengths in the range of 23 nt-29 nt can be used for the novel circular RNA purification/enrichment method.

In summary, probes with a length of 10 nt or less have no effect on circular RNA purification or enrichment, while probes with the length in the range of 20-29 nt can be used to purify or enrich circular RNA.

### Example 15. The Circular RNA Purified by the Novel Purification Method can be Translated

This example demonstrated that the circular mCherry purified by the novel circular RNA purification method described above can be translated into red fluorescent protein in the cells.

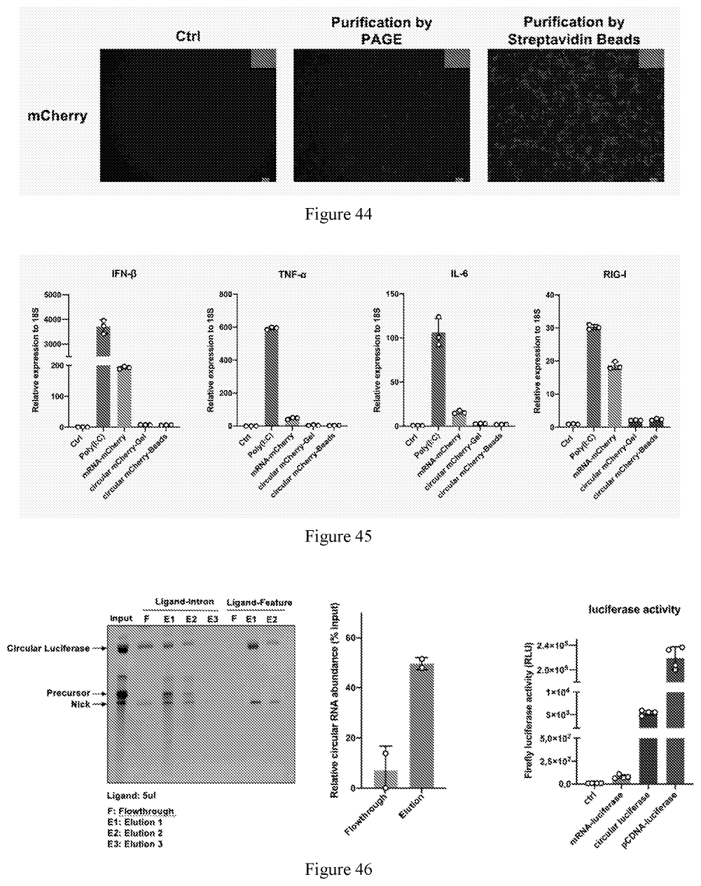

As mentioned above, the novel circular RNA purification method can efficiently enrich circular RNA and improve the specificity of enrichment and purification of circular RNA. In order to further test whether circular mCherry purified by the novel circular RNA purification method described above, can be translated into proteins in cells, 200 ng of circular mCherry purified by this method was introduced into the human HEK293FT cells using Lipofectamine MessengerMAX™ (Thermo Fisher) RNA transfection reagent. 200 ng of circular RNA purified by denature PAGE gel was used as a control and introduced into human HEK293FT cells at the same time. The red fluorescent signal of the translation product red fluorescent protein was detected by fluorescence imaging microscope (FIG. 44), confirming that the circular RNA product purified by this novel circular RNA purification method can be translated into the red fluorescent protein, and it is better than that obtained by purification via gel excision from denature PAGE gel.

### Example 16. Circular RNA Purified by the Novel Purification Method has Low Immunogenicity

This example demonstrated that the circular mCherry purified by the novel circular RNA purification method described above has low immunogenicity when introduced into human A549 cells.

As mentioned above, the novel circular RNA purification method can efficiently enrich circular RNA and improve the specificity of enrichment and purification of circular RNA. In order to further test the immunogenicity of circular mCherry purified by the novel circular RNA purification method described above, the same amount (200 ng) of circular mCherry purified by this method, circular mCherry and linear mCherry mRNAs purified by traditional gel excision from denature PAGE gel were transfected into human A549 cells, respectively. After 6 hours of transfection, the cells were collected, and Trizol reagent was added to extract the total RNA of cells for RT-qPCR detection of mRNA expression levels of cytokines IFNβ, TNFα, IL6 and RIG-I. As shown in FIG. 45, the immunogenicity of circular mCherry obtained by this novel purification method is similar with the circular mCherry purified by traditional denature PAGE, and both are significantly lower than linear mCherry mRNA. Among them, IFNβ, TNFα, IL6 and RIG-I are commonly used intracellular immunogenicity marker genes, and the positive control Poly (I:C) is a commonly used long double-stranded RNA virus mimic.

### Example 17. Using the Novel Circular RNA Purification Method to Purify Circular Luciferase RNA and the Purified RNA can be Translated

This example demonstrated that the novel circular RNA purification method described above can effectively enrich and purify circular Luciferase RNA, indicating that the method is not limited to the sequence of interest of the circular RNA.

As mentioned above, the novel circular RNA purification method can effectively enrich and purify circular RNA. The circular mCherry RNA purified by the novel circular RNA purification method, can be translated into proteins in the cells. In order to further exam whether the novel circular RNA purification method described above is limited by the sequence of interest of the circular RNA, another circular RNA with different sequence of interest, circular Luciferase RNA, was purified by this method. The specific experimental steps are similar to those in the previous example.

In order to test the efficiency of purifying circular Luciferase RNA by the improved new circular RNA purification method, the above-mentioned circular Luciferase RNA preparation primary product (Input), as well as Flow through and Elution after Ligand-Feature probe enrichment and elution were collected respectively. Components were subjected to the analysis of denaturing PAGE gel electrophoresis. The results are shown in FIG. 46, indicating that the method can effectively enrich circular Luciferase RNA and improve the specificity of enriching and purifying circular RNA (FIG. 46 shows the statistical results of purification efficiency).

Further, to test whether the circular Luciferase RNA purified by the novel circular RNA purification method described above can be translated into proteins in the cells, Lipofectamine MessengerMAX™ (Thermo Fisher) RNA transfection reagent was used to transfect equal amount (200 ng) of the purified circular RNA, the linear Luciferase mRNA and Luciferase plasmid into human HEK293FT cells. The luminescence intensity of the luciferase protein reacting with the substrate was used to reflect the protein expression level and the RNA translation efficiency. The results confirmed that the circular RNA product purified by this novel circular RNA purification method can be translated into the luciferase protein, and the translation efficiency is higher than that of the linear Luciferase mRNA (FIG. 46).

### Example 18. Using the Novel Circular RNA Purification Method to Purify Circular RNA Generated by T4 Ligase

This example demonstrated that the novel circular RNA purification method described above can effectively enrich and purify circular RNA synthesized by T4 Ligase, indicating that the method is not limited to the circularization method of generating circular RNA.

Taking circular POLR2A that generated by T4 Ligase circularization in vitro as an example. The primary product of circular POLR2A prepared by this method comprises circular POLR2A, unlinked linear precursor RNA and intermolecularly linked linear RNA. Complementary pairing DNA probe was designed for the circular RNA ligation site, named Ligand-Feature/ligase (SEQ ID No. 102), which was synthesized by Shanghai Sangon Biotech and modified with Biotin. 20 μg of the primary product of circular POLR2A was placed into a 1.5 ml RNA-free centrifuge tube, 5 μL (100 mM) of the above biotin-modified DNA probes was added. The mixture was incubated at 68° C. for 10 minutes, and placed at room temperature (˜25° C.) for naturally cooling down to allow the biotin-modified DNA probe to bind efficiently to the target circular RNA. 200 μL of streptavidin beads were added to the above mixture, placed on a rotating shaker and incubated at room temperature (˜25° C.) for 15 minutes. After incubation, the supernatant was collected and placed in a new RNase-free centrifuge tube as the flow-through fraction. Streptavidin beads were wash three times with BW buffer (5 mM Tris-HCl, 0.5 mM EDTA, 1 M NaCl, 0.01% Tween-20), 100 μL RNase-free ddH₂O was added and placed in a water bath at 68° C. for 2 minutes to collect the elution fraction (the two elution fractions were named E1 and E2, respectively).

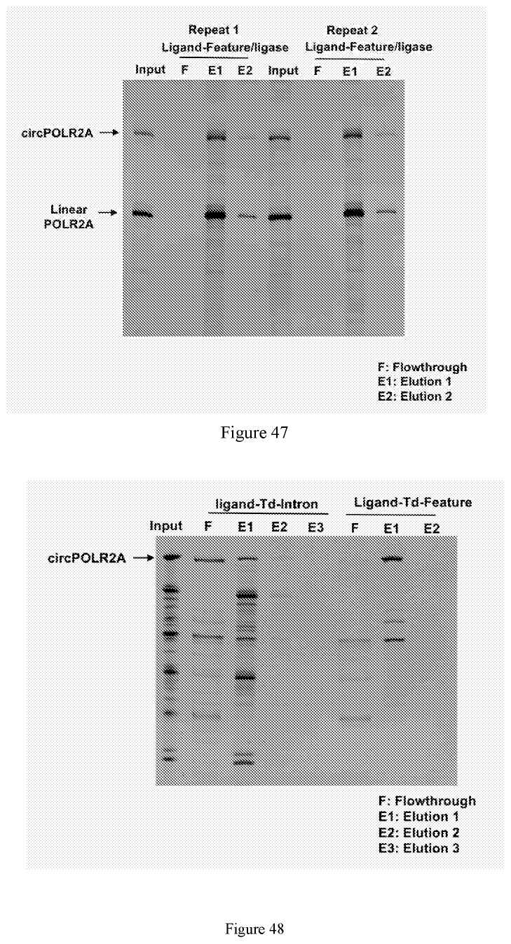

In order to test the purification efficiency of the novel circular RNA purification method on the circular RNA synthesized by T4 ligase, the above circular RNA preparation primary product (Input), flow-through (FT) and Elution fractions (Elution) were collected, respectively, and were subjected to denaturing PAGE gel electrophoresis. As shown in FIG. 47, the Ligand-Feature/ligase probe can enrich and purify circular POLR2A, but the enriched and purified product also contains a certain linear precursor RNA and intermolecularly linked linear RNA.

In conclusion, the novel circular RNA purification method described above has a certain enrichment or purification effect on circular RNA synthesized by T4 ligase.

### Example 19. Using the Novel Circular RNA Purification Method to Purify Circular RNA Generated by Td Group I Intron

This example demonstrated that the novel circular RNA purification method described above can effectively enrich and purify the circular RNA generated by using self-splicing Td group I intron for circularization, indicating that the method is not limited by the circularization method of generating circular RNA.

Taking circular POLR2A that generated by Td group I intron circularization in vitro as an example. The circular POLR2A primary product prepared by this method comprises circular POLR2A, a series of linear precursor RNAs, cleaved-intronic RNAs and linear nicked RNAs. Complementary paired DNA probes were designed for introns in linear precursor RNAs and residual circularizing elements of circular RNAs, named Ligand-Td-Intron (SEQ ID NO. 103), Ligand-Td-Feature (SEQ ID NO.104), respectively. They were synthesized by Shanghai Sangon Biotech and modified with Biotin (biotin). 20 μg of the primary product of circularized POLR2A was added into a 1.5 ml RNAse-free centrifuge tube, and 5 μL (100 mM) of the above biotin-modified Ligand-Td-Intron probe (the molar ratio of DNA probe to target RNA is 10⁰˜10⁵:1) was then added. Then the reaction was carried out at 68° C. for 10 minutes, then placed at room temperature (˜25° C.) to cool down naturally for annealing, so that the biotin-modified DNA probe can effectively bind to the target precursor RNA. 200 μL of streptavidin beads (1 mg of beads can bind 200 pmol of biotin-modified DNA probes) were added to the above mixture, placed on a rotating shaker and incubated at room temperature (˜25° C.) for 15 minutes. The supernatant was placed in a new RNase-free centrifuge tube as the flow-through fraction. Streptavidin beads were washed three times with BW buffer (5 mM Tris-HCl, 0.5 mM EDTA, 1 M NaCl, 0.01% Tween-20), 100 μL of RNase-free ddH₂O was added and the tube was placed in a water bath at 68° C. for 2 minutes to collect the elution fraction (three elution fractions were named E1, E2 and E3, respectively).

After the Ligand-Td-Intron probe is used to remove the precursor RNA, the previously collected flow-through fraction was taken to further enrich and purify the circular RNA. 5 μL (100 mM) of the biotin-modified Ligand-Td-Feature probes were added to the above flow-through fractions, incubated at 68° C. for 10 minutes, and placed at room temperature (˜25° C.) to naturally cool down for annealing, so that the biotin-modified DNA probe can effectively bind to the target precursor RNA. 200 μL of streptavidin beads were added to the above mixture, placed on a rotating shaker and incubated at room temperature (˜25° C.) for 15 minutes. After incubation, the supernatant was collected and placed it in a new RNase-free centrifuge tube as flow-through components. Streptavidin beads were washed three times with BW buffer (5 mM Tris-HCl, 0.5 mM EDTA, 1 M NaCl, 0.01% Tween-20), 100 L of RNase-free ddH₂O was added and the tube was placed in a water bath at 68° C. for 2 minutes to collect the elution fractions (two elution fractions were named E1 and E2, respectively).

In order to detect the purification efficiency of the novel circular RNA purification method on the circular RNA synthesized by the self-splicing of Td group I introns, the above-mentioned circular RNA preparation initial products (Input), flow-through (FT) and elution fractions were collected respectively, and analyzed by denaturing PAGE gel electrophoresis. As shown in FIG. 48, the Ligand-Td-Feature probe can be used to enrich and purify circular POLR2A, but the enriched and purified product still contained a certain portion of linear precursor RNAs and nick RNA.

In conclusion, the novel circular RNA purification method described above has a certain enrichment effect on the circular RNA synthesized by the self-splicing of Td group I introns.

### Example 20. Novel Circular RNA Purification Method Combined with Chromatography Column to Purify Circular RNA

This example demonstrated that the use of the novel circular RNA purification method described above combined with chromatography column can effectively remove linear precursor RNA, enrich and purify circular RNA, indicating this affinity-based purification can be realized without the limitation of the immobilization method and material.

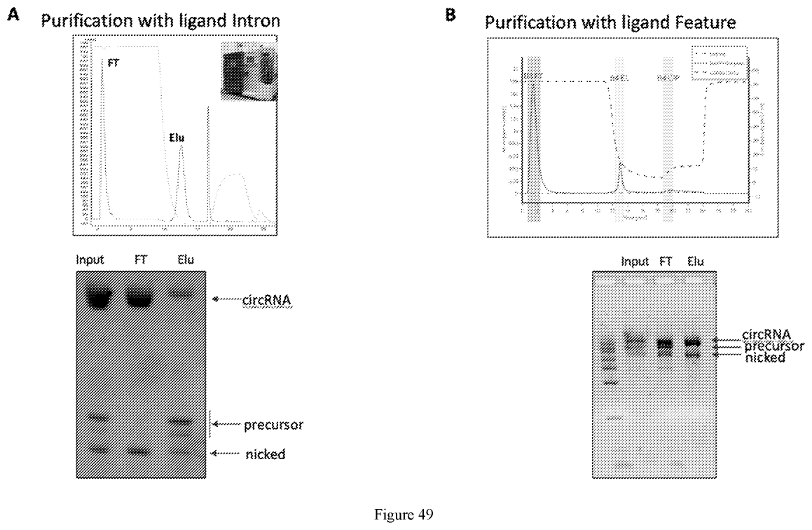

DNA probes targeting introns in linear precursor RNAs and residual circularizing elements of circular RNAs were immobilized on the chromatography column for removing precursor RNAs and enriching circular RNAs. The ligands immobilized to columns were named Ligand-Feature and Ligand-Intron, respectively. Taking the circular IL2 synthesized in vitro as an example, the removal efficiency of precursor RNA by Ligand-Intron chromatography column was examed. Circular IL2 was prepared by in vitro transcription followed by circularization reaction using AnaX self-splicing intron. The circular IL2 primary product prepared by the in vitro transcription comprises circular IL2, a series of linear precursor RNAs, cleaved-intronic RNA and nick RNAs in the process of self-splicing and circularization. 100 μg of circular IL2 circularization product (Input) was loaded to the Ligand-Intron chromatography column, flow-through (FT) was collected. TE buffer was then used to elute the Ligand-Intron chromatography column and the corresponding elution (Elution) was collect. Input, FT and Elution were loaded on denaturing PAGE gel for electrophoresis and the results showed that the Ligand-Intron column could effectively remove the precursor RNA (FIG. 49A).

Taking the circular RNA circular Luciferase synthesized in vitro as an example, the efficiency of enriching circular RNA by Ligand-Feature chromatography column was tested. Firstly, circular Luciferase was prepared by in vitro transcription using AnaX self-splicing method. The primary product of circular Luciferase prepared by in vitro transcription and circularization comprises circular Luciferase, a series of linear precursor RNAs, cleaved-intronic RNA and nick RNAs. 100 μg of the primary product of circular Luciferase (Input) via circularization was loaded the Ligand-Feature chromatography column, and the flow-through fraction (FT), elution fraction (Elution) were collected separately. Input, FT and Elution were loaded on agarose gel electrophoresis and the results showed that the Ligand-Feature column could effectively enrich circular RNAs (FIG. 49B).

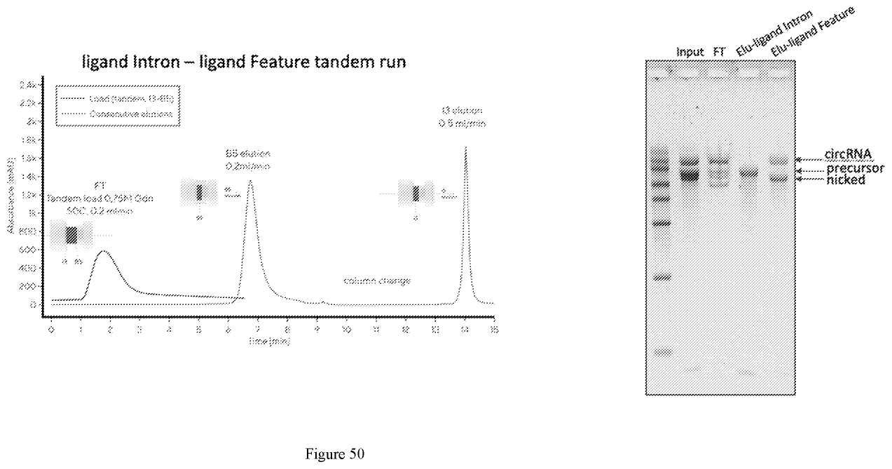

Taking the preparation of circular Luciferase using the self-splicing intron AnaX via in vitro transcription as an example, the efficiency of removing precursor RNA and enriching circular RNA after the combination of Ligand-Intron and Ligand-Feature columns was tested. Firstly, circular Luciferase was prepared by in vitro transcription and circularization. The primary product of circular Luciferase (Input) prepared by in vitro transcription and circularization comprises circular Luciferase, a series of linear precursor RNAs, cleaved-intronic RNA and nick RNAs. 100 μg of the primary product of circular Luciferase circularization was loaded to Ligand-Intron and the flow through directly run through Ligand-Feature chromatography columns. The final flow-through (FT) and elution fraction (Elution) were collected. Input, FT and Elution were analyzed by agarose gel electrophoresis. The Ligand-Intron column can effectively remove the precursor RNA, and the Ligand-Feature column can further enrich and purify the circular Luciferase (FIG. 50). With this new circular RNA purification method, the circular RNA can be efficiently enriched, and the rest of the linear components generated during IVT and circularization can be efficiently removed. Such ligands can be immobilized on solid supporting materials such as chromatography columns.

### Example 21. Purification of Circular RNA by Using Affinity-Based Purification with Oligo-dT as the Ligand and the Purified RNA can be Translated

### 1. Removal of Linear RNA Based on Oligo-dT Affinity Purification

This Example demonstrated the purification of circular RNA synthesized in vitro using the affinity purification method of oligo-dT. After the circularization reaction using of any circularization method, polyA can be added to the 3′-terminus of all linear RNAs in the circularization system using Poly A polymerase (e.g., E. coli. Poly A polymerase, yeast poly A polymerase, etc.). The oligo-dT affinity purification method removes all linear RNAs added with polyA tails and achieves the purpose of enriching and purifying circular RNAs.

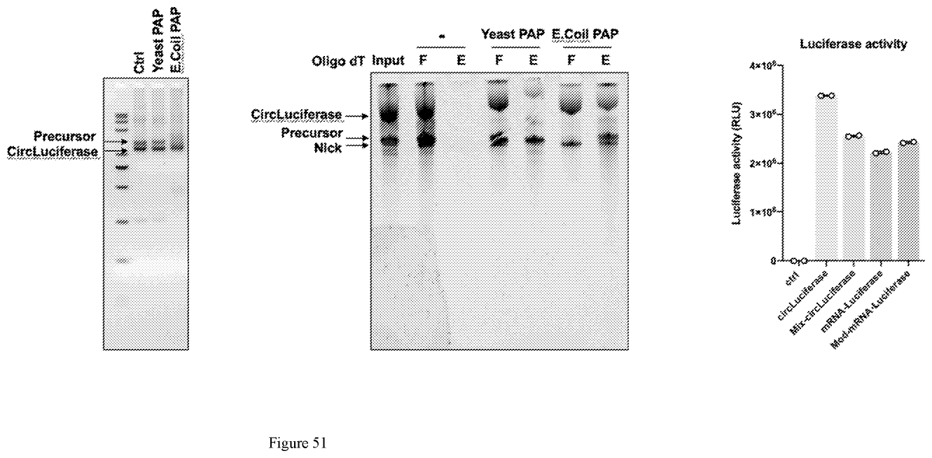

Taking the preparation of circular Luciferase RNA using the self-splicing intron AnaX via in vitro transcription as an example, after the circularization reaction is completed, poly A (20-500 nt) tail was added via E. coli. Poly A polymerase onto the 3′-termini of all linear RNAs in the reaction system (including the main linear precursor RNA, cleaved-intronic RNA and linear nick RNA). Agarose gel was used to analyze the result of poly A tailing of linear RNA, and the results show that compared with the sample without tailing reaction, the bands of all types of linear RNAs are significantly shifted upward. The results indicated that the majority of the linear RNAs were added with poly A tails (FIG. 51). Then, the Oligo-dT chromatography column was used to capture all the tailed linear RNAs via complimentary sequences to polyA tails. The circular RNAs flowed through the column, thus, got enriched and purified. The collected circular RNA from circularization reaction (Input), Flow-through and Elution components were analyzed by denature PAGE gel for the efficiency of enrichment and purification of circular RNA. The results showed that this method can effectively remove all linear RNAs containing Poly A sequences to enrich and purify the circular RNA (FIG. 51).

In summary, after the intron self-splicing and circularization reaction, Poly A tailing is performed, and the oligo-dT affinity purification method can be used to remove linear RNAs and achieve the effect of enriching and purifying circular RNA.

### 2. The Circular RNA Obtained by Oligo-dT Affinity Purification is Expressed in a High Level in Cells

Further, to test whether the circular Luciferase RNA purified by the purification method described above leads to an efficient translation to proteins in cells, Lipofectamine MessengerMAX™ (Thermo Fisher) RNA transfection reagent was used to transfect the RNAs into the cells. The equal amount (200 ng) of the purified circular Luciferase RNA by this method, unpurified circular Luciferase RNA, linear Luciferase mRNA and m1Ψ modified linear Luciferase mRNA were transfected into human HEK293FT cells, respectively. The luminescence signal of the luciferase protein reacting with the substrate was used to reflect and quantify the protein expression level and the RNA translation efficiency (FIG. 51). It is shown that the circular Luciferase RNA purified by this novel circular RNA purification method can be translated efficiently into the Luciferase protein, and the translation efficiency is higher than that of the linear Luciferase mRNA with either modified or unmodified nucleotides.

### Example 22. Advantages of Circular RNA Over Linear RNA

### 1. High Stability of Circular RNA In Vitro and In Vivo

This example demonstrated that circular RNAs exhibit better in vitro and in vivo stability than linear RNAs.

Taking the human endogenously expressed circular POLR2A RNA as an example, the in vitro synthesis and purification of circular RNA were carried out. The circular POLR2A sequence was obtained from the circexplorer database (http://yanglab.github.io/CIRCexplorer/), and the plasmids and related primers used to construct the template were synthesized by Shanghai Boshang Biological Co., Ltd. Circular RNA was prepared in vitro by in vitro transcription and Anabaena Group I intron autocatalytic circularization (Wesselhoeft et al., 2018 A. R., et al., (2018) Engineering circular RNA for potent and stable translation in eukaryotic cells.). High-purity circular POLR2A and linear POLR2A with the same sequence were purified by gel excision method from denature PAGE gel.

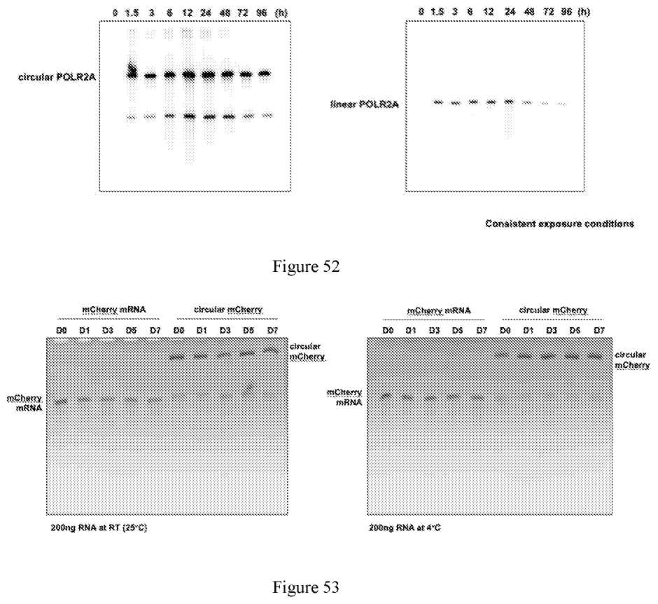

In order to test the intracellular stability of in vitro synthesized circular RNA and linear RNA, an equal amount (200 ng) of circular POLR2A and linear POLR2A were transfected into human HEK293FT cells using Lipofectamine MessengerMAX™ (Thermo Fisher) RNA transfection reagent, respectively. Next, at the time points shown in FIG. 52, HEK293FT cells transfected with circular POLR2A and linear POLR2A were collected, and Trizol reagent was added to extract the total RNA from the cells. Northern Blot was used to detect the amount of circular POLR2A and linear POLR2A in cells. The results (FIG. 52) showed that the linear POLR2A was degraded within 48 hours, and the circular POLR2A was more stable in cells and was not easily degraded.

To test the shelf-life of in vitro synthesized circular RNA and linear RNA at different temperatures, 200 ng of circular mCherry and 200 ng linear mCherry mRNA dissolved in RNase free ddH₂O and stored at 25° C. or 4° C., and the stability of the corresponding RNA after different day during was analyzed by denaturing PAGE gel electrophoresis. As shown in the results in FIG. 53, compared with day 0 (DO), the amount of RNA did not change significantly up till 7 days, indicating that RNAs in RNase-free ddH₂O are relatively stable at 25° C. or 4° C.

### 2. Long Half-Life and High Translation Efficiency of Circular RNA in Cells

This example demonstrated that in cells, circular RNAs have longer half-life and better translational efficiency than linear RNAs.

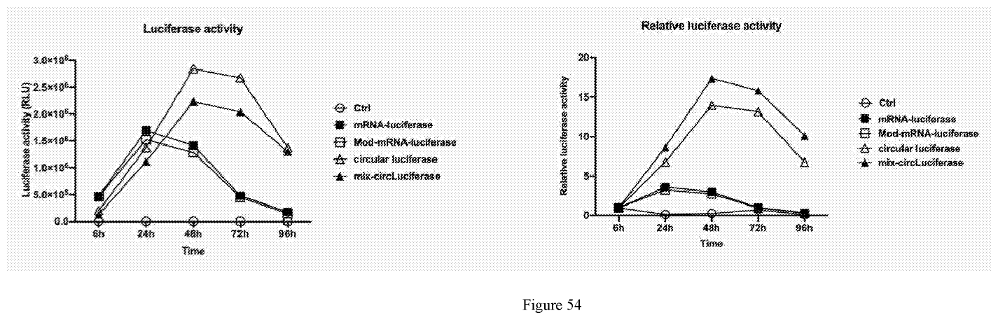

In order to test the translation efficiency of in vitro synthesis of circular RNA and linear mRNA in cells, same amount of purified circular Luciferase RNA, unpurified circular Luciferase RNA, m1Ψ modified linear Luciferase mRNA and unmodified linear Luciferase mRNA were transfected into human HEK293FT cells, respectively, using Lipofectamine MessengerMAX™ (Thermo Fisher) RNA transfection reagent. The elicited luminescence signal of luciferase upon adding the luciferin substrate was used to indicate the luciferase expression level thus to quantify RNA translational efficiency (FIG. 54). The luminescence signal was measured at different time points. As shown in FIG. 54, at 6 hours post transfection, the expression of linear RNA was higher than circular RNA, indicating translation initiation speed of linear RNA might be faster than that of circular RNA. At 24 hours post translation, the expression level of circular RNA was comparable to that of linear RNA. The circular RNA expression level continued to increase and was expressed stably until 72 hours. The expression level linear RNA peaked at 24 hours and decreased quickly to baseline at 72 hours. The results demonstrated that the circular Luciferase RNA was translated more efficiently thus produced more proteins in the cells and the protein expression is more stable than that of the linear mRNA.

3. Circular RNAs have Low Intracellular Immunogenicity

This example demonstrated the low immunogenicity of circular RNAs in cells.

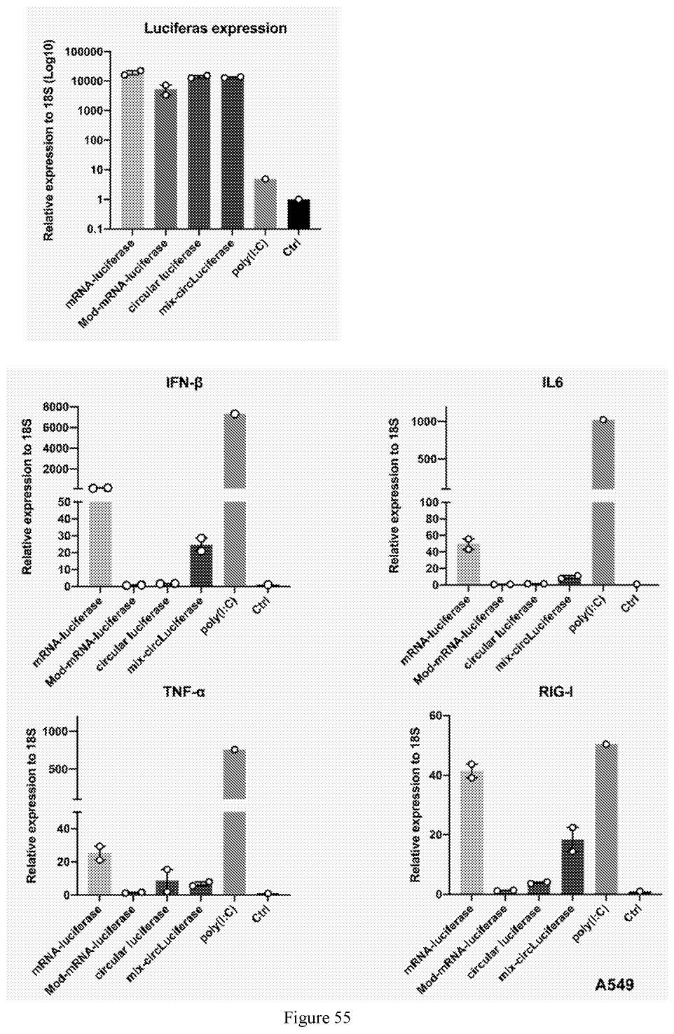

In order to measure the intracellular innate immune response caused by introduction of in vitro synthetic circular RNA and linear RNA into cells, the same amount of purified circular Luciferase (˜70% purity), unpurified circular Luciferase, m1Ψ modified linear Luciferase mRNA (>95% purity) and unmodified linear Luciferase mRNA (>95% purity) were transfected into human A549 cells using Lipofectamine MessengerMAX™ (Thermo Fisher) RNA transfection reagent. Six hours after transfection, the cells were collected and Trizol reagent was added to extract the total RNA of the cells for RT-qPCR to determine the mRNA levels of cytokines IFNβ, TNFα, IL6 and RIG-I. The transfection efficiency was monitored via measuring the level of the transfected Luciferase RNA by RT-qPCR. As shown in FIG. 55, compared with unmodified linear Luciferase mRNA, regardless of purification of circular RNA, the expression level of corresponding detected cytokines in A549 cells is lower. The cytokine level elicited by purified circular RNA was comparable to m1Ψ modified linear Luciferase mRNA, indicating that circular RNA has lower immunogenicity and better safety than linear Luciferase mRNA. Among them, IFNβ, TNFα, IL6 and RIG-I are commonly used intracellular immunogenicity detection marker genes, and the positive control Poly (I:C) is a commonly used double-stranded RNA virus mimic.

### 4. Circular RNAs Lead to Stable Expression In Vivo

This example demonstrated that more stable protein expression of circular RNA than linear mRNA when injected via intradermal delivery to mice in a naked form.

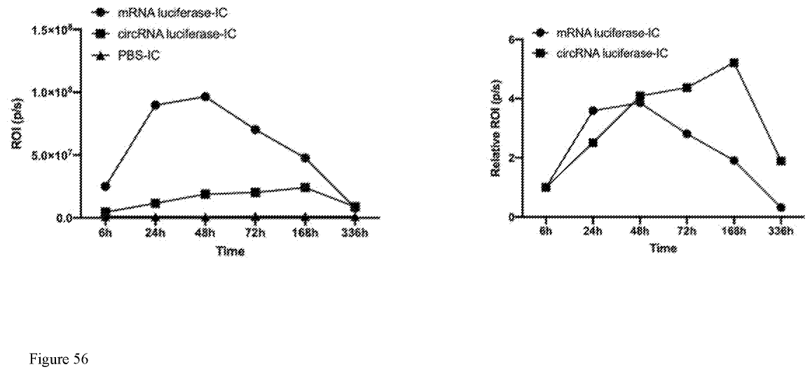

In order to test the in vivo performance of in vitro synthesized circular RNA and linear RNA, 5 ug of m1Ψ modified linear Luciferase mRNA and 5 ug of circular Luciferase RNAs in PBS solution were injected to mice via intradermal delivery. Live imaging by IVIS was taken to measure the luminescence signal elicited upon the injection of luciferin at different timepoints post injection of the RNAs to reflect protein expression level. The intensity of the luminescence signal was used to reflect the luciferase expression. At 6 hours post RNA injection, luminescence signal has been observed indicating the expression of the RNA. Additional time points were measured till 336 hours. The results indicated that linear Luciferase mRNA and circular Luciferase could be delivered intradermally without special formulation but with only PBS and the expression was sustainable in vivo for at least 168 hours, and circular RNA expression is more stable for at least 336 hours. (FIG. 56).

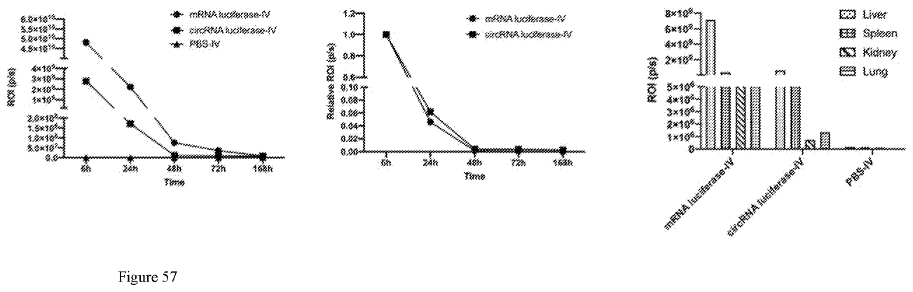

In order to detect the expression of lipid-nanoparticle (LNP)-encapsulated circular RNA in tissues and organs, 5 ug of m1Ψ-modified linear Luciferase mRNA and 5 ug of circular Luciferase encapsulated by MC3-LNP were injected into mice through tail vein delivery. Live imaging by IVIS was taken to measure the luminescence signal elicited upon the injection of luciferin at different timepoints post injection of the RNAs to reflect protein expression level. The results showed that at the 6-hour time point, the expression of linear Luciferase mRNA and circular Luciferase reached the highest point in the tissue. Over time, the expression of linear Luciferase mRNA and circular Luciferase gradually decreased, but compared with linear Luciferase mRNA, signal of circular Luciferase RNA declined more slowly (FIG. 57).

Taken together, this series of examples demonstrates that circular RNAs have at least several advantages over linear RNAs, including higher stability, lower immunogenicity higher translation efficiency and longer duration in vivo than linear RNA.

### Example 23. Chemically Modified Nucleotides in Preparation of Circular RNA Reduces Circularization Efficiency

This Example demonstrated that introducing chemically modified nucleotides during the in vitro synthesis of circular RNA reduces the circularization efficiency.

### 1. Introducing m5C Reduces the Circularization Efficiency

This Example demonstrated that introducing m5C reduces the circularization efficiency

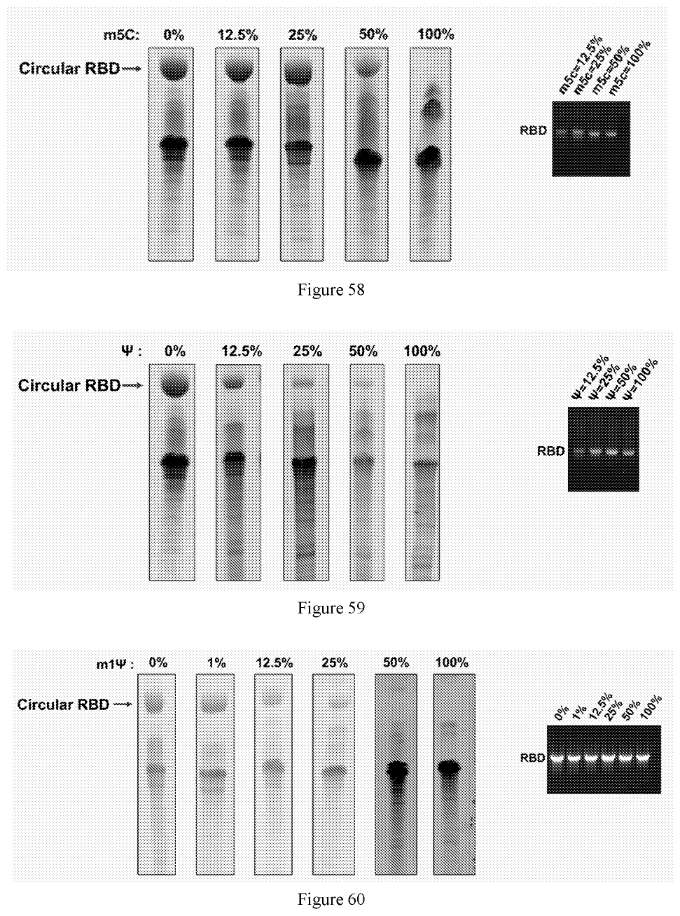

Taking the preparation of circular RBD using the self-splicing intron AnaX via in vitro transcription as an example, during the in vitro transcription, different percentages of cytosine were replaced with m5C-modified cytosine (0%, 12.5%, 25%, 50% and 100%). After 3.5 hours incubation at 37° C., 0.5 μl of the reaction product was taken out for native agarose gel electrophoresis to monitor the in vitro transcription efficiency. Subsequently, GTP and Mg²⁺ were added to for circularization at 55° C. for 15 minutes. The circularization efficiency was analyzed by denature PAGE gel electrophoresis. The results showed that introducing m5C-modified cytosine did not affect the in vitro transcription efficiency (FIG. 58), but the introduction of various amount of m5C significantly inhibited RBD RNA circularization. When 100% cytosine was replaced with m5C, RBD RNA circularization was almost completely abolished (FIG. 58).

### 2. Introducing Ψ Reduces the Circularization Efficiency

This Example demonstrated that introducing Ψ reduces the circularization efficiency.

Taking the preparation of circular RBD using the self-splicing intron AnaX via in vitro transcription as an example, during the in vitro transcription, different percentages of uridine were replaced with of Ψ (0%, 12.5%, 25%, 50% and 100%). After 3.5 hours incubation at 37° C., 0.5 ul of the reaction product was taken out for native agarose gel electrophoresis to monitor the in vitro transcription efficiency. Then, GTP and Mg²⁺ were added for circularization at 55° C. for 15 minutes. The circularization efficiency was analyzed by denature PAGE gel electrophoresis. The results showed that introducing Ψ did not affect the in vitro transcription efficiency (FIG. 59), but the introduction of various amount of Ψ significantly inhibited RNA circularization. When 100% uridine was replaced with Ψ, circularization was almost completely abolished. (FIG. 59).

3. The m1Ψ Modification Affects the Efficiency of Circular RNA Formation

This Example demonstrated that introducing m1Ψ reduces the circularization efficiency.

Taking the preparation of circular RBD using the self-splicing intron AnaX via in vitro transcription as an example, during the in vitro transcription, different percentages of uridine were replaced with m1Ψ (0%, 1%, 12.5%, 25%, 50% and 100%). After 3.5 hours incubation at 37° C., 0.5 μl of the reaction product was taken out for native agarose gel electrophoresis to monitor the in vitro transcription efficiency. Subsequently, GTP and Mg²⁺ were added to the remaining reaction system for circularization at 55° C. for 15 minutes. The circularization efficiency was analyzed by denature PAGE gel electrophoresis. The results showed that introducing m1Ψ did not affect the in vitro transcription efficiency (FIG. 60), but the introduction of various amount of m1Ψ significantly inhibited RNA circularization. When 100% uridine was replaced with m1Ψ, RNA circularization was completely abolished (FIG. 60).

### 4. m6A Modification Affects the Efficiency of Circular RNA Formation

This example demonstrated that introducing m6A reduces the circularization efficiency.

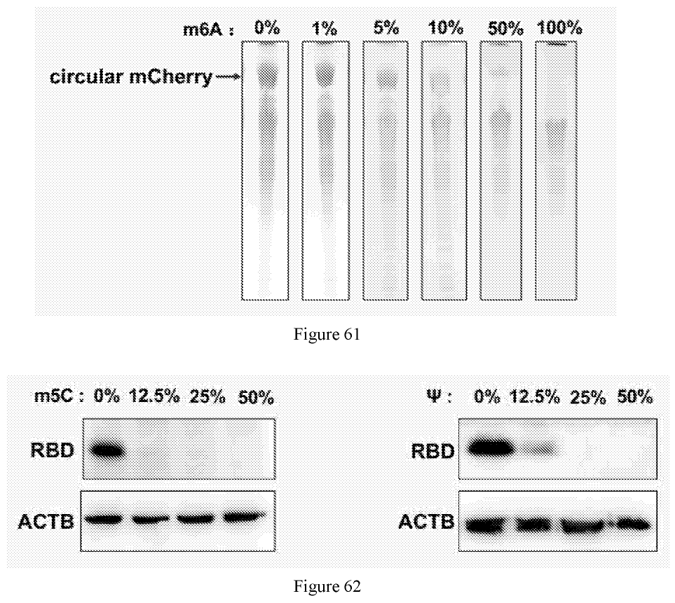

Taking the preparation of circular RBD using the self-splicing intron AnaX via in vitro transcription as an example, during the in vitro transcription, different percentages of adenine were replaced with m6A-modified adenine (0%, 1%, 5%, 10%, 50% and 100%). After 3.5 hours incubation at 37° C., 0.5 μl of the reaction product was taken out for native agarose gel electrophoresis to monitor the in vitro transcription efficiency. Subsequently, GTP and Mg²⁺ were added to the remaining reaction system for circularization at 55° C. for 15 minutes. The circularization efficiency was analyzed by denature PAGE gel electrophoresis. The results showed that introducing m6A did not affect the in vitro transcription efficiency (FIG. 61), but the introduction of various amount of m6A significantly inhibited RNA circularization. When 100% adenine was replaced with m6A-modified adenine, RNA circularization was almost completely abolished (FIG. 61).

### Example 24. Nucleotide Modification Affects Circular RNA Translation

This Example demonstrated that introducing chemically modified nucleotides in the circular RNA during the preparation via the in vitro synthesis of circular RNA can reduce the translation efficiency of the circular RNA.

Taking the preparation of circular RBD using the self-splicing intron AnaX via in vitro transcription as an example, circular RNAs with different percentages of cytosine replaced with m5C or different percentages of uridine replaced with Ψ were synthesized by the methods described in Examples 23.1 and 23.2. Using Lipofectamine MessengerMAX™ (Thermo Fisher) RNA transfection reagent, equal amounts of circular RBD with chemically modified nucleotides were transfected into human HEK293FT cells. Twenty-four hours after transfection, Western Blot was used to detect the expression levels of protein products translated from circular RBD with chemically modified nucleotides. The results showed that compared with the unmodified circular RBD, the introducing m5C or Ψ to circular RBDs led to very low to no expression in cells (FIG. 62).

In summary, introducing m5C or Ψ to circular RNA can significantly inhibit the translation and expression of circular RNA in cells.

### Example 25. IRES Element Affects the Translation Level of Circular RNA

This example demonstrates that IRES elements can affect the protein expression levels of in vitro synthesized circular RNA.

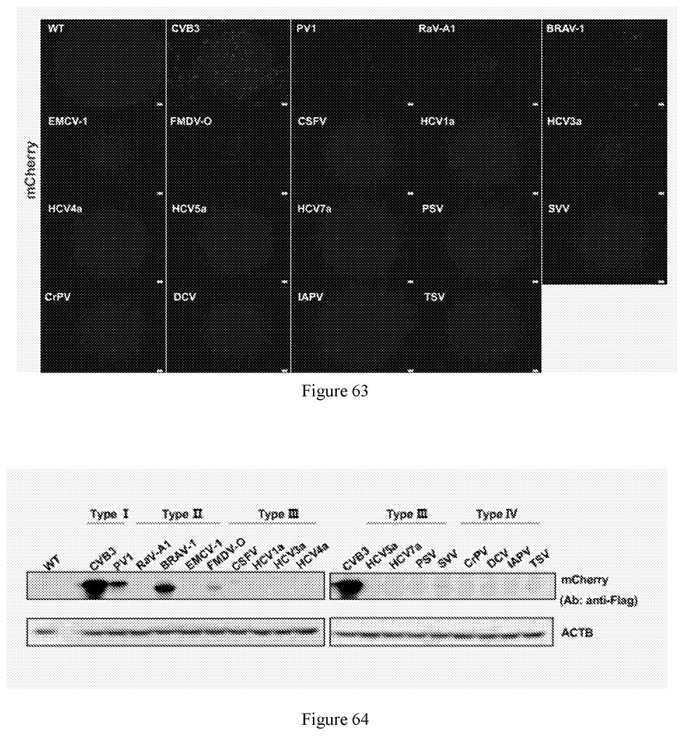

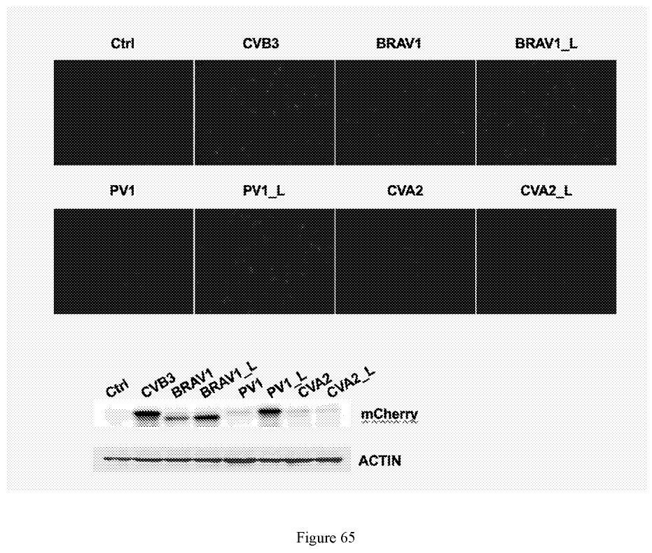

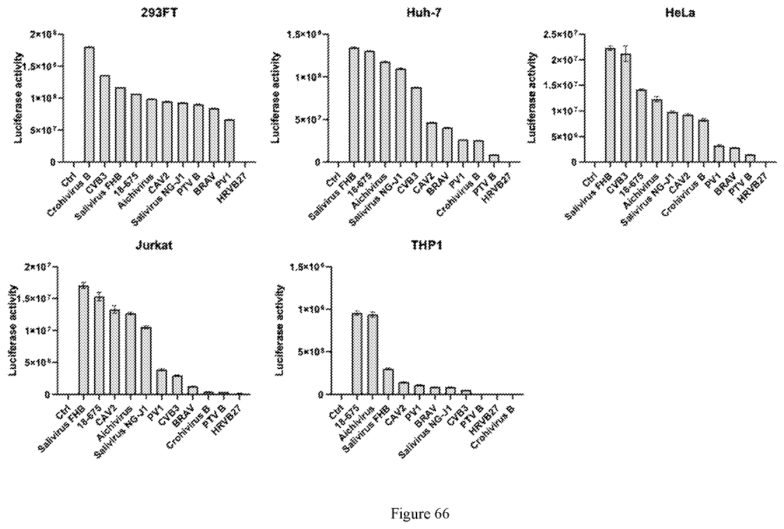

Type I, type II, type III and type IV IRES from different viruses were selected for the expression of circular mCherry. Circular RNA was prepared by in vitro transcription using the self-splicing method of AnaX system, and the same amount of circular RNA containing different IRES elements was introduced into human HEK293FT cells using Lipofectamine MessengerMAX™ (Thermo Fisher) RNA transfection reagent. 48 hours after transfection, the red fluorescent signal of the translation product red fluorescent protein mCherry was detected by fluorescence microscope (the upper panel of FIG. 63), and the expression level of the translated protein product was detected by Western Blot (the lower panel of FIG. 64). These IRES are from Poliovirus 1, Rhinovirus A1, Coxsackievirus A2, Coxsackievirus B3, Encephalomyocarditis virus, Foot-and-mouth disease virus, Bovine rhinitis A virus, Hepatitis C virus, Classical swine fever virus, Seneca Valley virus, Porcine sapelovirus, Cricket paralysis virus, Drosophila C virus, Taura syndrome virus, Israel acute paralysis virus. The results showed that among the four types of IRES, the translation efficiency of circular RNAs initiated by IRES elements of type I and type II was relatively higher.

In order to further explore the effect of the complete 5′ UTR and IRES sequences of the virus on the translation efficiency, the translation capability of the IRES sequences BRAV-1, PV1, CVA2 and their complete 5′ UTR sequences BRAV-1_L, PV1_L, and CVA2_L were tested. BRAV-1_L, PV1_L, and CVA2_L extend forward by 100 bases respectively in the genome position. The circular mCherry was prepared by in vitro transcription using the intron self-splicing of the AnaX system, and the same amount of circular RNA with different IRES elements was introduced into the HEK293FT cell using Lipofectamine MessengerMAX™ (Thermo Fisher) RNA transfection reagent. 48 hours after transfection, the red fluorescent signal of the translation product red fluorescent protein mCherry was detected by fluorescence imaging microscope, and the expression level of the translated protein product was detected by Western Blot (FIG. 65). The results showed that the complete 5′ UTR sequences BRAV-1_L, PV1_L, CVA2_L have stronger translation ability than the traditionally defined BRAV-1, PV1, CVA2.

To sum up, the IRES elements of type I and type II are more suitable for regulating the translation of circular RNA, and the complete 5′UTR sequence of the virus has stronger translation capability than the traditionally defined IRES, that is, a more complete IRES element.

### SEQUENCES MENTIONED HEREIN

| SEQ ID | | |
|---|---|---|
| NO. | Sequence | Description |
| 1 | AACAATAGATGACTTACAACTAATCGGAAGGTGCAGAGACTCGACGGGAG | 3′ Group I intron |
| | CTACCCTAACGTCAAGACGAGGGTAAAGAGAGAGTCCAATTCTCAAAGCC | fragment derived |
| | AATAGGCAGTAGCGAAAGCTGCAAGAGAATG | from Anabaena |
| | | pre-tRNA-Leu gene |
| | | GROUP I INTRON |
| 2 | AAATAATTGAGCCTTAAAGAAGAAATTCTTTAAGTGGATGCTCTCAAACT | 5′ Group I intron |
| | CAGGGAAACCTAAATCTAGTTATAGACAAGGCAATCCTGAGCCAAGCCGA | fragment derived |
| | AGTAGTAATTAGTAA | from Anabaena |
| | | pre-tRNA-Leu gene |
| | | GROUP I INTRON |
| 3 | TGCGCCGATGAAGGTGTAGAGACTAGACGGCACCCACCTAAGGCAAACGC | 3′ Group I intron |
| | TATGGTGAAGGCATAGTCCAGGGAGTGGCGAAAGTCACACAAACCGG | fragment derived |
| | | from Azoarcus sp. |
| | | BH72 |
| 4 | ATTTCGATGTGCCTTGCGCCGGGAAACCACGCAAGGGATGGTGTCAAATT | 5′ Group I intron |
| | CGGCGAAACCTAAGCGCCCGCCCGGGCGTATGGCAACGCCGAGCCAAGCT | fragment derived |
| | TCGGCGCC | from Azoarcus sp. |
| | | BH72 |
| 5 | AAAATTTCGTCTGGATTAGTTACTTATCGTGTAAAATCTGATAAATGGAA | 3′ Group I intron |
| | TTGGTTCTACATAAATGCCTAACGACTATCCCTTTGGGGAGTAGGGTCAA | fragment derived |
| | GTGACTCGAAACGATAGACAACTTGCTTTAACAAGTTGGAGATATAGTCT | from T4.v.td, 1 |
| | GCTCTGCATGGTGACATGCAGCTGGATATAATTCCGGGGTAAGATTAACG | |
| | ACCTTATCTGAACATAATG | |
| 6 | TAATTGAGGCCTGAGTATAAGGTGACTTATACTTGTAATCTATCTAAACG | 5′ Group I intron |
| | GGGAACCTCTCTAGTAGACAATCCCGTGCTAAATTGTAGGACTGCCCTTT | fragment derived |
| | AATAAATACTTCTATATTTAAAGAGGTATTTATGAAAAGCGGAATTTATC | from T4.v.td, 1 |
| | AGATTAAAAATACTTT | |
| 7 | AAAATCCGT | 3′ exon of Group |
| | | I intron from |
| | | Anabaena pre- |
| | | tRNA-Leu gene |
| 8 | CGCTACGGACTT | 5′ exon of Group |
| | | I intron from |
| | | Anabaena pre- |
| | | tRNA-Leu gene |
| 9 | AATCCGCCGGTG | 3′ exon of Group |
| | | I intron from |
| | | Azoarcus sp.BH72 |
| 10 | GAGCGGCGGACTCAT | 5′ exon of Group |
| | | I intron from |
| | | Azoarcus sp.BH72 |
| 11 | ctaccgtttaatatt | 3′ exon of Group |
| | | I intron from |
| | | T4.v.td, 1 |
| 12 | gatgttttcttgggt | 5′ exon of Group |
| | | I intron from |
| | | T4.v.td, 1 |
| 13 | AAAATCCGTTGA | first residual |
| | | circularizing |
| | | element, AnaX, |
| | | AnaXD1, AnaXD2, |
| | | AnaXD6, AnaXD 12, |
| | | AnaXD13, AnaXD14 |
| | | AnaXE1 |
| 14 | AAAAAGGCATGA | first residual |
| | | circularizing |
| | | element, AnaXD3, |
| | | AnaXRD2 |
| 15 | AAAATCTGTTGA | first residual |
| | | circularizing |
| | | element, AnaXRD1 |
| 16 | AAAGTCCGTTGA | first residual |
| | | circularizing |
| | | element, AnaXE2, |
| | | AnaXE3 |
| 17 | AAAATCCGTTGCGA | first residual |
| | | circularizing |
| | | element, AnaXE4 |
| 18 | AAAATCCGTTGCGTCA | first residual |
| | | circularizing |
| | | element, AnaXE5 |
| 19 | AAAAAAAAAAGG | first residual |
| | | circularizing |
| | | element, AnaXAU |
| 20 | AAAACCCCCTGA | first residual |
| | | circularizing |
| | | element, AnaXCGv1 |
| 21 | AAAATGGGGGGA | first residual |
| | | circularizing |
| | | element, AnaXCGv2 |
| 22 | AAAACCCCCCCA | first residual |
| | | circularizing |
| | | element, AnaXCGv3 |
| 23 | TTTTTCCGTTGA | first residual |
| | | circularizing |
| | | element, AnaXD5 |
| 24 | AATCCGTTGA | first residual |
| | | circularizing |
| | | element, AnaXD7 |
| 25 | TCCGTTGA | first residual |
| | | circularizing |
| | | element, AnaXD8 |
| 26 | GAAATCCGTTGA | first residual |
| | | circularizing |
| | | element, AnaXD9 |
| 27 | TAAATCCGTTGA | first residual |
| | | circularizing |
| | | element, AnaXD10 |
| 28 | CAAATCCGTTGA | first residual |
| | | circularizing |
| | | element, AnaXD11 |
| 29 | AAAATCCGTTGACCTTAAACGGTCGTGTGGGTTCAAGTCCCTCCACCCCC | first residual |
| | ACGCCGGAAACGCAATAGCCGAAAAACAAAAAACAAAAAAAACAAAAAAA | circularizing |
| | AAACCAAAAAAACAAAACACA | element, Ana3.0 |
| 30 | AAAATCCGTTGACCTTAAACGGTCGTGTGGGTTCAAGTCCCTCCACCCCC | first residual |
| | AGAAACCAACTTTATTACTATATTCCCCACAACC | circularizing |
| | | element, Ana1.0 |
| 31 | AAAATCCGTTGACCTTAAACGGTCGTGTGGGTTCAAGTCCCTCCACCCCC | first residual |
| | A | circularizing |
| | | element, Ana0.9 |
| 32 | AATCCGTTGGTG | first residual |
| | | circularizing |
| | | element, Azo |
| 33 | AATCCGCCGGTG | first residual |
| | | circularizing |
| | | element, AzoE |
| 34 | AAAA | first loop |
| | | sequence |
| 35 | TTTT | first loop |
| | | sequence |
| 36 | AA | first loop |
| | | sequence |
| 37 | TAAA | first loop |
| | | sequence |
| 38 | GAAA | first loop |
| | | sequence |
| 39 | CAAA | first loop |
| | | sequence |
| 40 | AAAAU | first loop |
| | | sequence |
| 41 | AAAAC | first loop |
| | | sequence |
| 42 | TCCGT | first pairing |
| | | sequence |
| 43 | GGC | first pairing |
| | | sequence |
| 44 | AGGCA | first pairing |
| | | sequence |
| 45 | CGTT | first pairing |
| | | sequence |
| 46 | TCTGT | first pairing |
| | | sequence |
| 47 | TCCGUUG | first pairing |
| | | sequence |
| 48 | AAGTCCGT | first pairing |
| | | sequence |
| 49 | AAGTCCGTTG | first pairing |
| | | sequence |
| 50 | TCCGTTGCG | first pairing |
| | | sequence |
| 51 | TCCGTTGCGTC | first pairing |
| | | sequence |
| 52 | AAAAAAGG | first pairing |
| | | sequence |
| 53 | CCCC | first pairing |
| | | sequence |
| 54 | GGGGGG | first pairing |
| | | sequence |
| 55 | CCCCCCC | first pairing |
| | | sequence |
| 56 | AGACGCTACGGACTT | second residual |
| | | circularizing |
| | | element, AnaX, |
| | | AnaXD3, AnaXE2, |
| | | AnaXD5, AnaXD7, |
| | | AnaXD8, AnaXD9, |
| | | AnaXD10, AnaXD11 |
| 57 | AGACGCTACAGACTT | second residual |
| | | circularizing |
| | | element, AnaXD1, |
| | | AnaXRD1 |
| 58 | AGACGCTTGCCTCTT | second residual |
| | | circularizing |
| | | element, AnaXD2, |
| | | AnaXRD2 |
| 59 | AGACGCAACGGACTT | second residual |
| | | circularizing |
| | | element, AnaXE1, |
| | | AnaXE3, AnaXE4, |
| | | AnaXE5 |
| 60 | AGACCCTTTTTTCTT | second residual |
| | | circularizing |
| | | element, AnaXAU |
| 61 | AGACGCTGGGGGCTT | second residual |
| | | circularizing |
| | | element, AnaXCGv1 |
| 62 | AGACGCCCCCCACTT | second residual |
| | | circularizing |
| | | element, AnaXCGv2 |
| 63 | AGACGGGGGGGGCTT | second residual |
| | | circularizing |
| | | element, AnaXCGv3 |
| 64 | AAAAAACAAAAAACAAAACGGCTATTATGCGTTACCGGCGAGACGCTACG | second residual |
| | GACTT | circularizing |
| | | element, Ana3.0 |
| 65 | AGACGCTACGGACTT | second residual |
| | | circularizing |
| | | element, Ana1.0, |
| | | Ana0.9 |
| 66 | AGACGCTACGGACAA | second residual |
| | | circularizing |
| | | element, AnaXD6 |
| 67 | AGACGCTACGGACTC | second residual |
| | | circularizing |
| | | element, AnaXD12 |
| 68 | AGACGCTACGGACTA | second residual |
| | | circularizing |
| | | element, AnaXD13 |
| 69 | AGACGCTACGGACTG | second residual |
| | | circularizing |
| | | element, AnaXD14 |
| 70 | GAGCAGCGGACTCAT | second residual |
| | | circularizing |
| | | element, Azo |
| 71 | GAGCGGCGGACTCAT | second residual |
| | | circularizing |
| | | element, AzoE |
| 72 | CTC | second loop |
| | | sequence |
| 73 | CTT | second loop |
| | | sequence |
| 74 | CTA | second loop |
| | | sequence |
| 75 | CTG | second loop |
| | | sequence |
| 76 | GCTT | second loop |
| | | sequence |
| 77 | ACTT | second loop |
| | | sequence |
| 78 | ACGGA | second pairing |
| | | sequence |
| 79 | GACG | second pairing |
| | | sequence |
| 80 | GACG | second pairing |
| | | sequence |
| 81 | GCT | second pairing |
| | | sequence |
| 82 | ACAGA | second pairing |
| | | sequence |
| 83 | TGCCT | second pairing |
| | | sequence |
| 84 | TGCCT | second pairing |
| | | sequence |
| 85 | CAACGGA | second pairing |
| | | sequence |
| 86 | ACGGACTT | second pairing |
| | | sequence |
| 87 | CAACGGACTT | second pairing |
| | | sequence |
| 88 | CGCAACGGA | second pairing |
| | | sequence |
| 89 | GACGCAACGGA | second pairing |
| | | sequence |
| 90 | CCTTTTTT | second pairing |
| | | sequence |
| 91 | GGGG | second pairing |
| | | sequence |
| 92 | CCCCCC | second pairing |
| | | sequence |
| 93 | GGGGGGG | second pairing |
| | | sequence |
| 94 | CCGATTAGTTGTAAGTCATCTATTG | Ligand Intron |
| 95 | TCAACGGATTTTAAGTCCGTAGCGTCT | Ligand Feature |
| 96 | ATTTTAAGTC | Ligand F10 |
| 97 | AACGGATTTTAAGTCCGTAG | Ligand F20 |
| 98 | CAACGGATTTTAAGTCCGTAGCG | Ligand F23 |
| 99 | TCAACGGATTTTAAGTCCGTAGCGT | Ligand F25 |
| 100 | TCAACGGATTTTAAGTCCGTAGCGTCT | Ligand F27 |
| 101 | ATCAACGGATTTTAAGTCCGTAGCGTCTT | Ligand F29 |
| 102 | TGCGAGGAACCATCCGGCCAGCTCCTG | Ligand- |
| | | Feature/ligase |
| 103 | GATAGTCGTTAGGCATTTATGTAGA | Ligand-Td-Intron |
| 104 | TATTAAACGGTAGACCCAAGAAAACAT | Ligand-Td-Feature |
| 105 | TTAAAACAGCCTGTGGGTTGATCCCACCCACAGGCCCATTGGGCGCTAGC | Coxsackievirus B3 |
| | ACTCTGGTATCACGGTACCTTTGTGCGCCTGTTTTATACCCCCTCCCCCA | (CVB3) |
| | ACTGTAACTTAGAAGTAACACACACCGATCAACAGTCAGCGTGGCACACC | |
| | AGCCACGTTTTGATCAAGCACTTCTGTTACCCCGGACTGAGTATCAATAG | |
| | ACTGCTCACGCGGTTGAAGGAGAAAGCGTTCGTTATCCGGCCAACTACTT | |
| | CGAAAAACCTAGTAACACCGTGGAAGTTGCAGAGTGTTTCGCTCAGCACT | |
| | ACCCCAGTGTAGATCAGGTCGATGAGTCACCGTATTCCCCACGGGCGACC | |
| | GTGGCGGTGGCTGCGTTGGCGGCCTGCCCATGGGGAAACCCATGGGACGC | |
| | TCTAATACAGACATGGTGCGAAGAGTCTATTGAGCTAGTTGGTAGTCCTC | |
| | CGGCCCCTGAATGCGGCTAATCCTAACTGCGGAGCACACACCCTCAAGCC | |
| | AGAGGGCAGTGTGTCGTAACGGGCAACTCTGCAGCGGAACCGACTACTTT | |
| | GGGTGTCCGTGTTTCATTTTATTCCTATACTGGCTGCTTATGGTGACAAT | |
| | TGAGAGATCGTTACCATATAGCTATTGGATTGGCCATCCGGTGACTAATA | |
| | GAGCTATTATATATCCCTTTGTTGGGTTTATACCACTTAGCTTGAAAGAG | |
| | GTTAAAACATTACAATTCATTGTTAAGTTGAATACAGCAAA | |
| 106 | TTTTGCGGCTCTGCCGCCGTTCGGGTTTTACCTGTTTTCACAGAGCAAAA | BRAV-1 L |
| | CAGGACCTCTAGTTTCGTGCTTAAACGAGATCATGCTCGAACTAGAACTA | |
| | TAACGCTGGTCACTGGACCCGTGCCGCGCCTTGCGGATCTTTGCGGGAAT | |
| | GGTGGCTAGTGGGCTGTGGAAGTGACTCTAACCACACGCCCCTCAAGTGT | |
| | GGGAAAACACGAACTGGTGTAGCGACGACGATAGGCCTTGGGACACCCTC | |
| | TCCAGTGATGGAGACCCAAGGGGCCAAAAGCCACGCCTTGTGCCCTGTCG | |
| | TTCACAACCCCAGTGCAGTTCGTGCCAGTACCTGCTTTTGGGAAGTGTGC | |
| | TTTGGACAGCTGAAAACAGTCCTAGTGGGAGACTAAGGATGCCCAGGAGG | |
| | TACCCGGAGGTAACAAGTGACACTCTGGATCTGACTTGGGGAGAGCGGGT | |
| | CTGCTTTACAGACGCCACTCTTTAAAAAACTTCTATGTCTCGTCAGGCAC | |
| | CGGAGGCCGGGCCTTTTCCTTTAAAACAATACACTTT | |
| 107 | ATAACGCTGGTCACTGGACCCGTGCCGCGCCTTGCGGATCTTTGCGGGAA | BRAV-1 |
| | TGGTGGCTAGTGGGCTGTGGAAGTGACTCTAACCACACGCCCCTCAAGTG | |
| | TGGGAAAACACGAACTGGTGTAGCGACGACGATAGGCCTTGGGACACCCT | |
| | CTCCAGTGATGGAGACCCAAGGGGCCAAAAGCCACGCCTTGTGCCCTGTC | |
| | GTTCACAACCCCAGTGCAGTTCGTGCCAGTACCTGCTTTTGGGAAGTGTG | |
| | CTTTGGACAGCTGAAAACAGTCCTAGTGGGAGACTAAGGATGCCCAGGAG | |
| | GTACCCGGAGGTAACAAGTGACACTCTGGATCTGACTTGGGGAGAGCGGG | |
| | TCTGCTTTACAGACGCCACTCTTTAAAAAACTTCTATGTCTCGTCAGGCA | |
| | CCGGAGGCCGGGCCTTTTCCTTTAAAACAATACACTTT | |
| 108 | CGTAACTTAGACGCACAAAACCAAGTTCAATAGAAGGGGGTACAAACCAG | PV1 |
| | TACCACCACGAACAAGCACTTCTGTTTCCCCGGTGATGTCGTATAGACTG | |
| | CTTGCGTGGTTGAAAGCGACGGATCCGTTATCCGCTTATGTACTTCGAGA | |
| | AGCCCAGTACCACCTCGGAATCTTCGATGCGTTGCGCTCAGCACTCAACC | |
| | CCAGAGTGTAGCTTAGGCTGATGAGTCTGGACATCCCTCACCGGTGACGG | |
| | TGGTCCAGGCTGCGTTGGCGGCCTACCTATGGCTAACGCCATGGGACGCT | |
| | AGTTGTGAACAAGGTGTGAAGAGCCTATTGAGCTACATAAGAATCCTCCG | |
| | GCCCCTGAATGCGGCTAATCCCAACCTCGGAGCAGGTGGTCACAAACCAG | |
| | TGATTGGCCTGTCGTAACGCGCAAGTCCGTGGCGGAACCGACTACTTTGG | |
| | GTGTCCGTGTTTCCTTTTATTTTATTGTGGCTGCTTATGGTGACAATCAC | |
| | AGATTGTTATCATAAAGCGAATTGGATTGGCCATCCGGTGAAAGTGAGAC | |
| | TCATTATCTATCTGTTTGCTGGATCCGCTCCATTGAGTGTGTTTACTCTA | |
| | AGTACAATTTCAACAGTTATTTCAATCAGACAATTGTATCATA | |
| 109 | TTAAAACAGCTCTGGGGTTGTACCCACCCCAGAGGCCCACGTGGCGGCTA | PV1_L |
| | GTACTCCGGTATTGCGGTACCCTTGTACGCCTGTTTTATACTCCCTTCCC | |
| | GTAACTTAGACGCACAAAACCAAGTTCAATAGAAGGGGGTACAAACCAGT | |
| | ACCACCACGAACAAGCACTTCTGTTTCCCCGGTGATGTCGTATAGACTGC | |
| | TTGCGTGGTTGAAAGCGACGGATCCGTTATCCGCTTATGTACTTCGAGAA | |
| | GCCCAGTACCACCTCGGAATCTTCGATGCGTTGCGCTCAGCACTCAACCC | |
| | CAGAGTGTAGCTTAGGCTGATGAGTCTGGACATCCCTCACCGGTGACGGT | |
| | GGTCCAGGCTGCGTTGGCGGCCTACCTATGGCTAACGCCATGGGACGCTA | |
| | GTTGTGAACAAGGTGTGAAGAGCCTATTGAGCTACATAAGAATCCTCCGG | |
| | CCCCTGAATGCGGCTAATCCCAACCTCGGAGCAGGTGGTCACAAACCAGT | |
| | GATTGGCCTGTCGTAACGCGCAAGTCCGTGGCGGAACCGACTACTTTGGG | |
| | TGTCCGTGTTTCCTTTTATTTTATTGTGGCTGCTTATGGTGACAATCACA | |
| | GATTGTTATCATAAAGCGAATTGGATTGGCCATCCGGTGAAAGTGAGACT | |
| | CATTATCTATCTGTTTGCTGGATCCGCTCCATTGAGTGTGTTTACTCTAA | |
| | GTACAATTTCAACAGTTATTTCAATCAGACAATTGTATCATAATGGGTGC | |
| 110 | TTAAAACAGCCTGTGGGTTGCACCCACCCACAGGGCCCACTGGGCGCTAG | CVA2 L |
| | CACTCTGGTATCACGATACCTTTGTGCGCCTGTTTTATATCCCCACCCCG | |
| | AGTAAACGTTAGAAGTTACGCAACCCCGATCAATAGTAGGTGTAGCACTC | |
| | CAGCTGCATCGAGATCAAGCACTTCTGTCTCCCCGGACCGAGTATCAATA | |
| | GACTGCTAACGCGGTTGAAGGAGAAAACGTTCGTTACCCGGCCAATTACT | |
| | TCGAGAAGCCCAGTAGTGCCGTGAAAGTTGCGGAGTGTTTCGCTCAGCAC | |
| | TTCCCCCGTGTAGATCAGGCTGATGAGTCACCGCGATCCCCACAGGTGAC | |
| | TGTGGCGGTGGCTGCGTTGGCGGCCTGCCTATGGGGCAACCCATAGGACG | |
| | CTCTAATACAGACATGGTGCGAAGAGCCTATTGAGCTAATTGGTAGTCCT | |
| | CCGGCCCCTGAATGCGGCTAATCCTAACTGCGGAGCACATGCCCTCAAAC | |
| | CAGGGGGTGGTGTGTCGTAACGGGTAACTCTGCAGCGGAACCGACTACTT | |
| | TGGGTGTCCGTGTTTCTTTTTATTCTTATAATGGCTGCTTATGGTGACAA | |
| | TTAAAGAATTGTTACCATATAGCTATTGGATTGGCCATCCGGTGACTAAC | |
| | AAATCGCTCATATACCAGTTTGTTGGTTTTGTTCCCTTATCACATACAGC | |
| | TCATAACACCCTCTTATATTTACTACAATTGAATAGCAAGAAATGGGGGC | |
| 111 | GAGTAAACGTTAGAAGTTACGCAACCCCGATCAATAGTAGGTGTAGCACT | CVA2 |
| | CCAGCTGCATCGAGATCAAGCACTTCTGTCTCCCCGGACCGAGTATCAAT | |
| | AGACTGCTAACGCGGTTGAAGGAGAAAACGTTCGTTACCCGGCCAATTAC | |
| | TTCGAGAAGCCCAGTAGTGCCGTGAAAGTTGCGGAGTGTTTCGCTCAGCA | |
| | CTTCCCCCGTGTAGATCAGGCTGATGAGTCACCGCGATCCCCACAGGTGA | |
| | CTGTGGCGGTGGCTGCGTTGGCGGCCTGCCTATGGGGCAACCCATAGGAC | |
| | GCTCTAATACAGACATGGTGCGAAGAGCCTATTGAGCTAATTGGTAGTCC | |
| | TCCGGCCCCTGAATGCGGCTAATCCTAACTGCGGAGCACATGCCCTCAAA | |
| | CCAGGGGGTGGTGTGTCGTAACGGGTAACTCTGCAGCGGAACCGACTACT | |
| | TTGGGTGTCCGTGTTTCTTTTTATTCTTATAATGGCTGCTTATGGTGACA | |
| | ATTAAAGAATTGTTACCATATAGCTATTGGATTGGCCATCCGGTGACTAA | |
| | CAAATCGCTCATATACCAGTTTGTTGGTTTTGTTCCCTTATCACATACAG | |
| | CTCATAACACCCTCTTATATTTACTACAATTGAATAGCAAGAA | |
| 112 | TAAGTAGGGAACATATTCAATTCATATTGTTCATCTCACTGAACCCGCAT | Rafivirus A1 |
| | GAAGGACTGCATTGCATATCCTGGACGAGGTGACGTGGAATATTTGGACA | (RaV-A1) |
| | TTTATGGATTGGACACTATAACGCTTTGTGCCTCTACGGAGATGTAACCA | |
| | TAATCTTAAGTAGTAGTACCCCAGCACAAGAGGATAAAGTGGCATACACG | |
| | ACAACGGGTGTTGCTCGCACCTTAGTAATGTGGATGTCCACCCTTGGAGC | |
| | GTGCTGAAACTCTGTGGGTAAAGACACATATTAGTACAAATGTGGGGGAA | |
| | CTCACTGAAAGGGCATGTCCCGTGTACTGGTGTGCCGGAAAGTGGGGGTC | |
| | GCTTTCTGGAGAACTTAGTAGTTCTTGTTATTGGGTGATAGCCTTGCGGC | |
| | GGATCAACCCACAGTTTTAATCCGTTGTTTTGCAT | |
| 113 | GAGTAAACGTTAGAAGTTACGCAACCCCGATCAATAGTAGGTGTAGCACT | Coxsackievirus A2 |
| | CCAGCTGCATCGAGATCAAGCACTTCTGTCTCCCCGGACCGAGTATCAAT | (CVA2) |
| | AGACTGCTAACGCGGTTGAAGGAGAAAACGTTCGTTACCCGGCCAATTAC | |
| | TTCGAGAAGCCCAGTAGTGCCGTGAAAGTTGCGGAGTGTTTCGCTCAGCA | |
| | CTTCCCCCGTGTAGATCAGGCTGATGAGTCACCGCGATCCCCACAGGTGA | |
| | CTGTGGCGGTGGCTGCGTTGGCGGCCTGCCTATGGGGCAACCCATAGGAC | |
| | GCTCTAATACAGACATGGTGCGAAGAGCCTATTGAGCTAATTGGTAGTCC | |
| | TCCGGCCCCTGAATGCGGCTAATCCTAACTGCGGAGCACATGCCCTCAAA | |
| | CCAGGGGGTGGTGTGTCGTAACGGGTAACTCTGCAGCGGAACCGACTACT | |
| | TTGGGTGTCCGTGTTTCTTTTTATTCTTATAATGGCTGCTTATGGTGACA | |
| | ATTAAAGAATTGTTACCATATAGCTATTGGATTGGCCATCCGGTGACTAA | |
| | CAAATCGCTCATATACCAGTTTGTTGGTTTTGTTCCCTTATCACATACAG | |
| | CTCATAACACCCTCTTATATTTACTACAATTGAATAGCAAGAA | |
| 114 | CCTGTCTTCTTGACGAGCATTCCTAGGGGTCTTTCCCCTCTCGCCAAAGG | Encephalomyocarditis |
| | AATGCAAGGTCTGTTGAATG | virus (EMCV- |
| | TCGTGAAGGAAGCAGTTCCTCTGGAAGCTTCTTGAAGACAAACAACGTCT | 1 |
| | GTAGCGACCCTTTGCAGGCAGCGGAACCCCCCACCTGGCGACAGGTGCCT | |
| | CTGCGGCCAAAAGCCACGTGTATAAGATACACCTGCAAAGGCGGCACAAC | |
| | CCCAGTGCCACGTTGTGAGTTGGATAGTTGTGGAAAGAGTCAAATGGCTC | |
| | TCCTCAAGCGTATTCAACAAGGGGCTGAAGGATGCCCAGAAGGTACCCCA | |
| | TTGTATGGGATCTGATCTGGGGCCTCGGTGCACATGCTTTACATGTGTTT | |
| | AGTCGAGGTTAAAAAACGTCTAGGCCCCCCGAACCACGGGGACGTGGTTT | |
| | TCCTTTGAAAAACACGATGATAAT | |
| 115 | TGACATACACCGTGCAATTTGAAACTCCGCCTGGTCTTTCCAGGTCTAGA | Foot-and-mouth |
| | GGGGTAACACTTTGTACTGTGCTTGACTCCACGCTCGGTCCACTGGCGAG | disease virus |
| | TGTTAGTAACAGCACTGGTGCTTCGTAGCGGAGCATGGTGGCCGTGGGAA | (FMDV-O) |
| | CTCCTCCTTGGTAACAAGGACCCACGGGGCCGAAAGCCACGTCCTGACGG | |
| | ACCCACCATGTGTGCAACCCCAGCACGGCAACTTTTCTGTGAAACTCACT | |
| | CTAAGGTGACACTGATACTGGTATTCAAGTACTGGTGACAGGCTAAGGAT | |
| | GCCCTTCAGGTACCCCGAGGTAACACGCGACACTCGGGATCTGAGAAGGG | |
| | GACTGGGGCTTCTGTAAAAGCGCCCAGTTTAAAAAGCTTCTATGCCTGGA | |
| | TAGGTGACCGGAGGCCGGCGCCTTTCCATTATAACTACTGACTTTA | |
| 116 | CCTGTGAGGAACTACTGTCTTCACGCAGAAAGCGTCTAGCCATGGCGTTA | Hepatitis C virus |
| | GTATGAGTGTCGTGCAGCCTCCAGGACCCCCCCTCCCGGGAGAGCCATAG | (HCV1a) |
| | TGGTCTGCGGAACCGGTGAGTACACCGGAATTGCCAGGACGACCGGGTCC | |
| | TTTCTTGGATAAACCCGCTCAATGCCTGGAGATTTGGGCGTGCCCCCGCA | |
| | AGACTGCTAGCCGAGTAGTGTTGGGTCGCGAAAGGCCTTGTGGTACTGCC | |
| | TGATAGGGTGCTTGCGAGTGCCCCGGGAGGTCTCGTAGACCGTGCACC | |
| 117 | ACCTGCCTCTTACGAGGCGACACTCCACCATGGATCACTCCCCTGTGAGG | Hepatitis C virus |
| | AACTTCTGTCTTCACGCGGAAAGCGCCTAGCCATGGCGTTAGTACGAGTG | (HCV3a) |
| | TCGTGCAGCCTCCAGGACCCCCCCTCCCGGGAGAGCCATAGTGGTCTGCG | |
| | GAACCGGTGAGTACACCGGAATCGCTGGGGTGACCGGGTCCTTTCTTGGA | |
| | GCAACCCGCTCAATACCCAGAAATTTGGGCGTGCCCCCGCGAGATCACTA | |
| | GCCGAGTAGTGTTGGGTCGCGAAAGGCCTTGTGGTACTGCCTGATAGGGT | |
| | GCTTGCGAGTGCCCCGGGAGGTCTCGTAGACCGTGCAAC | |
| 118 | TTCACGCAGAAAGCGTCTAGCCATGGCGTTAGTATGAGTGTTGTACAGCC | Hepatitis C virus |
| | TCCAGGACCCCCCCTCCCGGGAGAGCCATAGTGGTCTTCGGAACCGGTGA | (HCV4a) |
| | GTACACCGGAATCGCCGGGATGACCGGGTCCTTTCTTGGATTAACCCGCT | |
| | CAATGCCCGGAAATTTGGGCGTGCCCCCGCAAGACTGCTAGCCGAGTAGT | |
| | GTTGGGTCGCGAAAGGCCTTGCGGTACTGCCTGATAGGGTGCTTGCGAGT | |
| | GCCCCGGGAGGTCTCGTAGACCGTGCACC | |
| 119 | TTCACGCAGAAAGCGTCTAGCCATGGCGTTAGTATGAGTGTCGAACAGCC | Hepatitis C virus |
| | TCCAGGACCCCCCCTCCCGGGAGAGCCATAGTGGTCTGCGGAACCGGTGA | (HCV5a) |
| | GTACACCGGAATTGCCGGGATGACCGGGTCCTTTCTTGGATAAACCCGCT | |
| | CAATGCCCGGAGATTTGGGCGTGCCCCCGCGAGACTGCTAGCCGAGTAGT | |
| | GTTGGGTCGCGAAAGGCCTTGTGGTACTGCCTGATAGGGTGCTTGCGAGT | |
| | GCCCCGGGAGGTCTCGTAGACCGTGCACC | |
| 120 | GCCAGCCCCTTACGGGGCGACACTCCGCCATGAATCACTCCCCTGCGAGG | Hepatitis C virus |
| | AACCACTGTCCTCACGCAGAAAGCGTCTAGCCATGACGTTAGTATGAGTG | (HCV7a) |
| | TCGTACAGCCTCCAGGACCCCCCCTCCCGGGAGAGCCATAGTGGTCTGCG | |
| | GAACCGGTGAGTACACCGGAATTGCCGGGAAGACTGGGTCCTTTCTTGGA | |
| | TCAACCCACTCTATGCCCGGAGATTTGGGCGTGCCCCCGCGAGACTGCTA | |
| | GCCGAGTAGTGTTGGGTCGCGAAAGGCCTTGTGGTACTGCCTGATAGGGT | |
| | GCTTGCGAGTGCCCCGGGAGGTCTCGTAGACCGTGCACC | |
| 121 | GTATACGAGGTTAGTTCATTCTCGTATGCATGATTGGACAAATTAAAATT | Classical swine |
| | TCAATTTGGATCAGGGCCTCCCTCCAGCGACGGCCGAACTGGGCTAGCCA | fever virus |
| | TGCCCACAGTAGGACTAGCAAACGGAGGGACTAGCCGTAGTGGCGAGCTC | (CSFV) |
| | CCTGGGTGGTCTAAGTCCTGAGTACAGGACAGTCGTCAGTAGTTCGACGT | |
| | GAGCAGAAGCCCACCTCGATATGCTATGTGGACGAGGGCATGCCCAAGAC | |
| | ACACCTTAACCCTAGCGGGGGTCGCTAGGGTGAAATCACACCACGTGATG | |
| | GGAGTACGACCTGATAGGGTGCTGCAGAGGCCCACTATTAGGCTAGTATA | |
| | AAAATCTCTGCTGTACATGGCAC | |
| 122 | TAAGACTGGCTCAAGCGCGGAAAGCGCTGTAACCACATGCTGTTAGTCCC | Seneca Valley |
| | TTTATGGCTGCAAGATGGCTACCCACCTCGGATCACTGAACTGGAGCTCG | virus (SVV) |
| | ACCCTCCTTAGTAAGGGAACCGAGAGGCCTTCGTGCAACAAGCTCCGACA | |
| | CAGAGTCCACGTGACTGCTACCACCATGAGTACATGGTTCTCCCCTCTCG | |
| | ACCCAGGACTTCTTTTTGAATATCCACGGCTCGATCCAGAGGGTGGGGCA | |
| | TGACCCCTAGCATAGCGAGCTACAGCGGGAACTGTAGCTAGGCCTTAGCG | |
| | TGCCTTGGATACTGCCTGATAGGGCGACGGCCTAGTCGTGTCGGTTCTAT | |
| | AGGTAGCACATACAAAT | |
| 123 | ACACTCATTTCCCCCCTCCACCCTTAAGGTGGTTGTATCCCCTACACCCT | Porcine |
| | ACCCTCCCTTCCACATAGGACGAATAAACGGACTTGAGATTAAGGCAAGT | sapelovirus (PSV) |
| | ACATAAGGTATGGTTTTTGGATACACTTAAATGGCAGTAGCGTGGCGAGC | |
| | TATGGAAAAATCGCAATTGTCGATAGCCATGTTAGCGACGCGCTTCGGCG | |
| | TGCTCCTTTGGTGATTCGGCGACTGGTTACAGGAGAGTAGACAGTGAGCT | |
| | ATGGGCAAACCCCTACAGTATTACTTAGGGGAATGTGCAATTGAGACTTG | |
| | ACGAGCGTCTCTTTGAGATGTGGCGCATGCTCTTGGCATTACCATAGTGA | |
| | GCTTCCAGGTTGGGAAACCTGGACTGGGTCTATACTGCCTGATAGGGTCG | |
| | CGGCTGGCCGCCTGTAACTAGTATAGTCAGTTGAAAACCCCCC | |
| 124 | AAAAGCAAAAATGTGATCTTGCTTGTAAATACAATTTTGAGAGGTTAATA | Cricket paralysis |
| | AATTACAAGTAGTGCTATTTTTGTATTTAGGTTAGCTATTTAGCTTTACG | virus (CrPV) |
| | TTCCAGGATGCCTAGTGGCAGCCCCACAATATCCAGGAAGCCCTCTCTGC | |
| | GGTTTTTCAGATTAGGTAGTCGAAAAACCTAAGAAATTTACCT | |
| 125 | GTTAAGATGTGATCTTGCTTCCTTATACAATTTTGAGAGGTTAATAAGAA | Drosophila C |
| | GGAAGTAGTGCTATCTTAATAATTAGGTTAACTATTTAGTTTTACTGTTC | virus (DCV) |
| | AGGATGCCTATTGGCAGCCCCATAATATCCAGGACACCCTCTCTGCTTCT | |
| | TATATGATTAGGTTGTCATTTAGAATAAGAAAATAACCT | |
| 126 | ATAGCACCACCCGATCGTAAACTCCATGTATTGGTTACCCATCTGCATCG | Taura syndrome |
| | AAAACTCTCCGAACACTAGGTGCAGTAAGGCTTTCATGGAGTGGTTTGCT | virus (TSV) |
| | ATTTAGCGTACGTGTACCATAGGCAGCCCCAAAAACACGTGTGAGGAGAA | |
| | AGTCCCAGTCACTTTGGGCAAAGTAGACAGCCGCGCTTGCGTGGTGGGAC | |
| | TTAATTA | |
| 127 | AGTATAGTGTTCTGGAGGCATCATTCTATGGTTACCCATCATTAGAGGAA | Israeli acute |
| | ATTTCCAATAAACTCTGGTGTAAGGCTTAGAGTGATGGTCGAGGTGCCCT | paralysis virus |
| | ATTTAGGGTGAGGAGCCTCGGTGGCAGCCCCACCAAATCCTCTATTGGAT | (IAPV) |
| | AGGAACAGCTGTACTGGGCAGTTACAGCAGTCGTATGGTAACACATGCGG | |
| | CGTTCCGAAA | |
| 128 | TTAAAACTGGGTGTGGGTTGTTCCCACCCACACCACCCAATGGGTGTTGT | Rhinovirus A1 |
| | ACTCTGTTATTCCGGTAACTTTGTACGCCAGTTTTTCCCTCCCCTCCCCA | (RV-A1) |
| | TCCTTTTACGTAACTTAGAAGTTTTAAATACAAGACCAATAGTAGGCAAC | |
| | TCTCCAGGTTGTCTAAGGTCAAGCACTTCTGTTTCCCCGGTTGATGTTGA | |
| | TATGCTCCAACAGGGCAAAAACAACAGATACCGTTATCCGCAAAGTGCCT | |
| | ACACAGAGCTTAGTAGGATTCTGAAAGATCTTTGGTTGGTCGTTCAGCTG | |
| | CATACCCAGCAGTAGACCTTGCAGATGAGGCTGGACATTCCCCACTGGTA | |
| | ACAGTGGTCCAGCCTGCGTGGCTGCCTGCGCACCTCTCATGAGGTGTGAA | |
| | GCCAAAGATCGGACAGGGTGTGAAGAGCCGCGTGTGCTCACTTTGAGTCC | |
| | TCCGGCCCCTGAATGCGGCTAACCTTAAACCTGCAGCCATGGCTCATAAG | |
| | CCAATGAGTTTATGGTCGTAACGAGTAATTGCGGGATGGGACCGACTACT | |
| | TTGGGTGTCCGTGTTTCACTTTTTCCTTTATTAATTGCTTATGGTGACAA | |
| | TATATATATTGATATATATTGGCATC | |
| 129 | cccacagcaagaatgccatcatctgtcctcacccccaattttcccttttc | 18-675 |
| | ttcccctgcaaccattacgcttactcgcatgtgcattgagtggtgcatgt | |
| | gttgaacaaacagctacactcacatgggggcgggttttcccgccctacgg | |
| | cctctcgcgaggcccaccccttccctccccttataactacagtgctttgg | |
| | taggtaagcatcctgatcccccgcggaagctgctcacgtggcaactgtgg | |
| | ggacccagacaggttatcaaaggcacccggtctttccgccttcaggagta | |
| | tccctactagtgaattctagcggggctctgcttggtgccaacctccccca | |
| | aatgcgcgctgcgggagtgctcttccccaactcaccctagtatcctctca | |
| | tgtgtgtgcttggtcagcatatctgagacgatgttccgctgtcccagacc | |
| | agtccagtaatggacgggccagtgcgtgtagtcgtcttccggcttgtccg | |
| | gggcatgtttggtgaaccggtggggtaaggttggtgtgcccaacgcccgt | |
| | actttggtgacacctcaagaccacccaggaatgccagggaggtaccccac | |
| | ctcacggtgggatctgaccctgggctaattgtctacggtggttcttcttg | |
| | cttccacttctttcttctgttcacg | |
| 130 | Gtataagagacaggtgtttgccttgtcttcggactggcatcttgggacca | Crohivirus B |
| | accccccttttccccagccatgggttaaatggcaataaaggacgtaacaa | |
| | ctttgtaaccattaagctttgtaattttgtaaccactaagctttgtgcac | |
| | ataatgtaaccatcaagcttgttagtcccagcaggaggtttgcatgcttg | |
| | tagccgaaatggggctcgaccccccatagtaggatacttgattttgcatt | |
| | ccattgtggacctgcaaactctacacatagaggctttgtcttgcatctaa | |
| | acacctgagtacagtgtgtacctagaccctatagtacgggaggaccgttt | |
| | gtttcctcaataaccctacataataggctaggtgggcatgcccaatttgc | |
| | aagatcccagactgggggtcggtctgggcagggttagatccctgttagct | |
| | actgcctgatagggtggtgctcaaccatgtgtagtttaaattgagctgtt | |
| | catatacc | |
| 131 | Acatggggggtctgcggacggcttcggcccacccgcgacaagaatgccgt | Salivirus FHB |
| | catctgtcctcattacccgtattccttcccttcccccgcaaccaccacgc | |
| | ttactcgcgcacgtgttgagtggcacgtgcgttgtccaaacagctacacc | |
| | cacacccttcggggcgggtttgtcccgccctcgggttcctcgcggaaccc | |
| | ccccctccctctctctctttctatccgccctcacttcccataactacagt | |
| | gctttggtaggtgagcaccctgaccccccgcggaagctgctaacgtggca | |
| | actgtggggatccaggcaggttatcaaaggcacccggtctttccgccttc | |
| | aggagtatctctgccggtgaattccggtagggctctgcttggtgccaacc | |
| | tcccccaaatgcgcgctgcgggagtgctcttccccaactcatcttagtaa | |
| | cctctcatgtgtgtgcttggtcagcatatctgaggcgacgttccgctgtc | |
| | ccagaccagtccagcaatggacgggccagtgtgcgtagtcgctttccggt | |
| | tttccggcgcatgtttggcgaaacgctgaggtaaggttggtgtgcccaac | |
| | gcccgtaatttggtgatacctcaagaccacccaggaatgccagggaggta | |
| | ccccacttcggtgggatctgaccctgggctaattgtctacggtggttctt | |
| | cttgcttccacttctcttttttctggcatg | |
| 132 | Tttgaaaagggggtgggggggcctcggccccctcaccctcttttccggtg | Aichi Virus |
| | gtctggtcccggaccaccgttactccattcagcttcttcggaacctgttc | |
| | ggaggaattaaacgggcacccatactccccccaccccccttttgtaacta | |
| | agtatgtgtgctcgtgatcttgactcccacggaacggaccgatccgttgg | |
| | tgaacaaacagctaggtccacatcctcccttcccctgggagggcccccgc | |
| | cctcccacatcctccccccagcctgacgtatcacaggctgtgtgaagccc | |
| | ccgcgaaagctgctcacgtggcaattgtgggtccccccttcatcaagaca | |
| | ccaggtctttcctccttaaggctagccccggcgtgtgaattcacgttggg | |
| | caactagtggtgtcactgtgcgctcccaatctcggccgcggagtgctgtt | |
| | ccccaagccaaacccctggcccttcactatgtgcctggcaagcatatctg | |
| | agaaggtgttccgctgtggctgccaacctggtgacaggtgccccagtgtg | |
| | cgtaaccttcttccgtctccggacggtagtgattggttaagatttggtgt | |
| | aaggttcatgtgccaacgccctgtgcgggatgaaacctctactgccctag | |
| | gaatgccaggcaggtaccccacctccgggtgggatctgagcctgggctaa | |
| | ttgtctacgggtagtttcatttccaatccttttatgtcggagtc | |
| 133 | Tatggcaggcgggcttgtggacggcttcggcccacccacagcaagaatgc | Salivirus NG-J1 |
| | catcatctgtcctcacccccaattttcccttttcttcccctgcaaccatt | |
| | acgcttactcgcatgtgcattgagtggtgcatgtgttgaacaaacagcta | |
| | cactcacatgggggcgggttttcccgccctacggcctctcgcgaggccca | |
| | ccccttccctccccttataactacagtgctttggtaggtaagcatcctga | |
| | tcccccgcggaagctgctcacgtggcaactgtggggacccagacaggtta | |
| | tcaaaggcacccggtctttccgccttcaggagtatccctactagtgaatt | |
| | ctagcggggctctgcttggtgccaacctcccccaaatgcgcgctgcggga | |
| | gtgctcttccccaactcaccctagtatcctctcatgtgtgtgcttggtca | |
| | gcatatctgagacgatgttccgctgtcccagaccagtccagtaatggacg | |
| | ggccagtgcgtgtagtcgtcttccggcttgtccggggcatgtttggtgaa | |
| | ccggtggggtaaggttggtgtgcccaacgcccgtactttggtgacacctc | |
| | aagaccacccaggaatgccagggaggtaccccacctcacggtgggatctg | |
| | accctgggctaattgtctacggtggttcttcttgcttccacttctttctt | |
| | ctgttcacg | |
| 134 | Ttaaaacagcggatgggtatcccaccatccgacccacagggtgtagtgct | HRVB27 |
| | ctggtattttgtacctttgcacgcctgtttccccattgtacccctcctta | |
| | aatttcctccccaagtaacgttagaagtttaaggaaacaaatgtacaata | |
| | ggaagcatcacatccagtggtgttatgtacaagcacttctgtttccccgg | |
| | agcgaggtataagtggtacccaccgccgaaagcctttaaccgttatccgc | |
| | caatcaactacgtaatggctagtattaccatgtttgtgacttggtgttcg | |
| | atcaggtggttccccccactagtttggtcgatgaggctaggaactcccca | |
| | cgggtgaccgtgtcctagcctgcgtggcggccaacccagcttttgctggg | |
| | acgcctttttacagacatggtgtgaagacctgcatgtgcttgattgtgag | |
| | tcctcggcccctgaatgcggctaaccttaaccccggagccttgcaacata | |
| | atccaatgttgttgaggtcgtaatgagtaattctgggatgggaccgacta | |
| | ctttgggtgtccgtgtttccttttattctttatattgtcttatggtcaca | |
| | gcatatatagcatatatactgtgatc | |
| 135 | cttccttttaattcgtaactgataagtgatagtccttggaagctaggtat | PTV B |
| | ttgttacgctagttttggattatcttgtgcccaacatttgttttcgaaca | |
| | tatgttgtgtttaaacacagaaatctagtttctttggttatgagtttaat | |
| | ggaatatccttttgaaagacttgccttggcgcgggctagagcgcaattgt | |
| | caccaggtattgcaccaatggtggcgacagggtacagaagagcaagtact | |
| | cctgactgggtaatgggactgcattgcatatccctaggcatctattgaga | |
| | tttctctggagcccaccagcatggagttcctgtatgggaatgcaggactg | |
| | gacttgtgctgcctgacagggtcgcggctggccgtctgtactttgtatag | |
| | tcagttgaaactcatt | |
| 136 | CGGACTTAAATAATTGAGCCTTAAAGAAGAAATTCTTTAAGTGGATGCTC | GROUP I INTRON, |
| | TCAAACTCAGGGAAACCTAAATCTAGTTATAGACAAGGCAATCCTGAGCC | Anabaena sp.YBS01 |
| | AAGCCGAAGTAGTAATTAGTAAAACAATAGATGACTTACAACTAATCGGA | |
| | AGGTGCAGAGACTCGACGGGAGCTACCCTAACGTCAAGACGAGGGTAAAG | |
| | AGAGAGTCCAATTCTCAAAGCCAATAGGCAGTAGCGAAAGCTGCAAGAGA | |
| | ATGAAAATCCGT | |
| 137 | GAGCGGCGGACTCATATTTCGATGTGCCTTGCGCCGGGAAACCACGCAAG | GROUP I INTRON, |
| | GGATGGTGTCAAATTCGGCGAAACCTAAGCGCCCGCCCGGGCGTATGGCA | Azoarcus sp.BH72 |
| | ACGCCGAGCCAAGCTTCGGCGCCTGCGCCGATGAAGGTGTAGAGACTAGA | |
| | CGGCACCCACCTAAGGCAAACGCTATGGTGAAGGCATAGTCCAGGGAGTG | |
| | GCGAAAGTCACACAAACCGGAATCCGCCGGTG | |
| 138 | ctttcggacaggggttcgatTCAAAAGAGTCGCCTTATGAAGTGATTCAT | GROUP I INTRON, |
| | AAGTGAAAACTTGGCTTTATCGGTGAAACCGAAACGTAAGACGTCGGCAA | Clostridium |
| | TACCGAGTGGTATGTGGGGAAACCCAACAGCCGTATCGACTCATAGGTTT | botulinum |
| | CTAACCTTAAGAAATGAAATTTTCTTAAGTAAGAAATACTAGGATAGAAG | |
| | TTTTAAACTATAATATAGCTTCTATAACATGCCAAGGTACTTAAAACTTA | |
| | TAAGTTTTAAGCACGAGATATAGTCAGTGCCATTAGAGATAATGGAATAG | |
| | CATGtcccctcgcctccacca | |
| 139 | agacgctacggacttAATTGGATTGAGCCTTGGTATGGAAACCTACTAAG | GROUP I INTRON, |
| | TGATAACTTTCAAATTCAGAGAAACCCGGGAATTAACAATGGGCAATCCT | Ddu.c.trnL-1 |
| | GAGCCAAATCCTGGTTTACGCGAACAAACCAAAGTGTAGAAAGCGAGAAA | |
| | GAAGGGATAGGTGCAGAGACTCAATGGAAGCTGTTCTAACAAATGGAGTT | |
| | CACTACCTTGTATTGATCAAATGATTCACTTCATAGTCTGATAGATCCTT | |
| | GGTGGAACTGATTAATCGGACGAGAATAAAGATAGAGTCCCATTCTACAT | |
| | GTCAATACTGACAACAATGAAATTTATAGTAAGATGaaaatccgttgact | |
| | t | |
| 140 | agacgctacggacttAATTGGATTGAGCCTTGGTATGGAAACCTACTAAG | GROUP I INTRON, |
| | TGATAACTTTCAAATTCAGAGAAACCCGGGAATTAACAATGGGCAATCCT | Dla.c.trnL |
| | GAGCCAAATCCTGGTTTACGCGAACAAACCAAAGTGTAGAAAGCGAGAAA | |
| | GAAGGGATAGGTGCAGAGACTCAATGGAAGCTGTTCTAACAAATGGAGTT | |
| | CACTACCTTGTATTGATCAAATGATTCACTTCATAGTCTGATAGATCCTT | |
| | GGTGGAACTGATTAATCGGACGAGAATAAAGATAGAGTCCCATTCTACAT | |
| | GTCAATACTGACAACAATGAAATTTATAGTAAGATGaaaatccgttgact | |
| | t | |
| 141 | agacgctacggacttAATTGGATTGAGCCTTGGTATGGAAACCTACTAAG | GROUP I INTRON, |
| | TGATAACTTTCAAATTCAGAGAAACCCGGGAATTAACAATGGGCAATCCT | Dto.c.trnL-1 |
| | GAGCCAAATCCTGGTTTACGCGAACAAACCAAAGTGTAGAAAGCGAGAAA | |
| | GAAGGGATAGGTGCAGAGACTCAATGGAAGCTGTTCTAACAAATGGAGTT | |
| | CACTACCTTGTATTGATCAAATGATTCACTTCATAGTCTGATAGATCCTT | |
| | GGTGGAACTGATTAATCGGACGAGAATAAAGATAGAGTCCCATTCTACAT | |
| | GTCAATACTGACAACAATGAAATTTATAGTAAGATGaaaatccgttgact | |
| | t | |
| 142 | ctacggacttAATTGGATTGAGCCTTGGTATGGAAACCTACCAAGTGATA | GROUP I INTRON, |
| | ACTTTCAAATTCAGAGAAACCCTGGAATTAAAAATGGGCAATCCTGAGCC | Fan.c.trnL |
| | AAATCCCGTTTTATGAAAACAAACAAGGGTTTCAGAAAGCGAGAATAAAT | |
| | AAAGGATAGGTGCAGAGACTCAATGGAAGCTGTTCTAACAAATGGAGTTG | |
| | GCTGCATTGTGTTCGTAGTAAAGGAATCGAAACTTCCGAAAGGATGAAAG | |
| | ATAAACCTATATACATATGTATACTTACTGAAATACTATCGCCAAATAAT | |
| | TCAAAATGATTAATGACGACTCCGTCCGCTAAATCTTTTTATTTTTAAAA | |
| | ATTTTTAAATCGGATAAGAATAAAGATAGAGTCCCATTCTACATGTCAAT | |
| | ATCGACAACAATGAAATTTATAGTAAGAGGaaaatccgtc | |
| 143 | aatcgcagtgtggttTAACATTCGGGCCACACTATAATAAACTCCTTGAA | GROUP I INTRON, |
| | TTCGGTAAACCTCTCAACGAATAAAGTTCGCAGACAATACCGAGCTAAGC | LL-H.v.terL |
| | TTAATATGCTAAGATTTTAAATGTGTTTACTCCAACCAAACATGCTATAA | |
| | TAAACCTAAGAGGTGATAGCAATGGAGCATTGGAAATGGATAGATGGTTT | |
| | TGAGGGCAAATACGAAGTCAGTGATTTAGGCAGAGTAAGGTCTTATGCAA | |
| | CTGGCAAATGCGCTTATCTATCTGTTAATCGCCTAACTCGCGATGGCTAT | |
| | AGCCATATTGCTTTGCGAAAAAATGGCAAAGCATATGAGTTTAGACTAAA | |
| | CAGACTGGTGGCAGCAGCTTTTATTGGCCAACCATCTAAAGAGAAAAACA | |
| | CAGTTAATCACATTAATGGCATCAAAACTGATAATCGAGCAGTTAACCTT | |
| | GAATGGGCCTCACGATCAGAGCAAATGTATCATGCTTATCAACACCATTT | |
| | AAAATTACCCAGGAAAGGAAAACGCAGAACATTTGACGACTTCAGCTTTG | |
| | AAGAAAAGCAAGATATCTGGAATAATTATCAGCCATATAAAAAAGGGCAC | |
| | AGTAAAAGATATTTTTGCATTAAATATAATACCCACTATTCAGTGATTGA | |
| | CAAAATCTTAGCAGAAAAGAAAGTGTAACGACTATCGAAACACAAAAAGC | |
| | ATCAGCTGATTAGCTGATGCTTTTTGAATGGAGTAGAGTAGTCCCCAAGC | |
| | GGGGGCGAAGCAGGGAGCATCAGCAATATGATGAAGAGCTAGTCTGAACT | |
| | TGATAGAAATATCAAGAGCATGTATGGACGCGATACATGCGCAACAACAT | |
| | TAcgaagaatttgcaaa | |
| 144 | agacgctgcggacttAATTTAATTGAGCTTTAGTTGAGAAATTTACTAAA | GROUP I INTRON, |
| | TGATTGTTTTCAAATTCAGGGAAACCTAGGTTGCAAAAAAAAGATAAAAA | Mpo.c.trnL |
| | TTAGGTAATCCTGAGCCAAATTTTGTTTACTAAAACAAAAGAGGTGCAGA | |
| | GACTCAAAGAAAACTATCCTAACGAAATTTTTTATCATTTTTATAAAAAA | |
| | TTGGATTAATATATTAATTAATAATAATAAAATTATTAAATCATTTTTTC | |
| | ATTTTAAATATAGACGAGGATAAAGATAGAGTCCGTTTTTACAAGTTAAT | |
| | TTTAACAACAATGCAAATTGTAGTAAAATGaaaatccgttggctt | |
| 145 | ctacggacttGATCGAATTGAGCCTTGGTATGGAAACCTACCAAGTGATA | GROUP I INTRON, |
| | GCTTCCAAATCCAGGGAACCCTGGGATATTTTGAATGGGTAATCCTGAGC | Pth.c.trnL |
| | CAAATCCGGTTCATGAAGACAATGTTTCTTCTCCTAAGATAGGAAGGGGA | |
| | TAGGTGCAGAGACTCAATGGAAGCTATTCTAACGAATGAATCTCATTTGG | |
| | TCCAATACTGTAGTTACTGTAGTTATAGAACGATCTATTTACACCTCAAA | |
| | AATGGAGAGAGATGTTATATAACATCAGACAAAACTGGCGATCAGAACTC | |
| | GTTCCAAGCATTTTATTCATAAGATAGATGCCAGATTCGAGTTGAAGTAC | |
| | TGATTTGACATTAAGTAATCCAATTATGAACTTATCTACTCTAGATGAAT | |
| | AATTTAATTATTTTTTGGAATAAATGGTTGGACGAGGATAAAGATAGAGT | |
| | CCAATTCTACATGTCAATGTAAACAACAATGCAAATTGCAGTAGGAGGaa | |
| | aatccgtt | |
| 146 | agacgctacggacttGATTGTATTGAGCCTTGGTATGGAAACCTGCTAAG | GROUP I INTRON, |
| | TGGTAACTTCCAAATTCAGAGAAACCCTGGAATGAAAAATGGGCAATCCT | Sbi.c.trnL |
| | GAGCCAAATCCACTTTTTTCAAAAAAGTGGTTCTCAAACTAGAACCCAAA | |
| | GGAAAAGGATAGGTGCAGAGACTCAATGGAAGCTGTTCTAACGAATCGAA | |
| | GTAATAACGATTAATCACAGAACCCATATTATAATATAGGTTCTTTATTT | |
| | TATTTTGAGAATGAAATTAGGAATGATTATGAAATAGAAAATTCATAATT | |
| | TTTTTAGAATTATTGTGAATCTATTCCAATCGAATATTGAGTAATCAAAT | |
| | CCTTCAATTCATTGTTTTCGAGATCTTTTAAAAAGTGGATTAATCGGACG | |
| | AGGATAAAGAGAGAGTCCCATTCTACATGTCAATACTGACAACAATGAAA | |
| | TTTCTAGTAAAAGGaaaatccgtcgactt | |
| 147 | aacaggtTGACGACAACAGGCCTGTGACAGCGGGGCTTCGAACTTTCCGT | GROUP I INTRON, |
| | GTTTTTTTTTCTTCCGCTAGTCGATCATCCGTGACGGCCGGGCAAGCGCC | Scytalidium_ |
| | CGAGTACCAGGACCGTCGGGCCCCCGAGAAGGGGGGGCCTAGGATGCGGC | dimidiatum |
| | AAGACGACCCGGTTCGGGGAACGCCAGCGTGCGCTGGCCGATCCCGAGGC | |
| | GAGGTGCCGTAGCGGGCACCCGTCGTAACGCGCGGTAGGGCGTCGGTCCC | |
| | CCCTCCCACGGCGGAGGGGAGGCTTAAGGTACGTGCTAAACCCCCAGCGA | |
| | TGGGGCCTGTAGGAAAAGCCCTGGTACGGCGAAGCCTACGGGGACCGCCC | |
| | GATGGCGGTCGGATGCCGGCGGGCCACGGAGCCCCCGGCGTCGATTGctg | |
| | tta | |
| 148 | agacgcagcggacttAGAAAACTGGGCCTCGATCGCGAAAGGGATCGAGT | GROUP I INTRON, |
| | GGCAGCTCTCAAACTCAGGGAAACCTAAAACTTTAAACATTAAAGTCATG | Sel.b.trnL-2 |
| | GCAATCCTGAGCCAAGCTAAAGCTAAACAACTAACAGCTTTAGAAGGTGC | |
| | AGAGACTAGACGGGAGCTACCCTAACGGATTCAGCCGAGGGTAAAGGGAT | |
| | AGTCCAATTCTCAACATCGCGATTGTTGATGGCAGCGAAAGTTGCAGAGA | |
| | GAATGaaaatccgctgactg | |
| 149 | gatgttttcttgggtTAATTGAGGCCTGAGTATAAGGTGACTTATACTTG | GROUP I INTRON, |
| | TAATCTATCTAAACGGGGAACCTCTCTAGTAGACAATCCCGTGCTAAATT | T4.v.td, 1 |
| | GTAGGACTGCCCTTTAATAAATACTTCTATATTTAAAGAGGTATTTATGA | |
| | AAAGCGGAATTTATCAGATTAAAAATACTTTAAACAATAAAGTATATGTA | |
| | GGAAGTGCTAAAGATTTTGAAAAGAGATGGAAGAGGCATTTTAAAGATTT | |
| | AGAAAAAGGATGCCATTCTTCTATAAAACTTCAGAGGTCTTTTAACAAAC | |
| | ATGGTAATGTGTTTGAATGTTCTATTTTGGAAGAAATTCCATATGAGAAA | |
| | GATTTGATTATTGAACGAGAAAATTTTTGGATTAAAGAGCTTAATTCTAA | |
| | AATTAATGGATACAATATTGCTGATGCAACGTTTGGTGATACATGTTCTA | |
| | CGCATCCATTAAAAGAAGAAATTATTAAGAAACGTTCTGAAACTGTTAAA | |
| | GCTAAGATGCTTAAACTTGGACCTGATGGTCGGAAAGCTCTTTACAGTAA | |
| | ACCCGGAAGTAAAAACGGGCGTTGGAATCCAGAAACCCATAAGTTTTGTA | |
| | AGTGCGGTGTTCGCATACAAACTTCTGCTTATACTTGTAGTAAATGCAGA | |
| | AATCGTTCAGGTGAAAATAATTCATTCTTTAATCATAAGCATTCAGACAT | |
| | AACTAAATCTAAAATATCAGAAAAGATGAAAGGTAAAAAGCCTAGTAATA | |
| | TTAAAAAGATTTCATGTGATGGGGTTATTTTTGATTGTGCAGCAGATGCA | |
| | GCTAGACATTTTAAAATTTCGTCTGGATTAGTTACTTATCGTGTAAAATC | |
| | TGATAAATGGAATTGGTTCTACATAAATGCCTAACGACTATCCCTTTGGG | |
| | GAGTAGGGTCAAGTGACTCGAAACGATAGACAACTTGCTTTAACAAGTTG | |
| | GAGATATAGTCTGCTCTGCATGGTGACATGCAGCTGGATATAATTCCGGG | |
| | GTAAGATTAACGACCTTATCTGAACATAATGctaccgtttaatatt | |
| 150 | agacgctacggacttGATTGTATTGAGCCTTGGTATGGAAACCTGCTAAG | GROUP I INTRON, |
| | TGGTAACTTCCAAATTCAGAGAAACCCTGGAATGAAAAATGGGCAATCCT | Zma.c.trnL |
| | GAGCCAAATCCCTTTTTTGAAAAACAAGTGGTTCTCAAACTAGAACCCAA | |
| | AGGAAAAGGATAGGTGCAGAGACTCAATGGAAGCTGTTCTAACGAATCGA | |
| | AGTAATAACGATTAATCACAGAACCCATATTATAATATAGGTTCTTTATT | |
| | TTATTTTTAGAATGAAATTAGGAATGATTATGAAATAGAAAATTCATAAT | |
| | TTTTTTTTAGAATTATTGTGAATCTATTCCAATCAAATATTGAGTAATCA | |
| | AATCCTTCAATTCATTGTTTTCGAGATCTTTTAATTTTAAAAAGTGGATT | |
| | AATCGGACGAGGATAAAGAGAGAGTCCCATTCTACATGTCAATACTGACA | |
| | ACAATGAAATTTCTAGTAAAAGGaaaatccgtcgactt | |
| 151 | CCGTCGATTGTCCACTGGTC | 5′ homology arm- |
| | | original (20) |
| 152 | ccgtcgattgtccactggtcccgtcgattgtccactggtc | 5′ homology arm- |
| | | 2xoriginal (40) |
| 153 | aaaaaaaaaaaaaaaaaaaa | 5′ homology arm- |
| | | AU pair (20), 3′ |
| | | homology arm-UA |
| | | pair (20) |
| 154 | tttttttttttttttttttt | 5′ homology arm- |
| | | UA pair (20), 3′ |
| | | homology arm-AU |
| | | pair (20) |
| 155 | aaaaaaaaaaaaaaaaaaaaaaaaaaaaaaaaaaaaaaaa | 5′ homology arm- |
| | | AU pair (40), 3′ |
| | | homology arm-UA |
| | | pair (40) |
| 156 | tttttttttttttttttttttttttttttttttttttttt | 5′ homology arm- |
| | | UA pair (40), 3′ |
| | | homology arm-AU |
| | | pair (40) |
| 157 | CCCCCCCCCCCCCCCCCCCC | 5′ homology arm- |
| | | CG pair (20), |
| 158 | gggggggggggggggggggg | 3′ homology arm- |
| | | CG pair (20) |
| 159 | gggggggggggggggggggggggggggggggggggggggg | 5′ homology arm- |
| | | GC pair (40), 3′ |
| | | homology arm-CG |
| | | pair (40) |
| 160 | cccccccccccccccccccccccccccccccccccccccc | 5′ homology arm- |
| | | CG pair (40), 3′ |
| | | homology arm-GC |
| | | pair (40) |
| 161 | GACCAGTGGACAATCGACGG | 3′ homology arm- |
| | | original (20) |
| 162 | gaccagtggacaatcgacgggaccagtggacaatcgacgg | 3′ homology arm- |
| | | 2x original (40) |

What we claimed is:

1. A circular RNA precursor comprising the following elements from 5′ to 3′ direction in the following order:
a) a 3′ self-splicing intron fragment;
b) a first residual circularizing element;
c) a nucleotide sequence of interest;
d) a second residual circularizing element; and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 5 to about 100 nucleotides.

2. The circular RNA precursor of claim 1, wherein the self-splicing intron is selected from Group I introns and Group II introns, for example, the self-splicing intron is Group I introns.

3. The circular RNA precursor of claim 1 or 2, wherein the self-splicing intron is selected from IC3 subgroup of Group I introns.

4. The circular RNA precursor of any one of claims 1-3, wherein the self-splicing intron is selected from a Group I intron of the cyanobacterium Anabaena, a Group I intron from a T4 phage or a Group I intron from Azoarcus sp. BH72, for example, the self-splicing intron is a Group I intron of the cyanobacterium Anabaena.

5. The circular RNA precursor of any one of claims 1-4, wherein the self-splicing intron is selected from the Group I intron of the Anabaena pre-tRNA-Leu gene, the Group I intron of the td gene of T4 phage, or the Group I intron of the pre-tRNA-Ile gene of Azoarcus sp. BH72, for example, the self-splicing intron is the Group I intron of the Anabaena pre-tRNA-Leu gene.

6. The circular RNA precursor of any one of claims 1-5, wherein the 3′ self-splicing intron fragment is derived from a 3′ terminal portion of a native self-splicing intron starting from an internal split site to the 3′ end of the native self-splicing intron, and wherein the 5′ self-splicing intron fragment is derived from a 5′ terminal portion of the native self-splicing intron starting from the internal split site to the 5′ end of the native self-splicing intron, and, the 3′ self-splicing intron fragment and the 5′ self-splicing intron fragment in combination retain the self-splicing activity of the native self-splicing intron.

7. The circular RNA precursor of claim 6, wherein the internal split site is located within the P6, P2, P5, P8 or P9 region of the Group I intron or the D4 region of the Group II intron, preferably, the internal split site is located within the P6 region of the Group I intron.

8. The circular RNA precursor of claim 6 or 7, wherein the 3′ self-splicing intron fragment is a sequence having at least 75%, at least 80%, at least 85%, at least 90%, at least 95%, 100% sequence identity to the 3′ terminal portion of a native self-splicing intron, and the 5′ self-splicing intron fragment is a sequence having at least 75%, at least 80%, at least 85%, at least 90%, at least 95%, 100% sequence identity to the 5′ terminal portion of a native self-splicing intron.

9. The circular RNA precursor of any one of claims 1-8, wherein self-splicing intron is Group I intron of the Anabaena pre-tRNA-Leu gene, and the 3′ self-splicing intron fragment comprises or consists of a nucleotide sequence of SEQ ID NO: 1 or a nucleotide sequence having at least 75%, at least 80%, at least 85%, at least 90%, at least 95%, 100% identity to SEQ ID NO: 1, and the 5′ self-splicing intron fragment comprises or consists of a nucleotide sequence of SEQ ID NO: 2 or a nucleotide sequence having at least 75%, at least 80%, at least 85%, at least 90%, at least 95%, 100% identity to SEQ ID NO: 2.

10. The circular RNA precursor of any one of claims 1-8, wherein the self-splicing intron is the Group I intron of the td gene of T4 phage, and the 3′ self-splicing intron fragment comprises or consists of a nucleotide sequence of SEQ ID NO: 5 or a nucleotide sequence having at least 75%, at least 80%, at least 85%, at least 90%, at least 95%, 100% identity to SEQ ID NO: 5, and the 5′ self-splicing intron fragment comprises or consists of a nucleotide sequence of SEQ ID NO: 6 or a nucleotide sequence having at least 75%, at least 80%, at least 85%, at least 90%, at least 95%, 100% identity to SEQ ID NO: 6.

11. The circular RNA precursor of any one of claims 1-8, wherein the self-splicing intron is the Group I intron of the pre-tRNA-Ile gene of Azoarcus sp. BH72, and the 3′ self-splicing intron fragment comprises or consists of a nucleotide sequence of SEQ ID NO: 3 or a nucleotide sequence having at least 75%, at least 80%, at least 85%, at least 90%, at least 95%, 100% identity to SEQ ID NO: 3, and the 5′ self-splicing intron fragment comprises or consists of a nucleotide sequence of SEQ ID NO: 4 or a nucleotide sequence having at least 75%, at least 80%, at least 85%, at least 90%, at least 95%, 100% identity to SEQ ID NO: 4.

12. The circular RNA precursor of any one of claims 1-11, wherein the first residual circularizing element and the second residual circularizing element are configured such that the circular RNA comprising them has reduced immunogenicity relative to a control RNA, such as a control circular RNA comprising the circularizing elements of SEQ ID NO:29 and SEQ ID NO:64 or a control linear RNA comprising the same nucleotide sequence of interest.

13. The circular RNA precursor of any one of claims 1-12, wherein the first residual circularizing element and the second residual circularizing element are configured such that the circular RNA or circular RNA precursor comprising them has comparable or increased circularization efficiency relative to a control circular RNA or circular RNA precursor, such as a control circular RNA or circular RNA precursor comprising a first residual circularizing element and a second residual circularizing element of SEQ ID NO:29 and SEQ ID NO:64 (Ana 3.0), respectively.

14. The circular RNA precursor of any one of claims 1-13, wherein the total length of the first residual circularizing element and the second residual circularizing element is about 20 to about 35 nucleotides.

15. The circular RNA precursor of any one of claims 1-14, wherein the first residual circularizing element and the second residual circularizing element are configured to be capable of forming a stem-loop structure, e.g., upon self-splicing for circularization.

16. The circular RNA precursor of claim 15, wherein the loop of the stem-loop structure comprises the splicing junction.

17. The circular RNA precursor of any one of claims 15-16, wherein the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure, e.g., through self-splicing for circularization.

18. The circular RNA precursor of claim 17, wherein the first loop sequence comprises or consists of one or more nucleotides which can pair with the P1 region of the corresponding self-splicing intron to form a P10 duplex region during the circularization.

19. The circular RNA precursor of claim 17 or 18, wherein the first loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.

20. The circular RNA precursor of any one of claims 17-19, wherein the first loop sequence comprises or consists of the sequence of AAAA, AA, UUUU, UAAA, CAAAA, or GAAAA, preferably AAAA.

21. The circular RNA precursor of any one of claims 17-20, wherein the second loop sequence comprises or consists of one or more nucleotides which can pair with the internal guide sequence (IGS) of the corresponding self-splicing intron to form a P1 duplex region during the circularization.

22. The circular RNA precursor of any one of claims 17-21, wherein the second loop sequence comprises or consists of CUU or CUC, preferably CUU.

23. The circular RNA precursor of any one of claims 17-22, wherein the loop of the stem-loop structure has a sequence of CUUAAAA, CUUUUUU, CUUAA, CUUGAAA, CUUUAAA, CUUCAAA or CUCAAAA, preferably CUUAAAA.

24. The circular RNA precursor of any one of claims 17-23, wherein the stem portion of the stem-loop structure comprises 2-15 or more consecutive matched base pairs, preferably, the stem portion of the stem-loop structure comprises 5, 6 or 7 consecutive matched base pairs.

25. The circular RNA precursor of any one of claims 17-24, wherein the stem portion in the stem-loop structure comprises up to 2 base pair mismatches, or the stem portion comprises only 1 base pair mismatch, preferably, the stem portion comprises no base pair mismatches.

26. The circular RNA precursor of any one of claims 17-25, wherein the first pairing sequence comprises only Gs and the second pairing sequence comprises only Cs.

27. The circular RNA precursor of any one of claims 17-25, wherein the first pairing sequence comprises only Cs and the second pairing sequence comprises only Gs.

28. The circular RNA precursor of any one of claims 17-25, wherein the first pairing sequence includes only A and the second pairing sequence includes only U.

29. The circular RNA precursor of any one of claims 17-25, wherein the first pairing sequence comprises or consists of the sequence of any one of SEQ ID NO:42-55, and, the second pairing sequence comprises or consists of the sequence of any one of SEQ ID NO:78-93.

30. The circular RNA precursor of any one of claims 15-29, wherein the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower.

31. The circular RNA precursor of any one of claims 1-30, wherein the first residual circularizing element comprises or consists of from 5′ to 3′ direction a 3′ exon region and optionally a first spacer, the second residual circularizing element comprises or consists of from 3′ to 5′ direction a 5′ exon region and optionally a second spacer.

32. The circular RNA precursor of claim 31, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.

33. The circular RNA precursor of claim 32, wherein 3′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 5′ exon or a contiguous fragment of about 1-about 50 nucleotides starting from the 3′ terminal nucleotide of the native 5′ exon.

34. The circular RNA precursor of any one of claims 1-33, wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55.

35. The circular RNA precursor of any one of claims 1-34, wherein the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.

36. The circular RNA precursor of any one of claims 1-35, wherein the first residual circularizing element comprises or consists of a nucleotide sequence of any one of SEQ ID NOs: 13-55 and the second residual circularizing element comprises or consists of a nucleotide sequence of any one of SEQ ID NOs: 56-93.

37. The circular RNA precursor of any one of claims 1-36, wherein,
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 13 and the second residual circularizing element comprises or consists of a nucleotide sequence of SEQ ID NO: 56;
15) the first residual circularizing element comprises or consists of a nucleotide sequence of SEQ ID NO: 17 and the second residual circularizing element comprises or consists of a nucleotide sequence of SEQ ID NO: 59; or
16) the first residual circularizing element comprises or consists of a nucleotide sequence of SEQ ID NO: 22 and the second residual circularizing element comprises or consists of a nucleotide sequence of SEQ ID NO: 63.

38. A circular RNA precursor comprising the following elements from 5′ to 3′ direction in the following order:
a) a 3′ self-splicing intron fragment;
b) a first residual circularizing element;
c) a nucleotide sequence of interest;
d) a second residual circularizing element; and
e) a 5′ self-splicing intron fragment;
wherein the circular RNA precursor allows generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 5 to about 100 nucleotides,
wherein the self-splicing intron is selected from the Group I intron of the Anabaena pre-tRNA-Leu gene,
wherein the 3′ self-splicing intron fragment comprises or consists of a nucleotide sequence of SEQ ID NO: 1 or a nucleotide sequence having at least 75%, at least 80%, at least 85%, at least 90%, at least 95%, 100% identity to SEQ ID NO: 1, and the 5′ self-splicing intron fragment comprises or consists of a nucleotide sequence of SEQ ID NO: 2 or a nucleotide sequence having at least 75%, at least 80%, at least 85%, at least 90%, at least 95%, 100% identity to SEQ ID NO: 2,
wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55, and the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.

39. The circular RNA precursor of claim 38, wherein the 3′ self-splicing intron fragment comprises or consists of a nucleotide sequence of SEQ ID NO: 1 and 5′ self-splicing intron fragment comprises or consists of a nucleotide sequence of SEQ ID NO: 2.

40. The circular RNA precursor of claim 38 or 39, wherein
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 13 and the second residual circularizing element comprises or consists of a nucleotide sequence of SEQ ID NO: 56;
15) the first residual circularizing element comprises or consists of a nucleotide sequence of SEQ ID NO: 17 and the second residual circularizing element comprises or consists of a nucleotide sequence of SEQ ID NO: 59; or
16) the first residual circularizing element comprises or consists of a nucleotide sequence of SEQ ID NO: 22 and the second residual circularizing element comprises or consists of a nucleotide sequence of SEQ ID NO: 63.

41. The circular RNA precursor of any one of claims 1-40, wherein the circular RNA precursor further comprises a 5′ homology arm sequence and a 3′ homology arm sequence capable of complementary pairing to each other to form a homology arm double-stranded region.

42. The circular RNA precursor of claim 41, wherein the 5′ homology arm sequence is upstream of the 3′ self-splicing intron fragment and the 3′ homology arm sequence is downstream of the 5′ self-splicing intron fragment.

43. The circular RNA precursor of any one of claims 41-42, wherein the homology arm is about 5-50 nucleotides in length, preferably, about 40 nucleotides in length.

44. The circular RNA precursor of any one of claims 41-43, wherein the two homology arm sequences may be polyA and polyT, respectively, or polyG and polyC, respectively.

45. The circular RNA precursor of any one of claims 41-44, wherein one of the homology arm sequence has the nucleotide sequence of any one of SEQ ID NO: 151-161 or a nucleotide sequence having at least 50%, at least 60%, at least 70%, at least 80%, at least 90%, at least 95%, or at least 99% sequence identity with any one of SEQ ID NO:151-161, while the other homology arm sequence has the corresponding complementary sequence.

46. The circular RNA precursor of any one of claims 1-45, wherein the nucleotide sequence of interest comprises at least one protein-coding sequence and a translation initiation element such as an internal ribosome entry site (IRES) operably linked thereto.

47. The circular RNA precursor of claim 46, wherein the translation initiation element, such as an IRES, is located upstream to the 5′ end of the at least one protein-coding sequence.

48. The circular RNA precursor of claim 46, wherein the translation initiation element, such as an IRES, is located downstream to the 3′ end of the at least one protein-coding sequence.

49. The circular RNA precursor of any one of claims 46-48, wherein the protein-coding sequence encodes a protein of eukaryotic, prokaryotic or viral origin, for example, a protein for therapeutic or diagnostic use.

50. The circular RNA precursor of any one of claims 46-49, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is CVB3, BRAV-1_L, PV1_L, CAV2_L, BRAV-1, PV1, or CAV2.

51. The circular RNA precursor of any one of claims 46-50, wherein the IRES comprises a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.

52. The circular RNA precursor of any one of claims 1-45, wherein the nucleotide sequence of interest is a non-protein coding sequence, for example, the non-protein-coding sequence is selected from antisense RNA, aptamer, guide RNA, or a non-protein-coding RNA naturally existing in an organism.

53. The circular RNA precursor of any one of claims 1-52, wherein the nucleotide sequence of interest is about 10-about 20000 nucleotides in length.

54. The circular RNA precursor of any one of claims 1-53, wherein the circular RNA precursor does not contain nucleotide chemical modification.

55. A nucleic acid vector for generating a circular RNA molecule, said vector comprises a coding sequence of the circular RNA precursor of any one of claims 1-54.

56. The nucleic acid vector of claim 55, which further comprises an RNA polymerase promoter sequence operably linked to the coding sequence of the circular RNA precursor.

57. The nucleic acid vector of claim 56, wherein the promoter is a T7 RNA polymerase promoter, a T6 viral RNA polymerase promoter, a SP6 viral RNA polymerase promoter, a T3 viral RNA polymerase promoter or a T4 viral RNA polymerase promoter, preferably a T7 RNA polymerase promoter.

58. A circular RNA, which is prepared from the circular RNA precursor of any one of claims 1-54 or from the nucleic acid vector of any one of claims 55-57.

59. A circular RNA, which comprises a first residual circularizing element, a nucleotide sequence of interest, and a second residual circularizing element, wherein the first residual circularizing element, the nucleotide sequence of interest, and the second residual circularizing element are defined in any one of claims 1-54.

60. A circular RNA, which comprises a first residual circularizing element, a nucleotide sequence of interest, and a second residual circularizing element, wherein the total length of the first residual circularizing element and the second residual circularizing element is about 5 to about 100 nucleotides.

61. The circular RNA of claim 60, wherein the first residual circularizing element and the second residual circularizing element are involved in RNA circularization with a self-splicing intron, such as a Group I intron, preferably a Group I intron of the Anabaena pre-tRNA-Leu gene.

62. The circular RNA of claim 60 or 61, wherein the first residual circularizing element and the second residual circularizing element are covalently linked, for example, 5′ end of the first residual circularizing element is covalently linked to 3′ end of the second residual circularizing element.

63. The circular RNA of any one of claims 60-62, wherein the first residual circularizing element and the second residual circularizing element are configured such that the circular RNA comprising them has reduced immunogenicity relative to a control RNA, such as a control circular RNA comprising the circularizing elements of SEQ ID NO:29 and SEQ ID NO:64 or a control linear RNA comprising the same nucleotide sequence of interest.

64. The circular RNA of any one of claims 60-63, wherein the first residual circularizing element and the second residual circularizing element are configured such that the circular RNA comprising them can be generated with a comparable or increased circularization efficiency relative to a control circular RNA, such as a control circular RNA comprising the residual circularizing elements of SEQ ID NO:29 and SEQ ID NO:64 (Ana 3.0).

65. The circular RNA of any one of claims 60-64, wherein the total length of the first residual circularizing element and the second residual circularizing element is about 20 to about 35 nucleotides.

66. The circular RNA of any one of claims 60-65, wherein the first residual circularizing element and the second residual circularizing element are configured to be capable of forming a stem-loop structure.

67. The circular RNA of claim 66, wherein the loop of the stem-loop structure comprises the splicing junction between the first residual circularizing element and the second residual circularizing element.

68. The circular RNA of any one of claims 60-67, wherein the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure.

69. The circular RNA of claim 68, wherein the first loop sequence comprises or consists of one or more nucleotides which can pair with the P1 region of the corresponding self-splicing intron to form a P10 duplex region during the circularization.

70. The circular RNA of claim 68 or 69, wherein the first loop sequence comprises or consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.

71. The circular RNA of any one of claims 68-70, wherein the first loop sequence comprises or consists of the sequence of AAAA, AA, UUUU, UAAA, CAAAA, or GAAAA, preferably AAAA.

72. The circular RNA of any one of claims 68-71, wherein the second loop sequence comprises or consists of one or more nucleotides which can pair with the internal guide sequence (IGS) of the corresponding self-splicing intron to form a P1 duplex region during the circularization.

73. The circular RNA of any one of claims 68-72, wherein the second loop sequence comprises or consists of CUU or CUC, preferably CUU.

74. The circular RNA of any one of claims 68-73, wherein the loop of the stem-loop structure has a sequence of CUUAAAA, CUUUUUU, CUUAA, CUUGAAA, CUUUAAA, CUUCAAA or CUCAAAA, preferably CUUAAAA.

75. The circular RNA of any one of claims 68-74, wherein the stem portion of the stem-loop structure comprises 2-15 or more consecutive base pairs, preferably, the stem portion of the stem-loop structure comprises 5, 6 or 7 consecutive base pairs.

76. The circular RNA of any one of claims 68-75, wherein the stem portion in the stem-loop structure comprises up to 2 base mismatches, or the stem portion comprises only 1 base mismatch, preferably, the stem portion comprises no base mismatches.

77. The circular RNA of any one of claims 68-76, wherein the first pairing sequence comprises only Gs and the second pairing sequence comprises only Cs.

78. The circular RNA of any one of claims 68-76, wherein the first pairing sequence comprises only Cs and the second pairing sequence comprises only Gs.

79. The circular RNA of any one of claims 68-76, wherein the first pairing sequence includes only A and the second pairing sequence includes only U.

80. The circular RNA of any one of claims 68-76, wherein the first pairing sequence comprises or consists of the sequence of any one of SEQ ID NO:42-55, and, the first pairing sequence comprises or consists of the sequence of any one of SEQ ID NO:78-93.

81. The circular RNA of any one of claims 67-80, wherein the predicted free energy of the stem-loop structure is lower than about −1 kal/mol, lower than about −2 kal/mol, lower than about −3 kal/mol, lower than about −4 kal/mol, lower than about −5 kal/mol, lower than about −6 kal/mol, lower than about −7 kal/mol, lower than about −8 kal/mol, lower than about −9 kal/mol, lower than about −10 kal/mol, or lower.

82. The circular RNA of any one of claims 60-81, wherein the first residual circularizing element comprises or consists of from 5′ to 3′ direction a 3′ exon region and optionally a spacer, the second residual circularizing element comprises or consists of from 3′ to 5′ direction a 5′ exon region and optionally a spacer.

83. The circular RNA of claim 82, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.

84. The circular RNA of claim 83, wherein 3′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 90%, at least 91%, at least 92%, at least 93%, at least 94%, at least 95%, at least 96%, at least 97%, at least 99%, or at least 99% sequence identity with the native 5′ exon or a contiguous fragment of about 1-about 50 nucleotides starting from the 3′ terminal nucleotide of the native 5′ exon.

85. The circular RNA of any one of claims 60-84, wherein the first residual circularizing element comprises or consists of a nucleotide sequence having at least 50%, at least 60%, at least 70%, at least 80%, at least 90%, at least 95%, or 100% sequence identity to a nucleotide sequence selected from SEQ ID NOs: 13-55.

86. The circular RNA precursor of any one of claims 60-85, wherein the second residual circularizing element comprises or consists of a nucleotide sequence having at least 50%, at least 60%, at least 70%, at least 80%, at least 90%, at least 95% or 100% sequence identity to a nucleotide sequence selected from SEQ ID NOs: 56-93.

87. The circular RNA of any one of claims 60-86, wherein the first residual circularizing element comprises or consists of a nucleotide sequence of any one of SEQ ID NOs: 13-55 and the second residual circularizing element comprises or consists of a nucleotide sequence of any one of SEQ ID NOs: 56-93.

88. The circular RNA of any one of claims 60-87, wherein,
1) the first residual circularizing element comprises or consists of a nucleotide sequence of SEQ ID NO: 15 and the second residual circularizing element comprises or consists of a nucleotide sequence of SEQ ID NO: 57;
2) the first residual circularizing element comprises or consists of a nucleotide sequence of SEQ ID NO: 14 and the second residual circularizing element comprises or consists of a nucleotide sequence of SEQ ID NO: 58;
3) the first residual circularizing element comprises or consists of a nucleotide sequence of SEQ ID NO: 18 and the second residual circularizing element comprises or consists of a nucleotide sequence of SEQ ID NO: 59;
4) the first residual circularizing element comprises or consists of a nucleotide sequence of SEQ ID NO: 19 and the second residual circularizing element comprises or consists of a nucleotide sequence of SEQ ID NO: 60;
5) the first residual circularizing element comprises or consists of a nucleotide sequence of SEQ ID NO: 20 and the second residual circularizing element comprises or consists of a nucleotide sequence of SEQ ID NO: 61;
6) the first residual circularizing element comprises or consists of a nucleotide sequence of SEQ ID NO: 21 and the second residual circularizing element comprises or consists of a nucleotide sequence of SEQ ID NO: 62;
7) the first residual circularizing element comprises or consists of a nucleotide sequence of SEQ ID NO: 23 and the second residual circularizing element comprises or consists of a nucleotide sequence of SEQ ID NO: 56;
8), the first residual circularizing element comprises or consists of a nucleotide sequence of SEQ ID NO: 24 and the second residual circularizing element comprises or consists of a nucleotide sequence of SEQ ID NO: 56;
9) the first residual circularizing element comprises or consists of a nucleotide sequence of SEQ ID NO: 26 and the second residual circularizing element comprises or consists of a nucleotide sequence of SEQ ID NO: 56;
10) the first residual circularizing element comprises or consists of a nucleotide sequence of SEQ ID NO: 27 and the second residual circularizing element comprises or consists of a nucleotide sequence of SEQ ID NO: 56;
11) the first residual circularizing element comprises or consists of a nucleotide sequence of SEQ ID NO: 28 and the second residual circularizing element comprises or consists of a nucleotide sequence of SEQ ID NO: 56;
12) the first residual circularizing element comprises or consists of a nucleotide sequence of SEQ ID NO: 13 and the second residual circularizing element comprises or consists of a nucleotide sequence of SEQ ID NO: 67;
13) the first residual circularizing element comprises or consists of a nucleotide sequence of SEQ ID NO: 13 and the second residual circularizing element comprises or consists of a nucleotide sequence of SEQ ID NO: 59;
14) the first residual circularizing element comprises or consists of a nucleotide sequence of SEQ ID NO: 13 and the second residual circularizing element comprises or consists of a nucleotide sequence of SEQ ID NO: 56;
15) the first residual circularizing element comprises or consists of a nucleotide sequence of SEQ ID NO: 17 and the second residual circularizing element comprises or consists of a nucleotide sequence of SEQ ID NO: 59; or
16) the first residual circularizing element comprises or consists of a nucleotide sequence of SEQ ID NO: 22 and the second residual circularizing element comprises or consists of a nucleotide sequence of SEQ ID NO: 63.

89. The circular RNA of any one of claims 60-88, wherein the nucleotide sequence of interest comprises at least one protein-coding sequence and a translation initiation element such as an internal ribosome entry site (IRES) operably linked thereto.

90. The circular RNA of claim 89, wherein the translation initiation element, such as an IRES, is located upstream to the 5′ end of the at least one protein-coding sequence.

91. The circular RNA of claim 89, wherein the translation initiation element, such as an IRES, is located downstream to the 3′ end of the at least one protein-coding sequence.

92. The circular RNA of any one of claims 89-91, wherein the protein-coding sequence encodes a protein of eukaryotic, prokaryotic or viral origin, for example, a protein for therapeutic or diagnostic use.

93. The circular RNA of any one of claims 89-92, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is CVB3, BRAV-1_L, PV1_L, CAV2_L, BRAV-1, PV1, or CAV2.

94. The circular RNA of any one of claims 89-93, wherein the IRES comprises a nucleotide sequence set forth in one of SEQ ID NOs: 105-135, or comprise a nucleotide sequence having at least 75%, e.g., at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 105-135.

95. The circular RNA of any one of claims 60-88, wherein the nucleotide sequence of interest is a non-protein coding sequence, for example, the non-protein-coding sequence is selected from antisense RNA, aptamer, guide RNA, or a non-protein-coding RNA naturally existing in an organism.

96. The circular RNA of any one of claims 60-95, wherein the nucleotide sequence of interest is about 10-about 20000 nucleotides in length.

97. The circular RNA of any one of claims 60-96, wherein the circular RNA does not contain nucleotide chemical modification.

98. Use of the circular RNA precursor any one of claims 1-54 and/or circular RNA of any one of claims 58-97 as an expression vector.

99. A pharmaceutical composition comprising the circular RNA precursor of any one of claims 1-54 and/or the nucleic acid vector of any one of claims 55-57 and/or circular RNA of any one of claims 58-97, and a pharmaceutically acceptable carrier.

100. The pharmaceutical composition of claim 99, which is for use in treating a disease in a subject.

101. A method for preparing a circular RNA, the method comprises:
1) providing a circular RNA precursor of any one of claims 1-54 or obtaining a circular RNA precursor by transcribing from the nucleic acid vector of any one of claims 55-57;
2) incubating the circular RNA precursor in the presence of a divalent metal cation at a temperature at which RNA circularization occurs; and
3) harvesting the circular RNA obtained in step 2).

102. The method of claim 101, wherein the divalent metal cation is Mg²⁺ and/or Mn²⁺.

103. The method of claim 101 or 102, wherein the concentration of the divalent metal cation is about 5 mM to about 550 mM.

104. A method for preparing a circular RNA, the method comprises
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.

105. The method of claim 104, wherein the nucleic acid vector is the nucleic acid vector of any one of claims 55-57.

106. The method of claim 104 or 105, wherein the divalent metal cation in the in vitro transcription system is Mg²⁺.

107. The method of any one of claims 104-106, wherein the in vitro transcription system further comprises a monovalent metal cation and/or a monovalent anion.

108. The method of claim 107, wherein the monovalent metal cation is Na⁺ or K⁺.

109. The method of claim 107, wherein the monovalent metal anion is Cl⁻ or CH3COO⁻ (OAc).

110. The method of any one of claims 104-109, wherein the concentration of the divalent metal cation in the system during the first time period is from about 5 mM to about 50 mM.

111. The method of any one of claims 107-110, wherein the concentration of the monovalent metal cation in the system during the first time period is from about 5 mM to about 100 mM.

112. The method of any one of claims 107-111, wherein the concentration of the monovalent anion in the system during the first time period is from about 5 mM to about 150 mM.

113. The method of any one of claims 107-112, wherein the method does not include a step of isolating and/or purifying the linear RNA produced by the in vitro transcription.

114. The method of any one of claims 107-113, wherein the buffer of the in vitro transcription system is selected from Tris-HCl buffer, or HEPES buffer, or MES buffer, or citrate buffer, or phosphate buffer, preferably, the buffer of the in vitro transcription system is HEPES buffer.

115. The method of any one of claims 107-114, wherein the in vitro transcription system has a pH of about 5-about 8, preferably, the in vitro transcription system has a pH of about 7.5.

116. The method of any one of claims 107-115, wherein the RNA polymerase is selected from a T7 RNA polymerase, a T6 viral RNA polymerase, a SP6 viral RNA polymerase, a T3 viral RNA polymerase, or a T4 viral RNA polymerase, preferably, the RNA polymerase is T7 RNA polymerase.

117. The method of any one of claims 107-116, wherein the first time period is about 0.5 hours to about 24 hours, preferably, the first time period is 3 hours.

118. The method of any one of claims 107-117, wherein the incubation for the first time period is carried out at about 16° C. to about 60° C., preferably, the incubation for the first time period is carried out at about 37° C.

119. The method of any one of claims 107-118, wherein after incubation for the first time period in step b), the method further comprises step c):
adding an additional amount of metal cation to the in vitro transcription system and incubating for a second time period; or
changing the buffer of the system, adding a metal cation and incubating for a second time period.

120. The method of claim 119, wherein the metal cation added for the incubation of the second time period is a divalent metal cation, such as Mg²⁺ or Mn²⁺.

121. The method of claim 119 or 120, wherein during the incubation of the second time period, the metal cation is added to a final concentration of about 5 mM to about 550 mM.

122. The method of any one of claims 119-121, wherein the buffer of the system is changed to Tris-HCl buffer, or HEPES buffer, or MES buffer, or citrate buffer, or phosphate buffer during the second time period.

123. The method of any one of claims 119-122, wherein the pH of the system in the second time period is 5-8.

124. The method of any one of claims 119-123, wherein the second time period is about 5 minutes to about 2 hour.

125. The method of any one of claims 119-124, wherein the incubation for the second time period is carried out at about 25° C. to about 75° C.

126. The method of any one of claims 104-125, wherein the method further comprises a step of recovering or purifying the circular RNA as produced.

127. A method for purifying a circular RNA, the method comprises:
a) contacting a mixture comprising circular RNA and uncircularized linear circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.

128. The method of claim 127, wherein the circular RNA is prepared by circularizing a linear circular RNA precursor.

129. The method of claim 127 or 128, wherein the circular RNA is prepared by ligating both ends of a linear circular RNA precursor with an RNA ligase, or, the circular RNA is prepared by the self-splicing ribozyme activity of self-splicing intron-based circularizing elements contained in the linear circular RNA precursor.

130. The method of any one of claims 127-129, wherein the circular RNA-specific probe is a single-stranded DNA or RNA probe.

131. The method of any one of claims 127-130, wherein the circular RNA-specific probe specifically hybridizes to a region flanking the circularization junction of the circular RNA.

132. The method of any one of claims 127-131, wherein the circular RNA-specific probe is about 10 nucleotides-about 35 nucleotides in length or longer.

133. The method of any one of claims 127-132, wherein the circular RNA-specific probe is immobilized on a support such as a solid support, e.g., the circular RNA-specific probe is immobilized on the support after binding to the circular RNA or the circular RNA-specific probe is pre-immobilized on the support.

134. The method of any one of claims 127-133, wherein the condition in step a) include denaturing the RNA at between about 60° C. and about 95° C. for about 2 minutes to about 10 minutes, then gradually reducing the temperature to below about 40° C. to allow the circular RNA annealing to the circular RNA-specific probe.

135. The method of claim 134, wherein step c) is performed by increasing the temperature to about 60° C. to about 95° C. (e.g., about 60° C., about 62° C., about 64° C., about 66° C., about 68° C.) C, about 70° C., about 75° C., about 80° C., about 85° C., about 90° C., about 95° C.) to release the circular RNA.

136. The method of any one of claims 127-133, wherein the condition in step a) include a high salt concentration range of 0.25M-2M.

137. The method of claim 136, wherein the salt is NaCl or a guanidine salt.

138. The method of claim 136 or 137, wherein in step c) the circular RNA is released by elution with an elution buffer, such as a low salt buffer.

139. The method of claim 138, wherein the elution buffer is Tris-EDTA buffer (TE buffer) or water.

140. The method of any one of claims 127-139, wherein in step b), the one or more components are removed by washing the complex with a washing buffer.

141. The method of any one of claims 127-140, the method further includes the following steps:
i) contacting the mixture comprising circular RNA and uncircularized linear circular RNA precursor to a linear circular RNA precursor-specific probe in a condition that allows the linear circular RNA precursor-specific probe to specifically bind to and form a complex with the linear circular RNA precursor;
ii) removing the complex formed by the linear circular RNA precursor-specific probe with the linear circular RNA precursor from the mixture, and
iii) collecting the circular RNA-containing mixture obtained in step ii).

142. The method of claim 141, wherein steps i)-iii) are performed before step a), for example, steps i)-iii) may be performed multiple times before step a), e.g., 2, 3, 4 or more times.

143. The method of claim 141, wherein steps i)-iii) are performed concurrently with steps a)-c).

144. A method for purifying circular RNA, the method comprises:
i) contacting a mixture comprising circular RNA and uncircularized linear circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and
optionally, steps i)-iii) are performed multiple times, e.g., 2 times, 3 times, 4 times or more.

145. The method of claim 144, wherein the linear circular RNA precursor-specific probe specifically binds to the linear circular RNA precursor and does not substantially bind to the circular RNA.

146. The method of claim 144 or 145, wherein the linear circular RNA precursor-specific probe is immobilized on a support, such as a solid support, for example, the linear circular RNA precursor-specific probe is then immobilized on the support after binding to the linear circular RNA precursor, or the linear circular RNA precursor-specific probe is pre-immobilized on the support.

147. The method of any one of claims 127-146, wherein the linear circular RNA precursor comprises the following elements arranged in the following order from the 5′ to 3′ direction:
a) a 3′ self-splicing intron fragment;
b) a first residual circularizing element;
c) a nucleotide sequence of interest;
d) a second residual circularizing element; and
e) a 5′ self-splicing intron fragment;
wherein the linear circular RNA precursor is capable of removing the 3′ self-splicing intron fragment and the 5′ self-splicing intron fragment by self-splicing, generating a circular RNA comprising the first residual circularizing element, the nucleotide sequence of interest and the second residual circularizing element.

148. The method of claim 147, wherein the circular RNA-specific probe specifically hybridizes to at least a portion of the first residual circularizing element and a portion of the second residual circularizing element.

149. The method of claim 147 or 148, wherein the linear precursor RNA-specific probe hybridizes to a portion of the linear circular RNA precursor outside the first residual circularizing element, the nucleotide sequence of interest, and the second residual circularizing element.

150. The method of any one of claims 147-149, wherein the linear precursor RNA-specific probe hybridizes to the 3′ self-splicing intron fragment or a portion thereof or a 5′ flanking sequence thereof, or the 5′ self-splicing intron fragment or a portion thereof or a 3′ flanking sequence thereof.

151. The method of any one of claims 147-150, wherein the linear circular RNA precursor contains a sequence selected from SEQ ID NOs:96-101 or a complement sequence thereof, preferably, SEQ ID NO:100 or a complement sequence thereof outside the first residual circularizing element, the nucleotide sequence of interest, and the second residual circularizing element, to which the linear precursor RNA-specific probe specifically hybridizes.

152. The method of any one of claims 147-151, the molar ratio of the probe to the RNA molecules in the mixture is from about 1:1 to about 100,000:1.

153. A method for purifying circular RNA, the method comprises:
i) adding a linear RNA-specific tag to the linear RNA in a mixture comprising circular RNA and linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii),
optionally, steps ii)-iv) are performed multiple times, e.g., 2 times, 3 times, 4 times or more.

154. The method of claim 153, wherein the tag comprises a polyA, polyG, polyU, or polyC sequence.

155. The method of claim 153 or 154, wherein the tag is about 10-200 nt in length or the probe is about 10-200 nt in length.

156. The method of any one of claims 153-155, wherein the tag is added to the linear RNA by adding a PolyA/T/C/G polymerase or a ligase to the mixture.

157. The method of any one of claims 153-156, wherein the linear RNA probe specifically binds to the added tag without substantially binding to the circular RNA.

158. The method of any one of claims 153-157, wherein the linear RNA probe is a single-stranded DNA probe, or a single-stranded RNA probe.

159. The method of any one of claims 153-158, wherein the linear RNA probe is immobilized on a support such as a solid support, for example, the linear RNA probe is immobilized on the support after binding to the linear RNA, or, the linear RNA probe is pre-immobilized on the support.
